SEQUENCE LISTING (2 sequences)

SEQ ID NO: 1 - protein, 26 aa, Artificial sequence
  sequence is synthesized
DLRPEIRIAQELRRIGDEFNETYTRR

SEQ ID NO: 2 - protein, 26 aa, Artificial sequence
  sequence is synthesized
PSSTMGQVGRQLAIIGDDINRRYDSE

(19) United States
(12) Patent Application Publication
(10) Pub. No.: US 2011/0098305 A1
(43) Pub. Date: Apr. 28, 2011

(54) ARYLSULFONAMIDE COMPOUNDS, COMPOSITIONS AND METHODS OF USE

(76) Inventors: Jonathan Bayldon Baell, Victoria (AU); Guillaume Laurent Lessene, Victoria (AU); Brad Edmund Sleebs, Miranda (AU); Wayne Fairbrother, Burlingame, CA (US); John Flygare, Burlingame, CA (US); Michael F.T. Koehler, Palo Alto, CA (US)

(21) Appl. No.: 12/993,574

(22) PCT Filed: May 20, 2009
(86) PCT No.: PCT/US09/44680

§ 371 Date: Dec. 23, 2010

Related U.S. Application Data
(60) Provisional application No. 61/054,872, filed on May 21, 2008.

(51) Int. Cl. A61K 31/501; C07D 239/88; A61K 31/497; C07D 237/30; A61P 35/00

(52) U.S. Cl. 514/248; 544/283; 514/252.17; 544/237

(57) ABSTRACT

Disclosed are compounds which inhibit the activity of anti-apoptotic protein family members, compositions containing the compounds and uses of the compounds for preparing medicaments for treating diseases during which occurs expression one or more than one of an anti-apoptotic protein family member.

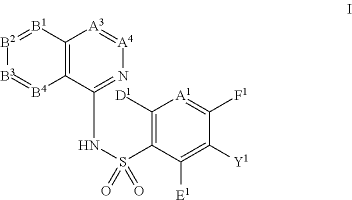

### CROSS REFERENCE TO RELATED APPLICATIONS

This application is a US National Stage of PCT/US09/44680, filed on May 20, 2009, which claims the benefit under 35 USC §119(e) of U.S. Provisional Application Ser. No. 61/054,872 filed on May 21, 2008. The entire disclosures of the foregoing applications are incorporated by reference herein.

### STATEMENT REGARDING FEDERALLY SPONSORED RESEARCH OR DEVELOPMENT

NONE

### BACKGROUND OF THE INVENTION

This invention pertains to compounds which inhibit the activity of anti-apoptotic protein family members, compositions containing the compounds, and methods of treating diseases during which are expressed of one or more than one of an anti-apoptotic protein family member.

Anti-apoptotic protein family members are associated with a number of diseases. There is therefore an existing need in the therapeutic arts for compounds which inhibit the activity of one or more than one of an anti-apoptotic protein family member.

### BRIEF DESCRIPTION OF THE FIGURES

NONE

### SUMMARY OF THE INVENTION

One embodiment of this invention, therefore, pertains to compounds or therapeutically acceptable salts, prodrugs or salts of prodrugs thereof, which are useful as inhibitors one or more than one anti-apoptotic protein family member, the compounds having Formula I

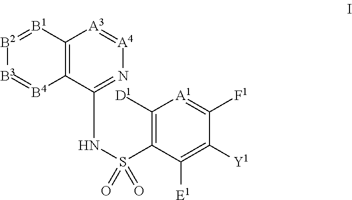

wherein

A¹ is N or C (A²);

one or two or three or each of A², and D¹ and E¹ are independently selected R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NR¹C(O)NHR¹, NR¹C(O)N(R¹)₂, SO₂NHR¹, SO₂N(R¹)₂, NHSO₂R¹, NHSO₂NHR¹ or N(CH₃) SO₂N(CH₃) R¹, and the remainder are independently selected H, F, Cl, Br, I, CN, CF₃, C(O)OH, C(O)NH₂ or C(O)OR^{1A}; and

Y¹ is H, CN, NO₂, C(O)OH, F, Cl, Br, I, CF₃, OCF₃, CF₂CF₃, OCF₂CF₃, R¹⁷, OR¹⁷, C(O)R¹⁷, C(O)OR¹⁷, SR¹⁷, NH₂, NHR¹⁷, N(R¹⁷)₂, NHC(O)R¹⁷, C(O)NH₂, C(O)NHR¹⁷, C(O)N(R¹⁷)₂, NHS(O)R¹⁷ or NHSO₂R¹⁷;

or

F¹ and Y¹, together with the atoms to which they are attached, are imidazole or triazole; and

one or two or each of A², D¹ and E¹ are independently selected R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NHC(O)NHR¹, N(CH₃) C(O)N(CH₃) R¹, SO₂NHR¹, SO₂N(R¹)₂, NHSO₂R¹, NHSO₂NHR¹ or N(CH₃) SO₂N(CH₃) R¹, and the remainder are independently selected H, F, Cl, Br, I, CF₃, C(O)OH, C(O)NH₂ or C(O)OR^{1A};

R¹ is R², R³, R⁴ or R⁵;

R^{1A} is C₁-C₆-alkyl, C₃-C₆-alkenyl or C₃-C₆-alkynyl;

R² is phenyl which is unfused or fused with arene, heteroarene or R^{2A}; R^{2A} is cycloalkane or heterocycloalkane;

R³ is heteroaryl which is unfused or fused with benzene, heteroarene or R^{3A}; R^{3A} is cycloalkane or heterocycloalkane;

R⁴ is cycloalkyl, cycloalkenyl, heterocycloalkyl or heterocycloalkenyl, each of which is unfused or fused with arene, heteroarene or R^{4A}; R^{4A} is cycloalkane, cycloalkene, heterocycloalkane or heterocycloalkene;

R⁵ is alkyl, alkenyl or alkynyl, each of which is unsubstituted or substituted with one or two or three independently selected R⁶, NC(R^{6A})(R^{6B}), OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂, NHC(O)NHR⁷, NHC(O)CH(CH₃) NHC(O)CH(CH₃) NH₂, NHC(O)CH(CH₃)NHC(O)CH(CH₃)NHR¹, OH, (O), C(O)OH, (O), N₃, CN, NH₂, CF₃, CF₂CF₃, F, Cl, Br or I substituents;

R⁶ is C₂-C₅-spiroalkyl, each of which is unsubstituted or substituted with OH, (O), N₃, CN, CF₃, CF₂CF₃, F, Cl, Br, I, NH₂, NH(CH₃) or N(CH₃)₂;

R^{6A} and R^{6B} are independently selected alkyl or, together with the N to which they are attached is R^{6C};

R^{6C} is aziridin-1-yl, azetidin-1-yl, pyrrolidin-1-yl or piperidin-1-yl, each having one CH₂ moiety unreplaced or replaced with O, C(O), CNOH, CNOCH₃, S, S(O), SO₂ or NH;

R⁷ is R⁸, R⁹, R¹⁰ or R¹¹;

R⁸ is phenyl which is unfused or fused with arene, heteroarene or R^{8A};

R^{8A} is cycloalkane, cycloalkene, heterocycloalkane or heterocycloalkene;

R⁹ is heteroaryl which is unfused or fused with arene, heteroarene or R^{9A}; R^{9A} is cycloalkane, cycloalkene, heterocycloalkane or heterocycloalkene;

R¹⁰ is C₃-C₁₀-cycloalkyl or C₄-C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{10A}; R^{10A} is cycloalkane, cycloalkene, heterocycloalkane or heterocycloalkene;

R¹¹ is alkyl, alkenyl or alkynyl, each of which is unsubstituted or substituted with one or two or three independently selected R¹², OR¹², NHR¹², N(R¹²)₂, C(O)NH₂, C(O)NHR¹², C(O)N(R¹²)₂, OH, (O), C(O)OH, N₃, CN, NH₂, CF₃, CF₂CF₃, F, Cl, Br or I substituents;

R¹² is R¹³, R¹⁴, R¹⁵ or R¹⁶;

R¹³ is phenyl which is unfused or fused with arene, heteroarene or R^{13A}; R^{13A} is cycloalkane, cycloalkene, heterocycloalkane or heterocycloalkene;

R¹⁴ is heteroaryl, each of which is unfused or fused with arene, heteroarene or R^{14A}; R^{14A} is cycloalkane, cycloalkene, heterocycloalkane or heterocycloalkene;

R¹⁵ is cycloalkane, cycloalkene, heterocycloalkane or heterocycloalkene, each of which is unfused or fused with arene, heteroarene or R^{15A}; R^{15A} is cycloalkane, cycloalkene, heterocycloalkane or heterocycloalkene;

R¹⁶ is alkyl, alkenyl or alkynyl;

R¹⁷ is R¹⁸, R¹⁹, R²⁰ or R²¹;

R¹⁸ is phenyl which is unfused or fused with arene, heteroarene or R^{18A}; R^{18A} is cycloalkane, cycloalkene, heterocycloalkane or heterocycloalkene;

R¹⁹ is heteroaryl which is unfused or fused with arene, heteroarene or R^{19A}; R^{19A} is cycloalkane, cycloalkene, heterocycloalkane or heterocycloalkene;

R²⁰ is C₃-C₁₀-cycloalkyl or C₄-C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{20A}; R^{20A} is cycloalkane, cycloalkene, heterocycloalkane or heterocycloalkene;

R²¹ is alkyl, alkenyl or alkynyl, each of which is unsubstituted or substituted with one or two or three independently selected R²², OR²², NHR²², N(R²²)₂, C(O)NH₂, C(O)NHR²², C(O)N(R²²)₂, OH, (O), C(O)OH, N₃, CN, NH₂, CF₃, CF₂CF₃, F, Cl, Br or I substituents;

R²² is R²³, R²⁴ or R²⁵;

R²³ is phenyl which is unfused or fused with arene, heteroarene or R^{23A}; R^{23A} is cycloalkane, cycloalkene, heterocycloalkane or heterocycloalkene;

R²⁴ is heteroarene which is unfused or fused with arene, heteroarene or R^{24A}; R^{24A} is cycloalkane, cycloalkene,

R²⁵ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{25A}; R^{25A} is cycloalkane, cycloalkene, heterocycloalkane or heterocycloalkene;

A³ and A⁴ are each independently N or C(R²⁶); each R²⁶ is independently hydrogen, halogen, C₁₋₆-alkyl, C₂₋₆-alkenyl, C₂₋₆-alkynyl, hydroxy, C₁₋₆-alkoxy, C₂₋₆-alkenyloxy, C₂₋₆-alkynyloxy, C(O)N(R^{26A})₂, acyl, N(R^{26B})₂, C(R^{26C})₃, wherein R^{26A} is independently C₁₋₆-alkyl, C₂₋₆-alkenyl or C₂₋₆-alkynyl; R^{26B} is independently hydrogen, C₁₋₆-alkyl, C₂₋₆-alkenyl, C₂₋₆-alkynyl or acyl; R^{26C} is independently hydrogen or halogen;

B¹, B², B³ and B⁴ are each independently N, C(Z) or C(R²⁷), wherein one of B¹, B², B³ or B⁴ is C(Z); each R²⁷ is independently selected H, F, Cl, Br, I, CH₂R^{27C}, CH(R^{27B})(R^{27C}) C(R^{27B})(R^{27A})(R^{27C}), C(O)R^{27C}, OR^{27C}, SR^{27C}, S(O)R^{27C}, SO₂R^{27C}, NHR^{27C} or N(R^{27D}) R^{27C};

R^{27A} and R^{27B} are independently F, Cl, Br or alkyl or are taken together and are C₂-C₅-spiroalkyl;

R^{27D} is R^{27E}, C(O)R^{27E} or C(O)OR^{27E};

R^{27E} is R^{27F} or R^{27G};

R^{27F} is phenyl which is unfused or fused with aryl, heteroaryl or R^{27FF}; R^{27FF} is cycloalkane, cycloalkene, heterocycloalkane or heterocycloalkene;

R^{27G} is alkyl which is unsubstituted or substituted with R^{27H};

R^{27H} is phenyl which is unfused or fused with arene, heteroarene or R^{27HH}; R^{27HH} is cycloalkane, cycloalkene, heterocycloalkane or heterocycloalkene;

R^{27C} is R^{27I}, R^{27J} or R^{27K}, each of which unsubstituted or is substituted with F, Cl, Br, I, R^{27L}, OR^{27L}, NHR^{27L}, N(R^{27L})₂, NHC(O)OR^{27L}, SR^{27L}, S(O)R^{27L} or SO₂R^{27L};

R^{27I} is phenyl which is unfused or fused with arene, heteroarene or R^{27II}; R^{27II} is cycloalkane, cycloalkene, heterocycloalkane or heterocycloalkene;

R^{27J} is heteroaryl which is unfused or fused with arene, heteroarene or R^{27JJ}; R^{27JJ} is cycloalkane, cycloalkene, heterocycloalkane or heterocycloalkene;

R^{27K} is C₃-C₈-cycloalkyl or C₄-C₈-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{27KK}; R^{27KK} cycloalkane, cycloalkene, heterocycloalkane or heterocycloalkene;

R^{27L} is R^{27M}, R^{27II}, R^{27P} or R^{27Q};

R^{27M} is phenyl which is unfused or fused with arene, heteroarene or R^{27MM}; R^{27MM} is cycloalkane, cycloalkene, heterocycloalkane or heterocycloalkene;

R^{27N} is heteroaryl which is unfused or fused with arene, heteroarene or R^{27NN}; R^{27NN} is cycloalkane, cycloalkene, heterocycloalkane or heterocycloalkene;

R^{27P} is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{27PP}; R^{27PP} is cycloalkane, cycloalkene, heterocycloalkane or heterocycloalkene;

R^{27Q} is alkyl, alkenyl or alkynyl, each of which is unsubstituted or substituted with one or two independently selected R^{27QQ}, OR^{27QQ}, NHR^{27QQ}, N(R^{27QQ})₂, C(O)NH₂, C(O)NHR^{27QQ}, C(O)N(R^{27QQ})₂, OH, (O), C(O)OH, N₃, CN, NH₂, CF₃, CF₂CF₃, F, Cl, Br or I substituents;

R^{27QQ} is R^{27R}, R^{27S} or R^{27T};

R^{27R} is phenyl which is unfused or fused with arene, heteroarene or R^{27R}; R^{27RR} is cycloalkane, cycloalkene, heterocycloalkane or heterocycloalkene;

R^{27S} is heteroaryl or R^{27SS}; R^{27SS} is cycloalkane, cycloalkene, heterocycloalkane or heterocycloalkene;

R^{27T} is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{27TT}; R^{27TT} is cycloalkane, cycloalkene, heterocycloalkane or heterocycloalkene;

Z is R²⁸, R²⁹ or R³⁰, wherein

R²⁸ is phenyl which is unfused or fused with arene, heteroarene or R^{28A}; R^{28A} is cycloalkane, cycloalkene, heterocycloalkane or heterocycloalkene

R²⁹ is heteroaryl or R^{29A}; R^{29A} is cycloalkane, cycloalkene, heterocycloalkane or heterocycloalkene;

R³⁰ is cycloalkyl or cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{30A}; R^{30A} is cycloalkane, cycloalkene, heterocycloalkane or heterocycloalkene; and

each of R²⁸, R²⁹ and R³⁰ is unsubstituted or is substituted with F, Cl, Br, I, CH₂R³⁷, CH(R³¹)(R³⁷), C(R³¹)(R^{31A})(R³⁷), C(O)R³⁷, OR³⁷, SR³⁷, S(O)R³⁷, SO₂R³⁷, NHR³⁷ or N(R³²)R³⁷;

R³¹ and R^{31A} are independently F, Cl, Br or alkyl or are taken together and are C₂-C₅-spiroalkyl;

R³² is R³³, C(O)R³³ or C(O)OR³³;

R³³ is R³⁴ or R³⁵;

R³⁴ is phenyl which is unfused or fused with aryl, heteroaryl or R^{34A}; R^{34-A} is cycloalkane, cycloalkene, heterocycloalkane or heterocycloalkene;

R³⁵ is alkyl which is unsubstituted or substituted with R³⁶;

R³⁶ is phenyl which is unfused or fused with arene, heteroarene or R^{36A}; R^{36A} is cycloalkane, cycloalkene, heterocycloalkane or heterocycloalkene;

R³⁷ is R³⁸, R³⁹ or R⁴⁰, each of which is unsubstituted or is substituted with F, Cl, Br, I, R⁴¹, OR⁴¹, NHR⁴¹, N(R⁴¹)₂, NHC(O)OR⁴¹, SR⁴¹, S(O)R⁴¹ or SO₂R⁴¹;

R³⁸ is phenyl which is unfused or fused with arene, heteroarene or R^{38A}; R^{38A} is cycloalkane, cycloalkene, i heterocycloalkane or heterocycloalkene;

R³⁹ is heteroaryl which is unfused or fused with arene, heteroarene or R^{39A}; R^{39A} is cycloalkane, cycloalkene, heterocycloalkane or heterocycloalkene;

R⁴⁰ is C₃-C₈-cycloalkyl or C₄-C₈-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{40A}; R^{40A} cycloalkane, cycloalkene, heterocycloalkane or heterocycloalkene;

R⁴¹ is R⁴², R⁴³, R⁴⁴ or R⁴⁵;

R⁴² is phenyl which is unfused or fused with arene, heteroarene or R^{42A}; R^{42A} is cycloalkane, cycloalkene, i heterocycloalkane or heterocycloalkene;

R⁴³ is heteroaryl which is unfused or fused with arene, heteroarene or R^{43A}; R^{43A} is cycloalkane, cycloalkene, heterocycloalkane or heterocycloalkene;

R⁴⁴ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{44A}; R^{44A} is cycloalkane, cycloalkene, heterocycloalkane or heterocycloalkene;

R⁴⁵ is alkyl, alkenyl or alkynyl, each of which is unsubstituted or substituted with one or two independently selected R⁴⁶, OR⁴⁶, NHR⁴⁶, N(R⁴⁶)₂, C(O)NH₂, C(O)NHR⁴⁶, C(O)N(R⁴⁶)₂, OH, (O)C(O)OH, N₃, CN, NH₂, CF₃, CF₂CF₃, F, Cl, Br or I substituents;

R⁴⁶ is R⁴⁷, R⁴⁸ or R⁴⁹;

R⁴⁷ is phenyl which is unfused or fused with arene, heteroarene or R^{47A}; R⁴⁷.p, is cycloalkane, cycloalkene, i heterocycloalkane or heterocycloalkene;

R⁴⁸ is heteroaryl or R^{48A}; R^{48A} is cycloalkane, cycloalkene, heterocycloalkane or heterocycloalkene;

R⁴⁹ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{49A}; R^{49A} is cycloalkane, cycloalkene, heterocycloalkane or heterocycloalkene;

wherein each foregoing cyclic moiety is independently unsubstituted, further unsubstituted, substituted or further substituted with one or two or three or four or five independently selected from R⁵⁰, OR⁵⁰, SR⁵⁰, S(O)R⁵⁰, SO₂R⁵⁰, C(O)R⁵⁰, CO(O)R⁵⁰, OC(O)R⁵⁰, OC(O)OR⁵⁰, NH₂, NHR⁵⁰, N(R⁵⁰)₂, C(O)NH₂, C(O)NHR⁵⁰)C(O)N(R⁵⁰)₂, C(O)NHOH, C(O)NHOR⁵⁰, C(O)NHSO₂R⁵⁰, C(O)NR⁵⁵SO₂R⁵⁰, SO₂NH₂, SO₂NHR⁵⁰, SO₂N(R⁵⁰)₂, CF₃, CF₂CF₃, C(O)H, C(O)OH, C(N)NH₂, C(N)NHR⁵⁰, C(N)N(R⁵⁰)₂ᵣ OH, (O) N₃, NO₂, CF₃, CF₂CF₃, OCF₃, OCF₂CF₃, F, Cl, Br or I substituents;

R⁵⁰ is R⁵¹, R⁵², R⁵³ or R⁵⁴;

R⁵¹ is phenyl which is unfused or fused with arene, heteroarene or R^{51A}; R^{51A} is cycloalkane, cycloalkene, heterocycloalkane or heterocycloalkene;

R⁵² is heteroaryl or R^{52A}; R^{52A} is cycloalkane, cycloalkene, heterocycloalkane or heterocycloalkene;

R⁵³ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{53A}; R^{53A} is cycloalkane, cycloalkene, heterocycloalkane or heterocycloalkene;

R⁵⁴ is alkyl, alkenyl or alkynyl, each of which is unsubstituted or substituted with one or two or three independently selected R⁵⁵, OR⁵⁵, SR⁵⁵, S(O)R⁵⁵, SO₂R⁵⁵, NHR⁵⁵, N(R⁵⁵)₂, C(O)R⁵⁵, C(O)NH₂, C(O)NHR⁵⁵, NHC(O)R⁵⁵, NHSO₂R⁵⁵, NHC(O)OR⁵⁵, SO₂NH₂, SO₂NHR⁵⁵, SO₂N(R⁵⁵)₂, NHC(O)NH₂, NHC(O)NHR⁵⁵, OH, (O), C(O)OH, (O), N₃, CN, NH₂, CF₃, OCF₃, CF₂CF₃, OCF₂CF₃, F, Cl, Br or I substituents;

R⁵⁵ is alkyl, alkenyl, alkynyl, phenyl, heteroaryl or R⁵⁶;

R⁵⁶ is C₃-C₆-cycloalkyl or C₄-C₆-cycloalkyl, each having one or two CH₂ moieties unreplaced or replaced with independently selected O, C(O), CNOH, CNOCH₃, S, S(O), SO₂ or NH and one or two CH moieties unreplaced or replaced with N; and

with the proviso that excluded from the presently claimed compounds of Formula I are the compounds of Formula I′:

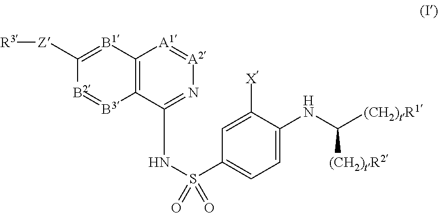

wherein X′ is NO₂ or —SO₂—C(X″)₂ wherein X″ is H or halo; A^{1′}, A^{2′}, B^{1′}, B^{2′} and B^{3′} are independently N or CR^{4′}; Z′ is a cycloalkyl, cycloalkenyl, aryl, heterocyclic or heteroaryl group; R^{1′} and R^{2′} are independently aryl, heteroaryl, —NR^{5′}R^{6′}, —CONR^{5′}R^{6′}, —O(CH₂)ᵣ-aryl, —O(CH₂)ᵣ-heteroaryl, —CO(CH₂)_{r′}aryl, —CO(CH₂)ᵣ-heteroaryl, —CO₂(CH₂)_{r′}aryl, —CO₂(CH₂)_{r′}heteroaryl, —OCO(CH₂)_{r′}aryl, —OCO(CH₂)_{r′}heteroaryl, —S(CH₂)_{r′}aryl, —S(CH₂)_{r′}heteroaryl, —SO(CH₂)^{r′}aryl, —SO(CH₂)_{r′}heteroaryl, —SO₂(CH₂)_{r′}aryl or —SO₂(CH₂)_{r′}heteroaryl; R^{3′} is alkyl, alkenyl, —(CH₂)_{t′}cycloalkyl, —(CH₂)_{t′}cycloalkenyl, —(CH₂)_{t′}aryl, —(CH₂)_{t′}heterocyclyl or —(CH₂)_{t′}heteroaryl, wherein each cycloalkyl, cycloalkenyl, aryl, heterocyclyl and heteroaryl may be optionally substituted with alkyl, alkenyl, halo, nitro, haloalkyl, or phenyl optionally substituted with 1, 2 or 3 alkyl, alkenyl, alkoxy, halo or nitro groups; R^{4′} is hydrogen, halogen, —C₂₋₆alkyl, —C₂₋₆alkenyl, —C₂₋₆alkynyl, hydroxy, —OC₂₋₆alkyl, —OC₂₋₆alkenyl, —OC₂₋₆alkynyl, —N(R^{7′})₂, acyl, —C(R^{8′})₂ or —CON(R^{9′})₂; R^{5′} and R^{6′} are independently hydrogen, alkyl or alkenyl or R^{5′} and R^{6′} taken together with the nitrogen to which they are attached form a heterocyclic or heteroaryl ring; each R^{7′} is independently hydrogen, —C₂₋₆alkyl, —C₂₋₆alkenyl, —C₂₋₆alkynyl or acyl; each R^{8′} is independently hydrogen or halogen; each R^{9′} is independently hydrogen, —C₁₋₆alkyl, —C₂₋₆alkenyl or —C₂₋₆alkynyl, t′ is 0 or an integer 1 to 6; and r′ is 0 or an integer 1 to 6; wherein each alkyl, alkenyl, alkynyl, cycloalkyl, cycloalkenyl, aryl, heterocyclyl and heteroaryl group may be optionally substituted.

In each of the additional embodiments set forth below, there is the proviso that the compounds of Formula I′ (vide supra) are excluded from these embodiments.

Still another embodiment pertains to compounds of Formula (I)-i

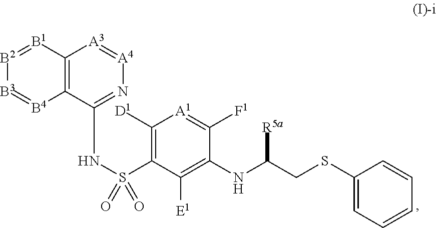

or therapeutically acceptable salts, prodrugs or salts of prodrugs thereof, wherein

R^{5A} is alkyl, alkenyl or alkynyl, each of which is unsubstituted or substituted with one or two or three independently selected R⁶, NC(R^{6A})(R^{6B}), R⁷, OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂. NHC(O)NHR⁷, NHC(O)CH(CH₃) NHC(O)CH(CH₃) NH₂, NHC(O)CH(CH₃)NHC(O)CH(CH₃)NHR¹, OH, (O), C(O)OH, (O), N₃, CN, NH₂, CF₃, CF₂CF₃, F, Cl, Br or I substituents.

Still another embodiment pertains to compounds having Formula I, or therapeutically acceptable salts, prodrugs or salts of prodrugs thereof, wherein A¹ is N or C(A²);

A² is H, F, CN, C(O)OH, C(O)NH₂ or C(O)OR^{1A};

F¹ is R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NR¹C(O)NHR¹, NR¹C(O)N(R¹)₂, SO₂NHR¹, SO₂N(R¹)₂, NHSO₂R¹, NHSO₂NHR¹ or N(CH₃)SO₂N(CH₃)R¹;

D¹ is H, F, C₁ or CF₃;

E¹ is H, F or Cl;

Y¹ is H, CN, NO₂, C(O)OH, F, Cl, Br, I, CF₃, OCF₃, CF₂CF₃, OCF₂CF₃, R¹⁷, OR¹⁷, C(O)R¹⁷, C(O)OR¹⁷, SR¹⁷, NH₂, NHR¹⁷, N(R¹⁷)₂. NHC(O)R¹⁷, C(O)NH₂, C(O)NHR¹⁷, C(O)N(R¹⁷)₂, NHS(O)R¹⁷ or NHSO₂R¹⁷;

or

F¹ and Y¹, together with the atoms to which they are attached, are imidazole or triazole;

R¹ is R², R³, R⁴ or R⁵;

R^{1A} is C₁-alkyl, C₂-alkyl, C₃-alkyl, C₄-alkyl, C₅-alkyl, C₆-alkyl;

R² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;

R⁵ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁶, R⁷, OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂, NHC(O)NHR⁷, NHC(O)CH(CH₃)NHC(O)CH(CH₃)NH₂, OH, (O), C(O)OH, (O), N₃, CN, NH₂, CF₃, CF₂CF₃, F, Cl, Br or I substituents;

R⁶ is C₂-spiroalkyl, C₃-spiroalkyl, C₄-spiroalkyl or C₅-spiroalkyl, each of which is unsubstituted or substituted with OH, (O), N₃, CN, CF₃, CF₂CF₃, F, Cl, Br, I, NH₂, NH(CH₃) or N(CH₃)₂;

R⁷ is R⁸, R⁹, R¹⁰ or R¹¹;

R⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine, 1,2,3-triazole or R^{8A};

R^{8A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), CNOH, CNOCH₃, S, S(O), SO₂ or NH and one or two CH moieties unreplaced or replaced with N;

R⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;

R¹¹ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two or three independently selected R¹², OR¹², NHR¹², N(R¹²)₂, C(O)NH₂, C(O)NHR¹², C(O)N(R¹²)₂, OH, (O), C(O)OH, N₃, CN, NH₂, CF₃, CF₂CF₃, F, Cl, Br or I substituents;

R¹² is R¹³, R¹⁴, R¹⁵ or R¹⁶;

R¹³ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{13A};

R^{13A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), CNOH, CNOCH₃, S, S(O), SO₂ or NH and one or two CH moieties unreplaced or replaced with N;

R¹⁴ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl, 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁵ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;

R¹⁶ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₃-alkynyl, C₄-alkynyl, C₅-alkynyl or C₆-alkynyl;

R¹⁷ is R¹⁸, R¹⁹, R²⁰ or R²¹;

R¹⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R¹⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;

R²¹ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two or three independently selected R²², OR²², NHR²², N(R²²)₂, C(O)NH₂, C(O)NHR²², C(O)N(R²²)₂, OH, (O), C(O)OH, N₃, CN, NH₂, CF₃, CF₂CF₃, F, Cl, Br or I substituents;

R²² is R²³, R²⁴ or R²⁵;

R²³ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²⁴ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl, 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine or 1,2,3-triazole;

R²⁵ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;

A³ and A⁴ are each independently N or C(R²⁶); each R²⁶ is independently hydrogen, halogen, C₂-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl, hydroxy, C₂-alkoxy, C₂-alkoxy, C₃-alkoxy, C₄-alkoxy, C₅-alkoxy, C₆-alkoxy, C₂-alkenyloxy, C₃-alkenyloxy, C₄-alkenyloxy, C₅-alkenyloxy, C₆-alkenyloxy, C₂-alkynyloxy, C₃-alkynyloxy, C₄-alkynyloxy, C₅-alkynyloxy, C₆-alkynyloxy, —C(O)N(R^{26A})₂, acyl, —N(R^{26B})₂, —C(R^{26C})₃, wherein R^{26A} is independently C₂-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl; R^{26B} is independently hydrogen, C₂-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl or acyl; R^{26C} is independently hydrogen, F, Cl, Br or I;

B¹, B², B³ and B⁴ are each independently N, C(Z) or C(R²⁷), wherein one of B², B², B³ or B⁴ is C(Z); each R²⁷ is independently selected from H, F, Cl, Br, I, CH₂R^{27C}, CH(R^{27B})(R^{27C}), C(R^{27B} (R^{27A})(R^{27C}), C(O)R^{27C}, OR^{27C}, SR^{27C}, S(O)R^{27C}, SO₂R^{27C}, NHR^{27C} or N(R^{27D})R^{27C};

R^{27A} and R^{27B} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;

R^{27D} is R^{27E}, C(O)R^{27E} or C(O)OR^{27E};

R^{27E} is R^{27F} or R^{27G};

R^{27F} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27G} is C₁-alkyl, alkyl, C₂-alkyl, C₃-alkyl, C₄-alkyl, C₅-alkyl or C₆-alkyl, each of which is unsubstituted or substituted with R^{27H};

R^{27H} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27C} is R^{27I}, R^{27J} or R^{27K}, each of which is substituted with F, Cl, Br, I, R^{27L}, OR^{27L}, NHR^{27L}, N(R^{27L})₂. NHC(O)OR^{27L}, SR^{27L}, S(O)R^{27L} or SO₂R^{27L};

R^{27I} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27J} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27K} is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;

R^{27L} is R^{27M}, R^{27N}, R^{27P} or R^{27Q};

R^{27M} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{27MM};

R^{27MM} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;

R^{27N} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27P} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27Q} is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R^{27QQ}, OR^{27QQ}, NHR^{27QQ}, N(R^{27QQ})₂, C(O)NH₂, C(O)NHR^{27QQ}, C(O)N(R^{27QQ})₂, OH, (O), C(O)OH, N₃, CN, NH₂, CF₃, CF₂CF₃, F, Cl, Br or I substituents;

R^{27QQ} is R^{27R}, R^{27S} or R^{T};

R^{27R} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27S} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27T} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

Z is R²⁸, R²⁹ or R³⁰;

R²⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R²⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R³⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₁₁-cycloalkyl, C₁₂-cycloalkyl, C₁₃-cycloalkyl, C₁₄-cycloalkyl, C₄-cycloalkenyl, C₅-cycloalkenyl, C₆-cycloalkenyl, C₇-cycloalkenyl, C₈-cycloalkenyl, C₉-cycloalkenyl or C₁₀-cycloalkenyl, each having one or two CH₂ moieties unreplaced or replaced with independently selected O, C(O), S, S(O), SO₂ or NH and one or two CH moieties unreplaced or replaced with N;

each of R²⁸, R²⁹ or R³⁰ is unsubstituted or substituted with F, Cl, Br, I, CH₂R³⁷, CH(R³¹)(R³⁷), C(R³¹)(R^{31A})(R³⁷), C(O)R³⁷, OR³⁷, SR³⁷, S(O)R³⁷, SO₂R³⁷, NHR³⁷ or N(R³²)R³⁷;

R³¹ and R^{31A} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;

R³² is R³³, C(O)R³³ or C(O)OR³³;

R³³ is R³⁴ or R³⁵:

R³⁴ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R³⁵ is C₁-alkyl, C₂-alkyl, C₃-alkyl, C₄-alkyl, C₅-alkyl or C₆-alkyl, each of which is unsubstituted or substituted with R³⁶;

R³⁶ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R³⁷ is R³⁸, R³⁹ or R⁴⁰, each of which is unsubstituted or is substituted with F, Cl, Br, I, R⁴¹, OR⁴¹, NHR⁴¹, NHC(O)OR⁴¹, SR⁴¹, S(O)R⁴¹ or SO₂R⁴¹;

R³⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R³⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁰ is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;

R⁴¹ is R⁴², R⁴³, R⁴⁴ or R⁴⁵;

R⁴² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{42A};

R^{42A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;

R⁴³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁵ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R⁴⁶, OR⁴⁶, NHR⁴⁶, N(R⁴⁶)₂. C(O)NH₂, C(O)NHR⁴⁶, C(O)N(R⁴⁶)₂, OH, (O), C(O)OH, N₃, CN, NH₂, CF₃, CF₂CF₃, F, Cl, Br or I substituents;

R⁴⁶ is R⁴⁷, R⁴⁸ or R⁴⁹;

R⁴⁷ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁸ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁹ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

wherein the moieties represented by F¹ and Y¹ together are substituted with C₁-alkyl, C₂-alkyl, C₃-alkyl, C₄-alkyl, C₅-alkyl or C₆-alkyl, each of which is substituted with one or two independently selected SR⁵⁵ or N(R⁵⁵)₂ substituents, wherein R⁵⁵ is independently selected phenyl, C₁-alkyl, C₂-alkyl, C₃-alkyl, C₄-alkyl, C₅-alkyl or C₆-alkyl;

the moieties represented by R², R³ and R⁴ are unsubstituted or substituted with one or two independently selected R⁵⁰, OR⁵⁰, SR⁵⁰, SO₂R⁵⁰, CO(O)R⁵⁰ or OCF₃ substituents, wherein R⁵⁰ is phenyl, C₁-alkyl, C₂-alkyl, C₃-alkyl, C₄-alkyl, C₅-alkyl or C₆-alkyl;

the moieties represented by R⁸, R⁹ and R¹⁰ are unsubstituted or substituted with one or two independently selected R⁵⁰, OR⁵⁰, C(O)NHSO₂R⁵⁰, CO(O)R⁵⁰, C(O)R⁵⁰, C(O)OH, C(O)NHOH, OH, NH₂, F, Cl, Br or I substituents, wherein R⁵⁰ is phenyl, tetrazolyl or R⁵⁴, wherein R⁵⁴ is C₁-alkyl, C₂-alkyl, C₃-alkyl, C₄-alkyl, C₅-alkyl or C₆-alkyl, each of which is unsubstituted or substituted with phenyl; the moieties represented by R²⁶ and R²⁷ are further unsubstituted or substituted with one or two independently selected F, Br, Cl or I substituents;

the moieties represented by R²⁸, R²⁹ and R³⁰ are further unsubstituted or substituted with OR⁵⁴, wherein R⁵⁴ is C₁-alkyl, C₂-alkyl, C₃-alkyl, C₄-alkyl, C₅-alkyl or C₆-alkyl, each of which is unsubstituted or substituted with R⁵⁶, wherein R⁵⁶ is C₃-cycloalkyl, C₄-cycloalkyl C₅-cycloalkyl or C₆-cycloalkyl, each having one or two CH₂ moieties replaced with independently selected O or NH and one CH moiety unreplaced or replaced with N;

the moieties represented by R³⁸, R³⁹ and R⁴⁰ are further unsubstituted or substituted with one or two independently selected R⁵⁴, F, Br, Cl or I substituents, wherein R⁵⁴ is C₁-alkyl, C₂-alkyl, C₃-alkyl, C₄-alkyl, C₅-alkyl or C₆-alkyl; and

the moieties represented by R⁴², R⁴³, R⁴⁴ and R⁴⁵ are unsubstituted or substituted with one or two independently selected R⁵⁰, OR⁵⁰, SR⁵⁰, N(R⁵⁰)₂, SO₂R⁵⁰, CN, CF₃, F, Cl, Br or I substituents, wherein R⁵⁰ is phenyl or R⁵⁴, wherein R⁵⁴ is C₁-alkyl, C₂-alkyl, C₃-alkyl, C₄-alkyl, C₅-alkyl or C₆-alkyl, each of which is unsubstituted or substituted with N(R⁵⁵)₂ or

R⁵⁶, wherein R⁵⁵ is C₁-alkyl, C₂-alkyl, C₃-alkyl, C₄-alkyl, C₅-alkyl or C₆-alkyl and R⁵⁶ is C₃-cycloalkyl, C₄-cycloalkyl, C₅-cycloalkyl or C₆-cycloalkyl, each having one CH₂ moiety unreplaced or replaced with independently selected O, C(O), S, S(O), SO₂ or NH and one or two CH moieties unreplaced or replaced with N.

Still another embodiment pertains to compounds having Formula I, or therapeutically acceptable salts, prodrugs or salts of prodrugs thereof, wherein A¹ is C(A²);

A² is H, F, CN, C(O)OH, C(O)NH₂ or C(O)OR^{1A};

F¹ is R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NR¹C(O)NHR¹, NR¹C(O)N(R¹)₂, SO₂NHR¹, SO₂N(R¹)₂, NHSO₂R¹, NHSO₂NHR¹ or N(CH₃)SO₂N(CH₃)R¹;

D¹ is H, F, C₁ or CF₃;

E¹ is H, F or Cl;

Y¹ is H, CN, NO₂, C(O)OH, F, Cl, Br, CF₃, 0CF₃, NH₂, C(O)NH₂, or

F¹ and Y¹, together with the atoms to which they are attached, are imidazole or triazole;

R¹ is R², R³, R⁴ or R⁵;

R^{1A} is C₁-alkyl, C₂-alkyl, C₃-alkyl, C₄-alkyl, C₅-alkyl, C₆-alkyl;

R² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine or 1,2,3-triazole;

R³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

R⁵ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁶, R⁷, OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂, NHC(O)NHR⁷, NHC(O)CH(CH₃)NHC(O)CH(CH₃)NH₂, OH, (O), C(O)OH, (O), N₃, CN, NH₂, CF₃, CF₂CF₃, F, Cl, Br or I substituents;

R⁶ is C₂-spiroalkyl, C₃-spiroalkyl, C₄-spiroalkyl or C₅-spiroalkyl, each of which is unsubstituted or substituted with OH, (O), N₃, CN, CF₃, CF₂CF₃, F, Cl, Br, I, NH₂, NH(CH₃) or N(CH₃)₂;

R⁷ is R⁸, R⁹, R¹⁰ or R¹¹;

R⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine, 1,2,3-triazole or R^{BA};

R^{8A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;

R⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R¹⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, each having one or two CH₂ moieties unreplaced or replaced with independently selected O, C(O), CNOCH₃, S, S(O), SO₂ or NH and one or two CH moieties unreplaced or replaced with N;

R¹¹ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two or three independently selected R¹², OR¹², NHR¹², N(R¹²)₂, C(O)NH₂, C(O)NHR¹², C(O)N(R¹²)₂, OH, (O)C(O)OH, N₃, CN, NH₂, CF₃, CF₂CF₃, F, Cl, Br or I substituents;

R¹² is R¹³, R¹⁴, R¹⁵ or R¹⁶;

R¹³ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine, 1,2,3-triazole or R^{13A};

R^{13A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;

R¹⁴ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl, 1,2,3-triazolyl, each of which is unfused or fused with benzene;

R¹⁵ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;

R¹⁶ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₃-alkynyl, C₄-alkynyl, C₅-alkynyl or C₆-alkynyl;

A³ and A⁴ are each independently N or C(R²⁶); each R²⁶ is independently hydrogen, halogen, C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl, hydroxy, C₁-alkoxy, C₂-alkoxy, C₃-alkoxy, C₄-alkoxy, C₅-alkoxy, C₆-alkoxy, C₂-alkenyloxy, C₃-alkenyloxy, C₄-alkenyloxy, C₅-alkenyloxy, C₆-alkenyloxy, alkynyloxy, C₃-alkynyloxy, C₄-alkynyloxy, C₅-alkynyloxy, C₆-alkynyloxy, —C(O)N(R^{26A})₂, acyl, —N(R^{26B})₂, —C(R^{26C})₃, wherein R^{26A} is independently C₁-alkyl, C₂-alkyl, C₃-alkyl, C₄-alkyl, alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl; R^{26B} is independently hydrogen, C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl or acyl; R^{26C} is independently hydrogen, F, Cl or Br;

B¹, B², B³ and B⁴ are each independently N, C (Z) or C(R²⁷) wherein one of B², B², B³ or B⁴ is C(Z); each R²⁷ is independently selected from H, Cl, Br, I, CH₂R^{27C}, C(R^{27B})(R^{27A})(R^{27C}), C(O)R^{27C}, OR^{27C}, SR^{27C}, S(O)R^{27C}, SO₂R^{27C} or NHR^{27C};

R^{27A} and R^{27B} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;

R^{27C} is R^{27I}, R^{27J} or R^{27K}, each of which is substituted with F, Cl, Br, I, R^{27L}, OR^{27L}, NHR^{27L}, N(R^{27L})₂, NHC(O)OR^{27L}, SR^{27L}S(O)R^{27L} or SO₂R^{27L};

R^{27I} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27J} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R^{27K} is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;

R^{27L} is R^{27M}, R^{27N}, R^{27P} or R^{27Q};

R^{27M} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{27MM};

R^{27MM} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;

R^{27N} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R^{27P} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27Q} is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R^{27QQ}, OR^{27QQ}, NHR^{27QQ}, N(R^{27QQ})₂, C(O)NH₂, C(O)NHR^{27QQ}, C(O)N(R^{27QQ})₂, OH, (O), C(O)OH, N₃, CN, NH₂, CF₃, CF₂CF₃, F, Cl, Br or I substituents;

R^{27QQ} is R^{27R}, R^{27S} or R^{T};

R^{27R} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R^{27S} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R^{27T} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

Z is R²⁸, R²⁹ or R³⁰;

R²⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine or 1,2,3-triazole;

R²⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R³⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₁₁-cycloalkyl, C₁₂-cycloalkyl, C₁₃-cycloalkyl, C₁₄-cycloalkyl, C₄-cycloalkenyl, C₅-cycloalkenyl, C₆-cycloalkenyl, C₇-cycloalkenyl, C₈-cycloalkenyl, C₉-cycloalkenyl or C₁₀-cycloalkenyl, each having one or two CH₂ moieties unreplaced or replaced with independently selected O, C(O), S, S(O), SO₂ or NH and one or two CH moieties unreplaced or replaced with N;

each of R²⁸, R²⁹ and R³⁰ is unsubstituted or substituted with Cl, Br, CH₂R³⁷, C(R³¹)(R^{31A})(O)R³⁷, C(O)R³⁷, OR³⁷, SR³⁷, S(O)R³⁷, SO₂R³⁷ or NHR³⁷;

R³¹ and R^{31A} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;

R³⁷ is R³⁸, R³⁹ or R⁴⁰, each of which is unsubstituted or substituted with F, Cl, Br, I, R⁴¹, OR⁴¹, NHR⁴¹, N(R⁴¹)₂, NHC(O)OR⁴¹, SR⁴¹, S(O)R⁴¹ or SO₂^{R41};

R³⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R³⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R⁴⁰ is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O). SO₂ or NH and one or two CH moieties unreplaced or replaced with N;

R⁴¹ is R⁴², R⁴³, R⁴⁴ or R⁴⁵;

R⁴² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{42A};

R^{42A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;

R⁴³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R⁴⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁵ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R⁴⁶, OR⁴⁶, NHR⁴⁶, N(R⁴⁶)₂. C(O)NH₂, C(O)NHR⁴⁶, C(O)N(R⁴⁶)₂, OH, (O), C(O)OH, N₃, CN, NH₂, CF₃, CF₂CF₃, F, Cl, Br or I substituents;

R⁴⁶ is R⁴⁷, R⁴⁸ or R⁴⁹;

R⁴⁷ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;

R⁴⁸ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;

R⁴⁹ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;

wherein the moieties represented by F¹ and Y¹ together are substituted with C₂-alkyl, C₃-alkyl or C₄-alkyl, each of which is substituted with one or two independently selected SR⁵⁵ or N(R⁵⁵)₂ substituents, wherein R⁵⁵ is independently selected phenyl or C₁-alkyl;

the moieties represented by R², R³ and R⁴ are unsubstituted or substituted with one or two independently selected R⁵⁰, OR⁵⁰, SR⁵⁰, SO₂R⁵⁰, CO(O)R⁵⁰ or OCF₃ substituents, wherein R⁵⁰ is phenyl, C₁-alkyl, C₂-alkyl, C₃-alkyl or C₄-alkyl;

the moieties represented by R⁸, R⁹ and R¹⁰ are unsubstituted or substituted with one or two independently selected R⁵⁰, OR⁵⁰, C(O)NHSO₂R⁵⁰, CO(O)R⁵⁰, C(O)R⁵⁰, C(O)OH, C(O)NHOH, OH, NH₂, F, Cl, Br or I substituents, wherein R⁵⁰ is phenyl, tetrazolyl or R⁵⁴, wherein R⁵⁴ is C₁-alkyl, C₂-alkyl or C₃-alkyl, each of which is unsubstituted or substituted with phenyl;

the moieties represented by R²⁸, R²⁹ and R³⁰ are further unsubstituted or substituted with OR⁵⁴, wherein R⁵⁴ is C₁-alkyl or C₂-alkyl, each of which is unsubstituted or substituted with N(R⁵⁵)₂ or R⁵⁶, wherein R⁵⁵ is C₁-alkyl, C₂-alkyl, C₃-alkyl, C₄-alkyl, C₅-alkyl or C₆-alkyl, and R⁵⁶ is C₅-cycloalkyl or C₆-cycloalkyl, each having one or two CH₂ moieties replaced with independently selected O or NH and one CH moiety unreplaced or replaced with N;

the moieties represented by R³⁸, R³⁹ and R⁴⁰ are further unsubstituted or substituted with one or two independently selected C₁-alkyl, F, Br, Cl or I substituents; and

the moieties represented by R⁴², R⁴³, R⁴⁴ and R⁴⁵ are unsubstituted or substituted with one or two independently selected R⁵⁰, OR⁵⁰, SR⁵⁰, SO₂R⁵⁰, CN, CF₃, F, Cl, Br or I substituents, wherein R⁵⁰ is phenyl or R⁵⁴, wherein R⁵⁴ is C₁-alkyl, C₂-alkyl or C₃-alkyl, each of which is unsubstituted or substituted with N(C₁-alkyl)₂ or C₆-cycloalkyl having one CH₂ moiety replaced with O and one CH moiety replaced with N.

Still in another embodiment, compounds of Formula I have the Subformulae Ia:

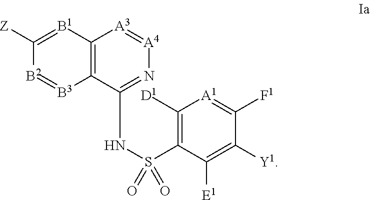

Still in another embodiment, in the compound of Formula I the moiety:

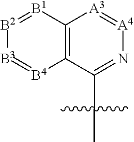

in Formula I is selected from the group consisting of:

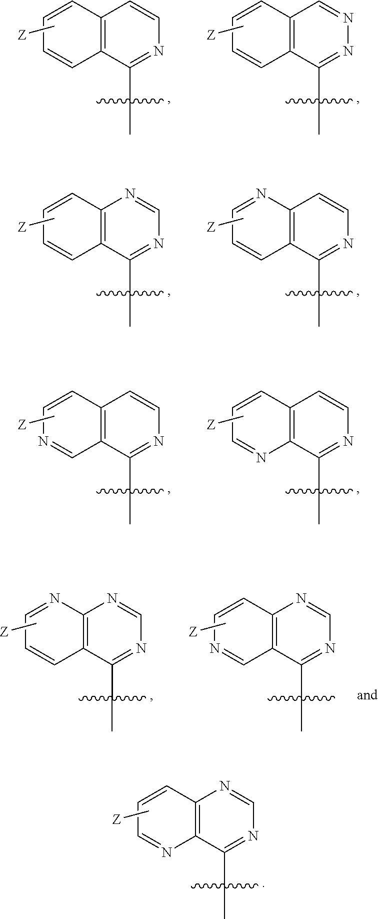

Still another embodiment pertains to compounds having Formula I, or therapeutically acceptable salts, prodrugs or salts of prodrugs thereof, wherein A¹ is C(A²);

A² is H, F, CN, C(O)OH, C(O)NH₂ or C(O)OCH₃;

F¹ is R¹, OR′, NHR¹, N(R¹)₂ or NR¹C(O)N(R¹)₂;

D¹ is H, F, C₁ or CF₃;

E¹ is H, F or Cl;

Y¹ is H, CN, NO₂, F, Cl, CF₃, OCF₃, NH₂, C(O)NH₂, or

F¹ and Y¹, together with the atoms to which they are attached, are imidazole or triazole;

R¹ is phenyl, pyrrolyl, C₅-cycloalkyl, C₆-cycloalkyl, piperidinyl, tetrahydrofuranyl, tetrahydropyranyl, tetrahydrothiopyranyl or R⁵;

R⁵ is C₁-alkyl, C₂-alkyl, C₃-alkyl, C₄-alkyl, C₅-alkyl or C₆-alkyl, each of which is unsubstituted or substituted with one or two or three independently selected C₄-spiroalkyl, C₅-spiroalkyl, R⁷, OR⁷, SR⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, NHC(O)NH₂, NHC(O)CH(CH₃)NHC(O)CH(CH₃)NH₂, OH, C(O)OH or NH₂ substituents;

R⁷ is phenyl, furanyl, imidazolyl, pyridinyl, tetrazolyl, thiazolyl, thienyl, 1,3-benzoxazolyl, 1,3-benzodioxolyl, 1,3-benzothiazole, C₃-cycloalkyl, C₄-cycloalkyl, C₅-cycloalkyl, C₆-cycloalkyl, azetidinyl, morpholinyl, piperazinyl, piperidinyl, thiomorpholinyl, thiomorpholinyl sulfone 7-azabicyclo[2.2.1]heptanyl, 8-azabicyclo[3.2.1]-octanyl, 4,5-dihydro-1H-imidazolyl 2-oxa-5-azabicyclo-[2.2.1]heptanyl, 1,4,5,6-tetrahydropyrimidinyl or R¹¹;

R¹¹ is C₁-alkyl, C₂-alkyl, C₃-alkyl or C₄-alkyl, each of which is unsubstituted or substituted with one or two or three independently selected R¹², OR¹²N(R¹²)₂, C(O)N(R¹²)₂, OH, C(O)OH, NH₂, CF₃, F, Cl, Br or I substituents;

R¹² is 1,3-benzodioxolyl, pyridinyl, morpholinyl or C₁-alkyl;

A³ and A⁴ are each independently N or C(R²⁶); each R²⁶ is independently hydrogen, halogen, C₁-alkyl, C₂-alkyl, C₃-alkyl, hydroxy, C₁-alkoxy, C₂-alkoxy, C₃-alkoxy, C₂-alkenyloxy, C₃-alkenyloxy, C₄-alkenyloxy, C₂-alkynyloxy, C₃-alkynyloxy, C₄-alkynyloxy, acyl, —N(R^{26B})₂, —C(R^{26C})₃, wherein R^{26A} is independently C₁-alkyl, C₂-alkyl, C₃-alkyl, C₄-alkyl, C₂-alkenyl, C₃-alkenyl, C₄-alkenyl, C₂-alkynyl, C₃-alkynyl or C₄-alkynyl; R^{26B} is independently hydrogen, C₁-alkyl, C₂-alkyl, C₃-alkyl, C₂-alkenyl, C₃-alkenyl, C₄-alkenyl, C₂-alkynyl, C₃-alkynyl, C₄-alkynyl or acyl; R^{26C} is independently hydrogen, F, Cl or Br;

B¹, B², B², B³ and B⁴ are each independently N, C(Z) or C(R²²), wherein one of B², B², B³ or B⁴ is C(Z) and two or three of B¹, B², B³ or B⁴ is C(R²²); R²⁷ is independently H, F, Cl, Br or I; Z is C₆-cycloalkenyl, piperazinyl, piperidinyl, 1,2,3,6-tetrahydropyridinyl or octahydropyrrolo[3,4-c]pyrrolyl, each of which is substituted with CH₂R³⁷, C(C₂-spiroalkyl) (R³⁷) or C(O)R³⁷;

R³⁷ is phenyl, naphthyl, imidazolyl, pyrazolyl, pyridinyl, C₅-cycloalkenyl, C₆-cycloalkenyl, C₇-cycloalkenyl, C₈-cycloalkenyl or 3,6-dihydro-2H-pyranyl, each of which is substituted with F, Cl, Br, I, R⁴¹, NHR⁴¹, N(R⁴¹)², NHC(O)OR⁴¹ or SR⁴¹;

R⁴¹ is phenyl, naphthyl, cyclohexyl, morpholinyl, piperidinyl, thienyl, pyridinyl, quinolinyl, benzofuranyl, 1,3-benzodioxolyl, isoindolinyl, 1,3-oxazolidin-2-onyl or R⁴⁵;

R⁴⁵ is C₁-alkyl, C₂-alkyl, C₃-alkyl or C₄-alkyl, each of which is unsubstituted or substituted with phenyl;

wherein the moieties represented by F¹ and Y¹ together are substituted with C₂-alkyl, C₃-alkyl or C₄-alkyl, each of which is substituted with one or two independently selected SR⁵⁵ or N(R⁵⁵)₂ substituents, wherein R⁵⁵ is independently selected phenyl or C₁-alkyl;

the moieties represented by R¹ are unsubstituted or substituted with one or two independently selected R⁵⁰, OR⁵⁰, SR⁵⁰, SO₂R⁵⁰, CO(O)R⁵⁰ or OCF₃ substituents, wherein R⁵⁰ is phenyl, C₁-alkyl, C₂-alkyl, C₃-alkyl or C₄-alkyl;

the moieties represented by R⁷ are unsubstituted or substituted with one or two independently selected R⁵⁰, OR⁵⁰, C(O)NHSO₂R⁵⁰, CO(O)R⁵⁰, C(O)R⁵⁰, C(O)OH, C(O)NHOH, OH, NH₂, F, Cl, Br or I substituents, wherein R⁵⁰ is phenyl, tetrazolyl or R⁵⁴, wherein R⁵⁴ is C₁-alkyl, C₂-alkyl or C₃-alkyl, each of which is unsubstituted or substituted with phenyl;

the C₆-cycloalkenyl, piperazinyl, piperidinyl, 1,2,3,6-tetrahydropyridinyl and octahydropyrrolo[3,4-c]pyrrolyl moieties of Z are further unsubstituted or substituted with OR⁵⁴, wherein R⁵⁴ is C₁-alkyl or C₂-alkyl, each of which is unsubstituted or substituted with N(C₁-alkyl)₂, morpholinyl, piperidinyl or piperidinyl; and

the moieties represented by R⁴¹ are unsubstituted or substituted with one or two independently selected R⁵⁰, OR⁵⁰, SR⁵⁰, N(R⁵⁰)₂, SO₂R⁵⁰, CN, CF₃, F, Cl, Br or I substituents, wherein R⁵⁰ is phenyl or R⁵⁴, wherein R⁵⁴ is C₁-alkyl, C₂-alkyl or C₃-alkyl, each of which is unsubstituted or substituted with N(C₁-alkyl)₂ or morpholinyl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(1,3-benzodioxol-5-yl)phenylmethyl)piperazin-4-yl, 1-(2-(benzofuran-2-ylphenyl)phenylmethyl)piperazin-4-yl, 1-(2-bromocyclohex-1-en-1-ylmethyl)piperazin-4-yl, 1-(2-bromocyclopent-1-en-1-ylmethyl)piperazin-4-yl, 1-(4-bromophenyl)phenylmethyl)piperazin-4-yl, 1-(2-(4-chlorophenyl)cyclohept-1-en-1-ylmethyl)piperazin-4-yl, 1-((4-chlorophenyl)cyclohex-1-en-1-ylmethyl)-1,2,3,6-tetrahydropyridin-4-yl, 1-(2-(4-chlorophenyl)cyclohex-1-en-1-ylmethyl)piperazin-4-yl, 1-(2-(4-chlorophenyl)cyclopent-1-en-1-ylmethyl)piperazin-4-yl, 1-(2-(4-chlorophenyl)cyclooct-1-en-1-ylmethyl)piperazin-4-yl, 1-((4-(4-chlorophenyl)-5,6-dihydro-2H-pyran-3-yl)methyl)piperazin-4-yl, 1-((4-(4-chlorophenyl)-5,6-dihydro-2H-pyran-3-yl)methyl)-piperazin-4-yl, 1-((2-(4-chlorophenyl)-5,5-dimethyl-1-cyclohex-1-en-1-yl)methyl)-1,2,3,6-tetrahydropyridin-4-yl, 1-(2-(4-chlorophenyl)naphth-3-ylmethyl)piperazin-4-yl, 1-(2-(4-chlorophenyl)pyridin-3-ylmethyl)piperazin-4-yl, 1-(3-(4-chlorophenyl)pyridin-4-ylmethyl)piperazin-4-yl, 1-((4-(4-chlorophenyl)pyridin-5-yl)methyl)-piperazin-4-yl, 1-(2-(4-chlorophenyl)phenylcarbonyl)piperazin-4-yl, 1-(2-(4-chlorophenyl)phenylcycloprop-1-yl)piperazin-4-yl or 1-(2-(4-chlorophenyl)phenylmethyl)cyclohex-1-en-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethyl-pyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 2-((4′-chlorobiphenyl-2-yl)methyl)-octahydropyrrolo[3,4-c]pyrrol-5-yl, 1-(2-(4-chlorophenyl)phenylmethyl)piperazin-4-yl, 1-(2-(4-chlorophenyl)phenylmethyl)-4-methoxypiperidin-4-yl, 1-(2-(4-chlorophenyl)phenylmethyl)-piperazin-4-yl, 1-(2-(4-chlorophenyl)phenylmethyl)piperazin-4-yl, 1-(2-(4-chlorophenyl)phenylmethyl)-1,2,3,6-tetrahydropyridin-4-yl, 1-(2-(4-chlorophenyl)phenylmethyl)piperidin-4-yl, 1-(2-(cyclohex-1-ylamino)phenylmethyl)piperazin-4-yl, 1-(2-cyclohex-1-ylphenylmethyl)piperazin-4-yl, 1-(2-(3-cyanophenyl)phenylmethyl)piperazin-4-yl, 1-(2-(2,4-dichlorophenyl)phenylmethyl)piperazin-4-yl, 1-(2-(3,4-dichlorophenyl)phenylmethyl)piperazin-4-yl, 1-(2-(2,4-difluorophenyl)phenylmethyl)piperazin-4-yl, 1-(2-(1,3-dihydro-2H-isoindol-2-yl)phenylmethyl)piperazin-4-yl, 1-(3-(1,1-dimethylethoxycarbonylamino)phenyl)piperazin-4-yl, 1-(4-(2-(dimethylamino)ethoxy)phenyl)phenylmethyl)piperazin-4-yl, or 1-(3-(dimethylamino)phenyl)piperazin-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethyl-pyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(4-(dimethylamino)phenyl)phenylcarbonyl)piperazin-4-yl, 1-(2-(4-(dimethylamino)phenyl)phenylmethyl)piperazin-4-yl, 1-((2-(4-(dimethylamino)phenyl)pyridin-3-yl)methyl)piperazin-4-yl, 1-(2-(5,5-dimethyl-2-oxo-1,3-oxazolidin-3-yl)phenylmethyl)piperazin-4-yl, 1-(2-(4-fluorophenyl)cyclopent-1-en-1-ylmethyl)piperazin-4-yl, 1-(2-(4-fluorophenyl)-3-fluorophenylmethyl)piperazin-4-yl, 1-(2-(4-fluorophenyl)phenylmethyl)piperazin-4-yl, 1-((2-(4-fluorophenyl)pyridin-3-yl)methyl)piperazin-4-yl, 1-(2-(isopropylamino)phenylmethyl)piperazin-4-yl, 1-(2-(4-(isopropylsulfanyl)phenyl)phenylmethyl)piperazin-4-yl, 1-(2-(4-methoxyphenyl)cyclopent-1-en-1-ylmethyl)piperazin-4-yl, 1-(2-(3-methoxyphenyl)phenylmethyl)piperazin-4-yl, 1-(2-(4-methoxyphenyl)phenylmethyl)piperazin-4-yl, 1-((2-(4-methoxyphenyl)pyridin-3-yl)methyl)piperazin-4-yl, (4-methoxy)-4-(2-(pyridin-3-yl)phenylmethyl)piperidin-1-yl, 1-(2-(2-methyl-4-dichlorophenyl)phenylmethyl)piperazin-4-yl, 1-(2-(2-methylphenyl)phenylmethyl)piperazin-4-yl, 1-(2-(4-(methylsulfonyl)phenyl)methyl)piperazin-4-yl, 1-(2-(4-methylsulfonylphenyl)phenylmethyl)piperazin-4-yl, or 1-((2-(4-methylsulfonylphenyl)pyridin-3-yl)methyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl) (methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethyl-pyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(5-methylthien-2-yl)phenylmethyl)piperazin-4-yl, 1-(2-(4-methylsulfanylphenyl)cyclohex-1-en-1-ylmethyl)piperazin-4-yl, 1-(2-(4-methylsulfanylphenyl)phenylcarbonyl)piperazin-4-yl, 1-((2-(4-methylsulfanylphenyl)pyridin-3-yl)methyl)piperazin-4-yl, 1-(2-(4-methylsulfanylphenyl)phenylmethyl)piperazin-4-yl, 1-(2-(4-(2-(morpholin-1-yl)ethoxy)phenyl)phenylmethyl)piperazin-4-yl, 1-(2-(morpholin-1-yl)phenylmethyl)piperazin-4-yl, 1-(2-(naphth-1-yl)phenylmethyl)piperazin-4-yl, 1-(2-(naphth-2-yl)phenylmethyl)-piperazin-4-yl, 1-(2-(4-phenoxyphenyl)phenylmethyl)piperazin-4-yl, 1-((1-phenyl-1H-imidazol-2-yl)methyl)piperazin-4-yl, 1-((1-phenyl-1H-imidazol-5-yl)methyl)piperazin-4-yl, 1-(2-((phenylmethyl)amino)phenylmethyl)piperazin-4-yl, 1-(2-(phenyl)phenylmethyl)-4-(2-(dimethylamino)ethoxy))piperidin-4-yl, 2-(phenyl)phenylmethyl-4-methoxypiperidin-1-yl, 4-((2-(phenyl)phenylmethyl)-4-(2-(morpholin-4-yl)ethoxy))piperidin-1-yl, 1-(2-(phenyl)phenylmethyl)piperazin-4-yl, 1-(3-(phenyl)phenylmethyl)piperazin-4-yl, 1-(2-(4-(phenyl)phenyl)phenylmethyl)piperazin-4-yl, or 4-(2-(phenyl)phenylmethyl)-4-(2-(piperidin-1-yl)ethoxy)piperidin-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl) (methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 4-(2-(phenyl)phenylmethyl)-4-(2-(pyrrolidin-1-yl)ethoxy)piperidin-1-yl, 1-((2-(phenyl)pyridin-3-yl)methyl)piperazin-4-yl, 1-((1-phenyl-1H-pyrazol-5-yl)methyl)piperazin-4-yl, 1-(2-(piperidin-1-yl)phenylmethyl)piperazin-4-yl, 1-(2-(pyrid-3-yl)phenyl)phenylmethyl)piperazin-4-yl, 1-(2-(quinolin-3-ylphenyl)phenylmethyl)piperazin-4-yl, 1-(2-(quinolin-8-ylphenyl)phenylmethyl)piperazin-4-yl, 4-(2-(thien-2-yl)phenylmethyl)-4-methoxypiperazin-1-yl, 1-(2-(thien-2-yl)phenylmethyl)-piperazin-4-yl, 1-(2-(4-trifluoromethoxyphenyl)phenylmethyl)piperazin-4-yl, or 1-(2-(4-trifluoromethylphenyl)phenylmethyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)1-((phenylsulfanyl)methyl)propylamino, (1R)-2-((2-(dimethylamino)ethyl) (methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(1,3-benzodioxol-5-yl)phenylmethyl)piperazin-4-yl, 1-(2-(benzofuran-2-ylphenyl)phenylmethyl)piperazin-4-yl, 1-(2-bromocyclohex-1-en-1-ylmethyl)piperazin-4-yl, 1-(2-bromocyclopent-1-en-1-ylmethyl)piperazin-4-yl, 1-(4-bromophenyl)phenylmethyl)piperazin-4-yl, 1-(2-(4-chlorophenyl)cyclohept-1-en-1-ylmethyl)piperazin-4-yl, 1-(2-(4-chlorophenyl)cyclohex-1-en-1-ylmethyl)-1,2,3,6-tetrahydropyridin-4-yl, 1-(2-(4-chlorophenyl)cyclohex-1-en-1-ylmethyl)piperazin-4-yl, 1-(2-(4-chlorophenyl)cyclopent-1-en-1-ylmethyl)piperazin-4-yl, 1-(2-(4-chlorophenyl)cyclooct-1-en-1-ylmethyl)piperazin-4-yl, 1-((4-(4-chlorophenyl)-5,6-dihydro-2H-pyran-3-yl)methyl)piperazin-4-yl, 1-((4-(4-chlorophenyl)-5,6-dihydro-2H-pyran-3-yl)methyl)piperazin-4-yl, 1-((2-(4-chlorophenyl)-5,5-dimethyl-1-cyclohex-1-en-1-yl)methyl)-1,2,3,6-tetrahydropyridin-4-yl, 1-(2-(4-chlorophenyl)naphth-3-ylmethyl)piperazin-4-yl, 1-(2-(4-chlorophenyl)pyridin-3-ylmethyl)piperazin-4-yl, 1-(3-(4-chlorophenyl)pyridin-4-ylmethyl)piperazin-4-yl, 1-((4-(4-chlorophenyl)pyridin-5-yl)methyl)piperazin-4-yl, 1-(2-(4-chlorophenyl)phenylcarbonyl)piperazin-4-yl, 1-(2-(4-chlorophenyl)phenylcycloprop-1-yl)piperazin-4-yl, or 4-(2-(4-chlorophenyl)phenylmethyl)cyclohex-1-en-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 5-(2-(4-chlorophenyl)phenylmethyl)hexahydropyrrolo[3,4-c]pyrrol-2(1H)-yl, 1-(2-(4-chlorophenyl)phenylmethyl)piperazin-4-yl, 4-(2-(4-chlorophenyl)phenylmethyl)-4-methoxypiperidin-1-yl, 1-(2-(4-chlorophenyl)phenylmethyl)-piperazin-4-yl, 1-(2-(4-chlorophenyl)phenylmethyl)-1,2,3,6-tetrahydropyridin-4-yl, 1-(2-(cyclohex-1-ylamino)phenylmethyl)piperazin-4-yl, 1-(2-(cyclohex-1-ylamino)phenylmethyl)piperazin-4-yl, 1-(2-(3-cyanophenyl)phenylmethyl)piperazin-4-yl, 1-(2-(2,4-dichlorophenyl)phenylmethyl)piperazin-4-yl, 1-(2-(3,4-dichlorophenyl)phenylmethyl)piperazin-4-yl, 1-(2-(2,4-difluorophenyl)phenylmethyl)piperazin-4-yl, 1-(2-(1,3-dihydro-2H-isoindol-2-yl)phenylmethyl)piperazin-4-yl, 1-(3-(1,1-dimethylethoxycarbonylamino)phenyl)piperazin-4-yl, 1-(4-(2-(dimethylamino)ethoxy)phenyl)phenylmethyl)piperazin-4-yl, or 1-(3-(dimethylamino)phenyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(4-(dimethylamino)phenyl)phenylcarbonyl)piperazin-4-yl, 1-(2-(4-(dimethylamino)phenyl)phenylmethyl)piperazin-4-yl, 1-((2-(4-(dimethylamino)phenyl)pyridin-3-yl)methyl)piperazin-4-yl, 1-(2-(5,5-dimethyl-2-oxo-1,3-oxazolidin-3-yl)phenylmethyl)piperazin-4-yl, 1-(2-(4-fluorophenyl)cyclopent-1-en-1-ylmethyl)piperazin-4-yl, 1-(2-(4-fluorophenyl)-3-fluorophenylmethyl)piperazin-4-yl, 1-(2-(4-fluorophenyl)phenylmethyl)piperazin-4-yl, 1-((2-(4-fluorophenyl)pyridin-3-yl)methyl)piperazin-4-yl, 4-(2-(isopropylamino)phenylmethyl)-piperazin-1-yl)phenylcarbonyl, 4-(2-(isopropylamino)phenylmethyl)-piperazin-1-yl, 1-(2-(4-(isopropylsulfanyl)phenyl)phenylmethyl)piperazin-4-yl, 1-(2-(4-methoxyphenyl)cyclopent-1-en-1-ylmethyl)piperazin-4-yl, 1-(2-(3-methoxyphenyl)phenylmethyl)piperazin-4-yl, 1-(2-(4-methoxyphenyl)phenylmethyl)piperazin-4-yl, 1-((2-(4-methoxyphenyl)pyridin-3-yl)methyl)piperazin-4-yl, (4-methoxy)-4-(2-(pyridin-3-yl)phenylmethyl)piperidin-1-yl, 1-(2-(2-methyl-4-dichlorophenyl)phenylmethyl)piperazin-4-yl, 1-(2-(2-methylphenyl)phenylmethyl)piperazin-4-yl, 1-(2-(4-(methylsulfonyl)-phenyl)methyl)piperazin-4-yl, 1-(2-(4-methylsulfonylphenyl)phenylmethyl)piperazin-4-yl, or 1-((2-(4-methylsulfonylphenyl)pyridin-3-yl)methyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₂ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(5-methylthien-2-yl)phenylmethyl)piperazin-4-yl, 1-(2-(4-methylsulfanylphenyl)cyclohex-1-en-1-ylmethyl)piperazin-4-yl, 1-(2-(4-methylsulfanylphenyl)phenylcarbonyl)piperazin-4-yl, 1-(2-(4-methylsulfanylphenyl)phenylcarbonyl)piperazin-4-yl, 1-((2-(4-methylsulfanylphenyl)pyridin-3-yl)methyl)piperazin-4-yl, 1-(2-(4-methylsulfanylphenyl)phenylmethyl)piperazin-4-yl, 1-(2-(4-(2-(morpholin-1-yl)ethoxy)phenyl)phenylmethyl)piperazin-4-yl, 1-(2-(morpholin-1-yl)phenylmethyl)piperazin-4-yl, 1-(2-(naphth-1-yl)phenylmethyl)piperazin-4-yl, 4-(2-(naphth-2-yl)phenylmethyl)piperazin-1-yl, 1-(2-(4-phenoxyphenyl)phenylmethyl)piperazin-4-yl, 1-((1-phenyl-1H-imidazol-2-yl)methyl)piperazin-4-yl, 1-((1-phenyl-1H-imidazol-5-yl)methyl)piperazin-4-yl, 1-(2-((phenylmethyl)amino)phenylmethyl)piperazin-4-yl, 1-(2-(phenyl)phenylmethyl)-4-(2-(dimethylamino)ethoxy))piperidin-4-yl, 2-(phenyl)phenylmethyl-4-methoxypiperidin-1-yl, 4-((2-(phenyl)phenylmethyl)-4-(2-(morpholin-4-yl)ethoxy))piperidin-1-yl, 1-(2-(phenyl)phenylmethyl)piperazin-4-yl, 1-(3-(phenyl)phenylmethyl)piperazin-4-yl, 1-(2-(4-(phenyl)phenyl)phenylmethyl)piperazin-4-yl, or 4-(2-(phenyl)phenylmethyl)-4-(2-(piperidin-1-yl)ethoxy)piperidin-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-2-(diethylamino)-1-((phenylsulfanyl)methyl)ethylamino, (1R)-3-(diethylamino)-1-((phenylsulfanyl)methyl)propylamino, (1R)-3-((2,2-difluoroethyl)amino)-1-((phenylsulfanyl)methyl)propylamino, (1R)-3-(5,6-dihydro-1(4H)-pyrimidin-1-yl)-1-((phenylsulfanyl)methyl)propylamino, (1R)-3-(diisopropylamino)-1-((phenylsulfanyl)methyl)propylamino, (1R)-2-((2-(dimethylamino)ethyl)(methyl)amino)-1-((phenylsulfanyl)methyl)ethylamino, (1R)-2-(2-(dimethylamino)ethoxy)-1-((phenylsulfanyl)methyl)ethylamino, (3R)-5-(N-((dimethylamino)methylcarbonyl)amino)-1-((phenylsulfanyl)methyl)pentylamino, (1R)-3-(dimethylamino)-1-((phenylsulfanyl)methyl)propoxy, (1R)-3-(dimethylamino)-1-((phenylsulfanyl)methyl)propylamino, (1S)-3-(dimethylamino)-1-((phenylsulfanyl)methyl)propylamino, (1R)-3-(dimethylamino)-3-oxo-1-((pyrimidin-2-ylsulfanyl)methyl)propylamino, (1R)-3-(dimethylamino)-3-oxo-1-((1,3-thiazol-2-yl)methyl)propylamino, (1R)-3-(dimethylamino)-1-((thien-2-ylsulfanyl)methyl)propylamino, (1R)-3-(dimethylamino)-1-(((4-(trifluoromethoxy)phenyl)sulfanyl)methyl)propylamino, (1R)-3-(2,4-dimethyl-4,5-dihydro-1H-imidazol-1-yl)-1-((phenylsulfanyl)methyl)propylamino, (1R)-3-(4,4-dimethyl-4,5-dihydro-1H-imidazol-1-yl)-1-((phenylsulfanyl)methyl)propylamino, (1R)-5-((1,1-dimethylethoxy)carbonylamino)-1-((phenylsulfanyl)methyl)pentylamino, 1-(1,1-dimethylethoxycarbonyl)piperidin-4-yloxy, 1,1-dimethyl-2-(phenylsulfonyl)ethylamino, (1,1-dimethyl-2-(phenylsulfanyl)ethyl)amino, 1,1-dimethyl-2-(phenylsulfanyl)ethyl, 4,4-dimethylpiperidin-1-yl, (1R)-3-(2,6-dimethylpiperidin-1-yl)-1-((phenylsulfanyl)methyl)propylamino, (1R)-3-((2R,6S)-2,6-dimethylpiperidin-1-yl)-1-((phenylsulfanyl)methyl)propylamino, 1,1-dimethyl-2-(pyrimidin-2-ylsulfanyl)ethylamino, (1R)-4-((2R,5S)-2,5-dimethylpyrrolidin-1-yl)-1-((phenylsulfanyl)methyl)butylamino, (1R)-3-((2R,5R)-2,5-dimethylpyrrolidin-1-yl)-1-((phenylsulfanyl)methyl)propylamino, (1R)-3-(2,5-dimethylpyrrolidin-1-yl)-1-((phenylsulfanyl)methyl)propylamino, (1R)-3-((2R,5S)-2,5-dimethylpyrrolidin-1-yl)-1-((phenylsulfanyl)methyl)propylamino, (1R)-3-((2S,5S)-2,5-dimethylpyrrolidin-1-yl)-1-((phenylsulfanyl)methyl)propylamino, 1,1-dimethyl-2-(thien-2-ylsulfanyl)ethylamino, (1R)-3-(1,1-dioxothiomorpholin-4-yl)-1-((phenylsulfanyl)methyl)propylamino, (1R)-3-(dipropylamino)-1-((phenylsulfanyl)methyl)propylamino, (1R)-3-(dipropylamino)-1-((phenylsulfanyl)methyl)propylamino, (1R)-3-(ethyl(2,2,2-trifluoroethyl)amino)-1-((phenylsulfanyl)methyl)propylamino, 1-(ethoxycarbonyl)piperidin-4-yloxy, (1R)-3-((2-fluoroethyl)amino)-1-((phenylsulfanyl)methyl)propylamino, (1R)-1-(((4-fluorophenyl)sulfanyl)methyl)-3-(morpholin-4-yl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 4-(2-(phenyl)phenylmethyl)-4-(2-(pyrrolidin-1-yl)ethoxy)piperidin-1-yl, 1-((2-(phenyl)pyridin-3-yl)methyl)piperazin-4-yl, 1-((1-phenyl-1H-pyrazol-5-yl)methyl)piperazin-4-yl, 1-(2-(piperidin-1-yl)phenylmethyl)piperazin-4-yl, 1-(2-(pyrid-3-yl)phenyl)phenylmethyl)piperazin-4-yl, 1-(2-(quinolin-3-ylphenyl)phenylmethyl)piperazin-4-yl, 1-(2-(quinolin-8-ylphenyl)phenylmethyl)piperazin-4-yl, 4-(2-(thien-2-yl)phenylmethyl)-4-methoxypiperazin-1-yl, 1-(2-(thien-2-yl)phenylmethyl)piperazin-4-yl, 1-(2-(4-trifluoromethoxyphenyl)phenylmethyl)piperazin-4-yl, or 1-(2-(4-trifluoromethylphenyl)phenylmethyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-(4-(hydroxyaminocarbonyl)piperidin-1-yl)-1-((phenylsulfanyl)methyl)propylamino, (1R)-3-(2-hydroxy-2-methylpropyl)amino)-1-((phenylsulfanyl)methyl)propylamino, (1R)-3-(4-hydroxypiperidin-1-yl)-1-((phenylsulfanyl)methyl)propylamino, (3R)-3-(3-hydroxypyrrolidin-1-yl)-1-((phenylsulfanyl)methyl)propylamino, isopropylamino, (1R)-3-(isopropylamino)-1-((phenylsulfanyl)methyl)propylamino, (1R)-3-(1H-imidazol-1-yl)-1-((phenylsulfanyl)methyl)propylamino, (1R)-3-(isopropyl(methyl)amino)-1-((phenylsulfanyl)methyl)propylamino, (4-methoxycyclohex-1-yl)methyl)amino, (1R)-3-(4-(methoxyimino)piperidin-1-yl)-1-((phenylsulfanyl)methyl)propylamino, (1R)-3-(N-methyl-N-carboxymethyl)amino)-1-((phenylsulfanyl)methyl)propylamino, (methyl) (cyclohexyl)amino, (methyl) (cyclohexylmethyl)amino, (1R)-3-(2-methyl-4,5-dihydro-1H-imidazol-1-yl)-1-((phenylsulfanyl)methyl)propylamino, (3R)-3-(N-methyl-N-(dimethylcarbonylmethyl))-1-((phenylsulfanyl)methyl)propylamino, (1R)-3-(N-methyl-N-(1,1-dimethylethyl)amino)-1-((phenylsulfanyl)methyl)propylamino, (N-methyl-N-(1,2-diphenyl)amino)carbonyl)-N-methylamino, (N-methyl-N-((diphenylmethyl)amino)carbonyl)-N-methylamino, (2-methylfuran-3-yl)sulfanyl) (1,1-spirobutyl)ethylamino, (1R)-3-(N-methyl-N-(2-hydroxyethyl))amino)-1-((phenylsulfanyl)methyl)propylamino, (1R)-3-(N-methyl-N-isopropylamino)-1-((phenylsulfanyl)methyl)propylamino, (N-methyl-N-(4-methoxyphenyl)amino)carbonyl)-N-methylamino, 1-methyl-4-(phenylsulfanyl)pyrrolidin-3-ylamino, (N-methyl-N-(4-methylphenyl)amino)carbonyl)-N-methylamino, (N-methyl-N-(2-methylphenyl)amino)carbonyl)-N-methylamino, (1R)-3-(4-methylpiperazin-1-yl)-1-((phenylsulfanyl)methyl)propylamino, 1-methylpiperidin-4-yloxy, (N-methyl-N—((S)-1-phenylethyl)amino)carbonyl)-N-methylamino, (N-methyl-N-(1-phenyl-2-(4-methylpiperazin-4-yl))amino)carbonyl)-N-methylamino, (N-methyl-N-(1-phenyl-2-(morpholin-1-yl))amino)carbonyl)-N-methylamino, (N-methyl-N-(1-phenyl-2-(N,N-dimethylamino)amino)carbonyl)-N-methylamino, (1R)-1-methyl-2-((phenylsulfanyl)methyl)ethylamino, (1S)-1-methyl-2-((phenylsulfanyl)methyl)ethylamino, (1R)-4-(4-methylpiperazin-1-yl)-1-((phenylsulfanyl)methyl)butylamino, (1R)-3-(methyl(pyridin-4-yl)amino)-1-((phenylsulfanyl)methyl)propylamino, (1R)-5-((methylsulfonylamino)-1-((phenylsulfanyl)methyl)pentylamino, (1R)-3-(4-(methylsulfonylaminocarbonyl)piperidin-1-yl)-1-((phenylsulfanyl)methyl)-propylamino, 2-((4-methyl-1,3-thiazol-2-yl)sulfanyl)ethylamino, (N-methyl-N-(4-trifluoromethoxyphenyl)amino)carbonyl)-N-methylamino, (1R)-3-(morpholin-4-ylamino)-1-((phenylsulfanyl)methyl)propylamino, (1R)-3-((2-(morpholin-4-yl)ethyl)amino)-1-((phenylsulfanyl)methyl)propylamino, (1R)-3-(morpholin-4-yl)-3-oxo-1-((2-thienylsulfanyl)methyl)-propylamino, (1R)-3-(morpholin-4-yl)-1-((1,3-thiazol-2-ylsulfanyl)methyl)propylamino, (1R)-3-(morpholin-4-yl)-1-((thien-2-ylsulfanyl)methyl)propylamino, (1R)-3-(morpholin-4-yl)-1-((phenylsulfanyl)methyl)propylamino, (1R)-3-(morpholin-4-yl)-1-(((4-(methoxy)phenyl) sulfanyl)methyl)propylamino, (1R)-3-(morpholin-4-yl)-1-(((4-((methyl)phenyl)sulfanyl)methyl)propylamino, (1R)-3-(morpholin-4-yl)-1-((phenylsulfonyl)methyl)propylamino, (1R)-3-(morpholin-4-yl)-1-(((4-(trifluoromethoxy)phenyl)sulfanyl)methyl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(1,3-benzodioxol-5-yl)phenylmethyl)piperazin-4-yl, 1-(2-(benzofuran-2-ylphenyl)phenylmethyl)piperazin-4-yl, 1-(2-bromocyclohex-1-en-1-ylmethyl)piperazin-4-yl, 1-(2-bromocyclopent-1-en-1-ylmethyl)piperazin-4-yl, 1-(4-bromophenyl)phenylmethyl)piperazin-4-yl, 1-(2-(4-chlorophenyl)cyclohept-1-en-1-ylmethyl)piperazin-4-yl, 1-(2-(4-chlorophenyl)cyclohex-1-en-1-ylmethyl)-1,2,3,6-tetrahydropyridin-4-yl, 1-(2-(4-chlorophenyl)cyclohex-1-en-1-ylmethyl)piperazin-4-yl, 1-(2-(4-chlorophenyl)cyclopent-1-en-1-ylmethyl)piperazin-4-yl, 1-(2-(4-chlorophenyl)cyclooct-1-en-1-ylmethyl)piperazin-4-yl, 1-((4-(4-chlorophenyl)-5,6-dihydro-2H-pyran-3-yl)methyl)piperazin-4-yl, 1-((4-(4-chlorophenyl)-5,6-dihydro-2H-pyran-3-yl)methyl)piperazin-4-yl, 1-((2-(4-chlorophenyl)-5,5-dimethyl-1-cyclohex-1-en-1-yl)methyl)-1,2,3,6-tetrahydropyridin-4-yl, 1-(2-(4-chlorophenyl)naphth-3-ylmethyl)piperazin-4-yl, 1-(2-(4-chlorophenyl)pyridin-3-ylmethyl)piperazin-4-yl, 1-(3-(4-chlorophenyl)pyridin-4-ylmethyl)piperazin-4-yl, 1-((4-(4-chlorophenyl)pyridin-5-yl)methyl)piperazin-4-yl, 1-(2-(4-chlorophenyl)phenylcarbonyl)piperazin-4-yl, 1-(2-(4-chlorophenyl)phenylcycloprop-1-yl)piperazin-4-yl, or 4-(2-(4-chlorophenyl)phenylmethyl)cyclohex-1-en-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-(4-(hydroxyaminocarbonyl)piperidin-1-yl)-1-((phenylsulfanyl)methyl)propylamino, (1R)-3-(2-hydroxy-2-methylpropyl)amino)-1-((phenylsulfanyl)methyl)propylamino, (1R)-3-(4-hydroxypiperidin-1-yl)-1-((phenylsulfanyl)methyl)propylamino, (3R)-3-(3-hydroxypyrrolidin-1-yl)-1-((phenylsulfanyl)methyl)propylamino, isopropylamino, (1R)-3-(isopropylamino)-1-((phenylsulfanyl)methyl)propylamino, (1R)-3-(1H-imidazol-1-yl)-1-((phenylsulfanyl)methyl)propylamino, (1R)-3-(isopropyl(methyl)amino)-1-((phenylsulfanyl)methyl)propylamino, (4-methoxycyclohex-1-yl)methyl)amino, (1R)-3-(4-(methoxyimino)piperidin-1-yl)-1-((phenylsulfanyl)methyl)propylamino, (1R)-3-(N-methyl-N-carboxymethyl)amino)-1-((phenylsulfanyl)methyl)propylamino, (methyl) (cyclohexyl)amino, (methyl) (cyclohexylmethyl)amino, (1R)-3-(2-methyl-4,5-dihydro-1H-imidazol-1-yl)-1-((phenylsulfanyl)methyl)propylamino, (3R)-3-(N-methyl-N-(dimethylcarbonylmethyl))-1-((phenylsulfanyl)methyl)propylamino, (1R)-3-(N-methyl-N-(1,1-dimethylethyl)amino)-1-((phenylsulfanyl)methyl)propylamino, (N-methyl-N-(1,2-diphenyl)amino)carbonyl)-N-methylamino, (N-methyl-N-((diphenylmethyl)amino)carbonyl)-N-methylamino, (2-methylfuran-3-yl)sulfanyl)-(1,1-spirobutyl)ethylamino, (1R)-3-(N-methyl-N-(2-hydroxyethyl))amino)-1-((phenylsulfanyl)methyl)propylamino, (1R)-3-(N-methyl-N-isopropylamino)-1-((phenylsulfanyl)methyl)propylamino, (N-methyl-N-(4-methoxyphenyl)amino)carbonyl)-N-methylamino, 1-methyl-4-(phenylsulfanyl)pyrrolidin-3-ylamino, (N-methyl-N-(4-methylphenyl)amino)carbonyl)-N-methylamino, (N-methyl-N-(2-methylphenyl)amino)carbonyl)-N-methylamino, (1R)-3-(4-methylpiperazin-1-yl)-1-((phenylsulfanyl)methyl)propylamino, 1-methylpiperidin-4-yloxy, (N-methyl-N—((S)-1-phenylethyl)amino)carbonyl)-N-methylamino, (N-methyl-N-(1-phenyl-2-(4-methylpiperazin-4-yl))amino)carbonyl)-N-methylamino, (N-methyl-N-(1-phenyl-2-(morpholin-1-yl))amino)carbonyl)-N-methylamino, (N-methyl-N-(1-phenyl-2-(N,N-dimethylamino))amino)carbonyl)-N-methylamino, (1R)-1-methyl-2-((phenylsulfanyl)methyl)ethylamino, (1S)-1-methyl-2-((phenylsulfanyl)methyl)ethylamino, (1R)-4-(4-methylpiperazin-1-yl)-1-((phenylsulfanyl)methyl)butylamino, (1R)-3-(methyl(pyridin-4-yl)amino)-1-((phenylsulfanyl)methyl)propylamino, (1R)-5-((methylsulfonylamino)-1-((phenylsulfanyl)methyl)pentylamino, (1R)-3-(4-(methylsulfonylaminocarbonyl)piperidin-1-yl)-1-((phenylsulfanyl)methyl)-propylamino, 2-((4-methyl-1,3-thiazol-2-yl)sulfanyl)ethylamino, (N-methyl-N-(4-trifluoromethoxyphenyl)amino)carbonyl)-N-methylamino, (1R)-3-(morpholin-4-ylamino)-1-((phenylsulfanyl)methyl)propylamino, (1R)-3-((2-(morpholin-4-yl)ethyl)amino)-1-((phenylsulfanyl)methyl)propylamino, (1R)-3-(morpholin-4-yl)-3-oxo-1-((2-thienylsulfanyl)methyl)-propylamino, (1R)-3-(morpholin-4-yl)-1-((1,3-thiazol-2-ylsulfanyl)methyl)propylamino, (1R)-3-(morpholin-4-yl)-1-((thien-2-ylsulfanyl)methyl)propylamino, (1R)-3-(morpholin-4-yl)-1-((phenylsulfanyl)methyl)propylamino, (1R)-3-(morpholin-4-yl)-1-(((4-(methoxy)phenyl) sulfanyl)methyl)propylamino, (1R)-3-(morpholin-4-yl)-1-(((4-(methyl)phenyl)sulfanyl)methyl)propylamino, (1R)-3-(morpholin-4-yl)-1-((phenylsulfonyl)-methyl)propylamino, (1R)-3-(morpholin-4-yl)-1-(((4-(trifluoromethoxy)phenyl)sulfanyl)methyl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 5-(2-(4-chlorophenyl)phenylmethyl)hexahydropyrrolo[3,4-c]pyrrol-2(1H)-yl, 1-(2-(4-chlorophenyl)phenylmethyl)-piperazin-4-yl, 1-(2-(4-chlorophenyl)phenylmethyl)-piperazin-4-yl, 1-(2-(4-chlorophenyl)phenylmethyl)piperazin-4-yl, 1-(2-(4-chlorophenyl)phenylmethyl)piperazin-4-yl, 1-(2-(4-chlorophenyl)phenylmethyl)piperazin-4-yl, 1-(2-(4-chlorophenyl)phenylmethyl)piperazin-4-yl, 1-(2-(4-chlorophenyl)phenylmethyl)piperidin-4-yl, or 1-(2-(4-chlorophenyl)phenylmethyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-(4-(hydroxyaminocarbonyl)piperidin-1-yl)-1-((phenylsulfanyl)methyl)propylamino, (1R)-3-(2-hydroxy-2-methylpropyl)amino)-1-((phenylsulfanyl)methyl)propylamino, (1R)-3-(4-hydroxypiperidin-1-yl)-1-((phenylsulfanyl)methyl)propylamino, (3R)-3-(3-hydroxypyrrolidin-1-yl)-1-((phenylsulfanyl)methyl)propylamino, isopropylamino, (1R)-3-(isopropylamino)-1-((phenylsulfanyl)methyl)propylamino, (1R)-3-(1H-imidazol-1-yl)-1-((phenylsulfanyl)methyl)propylamino, (1R)-3-(isopropyl (methyl)amino)-1-((phenylsulfanyl)methyl)propylamino, (4-methoxycyclohex-1-yl)methyl)amino, (1R)-3-(4-(methoxyimino)piperidin-1-yl)-1-((phenylsulfanyl)methyl)propylamino, (1R)-3-(N-methyl-N-carboxymethyl)amino)-1-((phenylsulfanyl)methyl)propylamino, (methyl) (cyclohexyl)amino, (methyl) (cyclohexylmethyl)amino, (1R)-3-(2-methyl-4,5-dihydro-1H-imidazol-1-yl)-1-((phenylsulfanyl)methyl)propylamino, (3R)-3-(N-methyl-N-(dimethylcarbonylmethyl))-1-((phenylsulfanyl)methyl)propylamino, (1R)-3-(N-methyl-N-(1,1-dimethylethyl)amino)-1-((phenylsulfanyl)methyl)propylamino, (N-methyl-N-(1,2-diphenyl)amino)carbonyl)-N-methylamino, (N-methyl-N-((diphenylmethyl)amino)carbonyl)-N-methylamino, (2-methylfuran-3-yl)sulfanyl)-(1,1-spirobutyl)ethylamino, (1R)-3-(N-methyl-N-(2-hydroxyethyl))amino)-1-((phenylsulfanyl)methyl)propylamino, (1R)-3-(N-methyl-N-isopropylamino)-1-((phenylsulfanyl)methyl)propylamino, (N-methyl-N-(4-methoxyphenyl)amino)carbonyl)-N-methylamino, 1-methyl-4-(phenylsulfanyl)pyrrolidin-3-ylamino, (N-methyl-N-(4-methylphenyl)amino)carbonyl)-N-methylamino, (N-methyl-N-(2-methylphenyl)amino)carbonyl)-N-methylamino, (1R)-3-(4-methylpiperazin-1-yl)-1-((phenylsulfanyl)methyl)propylamino, 1-methylpiperidin-4-yloxy, (N-methyl-N—((S)-1-phenylethyl)amino)carbonyl)-N-methylamino, (N-methyl-N-(1-phenyl-2-(4-methylpiperazin-4-yl))amino)carbonyl)-N-methylamino, (N-methyl-N-(1-phenyl-2-(morpholin-1-yl))amino)carbonyl)-N-methylamino, (N-methyl-N-(1-phenyl-2-(N,N-dimethylamino))amino)carbonyl)-N-methylamino, (1R)-1-methyl-2-((phenylsulfanyl)methyl)ethylamino, (1S)-1-methyl-2-((phenylsulfanyl)methyl)ethylamino, (1R)-4-(4-methylpiperazin-1-yl)-1-((phenylsulfanyl)methyl)butylamino, (1R)-3-(methyl(pyridin-4-yl)amino)-1-((phenylsulfanyl)methyl)propylamino, (1R)-5-((methylsulfonylamino)-1-((phenylsulfanyl)methyl)pentylamino, (1R)-3-(4-(methylsulfonylaminocarbonyl)piperidin-1-yl)-1-((phenylsulfanyl)methyl)propylamino, 2-((4-methyl-1,3-thiazol-2-yl)sulfanyl)ethylamino, (N-methyl-N-(4-trifluoromethoxyphenyl)amino)carbonyl)-N-methylamino, (1R)-3-(morpholin-4-ylamino)-1-((phenylsulfanyl)methyl)propylamino, (1R)-3-((2-(morpholin-4-yl)ethyl)amino)-1-((phenylsulfanyl)methyl)propylamino, (1R)-3-(morpholin-4-yl)-3-oxo-1-((2-thienylsulfanyl)methyl)propylamino, (1R)-3-(morpholin-4-yl)-1-((1,3-thiazol-2-ylsulfanyl)methyl)propylamino, (1R)-3-(morpholin-4-yl)-1-((thien-2-ylsulfanyl)methyl)propylamino, (1R)-3-(morpholin-4-yl)-1-((phenylsulfanyl)methyl)propylamino, (1R)-3-(morpholin-4-yl)-1-(((4-(methoxy)phenyl) sulfanyl)methyl)propylamino, (1R)-3-(morpholin-4-yl)-1-(((4-(methyl)phenyl)sulfanyl)methyl)propylamino, (1R)-3-(morpholin-4-yl)-1-((phenylsulfonyl)methyl)propylamino, (1R)-3-(morpholin-4-yl)-1-(((4-(trifluoromethoxy)phenyl)sulfanyl)methyl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(4-(dimethylamino)phenyl)phenylcarbonyl)piperazin-4-yl, 1-(2-(4-(dimethylamino)phenyl)phenylmethyl)piperazin-4-yl, 1-((2-(4-(dimethylamino)phenyl)pyridin-3-yl)methyl)piperazin-4-yl, 1-(2-(5,5-dimethyl-2-oxo-1,3-oxazolidin-3-yl)phenylmethyl)piperazin-4-yl, 1-(2-(4-fluorophenyl)cyclopent-1-en-1-ylmethyl)piperazin-4-yl, 1-(2-(4-fluorophenyl)-3-fluorophenylmethyl)piperazin-4-yl, 1-(2-(4-fluorophenyl)phenylmethyl)piperazin-4-yl, 1-((2-(4-fluorophenyl)pyridin-3-yl)methyl)piperazin-4-yl, 4-(2-(isopropylamino)phenylmethyl)-piperazin-1-yl, 1-(2-(4-(isopropylsulfanyl)phenyl)phenylmethyl)piperazin-4-yl, 1-(2-(4-methoxyphenyl)cyclopent-1-en-1-ylmethyl)piperazin-4-yl, 1-(2-(3-methoxyphenyl)phenylmethyl)piperazin-4-yl, 1-(2-(4-methoxyphenyl)phenylmethyl)piperazin-4-yl, 1-((2-(4-methoxyphenyl)pyridin-3-yl)methyl)piperazin-4-yl, (4-methoxy)-4-(2-(pyridin-3-yl)phenylmethyl)piperidin-1-yl, 1-(2-(2-methyl-4-dichlorophenyl)phenylmethyl)piperazin-4-yl, 1-(2-(2-methylphenyl)phenylmethyl)-piperazin-4-yl, 1-(2-(4-(methylsulfonyl)-phenyl)methyl)piperazin-4-yl, 1-(2-(4-methylsulfonylphenyl)phenylmethyl)piperazin-4-yl, 1-((2-(4-methylsulfonylphenyl)pyridin-3-yl)methyl)piperazin-4-yl, 1-(2-(4-chlorophenyl)phenylmethyl)-1,2,3,6-tetrahydropyridin-4-yl, 1-(2-(cyclohex-1-ylamino)phenylmethyl)piperazin-4-yl, 1-(2-(cyclohex-1-ylamino)phenylmethyl)piperazin-4-yl, 1-(2-(3-cyanophenyl)phenylmethyl)piperazin-4-yl, 1-(2-(2,4-dichlorophenyl)phenylmethyl)piperazin-4-yl, 1-(2-(3,4-dichlorophenyl)phenylmethyl)piperazin-4-yl, 1-(2-(2,4-difluorophenyl)phenylmethyl)piperazin-4-yl, 1-(2-(1,3-dihydro-2H-isoindol-2-yl)phenylmethyl)piperazin-4-yl, 1-(3-(1,1-dimethylethoxycarbonylamino)phenyl)piperazin-4-yl, 1-(4-(2-(dimethylamino)ethoxy)phenyl)phenylmethyl)piperazin-4-yl, or 1-(3-(dimethylamino)phenyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-(4-(hydroxyaminocarbonyl)piperidin-1-yl)-1-((phenylsulfanyl)methyl)propylamino, (1R)-3-(2-hydroxy-2-methylpropyl)amino)-1-((phenylsulfanyl)methyl)propylamino, (1R)-3-(4-hydroxypiperidin-1-yl)-1-((phenylsulfanyl)methyl)propylamino, (3R)-3-(3-hydroxypyrrolidin-1-yl)-1-((phenylsulfanyl)methyl)propylamino, isopropylamino, (1R)-3-(isopropylamino)-1-((phenylsulfanyl)methyl)propylamino, (1R)-3-(1H-imidazol-1-yl)-1-((phenylsulfanyl)methyl)propylamino, (1R)-3-(isopropyl (methyl)amino)-1-((phenylsulfanyl)methyl)propylamino, (4-methoxycyclohex-1-yl)methyl)amino, (1R)-3-(4-(methoxyimino)piperidin-1-yl)-1-((phenylsulfanyl)methyl)propylamino, (1R)-3-(N-methyl-N-carboxymethyl)amino)-1-((phenylsulfanyl)methyl)propylamino, (methyl) (cyclohexyl)amino, (methyl) (cyclohexylmethyl)amino, (1R)-3-(2-methyl-4,5-dihydro-1H-imidazol-1-yl)-1-((phenylsulfanyl)methyl)propylamino, (3R)-3-(N-methyl-N-(dimethylcarbonylmethyl))-1-((phenylsulfanyl)methyl)propylamino, (1R)-3-(N-methyl-N-(1,1-dimethylethyl)amino)-1-((phenylsulfanyl)methyl)propylamino, (N-methyl-N-(1,2-diphenyl)amino)carbonyl)-N-methylamino, (N-methyl-N-((diphenylmethyl)amino)carbonyl)-N-methylamino, (2-methylfuran-3-yl)sulfanyl)-(1,1-spirobutyl)ethylamino, (1R)-3-(N-methyl-N-(2-hydroxyethyl))amino)-1-((phenylsulfanyl)methyl)propylamino, (1R)-3-(N-methyl-N-isopropylamino)-1-((phenylsulfanyl)methyl)propylamino, (N-methyl-N-(4-methoxyphenyl)amino)carbonyl)-N-methylamino, 1-methyl-4-(phenylsulfanyl)pyrrolidin-3-ylamino, (N-methyl-N-(4-methylphenyl)amino)carbonyl)-N-methylamino, (N-methyl-N-(2-methylphenyl)amino)carbonyl)-N-methylamino, (1R)-3-(4-methylpiperazin-1-yl)-1-((phenylsulfanyl)methyl)propylamino, 1-methylpiperidin-4-yloxy, (N-methyl-N—((S)-1-phenylethyl)amino)carbonyl)-N-methylamino, (N-methyl-N-(1-phenyl-2-(4-methylpiperazin-4-yl))amino)carbonyl)-N-methylamino, (N-methyl-N-(1-phenyl-2-(morpholin-1-yl))amino)carbonyl)-N-methylamino, (N-methyl-N-(1-phenyl-2-(N,N-dimethylamino))amino)carbonyl)-N-methylamino, (1R)-1-methyl-2-((phenylsulfanyl)methyl)ethylamino, (1S)-1-methyl-2-((phenylsulfanyl)methyl)ethylamino, (1R)-4-(4-methylpiperazin-1-yl)-1-((phenylsulfanyl)methyl)butylamino, (1R)-3-(methyl(pyridin-4-yl)amino)-1-((phenylsulfanyl)methyl)propylamino, (1R)-5-((methylsulfonylamino)-1-((phenylsulfanyl)methyl)pentylamino, (1R)-3-(4-(methylsulfonylaminocarbonyl)piperidin-1-yl)-1-((phenylsulfanyl)methyl)propylamino, 2-((4-methyl-1,3-thiazol-2-yl)sulfanyl)ethylamino, (N-methyl-N-(4-trifluoromethoxyphenyl)amino)carbonyl)-N-methylamino, (1R)-3-(morpholin-4-ylamino)-1-((phenylsulfanyl)methyl)propylamino, (1R)-3-((2-(morpholin-4-yl)ethyl)amino)-1-((phenylsulfanyl)methyl)propylamino, (1R)-3-(morpholin-4-yl)-3-oxo-1-((2-thienylsulfanyl)methyl)propylamino, (1R)-3-(morpholin-4-yl)-1-((1,3-thiazol-2-ylsulfanyl)methyl)propylamino, (1R)-3-(morpholin-4-yl)-1-((thien-2-ylsulfanyl)methyl)propylamino, (1R)-3-(morpholin-4-yl)-1-((phenylsulfanyl)methyl)propylamino, (1R)-3-(morpholin-4-yl)-1-(((4-(methoxy)phenyl)sulfanyl)methyl)propylamino, (1R)-3-(morpholin-4-yl)-1-(((4-(methyl)phenyl)sulfanyl)methylpropylamino, (1R)-3-(morpholin-4-yl)-1-((phenylsulfonyl)methyl)propylamino, (1R)-3-(morpholin-4-yl)-1-(((4-(trifluoromethoxy)phenyl)sulfanyl)methyl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(5-methylthien-2-yl)phenylmethyl)piperazin-4-yl, 1-(2-(4-methylsulfanylphenyl)cyclohex-1-en-1-ylmethyl)piperazin-4-yl, 1-(2-(4-methylsulfanylphenyl)phenylcarbonyl)piperazin-4-yl, 1-(2-(4-methylsulfanylphenyl)phenylcarbonyl)piperazin-4-yl, 1-((2-(4-methylsulfanylphenyl)pyridin-3-yl)methyl)piperazin-4-yl. 1-(2-(4-methylsulfanylphenyl)phenylmethyl)piperazin-1-yl, 1-(2-(4-(2-(morpholin-1-yl)ethoxy)phenyl)phenylmethyl)piperazin-1-yl, 1-(2-(morpholin-1-yl)phenylmethyl)piperazin-4-yl, 1-(2-(naphth-1-yl)phenylmethyl)piperazin-4-yl, 4-(2-(naphth-2-yl)phenylmethyl)piperazin-1-yl, 1-(2-(4-phenoxyphenyl)phenylmethyl)piperazin-4-yl, 1-((1-phenyl-1H-imidazol-2-yl)methyl)piperazin-4-yl, 1-((1-phenyl-1H-imidazol-5-yl)methyl)piperazin-4-yl, 1-(2-((phenylmethyl)amino)phenylmethyl)piperazin-4-yl, 1-(2-(phenyl)phenylmethyl)-4-(2-(dimethylamino)ethoxy))piperidin-4-y-1,2-(phenyl)phenylmethyl-4-methoxypiperidin-1-yl, 4-((2-(phenyl)phenylmethyl)-4-(2-(morpholin-4-yl)ethoxy))piperidin-1-yl, 1-(2-(phenyl)phenylmethyl)piperazin-4-yl, 1-(3-(phenyl)phenylmethyl)piperazin-4-yl, 1-(2-(4-(phenyl)phenyl)phenylmethyl)piperazin-4-yl, or 4-(2-(phenyl)phenylmethyl)-4-(2-(piperidin-1-yl)ethoxy)piperidin-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-(4-(hydroxyaminocarbonyl)piperidin-1-yl)-1-((phenylsulfanyl)methyl)propylamino, (1R)-3-(2-hydroxy-2-methylpropyl)amino)-1-((phenylsulfanyl)methyl)propylamino, (1R)-3-(4-hydroxypiperidin-1-yl)-1-((phenylsulfanyl)methyl)propylamino, (3R)-3-(3-hydroxypyrrolidin-1-yl)-1-((phenylsulfanyl)methyl)propylamino, isopropylamino, (1R)-3-(isopropylamino)-1-((phenylsulfanyl)methyl)propylamino, (1R)-3-(1H-imidazol-1-yl)-1-((phenylsulfanyl)methyl)propylamino, (1R)-3-(isopropyl(methyl)amino)-1-((phenylsulfanyl)methyl)propylamino, (4-methoxycyclohex-1-yl)methyl)amino, (1R)-3-(4-(methoxyimino)piperidin-1-yl)-1-((phenylsulfanyl)methyl)propylamino, (1R)-3-(N-methyl-N-carboxymethyl)amino)-1-((phenylsulfanyl)methyl)propylamino, (methyl) (cyclohexyl)amino, (methyl) (cyclohexylmethyl)amino, (1R)-3-(2-methyl-4,5-dihydro-1H-imidazol-1-yl)-1-((phenylsulfanyl)methyl)propylamino, (3R)-3-(N-methyl-N-(dimethylcarbonylmethyl))-1-((phenylsulfanyl)methyl)propylamino, (1R)-3-(N-methyl-N-(1,1-dimethylethyl)amino)-1-((phenylsulfanyl)methyl)propylamino, (N-methyl-N-(1,2-diphenyl)amino)carbonyl)-N-methylamino, (N-methyl-N-((diphenylmethyl)amino)carbonyl)-N-methylamino, (2-methylfuran-3-yl)sulfanyl)-(1,1-spirobutyl)ethylamino, (1R)-3-(N-methyl-N-(2-hydroxyethyl))amino)-1-((phenylsulfanyl)methyl)propylamino, (1R)-3-(N-methyl-N-isopropylamino)-1-((phenylsulfanyl)methyl)propylamino, (N-methyl-N-(4-methoxyphenyl)amino)carbonyl)-N-methylamino, 1-methyl-4-(phenylsulfanyl)pyrrolidin-3-ylamino, (N-methyl-N-(4-methylphenyl)amino)carbonyl)-N-methylamino, (N-methyl-N-(2-methylphenyl)amino)carbonyl)-N-methylamino, (1R)-3-(4-methylpiperazin-1-yl)-1-((phenylsulfanyl)methyl)propylamino, 1-methylpiperidin-4-yloxy, (N-methyl-N—((S)-1-phenylethyl)amino)carbonyl)-N-methylamino, (N-methyl-N-(1-phenyl-2-(4-methylpiperazin-4-yl))amino)carbonyl)-N-methylamino, (N-methyl-N-(1-phenyl-2-(morpholin-1-yl))amino)carbonyl)-N-methylamino, (N-methyl-N-(1-phenyl-2-(N,N-dimethylamino))amino)carbonyl)-N-methylamino, (1R)-1-methyl-2-((phenylsulfanyl)methyl)ethylamino, (1S)-1-methyl-2-((phenylsulfanyl)methyl)ethylamino, (1R)-4-(4-methylpiperazin-1-yl)-1-((phenylsulfanyl)methyl)butylamino, (1R)-3-(methyl(pyridin-4-yl)amino)-1-((phenylsulfanyl)methyl)propylamino, (1R)-5-((methylsulfonylamino)-1-((phenylsulfanyl)methyl)pentylamino, (1R)-3-(4-(methylsulfonylaminocarbonyl)piperidin-1-yl)-1-((phenylsulfanyl)methyl)propylamino, 2-((4-methyl-1,3-thiazol-2-yl)sulfanyl)ethylamino, (N-methyl-N-(4-trifluoromethoxyphenyl)amino)carbonyl)-N-methylamino, (1R)-3-(morpholin-4-ylamino)-1-((phenylsulfanyl)methyl)propylamino, (1R)-3-((2-(morpholin-4-yl)ethyl)amino)-1-((phenylsulfanyl)methyl)propylamino, (1R)-3-(morpholin-4-yl)-3-oxo-1-((2-thienylsulfanyl)methyl)propylamino, (1R)-3-(morpholin-4-yl)-1-((1,3-thiazol-2-ylsulfanyl)methyl)propylamino, (1R)-3-(morpholin-4-yl)-1-((thien-2-ylsulfanyl)methyl)propylamino, (1R)-3-(morpholin-4-yl)-1-((phenylsulfanyl)methyl)propylamino, (1R)-3-(morpholin-4-yl)-1-(((4-(methoxy)phenyl) sulfanyl)methyl)propylamino, (1R)-3-(morpholin-4-yl)-1-(((4-(methyl)phenyl)sulfanyl)methyl)propylamino, (1R)-3-(morpholin-4-yl)-1-((phenylsulfonyl)methyl)propylamino, (1R)-3-(morpholin-4-yl)-1-(((4-(trifluoromethoxy)phenyl)sulfanyl)methyl)propylamino; D¹ is H, F, C₁ or CF₃; E¹ is H, F or Cl; Y¹ is H, CN, NO₂, F, Cl, CF₃, OCF₃, NH₂ or C(O)NH₂; and Z is 4-(2-(phenyl)phenylmethyl)-4-(2-(pyrrolidin-1-yl)ethoxy)piperidin-1-yl, 1-((2-(phenyl)pyridin-3-yl)methyl)piperazin-4-yl, 1-((1-phenyl-1H-pyrazol-5-yl)methyl)piperazin-4-yl, 1-(2-(piperidin-1-yl)phenylmethyl)piperazin-4-yl, 1-(2-(pyrid-3-yl)phenyl)phenylmethyl)piperazin-4-yl, 1-(2-(quinolin-3-ylphenyl)phenylmethyl)piperazin-4-yl, 1-(2-(quinolin-8-ylphenyl)phenylmethyl)piperazin-4-yl, 4-(2-(thien-2-yl)phenylmethyl)-4-methoxypiperazin-1-yl, 1-(2-(thien-2-yl)phenylmethyl)piperazin-4-yl, 1-(2-(4-trifluoromethoxyphenyl)phenylmethyl)piperazin-4-yl, or 1-(2-(4-trifluoromethyl-phenyl)phenylmethyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-((1S,4S)-2-oxa-5-azabicyclo[2.2.1]hept-5-yl)-1-((phenylsulfanyl)methyl)propylamino, (1R)-3-((1R,4R)-2-oxa-5-azabicyclo[2.2.1]hept-5-yl)-1-((phenylsulfanyl)methyl)propylamino, (1R)-3-oxo-3-(azetidin-1-yl)-1-((phenylsulfanyl)methyl)propylamino, (1R)-3-oxo-3-(cyclobutylamino)-1-((phenylsulfanyl)methyl)propylamino, (1R)-3-oxo-3-(cyclopropylamino)-1-((phenylsulfanyl)methyl)propylamino, (1R)-3-oxo-3-(dimethylamino)-1-((phenylsulfanyl)methyl)propylamino, (1R)-3-oxo-3-(dimethylamino)-1-((phenylsulfonylmethyl)methyl)propylamino, (1R)-3-oxo-3-(dimethylamino)-1-((pyrimidin-1-ylsulfanyl)methyl)propylamino, (1R)-3-oxo-3-((1,1-dimethylethyl)amino)-1-((phenylsulfanyl)methyl)propylamino, (1R)-3-oxo-3-(diisopropylamino)-1-((phenylsulfanyl)methyl)-propylamino, (1R)-3-oxo-3-(1,1-dioxothiomorpholin-4-yl)-1-((phenylsulfanyl)methyl)propylamino, (1R)-3-oxo-3-(N-methyl-N-(1,1-dimethylethyl)amino)-1-((phenylsulfanyl)methyl)propylamino, (1R)-3-oxo-3-(piperidin-1-yl)-1-((phenylsulfanyl)methyl)propylamino, (1R)-3-oxo-3-amino-1-((phenylsulfanyl)methyl)propylamino, (1R)-3-oxo-3-(methylamino)-1-((phenylsulfanyl)methyl)propylamino, (1R)-3-oxo-3-(4-methylpiperazin-1-yl)-1-((phenylsulfanyl)methyl)propylamino, (1R)-3-oxo-3-(morpholin-1-yl)-1-((phenylsulfanyl)methyl)propylamino, (1R)-3-oxo-3-(2-(morpholin-1-yl)ethyl)-1-((phenylsulfanyl)methyl)propylamino, (2-phenoxyethyl)amino, 4-(1-(phenylmethyl)piperidin-4-yl)amino, 4-(1-(phenylmethyl)piperidin-4-yl)methylamino, (4-phenyl-1,3-thiazol-2-ylsulfanyl)ethylamino, (1R,2S)-2-(phenylsulfanyl)cyclohex-1-ylamino, (1S,2R)-2-(phenylsulfanyl)cyclohex-1-ylamino, 2-(phenylsulfanyl)cyclopentylamino, 2-(phenylsulfanyl)ethoxy, 2-(phenylsulfanyl)ethylamino, 2-(phenylsulfonyl)ethylamino, (1R)-1-((phenylsulfanyl)methyl)-3-(morpholin-4-yl)propylamino, (1R)-1-((phenylsulfanyl)methyl)-3-((2,2,2-trifluoroethyl)amino)propylamino, 4-(phenylsulfonyl)tetrahydrofuran-3-ylamino, 4-(phenylsulfanyl)tetrahydrofuran-3-ylamino, (1R)-1-((phenylsulfanyl)methyl)-3-((2,2,2-trifluoroethyl)amino)propylamino, (1S)-1-((phenylsulfanyl)methyl)propylamino, (1R)-1-((phenylsulfanyl)methyl)-3-(pyridin-4-ylsulfanyl)propylamino, (1R)-1-((phenylsulfanyl)methyl)-3-(thiomorpholin-4-yl)propylamino, (1R)-1-((phenylsulfanyl)methyl)-3-(piperazin-1-yl)propylamino, (1R)-1-((phenylsulfanyl)methyl)-3-((2-(pyridin-2-yl)ethyl)amino)propylamino, (1R)-1-((phenylsulfanyl)methyl)-3-((pyridin-4-ylmethyl)amino)propylamino, (1R)-1-((phenylsulfanyl)methyl)-3-(pyridin-3-ylamino)propylamino, (1R)-1-((phenylsulfanyl)methyl)-3-(pyrrolidin-1-ylamino)propylamino), (1R)-1-((phenylsulfanyl)methyl)-3-(2H-tetrazol-5-yl)propylamino, (1R)-1-((phenylsulfanyl)methyl)-3-(pyrrolidin-1-yl)propylamino, (1R)-1-((phenylsulfanyl)methyl)-3-((pyridin-2-ylmethyl)amino)propylamino, (1S)-2-(phenylsulfanyl)-1-(pyridin-3-ylmethyl)ethylamino, (3S,4R)(phenylsulfanyl)pyrrolidin-4-ylamino, 2-(phenylsulfanyl)-1,1-spirobutylethylamino, 2-(phenylsulfanyl)-1,1-spiroethylethylamino, 2-(phenylsulfanyl)-1,1-spiropentylethylamino, piperidin-4-yloxy, (1-propylpiperidin-4-yl)methylamino, pyran-4-ylamino, 2-(pyridin-4-ylsulfanyl)ethylamino, 2-(pyrimidin-2-ylsulfanyl)ethylamino, 1,1-spirobutyl-2-(phenylsulfanyl)ethyl, 2-(thien-2-ylsulfanyl)ethylamino, sulfanylpyran-4-ylamino, (1R)-3-(2-(2H-tetrazol-3-yl)pyrrolidin-1-yl)-1-((phenylsulfanyl)methyl)propylamino, (1R)-3-(3-(2H-tetrazol-3-yl)azetidin-1-yl)-1-((phenylsulfanyl)methyl)propylamino or 2-(1,3-thiazol-2-ylsulfanyl)ethylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(1,3-benzodioxol-5-yl)phenylmethyl)piperazin-4-yl, 1-(2-(benzofuran-2-ylphenyl)phenylmethyl)piperazin-4-yl, 1-(2-bromocyclohex-1-en-1-ylmethyl)piperazin-4-yl, 1-(2-bromocyclopent-1-en-1-ylmethyl)piperazin-4-yl, 1-(4-bromophenyl)phenylmethyl)piperazin-4-yl, 1-(2-(4-chlorophenyl)cyclohept-1-en-1-ylmethyl)piperazin-4-yl, 1-(2-(4-chlorophenyl)cyclohex-1-en-1-ylmethyl)-1,2,3,6-tetrahydropyridin-4-yl, 1-(2-(4-chlorophenyl)cyclohex-1-en-1-ylmethyl)piperazin-4-yl, 1-(2-(4-chlorophenyl)cyclopent-1-en-1-ylmethyl)piperazin-4-yl, 1-(2-(4-chlorophenyl)cyclooct-1-en-1-ylmethyl)piperazin-4-yl, 1-((4-(4-chlorophenyl)-5,6-dihydro-2H-pyran-3-yl)methyl)piperazin-4-yl, 1-((4-(4-chlorophenyl)-5,6-dihydro-2H-pyran-3-yl)methyl)piperazin-4-yl, 1-((2-(4-chlorophenyl)-5,5-dimethyl-1-cyclohex-1-en-1-yl)methyl)-1,2,3,6-tetrahydropyridin-4-yl, 1-(2-(4-chlorophenyl)naphth-3-ylmethyl)piperazin-4-yl, 1-(2-(4-chlorophenyl)pyridin-3-ylmethyl)piperazin-4-yl, 1-(3-(4-chlorophenyl)pyridin-4-ylmethyl)piperazin-4-yl, 1-((4-(4-chlorophenyl)pyridin-5-yl)methyl)piperazin-4-yl, 1-(2-(4-chlorophenyl)phenylcarbonyl)piperazin-4-yl, 1-(2-(4-chlorophenyl)phenylcycloprop-1-yl)piperazin-4-yl, or 4-(2-(4-chlorophenyl)phenylmethyl)cyclohex-1-en-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-((1S,4S)-2-oxa-5-azabicyclo[2.2.1]hept-5-yl)-1-((phenylsulfanyl)methyl)propylamino, (1R)-3-((1R,4R)-2-oxa-5-azabicyclo[2.2.1]hept-5-yl)-1-((phenylsulfanyl)methyl)propylamino, (1R)-3-oxo-3-(azetidin-1-yl)-1-((phenylsulfanyl)methyl)propylamino, (1R)-3-oxo-3-(cyclobutylamino)-1-((phenylsulfanyl)methyl)propylamino, (1R)-3-oxo-3-(cyclopropylamino)-1-((phenylsulfanyl)methyl)propylamino, (1R)-3-oxo-3-(dimethylamino)-1-((phenylsulfanyl)methyl)propylamino, (1R)-3-oxo-3-(dimethylamino)-1-((phenylsulfonylmethyl)methyl)propylamino, (1R)-3-oxo-3-(dimethylamino)-1-((pyrimidin-1-ylsulfanyl)methyl)propylamino, (1R)-3-oxo-3-((1,1-dimethylethyl)amino)-1-((phenylsulfanyl)methyl)propylamino, (1R)-3-oxo-3-(diisopropylamino)-1-((phenylsulfanyl)methyl)-propylamino, (1R)-3-oxo-3-(1,1-dioxothiomorpholin-4-yl)-1-((phenylsulfanyl)methyl)propylamino, (1R)-3-oxo-3-(N-methyl-N-(1,1-dimethylethyl)amino)-1-((phenylsulfanyl)methyl)propylamino, (1R)-3-oxo-3-(piperidin-1-yl)-1-((phenylsulfanyl)methyl)propylamino, (1R)-3-oxo-3-amino-1-((phenylsulfanyl)methyl)propylamino, (1R)-3-oxo-3-(methylamino)-1-((phenylsulfanyl)methyl)propylamino, (1R)-3-oxo-3-(4-methylpiperazin-1-yl)-1-((phenylsulfanyl)methyl)propylamino, (1R)-3-oxo-3-(morpholin-1-yl)-1-((phenylsulfanyl)methyl)propylamino, (1R)-3-oxo-3-(2-(morpholin-1-yl)ethyl)-1-((phenylsulfanyl)methyl)propylamino, (2-phenoxyethyl)amino, 4-(1-(phenylmethyl)piperidin-4-yl)amino, 4-(1-(phenylmethyl)piperidin-4-yl)methylamino, (4-phenyl-1,3-thiazol-2-ylsulfanyl)ethylamino, (1R,2S)-2-(phenylsulfanyl)cyclohex-1-ylamino, (1S,2R)-2-(phenylsulfanyl)cyclohex-1-ylamino, 2-(phenylsulfanyl)cyclopentylamino, 2-(phenylsulfanyl)ethoxy, 2-(phenylsulfanyl)ethylamino, 2-(phenylsulfonyl)ethylamino, (1R)-1-((phenylsulfanyl)methyl)-3-(morpholin-4-yl)propylamino, (1R)-1-((phenylsulfanyl)methyl)-3-((2,2,2-trifluoroethyl)amino)propylamino, 4-(phenylsulfonyl)tetrahydrofuran-3-ylamino, 4-(phenylsulfanyl)tetrahydrofuran-3-ylamino, (1R)-1-((phenylsulfanyl)methyl)-3-((2,2,2-trifluoroethyl)amino)propylamino, (1S)-1-((phenylsulfanyl)methyl)propylamino, (1R)-1-((phenylsulfanyl)methyl)-3-(pyridin-4-ylsulfanyl)propylamino, (1R)-1-((phenylsulfanyl)methyl)-3-(thiomorpholin-4-yl)propylamino, (1R)-1-((phenylsulfanyl)methyl)-3-(piperazin-1-yl)propylamino, (1R)-1-((phenylsulfanyl)methyl)-3-((2-(pyridin-2-yl)ethyl)amino)propylamino, (1R)-1-((phenylsulfanyl)methyl)-3-((pyridin-4-ylmethyl)amino)propylamino, (1R)-1-((phenylsulfanyl)methyl)-3-(pyridin-3-ylamino)propylamino, (1R)-1-((phenylsulfanyl)methyl)-3-(pyrrolidin-1-ylamino)propylamino), (1R)-1-((phenylsulfanyl)methyl)-3-(2H-tetrazol-5-yl)propylamino, (1R)-1-((phenylsulfanyl)methyl)-3-(pyrrolidin-1-yl)propylamino, (1R)-1-((phenylsulfanyl)methyl)-3-((pyridin-2-ylmethyl)amino)propylamino, (1S)-2-(phenylsulfanyl)-1-(pyridin-3-ylmethyl)ethylamino, (3S,4R)-(phenylsulfanyl)pyrrolidin-4-ylamino, 2-(phenylsulfanyl)-1,1-spirobutylethylamino, 2-(phenylsulfanyl)-1,1-spiroethylethylamino, 2-(phenylsulfanyl)-1,1-spiropentylethylamino, piperidin-4-yloxy, (1-propylpiperidin-4-yl)methylamino, pyran-4-ylamino, 2-(pyridin-4-ylsulfanyl)ethylamino, 2-(pyrimidin-2-ylsulfanyl)ethylamino, 1,1-spirobutyl-2-(phenylsulfanyl)ethyl, 2-(thien-2-ylsulfanyl)ethylamino, sulfanylpyran-4-ylamino, (1R)-3-(2-(2H-tetrazol-3-yl)pyrrolidin-1-yl)-1-((phenylsulfanyl)methyl)propylamino, (1R)-3-(3-(2H-tetrazol-3-yl)azetidin-1-yl)-1-((phenylsulfanyl)methyl)propylamino or 2-(1,3-thiazol-2-ylsulfanyl)ethylamino; D¹ is H, F, C₁ or CF₃; E¹ is H, F or Cl; Y¹ is H, CN, NO₂, F, Cl, CF₃, OCF₃, NH₂ or C(O)NH₂; and Z is 5-(2-(4-chlorophenyl)phenylmethyl)hexahydropyrrolo[3,4-c]pyrrol-2(1H)-yl, 1-(2-(4-chlorophenyl)phenylmethyl)piperazin-4-yl, 1-(2-(4-chlorophenyl)phenylmethyl)-piperazin-4-yl, 1-(2-(4-chlorophenyl)phenylmethyl)-piperazin-4-yl, 1-(2-(4-chlorophenyl)phenylmethyl)piperazin-4-yl, 1-(2-(4-chlorophenyl)phenylmethyl)piperazin-4-yl, 1-(2-(4-chlorophenyl)phenylmethyl)piperazin-4-yl, 1-(2-(4-chlorophenyl)phenylmethyl)piperidin-4-yl, 1-(2-(4-chlorophenyl)phenylmethyl)piperazin-4-yl, 1-(2-(4-chlorophenyl)phenylmethyl)-1,2,3,6-tetrahydropyridin-4-yl, 1-(2-(cyclohex-1-ylamino)phenylmethyl)piperazin-4-yl, 1-(2-(cyclohex-1-ylamino)phenylmethyl)piperazin-4-yl, 1-(2-(3-cyanophenyl)phenylmethyl)piperazin-4-yl, 1-(2-(2,4-dichlorophenyl)phenylmethyl)piperazin-4-yl, 1-(2-(3,4-dichlorophenyl)phenylmethyl)piperazin-4-yl, 1-(2-(2,4-difluorophenyl)phenylmethyl)piperazin-4-yl, 1-(2-(1,3-dihydro-2H-isoindol-2-yl)phenylmethyl)piperazin-4-yl, 1-(3-(1,1-dimethylethoxycarbonylamino)phenyl)piperazin-4-yl, 1-(4-(2-(dimethylamino)ethoxy)phenyl)phenylmethyl)piperazin-4-yl, or 1-(3-(dimethylamino)phenyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; B¹ is (1R)-3-((1S,4S)-2-oxa-5-azabicyclo[2.2.1]hept-5-yl)-1-((phenylsulfanyl)methyl)propylamino, (1R)-3-((1R,4R)-2-oxa-5-azabicyclo[2.2.1]hept-5-yl)-1-((phenylsulfanyl)methyl)propylamino, (1R)-3-oxo-3-(azetidin-1-yl)-1-((phenylsulfanyl)methyl)propylamino, (1R)-3-oxo-3-(cyclobutylamino)-1-((phenylsulfanyl)methyl)propylamino, (1R)-3-oxo-3-(cyclopropylamino)-1-((phenylsulfanyl)methyl)propylamino, (1R)-3-oxo-3-(dimethylamino)-1-((phenylsulfanyl)methyl)propylamino, (1R)-3-oxo-3-(dimethylamino)-1-((phenylsulfonylmethyl)methyl)propylamino, (1R)-3-oxo-3-(dimethylamino)-1-((pyrimidin-1-ylsulfanyl)methyl)propylamino, (1R)-3-oxo-3-((1,1-dimethylethyl)amino)-1-((phenylsulfanyl)methyl)propylamino, (1R)-3-oxo-3-(diisopropylamino)-1-((phenylsulfanyl)methyl)-propylamino, (1R)-3-oxo-3-(1,1-dioxothiomorpholin-4-yl)-1-((phenylsulfanyl)methyl)propylamino, (1R)-3-oxo-3-(N-methyl-N-(1,1-dimethylethyl)amino)-1-((phenylsulfanyl)methyl)propylamino, (1R)-3-oxo-3-(piperidin-1-yl)-1-((phenylsulfanyl)methyl)propylamino, (1R)-3-oxo-3-amino-1-((phenylsulfanyl)methyl)propylamino, (1R)-3-oxo-3-(methylamino)-1-((phenylsulfanyl)methyl)propylamino, (1R)-3-oxo-3-(4-methylpiperazin-1-yl)-1-((phenylsulfanyl)methyl)propylamino, (1R)-3-oxo-3-(morpholin-1-yl)-1-((phenylsulfanyl)methyl)propylamino, (1R)-3-oxo-3-(2-(morpholin-1-yl)ethyl)-1-((phenylsulfanyl)methyl)propylamino, (2-phenoxyethyl)amino, 4-(1-(phenylmethyl)piperidin-4-yl)amino, 4-(1-(phenylmethyl)piperidin-4-yl)methylamino, (4-phenyl-1,3-thiazol-2-ylsulfanyl)ethylamino, (1R,2S)-2-(phenylsulfanyl)cyclohex-1-ylamino, (1S,2R)-2-(phenylsulfanyl)cyclohex-1-ylamino, 2-(phenylsulfanyl)cyclopentylamino, 2-(phenylsulfanyl)ethoxy, 2-(phenylsulfanyl)ethylamino, 2-(phenylsulfonyl)ethylamino, (1R)-1-((phenylsulfanyl)methyl)-3-(morpholin-4-yl)propylamino, (1R)-1-((phenylsulfanyl)methyl)-3-((2,2,2-trifluoroethyl)amino)propylamino, 4-(phenylsulfonyl)tetrahydrofuran-3-ylamino, 4-(phenylsulfanyl)tetrahydrofuran-3-ylamino, (1R)-1-((phenylsulfanyl)methyl)-3-((2,2,2-trifluoroethyl)amino)propylamino, (1S)-1-((phenylsulfanyl)methyl)propylamino, (1R)-1-((phenylsulfanyl)methyl)-3-(pyridin-4-ylsulfanyl)propylamino, (1R)-1-((phenylsulfanyl)methyl)-3-(thiomorpholin-4-yl)propylamino, (1R)-1-((phenylsulfanyl)methyl)-3-(piperazin-1-yl)propylamino, (1R)-1-((phenylsulfanyl)methyl)-3-((2-(pyridin-2-yl)ethyl)amino)propylamino, (1R)-1-((phenylsulfanyl)methyl)-3-((pyridin-4-ylmethyl)amino)propylamino, (1R)-1-((phenylsulfanyl)methyl)-3-(pyridin-3-ylamino)propylamino, (1R)-1-((phenylsulfanyl)methyl)-3-(pyrrolidin-1-ylamino)propylamino), (1R)-1-((phenylsulfanyl)methyl)-3-(2H-tetrazol-5-yl)propylamino, (1R)-1-((phenylsulfanyl)methyl)-3-(pyrrolidin-1-yl)propylamino, (1R)-1-((phenylsulfanyl)methyl)-3-((pyridin-2-ylmethyl)amino)propylamino, (1S)-2-(phenylsulfanyl)-1-(pyridin-3-ylmethyl)ethylamino, (3S,4R)-(phenylsulfanyl)pyrrolidin-4-ylamino, 2-(phenylsulfanyl)-1,1-spirobutylethylamino, 2-(phenylsulfanyl)-1,1-spiroethylethylamino, 2-(phenylsulfanyl)-1,1-spiropentylethylamino, piperidin-4-yloxy, (1-propylpiperidin-4-yl)methylamino, pyran-4-ylamino, 2-(pyridin-4-ylsulfanyl)ethylamino, 2-(pyrimidin-2-ylsulfanyl)ethylamino, 1,1-spirobutyl-2-(phenylsulfanyl)ethyl, 2-(thien-2-ylsulfanyl)ethylamino, sulfanylpyran-4-ylamino, (1R)-3-(2-(2H-tetrazol-3-yl)pyrrolidin-1-yl)-1-((phenylsulfanyl)methyl)propylamino, (1R)-3-(3-(2H-tetrazol-3-yl)azetidin-1-yl)-1-((phenylsulfanyl)methyl)propylamino or 2-(1,3-thiazol-2-ylsulfanyl)ethylamino; D¹ is H, F, C₁ or CF₃; E¹ is Y¹ is H, F or Cl; Y¹ is H, CN, NO₂, F, Cl, CF₃, OCF₃, NH₂ or C(O)NH₂; and Z is 1-(4-(dimethylamino)phenyl)phenylcarbonyl)piperazin-4-yl, 1-(2-(4-(dimethylamino)phenyl)phenylmethyl)piperazin-4-yl, 1-((2-(4-(dimethylamino)phenyl)pyridin-3-yl)methyl)piperazin-4-yl, 1-(2-(5,5-dimethyl-2-oxo-1,3-oxazolidin-3-yl)phenylmethyl)piperazin-4-yl, 1-(2-(4-fluorophenyl)cyclopent-1-en-1-ylmethyl)piperazin-4-yl, 1-(2-(4-fluorophenyl)-3-fluorophenylmethyl)piperazin-4-yl, 1-(2-(4-fluorophenyl)phenylmethyl)piperazin-4-yl, 1-((2-(4-fluorophenyl)pyridin-3-yl)methyl)piperazin-4-yl, 1-(2-(isopropylamino)phenylmethyl)piperazin-4-yl, 1-(2-(4-(isopropylsulfanyl)phenyl)phenylmethyl)piperazin-4-yl, 1-(2-(4-methoxyphenyl)cyclopent-1-en-1-ylmethyl)piperazin-4-yl, 1-(2-(3-methoxyphenyl)phenylmethyl)piperazin-4-yl, 1-(2-(4-methoxyphenyl)phenylmethyl)piperazin-4-yl, 1-((2-(4-methoxyphenyl)pyridin-3-yl)methyl)piperazin-4-yl, (4-methoxy)-4-(2-(pyridin-3-yl)phenylmethyl)piperidin-1-yl, 1-(2-(2-methyl-4-dichlorophenyl)phenylmethyl)piperazin-4-yl, 1-(2-(2-methylphenyl)phenylmethyl)piperazin-4-yl, 1-(2-(4-(methylsulfonyl)phenyl)methyl)piperazin-4-yl, 1-(2-(4-methylsulfonylphenyl)phenylmethyl)piperazin-4-yl or 1-((2-(4-methylsulfonylphenyl)pyridin-3-yl)methyl)piperazin-4-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₃ or C(O)NH₂; F¹ is (1R)-3-((1S,4S)-2-oxa-5-azabicyclo[2.2.1]hept-5-yl)-1-((phenylsulfanyl)methyl)propylamino, (1R)-3-((1R,4R)-2-oxa-5-azabicyclo[2.2.1]hept-5-yl)-1-((phenylsulfanyl)methyl)propylamino, (1R)-3-oxo-3-(azetidin-1-yl)-1-((phenylsulfanyl)methyl)propylamino, (1R)-3-oxo-3-(cyclobutylamino)-1-((phenylsulfanyl)methyl)propylamino, (1R)-3-oxo-3-(cyclopropylamino)-1-((phenylsulfanyl)methyl)propylamino, (1R)-3-oxo-3-(dimethylamino)-1-((phenylsulfanyl)methyl)propylamino, (1R)-3-oxo-3-(dimethylamino)-1-((phenylsulfonylmethyl)methyl)propylamino, (1R)-3-oxo-3-(dimethylamino)-1-((pyrimidin-1-ylsulfanyl)methyl)propylamino, (1R)-3-oxo-3-((1,1-dimethylethyl)amino)-1-((phenylsulfanyl)methyl)propylamino, (1R)-3-oxo-3-(diisopropylamino)-1-((phenylsulfanyl)methyl)propylamino, (1R)-3-oxo-3-(1,1-dioxothiomorpholin-4-yl)-1-((phenylsulfanyl)methyl)propylamino, (1R)-3-oxo-3-(N-methyl-N-(1,1-dimethylethyl)amino)-1-((phenylsulfanyl)methyl)propylamino, (1R)-3-oxo-3-(piperidin-1-yl)-1-((phenylsulfanyl)methyl)propylamino, (1R)-3-oxo-3-amino-1-((phenylsulfanyl)methyl)propylamino, (1R)-3-oxo-3-(methylamino)-1-((phenylsulfanyl)methyl)propylamino, (1R)-3-oxo-3-(4-methylpiperazin-1-yl)-1-((phenylsulfanyl)methyl)propylamino, (1R)-3-oxo-3-(morpholin-1-yl)-1-((phenylsulfanyl)methyl)propylamino, (1R)-3-oxo-3-(2-(morpholin-1-yl)ethyl)-1-((phenylsulfanyl)methyl)propylamino, (2-phenoxyethyl)amino, 4-(1-(phenylmethyl)piperidin-4-yl)amino, 4-(1-(phenylmethyl)piperidin-4-yl)methylamino, (4-phenyl-1,3-thiazol-2-ylsulfanyl)ethylamino, (1R,2S)-2-(phenylsulfanyl)cyclohex-1-ylamino, (1S,2R)-2-(phenylsulfanyl)cyclohex-1-ylamino, 2-(phenylsulfanyl)cyclopentylamino, 2-(phenylsulfanyl)ethoxy, 2-(phenylsulfanyl)ethylamino, 2-(phenylsulfonyl)ethylamino, (1R)-1-((phenylsulfanyl)methyl)-3-(morpholin-4-yl)propylamino, (1R)-1-((phenylsulfanyl)methyl)-3-((2,2,2-trifluoroethyl)amino)propylamino, 4-(phenylsulfonyl)tetrahydrofuran-3-ylamino, 4-(phenylsulfanyl)tetrahydrofuran-3-ylamino, (1R)-1-((phenylsulfanyl)methyl)-3-((2,2,2-trifluoroethyl)amino)propylamino, (1S)-1-((phenylsulfanyl)methyl)propylamino, (1R)-1-((phenylsulfanyl)methyl)-3-(pyridin-4-ylsulfanyl)propylamino, (1R)-1-((phenylsulfanyl)methyl)-3-(thiomorpholin-4-yl)propylamino, (1R)-1-((phenylsulfanyl)methyl)-3-(piperazin-1-yl)propylamino, (1R)-1-((phenylsulfanyl)methyl)-3-((2-(pyridin-2-yl)ethyl)amino)propylamino, (1R)-1-((phenylsulfanyl)methyl)-3-((pyridin-4-ylmethyl)amino)propylamino, (1R)-1-((phenylsulfanyl)methyl)-3-(pyridin-3-ylamino)propylamino, (1R)-1-((phenylsulfanyl)methyl)-3-(pyrrolidin-1-ylamino)propylamino), (1R)-1-((phenylsulfanyl)methyl)-3-(2H-tetrazol-5-yl)propylamino, (1R)-1-((phenylsulfanyl)methyl)-3-(pyrrolidin-1-yl)propylamino, (1R)-1-((phenylsulfanyl)methyl)-3-((pyridin-2-ylmethyl)amino)propylamino, (1S)-2-(phenylsulfanyl)-1-(pyridin-3-ylmethyl)ethylamino, (3S,4R)-(phenylsulfanyl)pyrrolidin-4-ylamino, 2-(phenylsulfanyl)-1,1-spirobutylethylamino, 2-(phenylsulfanyl)-1,1-spiroethylethylamino, 2-(phenylsulfanyl)-1,1-spiropentylethylamino, piperidin-4-yloxy, (1-propyl-piperidin-4-yl)methylamino, pyran-4-ylamino, 2-(pyridin-4-ylsulfanyl)ethylamino, 2-(pyrimidin-2-ylsulfanyl)ethylamino, 1,1-spirobutyl-2-(phenylsulfanyl)ethyl, 2-(thien-2-ylsulfanyl)ethylamino, sulfanylpyran-4-ylamino, (1R)-3-(2-(2H-tetrazol-3-yl)pyrrolidin-1-yl)-1-((phenylsulfanyl)methyl)propylamino, (1R)-3-(3-(2H-tetrazol-3-yl)azetidin-1-yl)-1-((phenylsulfanyl)methyl)propylamino or 2-(1,3-thiazol-2-ylsulfanyl)ethylamino; D¹ is H, F, C₁ or CF₃; E¹ is H, F or Cl; Y¹ is H, CN, NO₂, F, Cl, CF₃, OCF₃, NH₂ or C(O)NH₂; and Z is 1-(2-(5-methylthien-2-yl)phenylmethyl)piperazin-4-yl, 1-(2-(4-methylsulfanylphenyl)cyclohex-1-en-1-ylmethyl)piperazin-4-yl, 1-(2-(4-methylsulfanylphenyl)phenylcarbonyl)piperazin-4-yl, 1-(2-(4-methylsulfanylphenyl)phenylcarbonyl)piperazin-4-yl, 1-((2-(4-methylsulfanylphenyl)pyridin-3-yl)methyl)piperazin-4-yl, 1-(2-(4-methylsulfanylphenyl)phenylmethyl)piperazin-1-yl, 1-(2-(4-(2-(morpholin-1-yl)ethoxy)phenyl)phenylmethyl)piperazin-1-yl, 1-(2-(morpholin-1-yl)phenylmethyl)piperazin-4-yl, 1-(2-(naphth-1-yl)phenylmethyl)piperazin-4-yl, 4-(2-(naphth-2-yl)phenylmethyl)piperazin-1-yl, 1-(2-(4-phenoxyphenyl)phenylmethyl)piperazin-4-yl, 1-((1-phenyl-1H-imidazol-2-yl)methyl)piperazin-4-yl, 1-((1-phenyl-1H-imidazol-5-yl)methyl)piperazin-4-yl, 1-(2-((phenylmethyl)amino)phenylmethyl)piperazin-4-yl, 1-(2-(phenyl)phenylmethyl)-4-(2-(dimethylamino)ethoxy))piperidin-4-y-1,2-(phenyl)phenylmethyl-4-methoxypiperidin-1-yl, 4-((2-(phenyl)phenylmethyl)-4-(2-(morpholin-4-yl)ethoxy))piperidin-1-yl, 1-(2-(phenyl)phenylmethyl)piperazin-4-yl, 1-(3-(phenyl)phenylmethyl)piperazin-4-yl, 1-(2-(4-(phenyl)phenyl)phenylmethyl)piperazin-4-yl, or 4-(2-(phenyl)phenylmethyl)-4-(2-(piperidin-1-yl)ethoxy)piperidin-1-yl.

Still another embodiment pertains to compounds having Formula I, or therapeutically acceptable salts, prodrugs or salts of prodrugs thereof, wherein A¹ is C(A²); A² is H, F, CN, C(O)OH, C(O)OCH₂ or C(O)NH₂; F¹ is (1R)-3-((1S,4S)-2-oxa-5-azabicyclo[2.2.1]hept-5-yl)-1-((phenylsulfanyl)methyl)propylamino, (1R)-3-((1R,4R)-2-oxa-5-azabicyclo[2.2.1]hept-5-yl)-1-((phenylsulfanyl)methyl)propylamino, (1R)-3-oxo-3-(azetidin-1-yl)-1-((phenylsulfanyl)methyl)propylamino, (1R)-3-oxo-3-(cyclobutylamino)-1-((phenylsulfanyl)methyl)propylamino, (1R)-3-oxo-3-(cyclopropylamino)-1-((phenylsulfanyl)methyl)propylamino, (1R)-3-oxo-3-(dimethylamino)-1-((phenylsulfanyl)methyl)propylamino, (1R)-3-oxo-3-(dimethylamino)-1-((phenylsulfonylmethyl)methyl)propylamino, (1R)-3-oxo-3-(dimethylamino)-1-((pyrimidin-1-ylsulfanyl)methyl)propylamino, (1R)-3-oxo-3-((1,1-dimethylethyl)amino)-1-((phenylsulfanyl)methyl)propylamino, (1R)-3-oxo-3-(diisopropylamino)-1-((phenylsulfanyl)methyl)propylamino, (1R)-3-oxo-3-(1,1-dioxothiomorpholin-4-yl)-1-((phenylsulfanyl)methyl)propylamino, (1R)-3-oxo-3-(N-methyl-N-(1,1-dimethylethyl)amino)-1-((phenylsulfanyl)methyl)propylamino, (1R)-3-oxo-3-(piperidin-1-yl)-1-((phenylsulfanyl)methyl)propylamino, (1R)-3-oxo-3-amino-1-((phenylsulfanyl)methyl)propylamino, (1R)-3-oxo-3-(methylamino)-1-((phenylsulfanyl)methyl)propylamino, (1R)-3-oxo-3-(4-methylpiperazin-1-yl)-1-((phenylsulfanyl)methyl)propylamino, (1R)-3-oxo-3-(morpholin-1-yl)-1-((phenylsulfanyl)methyl)propylamino, (1R)-3-oxo-3-(2-(morpholin-1-yl)ethyl)-1-((phenylsulfanyl)methyl)propylamino, (2-phenoxyethyl)amino, 4-(1-(phenylmethyl)piperidin-4-yl)amino, 4-(1-(phenylmethyl)piperidin-4-yl)methylamino, (4-phenyl-1,3-thiazol-2-ylsulfanyl)ethylamino, (1R,2S)-2-(phenylsulfanyl)cyclohex-1-ylamino, (1S,2R)-2-(phenylsulfanyl)cyclohex-1-ylamino, 2-(phenylsulfanyl)cyclopentylamino, 2-(phenylsulfanyl)ethoxy, 2-(phenylsulfanyl)ethylamino, 2-(phenylsulfonyl)ethylamino, (1R)-1-((phenylsulfanyl)methyl)-3-(morpholin-4-yl)propylamino, (1R)-1-((phenylsulfanyl)methyl)-3-((2,2,2-trifluoroethyl)amino)propylamino, 4-(phenylsulfonyl)tetrahydrofuran-3-ylamino, 4-(phenylsulfanyl)tetrahydrofuran-3-ylamino, (1R)-1-((phenylsulfanyl)methyl)-3-((2,2,2-trifluoroethyl)amino)propylamino, (1S)-1-((phenylsulfanyl)methyl)propylamino, (1R)-1-((phenylsulfanyl)methyl)-3-(pyridin-4-ylsulfanyl)propylamino, (1R)-1-((phenylsulfanyl)methyl)-3-(thiomorpholin-4-yl)propylamino, (1R)-1-((phenylsulfanyl)methyl)-3-(piperazin-1-yl)propylamino, (1R)-1-((phenylsulfanyl)methyl)-3-((2-(pyridin-2-yl)ethyl)amino)propylamino, (1R)-1-((phenylsulfanyl)methyl)-3-((pyridin-4-ylmethyl)amino)propylamino, (1R)-1-((phenylsulfanyl)methyl)-3-(pyridin-3-ylamino)propylamino, (1R)-1-((phenylsulfanyl)methyl)-3-(pyrrolidin-1-ylamino)propylamino), (1R)-1-((phenylsulfanyl)methyl)-3-(2H-tetrazol-5-yl)propylamino, (1R)-1-((phenylsulfanyl)methyl)-3-(pyrrolidin-1-yl)propylamino, (1R)-1-((phenylsulfanyl)methyl)-3-((pyridin-2-ylmethyl)amino)propylamino, (1S)-2-(phenylsulfanyl)-1-(pyridin-3-ylmethyl)ethylamino, (3S,4R)-(phenylsulfanyl)pyrrolidin-4-ylamino, 2-(phenylsulfanyl)-1,1-spirobutylethylamino, 2-(phenylsulfanyl)-1,1-spiroethylethylamino, 2-(phenylsulfanyl)-1,1-spiropentylethylamino, piperidin-4-yloxy, (1-propylpiperidin-4-yl)methylamino, pyran-4-ylamino, 2-(pyridin-4-ylsulfanyl)ethylamino, 2-(pyrimidin-2-ylsulfanyl)ethylamino, 1,1-spirobutyl-2-(phenylsulfanyl)ethyl, 2-(thien-2-ylsulfanyl)ethylamino, sulfanylpyran-4-ylamino, (1R)-3-(2-(2H-tetrazol-3-yl)pyrrolidin-1-yl)-1-((phenylsulfanyl)methyl)propylamino, (1R)-3-(3-(2H-tetrazol-3-yl)azetidin-1-yl)-1-((phenylsulfanyl)methyl)propylamino or 2-(1,3-thiazol-2-ylsulfanyl)ethylamino; D¹ is H, F, C₁ or CF₃; E¹ is H, F or Cl; Y¹ is H, CN, NO₂, F, Cl, CF₃, OCF₃, NH₂ or C(O)NH₂ and Z is 4-(2-(phenyl)phenylmethyl)-4-(2-(pyrrolidin-1-yl)ethoxy)piperidin-1-yl, 1-((2-(phenyl)pyridin-3-yl)methyl)piperazin-4-yl, 1-((1-phenyl-1H-pyrazol-5-yl)methyl)piperazin-4-yl, 1-(2-(piperidin-1-yl)phenylmethyl)piperazin-4-yl, 1-(2-(pyrid-3-yl)phenyl)phenylmethyl)piperazin-4-yl, 1-(2-(quinolin-3-ylphenyl)phenylmethyl)piperazin-4-yl, 1-(2-(quinolin-8-ylphenyl)phenylmethyl)piperazin-4-yl, 4-(2-(thien-2-yl)phenylmethyl)-4-methoxypiperazin-1-yl, 1-(2-(thien-2-yl)phenylmethyl)piperazin-4-yl, 1-(2-(4-trifluoromethoxyphenyl)phenylmethyl)piperazin-4-yl, or 1-(2-(4-trifluoromethyl-phenyl)phenylmethyl)piperazin-4-yl.

Still another embodiment pertains to compounds having Formula I selected from the compounds below:
(R)—N-(7-(4-(biphenyl-2-ylmethyl)piperazin-1-yl)quinazolin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-((4′-methoxybiphenyl-2-yl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((4′-fluorobiphenyl-2-yl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((4′-(methylthio)biphenyl-2-yl)methyl)piperazin-1-yl)quinazolin-4-yl)-3-nitrobenzenesulfonamide
(R)-4-(4-(dimethylamino)-1-(phenylthio)butan-2-ylamino)-n-(7-(4-((4′-phenylbiphenyl-2-yl)methyl)piperazin-1-yl)quinazolin-4-yl)-3-nitrobenzenesulfonamide
(R)-4-(4-(dimethylamino)-1-(phenylthio)butan-2-ylamino)-3-nitro-N-(7-(4-((4′-phenoxybiphenyl-2-yl)methyl)piperazin-1-yl)quinazolin-4-yl)benzenesulfonamide
N-(7-(4-(biphenyl-2-ylmethyl)piperazin-1-yl)quinazolin-4-yl)-4-(2-methyl-1-(phenylthio)propan-2-ylamino)-3-nitrobenzenesulfonamide
N-(7-(4-(biphenyl-2-ylmethyl)piperazin-1-yl)quinazolin-4-yl)-3-nitro-4-(2-(phenylthio) ethylamino)benzenesulfonamide
N-(7-(4-((4′-chlorobiphenyl-2-yl)methyl)piperazin-1-yl)quinazolin-4-yl)-3-nitro-4-(2-(phenylthio)ethylamino)benzenesulfonamide
(R)—N-(7-(4-((4′-chlorobiphenyl-2-yl)methyl)piperazin-1-yl)quinazolin-4-yl)-4-(4-morpholino-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-(2-methyl-1-(phenylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(5-(dimethylamino)-1-(phenylthio)pentan-2-ylamino)-3-nitrobenzenesulfonamide
(R)—N-(7-(4-((4′-chlorobiphenyl-2-yl)methyl)piperazin-1-yl)quinazolin-4-yl)-4-(6-(dimethylamino)-1-(phenylthio)hexan-2-ylamino)-3-nitrobenzenesulfonamide
(R)—N-(7-(4-((4′-fluorobiphenyl-2-yl)methyl)piperazin-1-yl)quinazolin-4-yl)-4-(4-morpholino-1-(phenylthio)butan-2-ylamino)-3-nitrobenzenesulfonamide
(R)—N-(7-(4-((4′-chloro-4-fluorobiphenyl-2-yl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-4-(4-morpholino-1-(phenylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-3-nitro-4-(1-(phenylthio)-4-(pyrrolidin-1-yl)butan-2-ylamino)benzenesulfonamide
N-(7-(4-((4′-chlorobiphenyl-2-yl)methyl)piperazin-1-yl)quinazolin-4-yl)-4-(2-methyl-1-(thiazol-2-ylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(thiazol-2-ylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(thiophen-2-ylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(1-(2-(dimethylamino)ethoxy)-3-(phenylthio)propan-2-ylamino)-3-nitrobenzenesulfonamide
(S)—N-(7-(4-((4′-chlorobiphenyl-2-yl)methyl)piperazin-1-yl)quinazolin-4-yl)-4-(4-(dimethylamino)-2-methyl-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(methylamino)-1-(phenylthio)butan-2-ylamino)-3-nitrobenzenesulfonamide
(R)-3-(4-(N-(7-(4-((4′-chlorobiphenyl-2-yl)methyl)piperazin-1-yl)quinazolin-4-yl)sulfamoyl)-2-nitrophenylamino)-4-(phenylthio)butanoic acid
(R)-3-(4-(N-(7-(4-((4′-chlorobiphenyl-2-yl)methyl)piperazin-1-yl)quinazolin-4-yl)sulfamoyl)-2-nitrophenylamino)-N-isopropyl-4-(phenylthio)butanamide
(R)—N-(7-(4-((4′-chlorobiphenyl-2-yl)methyl)piperazin-1-yl)quinazolin-4-yl)-4-(4-(diisopropylamino)-1-(phenylthio)butan-2-ylamino)-3-nitrobenzenesulfonamide
(R)-4-(4-(azetidin-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
N-(7-(4-((4′-chlorobiphenyl-2-yl)methyl)piperazin-1-yl)quinazolin-4-yl)-3-nitro-4-(4-(phenylthio)tetrahydrofuran-3-ylamino)benzenesulfonamide
(R)—N-(7-(4-((4′-chlorobiphenyl-2-yl)methyl)-4-methoxypiperidin-1-yl)quinazolin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)-4-methoxypiperidin-1-yl)quinazolin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-hydroxy-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(isopropylamino)-1-(phenylthio)butan-2-ylamino)-3-nitrobenzenesulfonamide
(R)-4-(4-(dimethylamino)-1-(phenylthio)butan-2-ylamino)-N-(6-(4-(2-(naphthalen-2-yl)benzyl)piperazin-1-yl)naphthalen-1-yl)-3-nitrobenzenesulfonamide
4-((R)-4-(dimethylamino)-1-(phenylthio)butan-2-ylamino)-N-(7-(4-(2-(naphthalen-1-yl)benzyl)piperazin-1-yl)quinazolin-4-yl)-3-nitrobenzenesulfonamide
(R)—N-(7-(4-((3′-cyanobiphenyl-2-yl)methyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-((3′-methoxybiphenyl-2-yl)methyl)piperazin-1-yl)quinazolin-4-yl)-3-nitrobenzenesulfonamide
(R)—N-(7-(4-((3′-chlorobiphenyl-2-yl)methyl)piperazin-1-yl)quinazolin-4-yl)-4-(4-(dimethylamino)-1-(phenylthio)butan-2-ylamino)-3-nitrobenzenesulfonamide
N-(7-(4-((2′-chlorobiphenyl-2-yl)methyl)piperazin-1-yl)quinazolin-4-yl)-4-((R)-4-(dimethylamino)-1-(phenylthio)butan-2-ylamino)-3-nitrobenzenesulfonamide
(R)—N-(7-(4-(2-(benzo[d][1,3]dioxol-5-yl)benzyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-3-nitro-N-(7-(4-(2-(thiophen-3-yl)benzyl)piperazin-1-yl)quinazolin-4-yl)benzenesulfonamide
(R)-4-(4-(dimethylamino)-1-(phenylthio)butan-2-ylamino)-3-nitro-N-(7-(4-(2-(pyridin-3-yl)benzyl)piperazin-1-yl)quinazolin-4-yl)benzenesulfonamide
4-((R)-4-(dimethylamino)-1-(phenylthio)butan-2-ylamino)-3-nitro-N-(7-(4-(2-(quinolin-8-yl)benzyl)piperazin-1-yl)quinazolin-4-yl)benzenesulfonamide
(R)—N-(7-(4-(2-(benzofuran-2-yl)benzyl)piperazin-1-yl)quinazolin-4-yl)-4-(4-(dimethylamino)-1-(phenylthio)butan-2-ylamino)-3-nitrobenzenesulfonamide
4-((R)-4-(dimethylamino)-1-(phenylthio)butan-2-ylamino)-N-(7-(4-((2′-methylbiphenyl-2-yl)methyl)piperazin-1-yl)quinazolin-4-yl)-3-nitrobenzenesulfonamide
(R)-4-(4-(dimethylamino)-1-(phenylthio)butan-2-ylamino)-3-nitro-N-(7-(4-(2-(quinolin-3-yl)benzyl)piperazin-1-yl)quinazolin-4-yl)benzenesulfonamide
N-(7-(4-((1-(4-chlorophenyl)naphthalen-2-yl)methyl)piperazin-1-yl)quinazolin-4-yl)-3-nitro-4-(2-(phenylthio)ethylamino)benzenesulfonamide
(R)—N-(7-(4-((1-(4-chlorophenyl)naphthalen-2-yl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((1-(4-chlorophenyl)naphthalen-2-yl)methyl)piperazin-1-yl)quinazolin-4-yl)-4-(4-morpholino-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-3-nitro-4-(2-(phenylthio)cyclopentylamino)benzenesulfonamide
N-(7-(4-(biphenyl-2-ylmethyl)-4-methoxypiperidin-1-yl)quinazolin-4-yl)-3-nitro-4-(2-(phenylthio)cyclopentylamino)benzenesulfonamide
N-(7-(4-((4′-fluorobiphenyl-2-yl)methyl)piperazin-1-yl)quinazolin-4-yl)-3-nitro-4-(2-(phenylthio)ethylamino)benzenesulfonamide
N-(7-(4-((3′,4′-dichlorobiphenyl-2-yl)methyl)piperazin-1-yl)quinazolin-4-yl)-3-nitro-4-(2-(phenylthio)ethylamino)benzenesulfonamide
(R)—N-(7-(4-((3′,4′-dichlorobiphenyl-2-yl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((3′,4′-dichlorobiphenyl-2-yl)methyl)piperazin-1-yl)quinazolin-4-yl)-4-(4-morpholino-1-(phenylthio)butan-2-ylamino)-3-nitrobenzenesulfonamide
3-nitro-4-(2-(phenylthio)ethylamino)-N-(7-(4-((4′-(trifluoromethyl)biphenyl-2-yl)methyl)piperazin-1-yl)quinazolin-4-yl)benzenesulfonamide
(R)-4-(4-(dimethylamino)-1-(phenylthio)butan-2-ylamino)-3-nitro-N-(7-(4-((4′-(trifluoromethyl)biphenyl-2-yl)methyl)piperazin-1-yl)quinazolin-4-yl)benzenesulfonamide
(R)-4-(4-morpholino-1-(phenylthio)butan-2-ylamino)-3-nitro-N-(7-(4-((4′-(trifluoromethyl)biphenyl-2-yl)methyl)piperazin-1-yl)quinazolin-4-yl)benzenesulfonamide
3-nitro-4-(2-(phenylthio)ethylamino)-N-(7-(4-((4′-(trifluoromethoxy)biphenyl-2-yl)methyl)piperazin-1-yl)quinazolin-4-yl)benzenesulfonamide
3-nitro-4-(2-(phenylthio)ethylamino)-N-(7-(4-((4′-(trifluoromethoxy)biphenyl-2-yl)methyl)piperazin-1-yl)quinazolin-4-yl)benzenesulfonamide
(R)-4-(4-morpholino-1-(phenylthio)butan-2-ylamino)-3-nitro-N-(7-(4-((4′-(trifluoromethoxy)biphenyl-2-yl)methyl)piperazin-1-yl)quinazolin-4-yl)benzenesulfonamide
3-nitro-N-(7-(4-((4′-phenoxybiphenyl-2-yl)methyl)piperazin-1-yl)quinazolin-4-yl)-4-(2-(phenylthio)ethylamino)benzenesulfonamide
(R)-4-(4-morpholino-1-(phenylthio)butan-2-ylamino)-3-nitro-N-(7-(4-((4′-phenoxybiphenyl-2-yl)methyl)piperazin-1-yl)quinazolin-4-yl)benzenesulfonamide
(S)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-(2-methyl-1-(phenylsulfonyl)propan-2-ylamino)-3-nitrobenzenesulfonamide
N-(7-(4-((2′,4′-dichlorobiphenyl-2-yl)methyl)piperazin-1-yl)quinazolin-4-yl)-4-((R)-4-(dimethylamino)-1-(phenylthio)butan-2-ylamino)-3-nitrobenzenesulfonamide
(R)-4-(4-(dimethylamino)-1-(phenylthio)butan-2-ylamino)-3-nitro-N-(7-(4-(2-(thiophen-2-yl)benzyl)piperazin-1-yl)quinazolin-4-yl)benzenesulfonamide
N-(7-(4-((4′-chloro-2′-methylbiphenyl-2-yl)methyl)piperazin-1-yl)quinazolin-4-yl)-4-((R)-4-(dimethylamino)-1-(phenylthio)butan-2-ylamino)-3-nitrobenzenesulfonamide
N-(7-(4-((2′,4′-difluorobiphenyl-2-yl)methyl)piperazin-1-yl)quinazolin-4-yl)-4-((R)-4-(dimethylamino)-1-(phenylthio)butan-2-ylamino)-3-nitrobenzenesulfonamide
N-(7-(4-(biphenyl-2-ylmethyl)piperazin-1-yl)quinazolin-4-yl)-3-nitro-4-(2-(phenylsulfonyl)ethylamino)benzenesulfonamide
N-(7-(4-((4′-chlorobiphenyl-2-yl)methyl)piperazin-1-yl)quinazolin-4-yl)-3-nitro-4-(2-(phenylsulfonyl)ethylamino)benzenesulfonamide
N-(7-(4-(biphenyl-2-ylmethyl)piperazin-1-yl)quinazolin-4-yl)-3-nitro-4-(4-(phenylthio)tetrahydrofuran-3-ylamino)benzenesulfonamide
(R)-4-(4-(dimethylamino)-1-(phenylthio)butan-2-ylamino)-N-(7-(4-(2-(5-methylthiophen-2-yl)benzyl)piperazin-1-yl)quinazolin-4-yl)-3-nitrobenzenesulfonamide
N-(7-(4-((4′-chlorobiphenyl-2-yl)methyl)piperazin-1-yl)quinazolin-4-yl)-3-nitro-4-(4-(phenylsulfonyl)tetrahydrofuran-3-ylamino)benzenesulfonamide
N-(7-(4-(biphenyl-2-ylmethyl)piperazin-1-yl)quinazolin-4-yl)-3-nitro-4-(4-(phenylsulfonyl)tetrahydrofuran-3-ylamino)benzenesulfonamide
N-(7-(4-((4′-chlorobiphenyl-2-yl)methyl)piperazin-1-yl)quinazolin-4-yl)-4-(1-methyl-4-(phenylthio)pyrrolidin-3-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-morpholino-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-morpholino-1-(phenylthio)butan-2-ylamino)-3-nitrobenzenesulfonamide
(R)—N-(7-(4-((4′-bromobiphenyl-2-yl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-(1-(4′-chlorobiphenyl-2-yl)cyclopropyl)piperazin-1-yl)quinazolin-4-yl)-4-(4-(dimethylamino)-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(1-(dimethylamino)-3-(phenylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(1-(dimethylamino)-3-(phenylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(1-(diethylamino)-3-(phenylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(1-morpholino-3-(phenylthio)propan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(1-(diethylamino)-3-(phenylthio)propan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(1-morpholino-3-(phenylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(4-(cyclopropyl(methyl)amino)-1-(phenylthio)butan-2-ylamino)-3-nitrobenzenesulfonamide
(R)—N-(7-(4-(biphenyl-2-ylmethyl)-4-methoxypiperidin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-methoxy-4-(2-(pyridin-3-yl)benzyl)piperidin-1-yl)quinazolin-4-yl)-3-nitrobenzenesulfonamide
(R)-4-(4-(dimethylamino)-1-(phenylthio)butan-2-ylamino)-N-(7-(4-methoxy-4-(2-(pyridin-4-yl)benzyl)piperidin-1-yl)quinazolin-4-yl)-3-nitrobenzenesulfonamide
(R)—N-(4-(4-(dimethylamino)-1-(phenylthio)butan-2-ylamino)-3-nitrophenylsulfonyl)-4-(4-methoxy-4-(2-(thiophen-2-yl)benzyl)piperidin-1-yl)benzamide
(R)-4-(4-(dimethylamino)-1-(phenylthio)butan-2-ylamino)-N-(7-(4-methoxy-4-(2-(thiophen-3-yl)benzyl)piperidin-1-yl)quinazolin-4-yl)-3-nitrobenzenesulfonamide
(R)-4-(4-(azetidin-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(2,2,2-trifluoroethylamino)butan-2-ylamino)benzenesulfonamide
(R)—N-(7-(4-((4′-chlorobiphenyl-2-yl)methyl)piperazin-1-yl)quinazolin-4-yl)-4-(4-(methyl(2,2,2-trifluoroethyl)amino)-1-(phenylthio)butan-2-ylamino)-3-nitrobenzenesulfonamide
(R)—N-(7-(4-((4′-chlorobiphenyl-2-yl)methyl)-4-methoxypiperidin-1-yl)quinazolin-4-yl)-4-(4-morpholino-1-(phenylthio)butan-2-ylamino)-3-nitrobenzenesulfonamide
N-(7-(4-((4′-chlorobiphenyl-2-yl)methyl)-4-methoxypiperidin-1-yl)quinazolin-4-yl)-3-nitro-4-(2-(phenylthio)ethylamino)benzenesulfonamide
(R)—N-(7-(4-((4′-chlorobiphenyl-2-yl)methyl)piperazin-1-yl)quinazolin-4-yl)-4-(4-(ethyl(2,2,2-trifluoroethyl)amino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(2-fluoroethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(2,2-difluoroethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-1-(4-(dimethylamino)-1-(phenylthio)butan-2-yl)-1H-benzo[d]imidazole-5-sulfonamide
(R)—N-(7-(4-((4′-chlorobiphenyl-2-yl)methyl)piperazin-1-yl)quinazolin-4-yl)-1-(4-(dimethylamino)-1-(phenylthio)butan-2-yl)-1H-benzo[d][1,2,3]triazole-5-sulfonamide
(R)-5-(N-(7-(4-((4′-chlorobiphenyl-2-yl)methyl)piperazin-1-yl)quinazolin-4-yl)sulfamoyl)-2-(4-(dimethylamino)-1-(phenylthio)butan-2-ylamino)benzamide
(R)-4-(4-(dimethylamino)-1-(phenylthio)butan-2-ylamino)-N-(7-(4-(4′-(dimethylamino)biphenylcarbonyl)piperazin-1-yl)quinazolin-4-yl)-3-nitrobenzenesulfonamide
(R)-4-(4-(dimethylamino)-1-(phenylthio)butan-2-ylamino)-N-(7-(4-(4′-(methylthio) biphenylcarbonyl)piperazin-1-yl)quinazolin-4-yl)-3-nitrobenzenesulfonamide
(R)-4-(4-(dimethylamino)-1-(phenylthio)butan-2-ylamino)-N-(7-(4-(4′-(methylthio) biphenylcarbonyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-3-cyano-4-(4-(dimethylamino)-1-(phenylthio)butan-2-ylamino)benzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-yloxy)-3-nitrobenzenesulfonamide
(R)—N-(7-(4-((4′-chlorobiphenyl-2-yl)methyl)piperazin-1-yl)quinazolin-4-yl)-4-(4-(4,4-dimethyl-4,5-dihydro-1H-imidazol-1-yl)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(2-methyl-4,5-dihydro-1H-imidazol-1-yl)-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-((2R)-4-(2,4-dimethyl-4,5-dihydro-1H-imidazol-1-yl)-1-(phenylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-4-(4-(2-methyl-4,5-dihydro-1H-imidazol-1-yl)-1-(phenylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-4-(4-(4,4-dimethyl-4,5-dihydro-1H-imidazol-1-yl)-1-(phenylthio)butan-2-ylamino)-3-(trifluoromethyl)benzenesulfonamide
(R)—N-(7-(4-((2-(4-chlorophenyl)cyclohex-1-enyl)methyl)piperazin-1-yl)quinazolin-4-yl)-4-(4-(dimethylamino)-1-(phenylthio)butan-2-yloxy)-3-(trifluoromethyl)benzenesulfonamide
(R,Z)—N-(7-(4-((2-(4-chlorophenyl)cyclohept-1-enyl)methyl)piperazin-1-yl)quinazolin-4-yl)-4-(4-(dimethylamino)-1-(phenylthio)butan-2-ylamino)-3-(trifluoromethyl)benzenesulfonamide
(R)-4-(4-(bis(2-methoxyethyl)amino)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)-4-(4-(bis(2-methoxyethyl)amino)-1-(phenylthio)butan-2-ylamino)-N-(7-(4-((4′-chlorobiphenyl-2-yl)methyl)piperazin-1-yl)quinazolin-4-yl)-3-(trifluoromethyl)benzenesulfonamide
(R)-4-(6-amino-1-(phenylthio)hexan-2-ylamino)-N-(7-(4-((4′-chlorobiphenyl-4-yl)methyl)piperazin-1-yl)quinazolin-4-yl)-3-nitrobenzenesulfonamide
(R)—N-(7-(4-(biphenyl-2-ylmethyl)piperazin-1-yl)quinazolin-4-yl)-4-(5-methyl-1-(phenylthio)hexan-2-ylamino)-3-nitrobenzenesulfonamide
(R)-tert-butyl 5-(4-(N-(7-(4-((4′-chlorobiphenyl-2-yl)methyl)piperazin-1-yl)quinazolin-4-yl)sulfamoyl)-2-nitrophenylamino)-6-(phenylthio)hexylcarbamate
(R)-4-(6-amino-1-(phenylthio)hexan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-4-(6-(methylsulfonamido)-1-(phenylthio)hexan-2-ylamino)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-6-ureidohexan-2-ylamino)benzenesulfonamide
(R)-4-(4-(dimethylamino)-1-(phenylthio)butan-2-ylamino)-N-(7-(4-(2-(methylthio)benzyl)piperazin-1-yl)quinazolin-4-yl)-3-nitrobenzenesulfonamide
(R)-4-(4-(dimethylamino)-1-(phenylthio)butan-2-ylamino)-N-(7-(4-(2-(methylsulfonyl)benzyl)piperazin-1-yl)quinazolin-4-yl)-3-nitrobenzenesulfonamide
(R)—N-(7-(4-(2-(5,5-dimethyl-2-oxooxazolidin-3-yl)benzyl)piperazin-1-yl)quinazolin-4-yl)-4-(4-(dimethylamino)-1-(phenylthio)butan-2-ylamino)-3-nitrobenzenesulfonamide
(R)—N-(7-(4-(2-cyclohexylbenzyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-(2-morpholinobenzyl)piperazin-1-yl)quinazolin-4-yl)-3-nitrobenzenesulfonamide
(R)-4-(4-(dimethylamino)-1-(phenylthio)butan-2-ylamino)-N-(7-(4-(2-(isopropylthio)benzyl)piperazin-1-yl)quinazolin-4-yl)-3-nitrobenzenesulfonamide
(R)—N-(7-(4-((2-(4-chlorophenyl)cyclohex-1-enyl)methyl)piperazin-1-yl)quinazolin-4-yl)-4-(4-(isopropyl(methyl)amino)-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-(dipropylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dipropylamino)-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-(diethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(diethylamino)-1-(phenylthio)butan-2-ylamino)-3-nitrobenzenesulfonamide
N-(7-(4-(biphenyl-3-ylmethyl)piperazin-1-yl)quinazolin-4-yl)-3-nitro-4-(2-(phenylthio)ethylamino)benzenesulfonamide
(R)—N-(7-(4-(biphenyl-3-ylmethyl)piperazin-1-yl)quinazolin-4-yl)-4-(4-(dimethylamino)-1-(phenylthio)butan-2-ylamino)-3-nitrobenzenesulfonamide
(R)—N-(7-(4-(biphenyl-3-ylmethyl)piperazin-1-yl)quinazolin-4-yl)-4-(4-morpholino-1-(phenylthio)butan-2-ylamino)-3-nitrobenzenesulfonamide
(R)—N-(7-(4-(biphenyl-2-ylmethyl)piperazin-1-yl)-8-fluoroquinazolin-4-yl)-4-(4-(dimethylamino)-1-(phenylthio)butan-2-ylamino)-3-nitrobenzenesulfonamide
N-(7-(4-(biphenyl-2-ylmethyl)piperazin-1-yl)-8-fluoroquinazolin-4-yl)-3-nitro-4-(2-(phenylthio)ethylamino)benzenesulfonamide; N-(7-(4-(biphenyl-2-ylmethyl)piperazin-1-yl)-6-fluoroquinazolin-4-yl)-3-nitro-4-(2-(phenylthio) ethylamino)benzenesulfonamide
(R)—N-(7-(4-(biphenyl-2-ylmethyl)piperazin-1-yl)-6-fluoroquinazolin-4-yl)-4-(4-morpholino-1-(phenylthio)butan-2-ylamino)-3-nitrobenzenesulfonamide; (R)—N-(7-(4-(biphenyl-2-ylmethyl)piperazin-1-yl)-8-fluoroquinazolin-4-yl)-4-(4-morpholino-1-(phenylthio)butan-2-ylamino)-3-nitrobenzenesulfonamide
(R)—N-(7-(4-(biphenyl-2-ylmethyl)piperazin-1-yl)-6,8-difluoroquinazolin-4-yl)-4-(4-(dimethylamino)-1-(phenylthio)butan-2-ylamino)-3-nitrobenzenesulfonamide
N-(7-(4-(biphenyl-2-ylmethyl)piperazin-1-yl)-6,8-difluoroquinazolin-4-yl)-3-nitro-4-(2-(phenylthio)ethylamino)benzenesulfonamide
(R)—N-(7-(4-(biphenyl-2-ylmethyl)piperazin-1-yl)-6,8-difluoroquinazolin-4-yl)-4-(4-morpholino-1-(phenylthio)butan-2-ylamino)-3-nitrobenzenesulfonamide
(R)—N-(7-(4-(biphenyl-2-ylmethyl)piperazin-1-yl)-6,8-difluoroquinazolin-4-yl)-4-(4-morpholino-1-(phenylthio)butan-2-ylamino)-3-nitrobenzenesulfonamide
(R)-4-(4-morpholino-1-(phenylthio)butan-2-ylamino)-3-nitro-N-(7-(4-((1-phenyl-1H-imidazol-2-yl)methyl)piperazin-1-yl)quinazolin-4-yl)benzenesulfonamide
3-nitro-N-(7-(4-((1-phenyl-1H-pyrazol-5-yl)methyl)piperazin-1-yl)quinazolin-4-yl)-4-(2-(phenylthio) ethylamino)benzenesulfonamide
(R)-4-(4-morpholino-1-(phenylthio)butan-2-ylamino)-3-nitro-N-(7-(4-((1-phenyl-1H-pyrazol-5-yl)methyl)piperazin-1-yl)quinazolin-4-yl)benzenesulfonamide
(R)-4-(4-morpholino-1-(phenylthio)butan-2-ylamino)-3-nitro-N-(7-(4-((1-phenyl-1H-pyrazol-5-yl)methyl)piperazin-1-yl)quinazolin-4-yl)benzenesulfonamide
(R)-4-(4-morpholino-1-(phenylthio)butan-2-ylamino)-3-nitro-N-(7-(4-((1-phenyl-1H-imidazol-5-yl)methyl)piperazin-1-yl)quinazolin-4-yl)benzenesulfonamide
(R)-1-(3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl)azetidine-3-carboxylic acid
(R)—N-(7-(4-((4′-chlorobiphenyl-2-yl)methyl)piperazin-1-yl)quinazolin-4-yl)-4-(4-(2-hydroxy-2-methylpropylamino)-1-(phenylthio)butan-2-ylamino)-3-nitrobenzenesulfonamide
(R)-2-((3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl) (methyl)amino)acetic acid
(R)-1-((R)-3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl)pyrrolidine-2-carboxylic acid
(R)-1-(3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl)piperidine-4-carboxylic acid
(R)—N-(7-(4-((4′-chlorobiphenyl-2-yl)methyl)piperazin-1-yl)quinazolin-4-yl)-4-(4-((2-hydroxyethyl)(methyl)amino)-1-(phenylthio)butan-2-ylamino)-3-nitrobenzenesulfonamide
(S)-1-((R)-3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl)pyrrolidine-2-carboxylic acid
(R)-4-(4-(3-(2H-tetrazol-5-yl)azetidin-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(S)-2-amino-N—((S)-1-((R)-3-(4-(N-(7-(4-((4′-chlorobiphenyl-2-yl)methyl)piperazin-1-yl)quinazolin-4-yl)sulfamoyl)-2-nitrophenylamino)-4-(phenylthio)butylamino)-1-oxopropan-2-yl)propanamide
4-((2R)-4-(2-(2H-tetrazol-5-yl)pyrrolidin-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)-1-(3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl)-N-(methylsulfonyl)piperidine-4-carboxamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)benzenesulfonamide
(R)-1-(3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl)-N-hydroxypiperidine-4-carboxamide
(R)-2-chloro-N-(7-(4-((4′-chlorobiphenyl-2-yl)methyl)piperazin-1-yl)quinazolin-4-yl)-4-(4-(dimethylamino)-1-(phenylthio)butan-2-ylamino)benzenesulfonamide
(R)-2,6-dichloro-N-(7-(4-((4′-chlorobiphenyl-2-yl)methyl)piperazin-1-yl)quinazolin-4-yl)-4-(4-(dimethylamino)-1-(phenylthio)butan-2-ylamino)benzenesulfonamide
4-(4-((1R,5S)-8-azabicyclo[3.2.1]octan-8-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)-4-(4-(7-azabicyclo[2.2.1]heptan-7-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
N-(7-(4-((4′-chlorobiphenyl-2-yl)methyl)piperazin-1-yl)quinazolin-4-yl)-3-nitro-4-(2-(phenylthio)ethoxy)benzenesulfonamide
N-(7-(4-((4′-chlorobiphenyl-2-yl)methyl)piperazin-1-yl)quinazolin-4-yl)-4-(2-(phenylthio)ethoxy)-3-(trifluoromethyl)benzenesulfonamide
4-(4-((1R,4S)-2-oxa-5-azabicyclo[2.2.1]heptan-5-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
4-(4-((1R,4R)-2-oxa-5-azabicyclo[2.2.1]heptan-5-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
4-(4-((1R,4S)-2-oxa-5-azabicyclo[2.2.1]heptan-5-yl)-1-(phenylthio)butan-2-ylamino)-N-(7-(4-((2-(4-chlorophenyl)cyclohex-1-enyl)methyl)piperazin-1-yl)quinazolin-4-yl)-3-nitrobenzenesulfonamide
4-(4-((1R,4R)-2-oxa-5-azabicyclo[2.2.1]heptan-5-yl)-1-(phenylthio)butan-2-ylamino)-N-(7-(4-((2-(4-chlorophenyl)cyclohex-1-enyl)methyl)piperazin-1-yl)quinazolin-4-yl)-3-nitrobenzenesulfonamide
(R)-4-(4-(7-azabicyclo[2.2.1]heptan-7-yl)-1-(phenylthio)butan-2-ylamino)-N-(7-(4-((2-(4-chlorophenyl)cyclohex-1-enyl)methyl)piperazin-1-yl)quinazolin-4-yl)-3-nitrobenzenesulfonamide
N-(7-(4-((2-(4-chlorophenyl)cyclohex-1-enyl)methyl)piperazin-1-yl)quinazolin-4-yl)-4-((R)-4-((2R,5S)-2,5-dimethylpyrrolidin-1-yl)-1-(phenylthio)butan-2-ylamino)-3-nitrobenzenesulfonamide
4-(4-((1R,4R)-2-oxa-5-azabicyclo[2.2.1]heptan-5-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
N-(7-(4-(biphenyl-2-ylmethyl)piperazin-1-yl)quinazolin-4-yl)-4-(cyclohexyloxy)-3-nitrobenzenesulfonamide
N-(7-(4-(biphenyl-2-ylmethyl)piperazin-1-yl)quinazolin-4-yl)-4-(cyclohexylmethoxy)-3-nitrobenzenesulfonamide
N-(7-(4-(biphenyl-2-ylmethyl)piperazin-1-yl)quinazolin-4-yl)-4-(2-cyclohexylethoxy)-3-nitrobenzenesulfonamide
N-(7-(4-(biphenyl-2-ylmethyl)piperazin-1-yl)quinazolin-4-yl)-3-nitro-4-(tetrahydro-2H-pyran-4-ylamino) benzenesulfonamide
N-(7-(4-(biphenyl-2-ylmethyl)piperazin-1-yl)quinazolin-4-yl)-4-(2-cyclohexylethylamino)-3-nitrobenzenesulfonamide
N-(7-(4-(biphenyl-2-ylmethyl)piperazin-1-yl)quinazolin-4-yl)-4-(cyclohexyl(methyl)amino)-3-nitrobenzenesulfonamide
N-(7-(4-(biphenyl-2-ylmethyl)piperazin-1-yl)quinazolin-4-yl)-4-(4,4-dimethylpiperidin-1-yl)-3-nitrobenzenesulfonamide
tert-butyl 4-(4-(N-(7-(4-(biphenyl-2-ylmethyl)piperazin-1-yl)quinazolin-4-yl)sulfamoyl)-2-nitrophenoxy)piperidine-1-carboxylate
N-(7-(4-(biphenyl-2-ylmethyl)piperazin-1-yl)quinazolin-4-yl)-3-nitro-4-(piperidin-4-yloxy)benzenesulfonamide
N-(7-(4-(biphenyl-2-ylmethyl)piperazin-1-yl)quinazolin-4-yl)-4-(1-methylpiperidin-4-yloxy)-3-nitrobenzenesulfonamide
N-(7-(4-((4′-chlorobiphenyl-2-yl)methyl)piperazin-1-yl)quinazolin-4-yl)-4-(cyclohexylmethylamino)-3-nitrobenzenesulfonamide
N-(7-(4-((4′-chlorobiphenyl-2-yl)methyl)piperazin-1-yl)quinazolin-4-yl)-4-((cyclohexylmethyl)(propyl)amino)-3-nitrobenzenesulfonamide
4-((1-benzylpiperidin-4-yl)methylamino)-N-(7-(4-(biphenyl-2-ylmethyl)piperazin-1-yl)quinazolin-4-yl)-3-nitrobenzenesulfonamide
N-(7-(4-(biphenyl-2-ylmethyl)piperazin-1-yl)quinazolin-4-yl)-4-((cyclohexylmethyl)(methyl)amino)-3-nitrobenzenesulfonamide
4-(1-benzylpiperidin-4-ylamino)-N-(7-(4-(biphenyl-2-ylmethyl)piperazin-1-yl)quinazolin-4-yl)-3-nitrobenzenesulfonamide
N-(7-(4-(biphenyl-2-ylmethyl)piperazin-1-yl)quinazolin-4-yl)-4-(2-mercaptotetrahydro-2H-pyran-4-ylamino)-3-nitrobenzenesulfonamide
ethyl 4-(4-(N-(7-(4-(biphenyl-2-ylmethyl)piperazin-1-yl)quinazolin-4-yl)sulfamoyl)-2-nitrophenylamino)piperidine-1-carboxylate
N-(7-(4-(biphenyl-2-ylmethyl)piperazin-1-yl)quinazolin-4-yl)-3-nitro-4-((1-propylpiperidin-4-yl)methylamino)benzenesulfonamide
N-(7-(4-(biphenyl-2-ylmethyl)piperazin-1-yl)quinazolin-4-yl)-4-(isopropylamino)-3-nitrobenzenesulfonamide
N-(7-(4-(biphenyl-2-ylmethyl)piperazin-1-yl)quinazolin-4-yl)-3-nitro-4-(2-(thiazol-2-ylthio)ethylamino)benzenesulfonamide
N-(7-(4-(biphenyl-2-ylmethyl)piperazin-1-yl)quinazolin-4-yl)-3-nitro-4-(2-(4-phenylthiazol-2-ylthio)ethylamino)benzenesulfonamide
4-(2-(benzo[d]thiazol-2-ylthio)ethylamino)-N-(7-(4-(biphenyl-2-ylmethyl)piperazin-1-yl)quinazolin-4-yl)-3-nitrobenzenesulfonamide
N-(7-(4-((4′-chlorobiphenyl-2-yl)methyl)piperazin-1-yl)quinazolin-4-yl)-3-nitro-4-(2-(thiazol-2-ylthio)ethylamino)benzenesulfonamide
4-(2-(benzo[d]oxazol-2-ylthio)ethylamino)-N-(7-(4-((4′-chlorobiphenyl-2-yl)methyl)piperazin-1-yl)quinazolin-4-yl)-3-nitrobenzenesulfonamide
4-(2-(benzo[d]thiazol-2-ylthio)ethylamino)-N-(7-(4-((4′-chlorobiphenyl-2-yl)methyl)piperazin-1-yl)quinazolin-4-yl)-3-nitrobenzenesulfonamide
N-(7-(4-((4′-chlorobiphenyl-2-yl)methyl)piperazin-1-yl)quinazolin-4-yl)-3-nitro-4-(2-(pyrimidin-2-ylthio)ethylamino)benzenesulfonamide
(R)-4-(4-morpholino-1-(phenylthio)butan-2-ylamino)-3-nitro-N-(7-(4-((1-phenyl-1H-pyrazol-5-yl)methyl)piperazin-1-yl)quinazolin-4-yl)benzenesulfonamide
4-((1-benzylpiperidin-4-yl)methylamino)-N-(7-(4-((4′-chlorobiphenyl-2-yl)methyl)piperazin-1-yl)quinazolin-4-yl)-3-nitrobenzenesulfonamide
N-(7-(4-(biphenyl-2-ylmethyl)piperazin-1-yl)quinazolin-4-yl)-4-(2-bromoethylamino)-3-nitrobenzenesulfonamide
N-(7-(4-((4′-chlorobiphenyl-2-yl)methyl)piperazin-1-yl)quinazolin-4-yl)-4-(2-(4-methylthiazol-2-ylthio)ethylamino)-3-nitrobenzenesulfonamide
N-(7-(4-((4′-chlorobiphenyl-2-yl)methyl)piperazin-1-yl)quinazolin-4-yl)-4-((4-methoxycyclohexyl)methylamino)-3-nitrobenzenesulfonamide
N-(7-(4-(biphenyl-2-ylmethyl)piperazin-1-yl)quinazolin-4-yl)-3-nitro-4-(2-(thiophen-2-ylthio)ethylamino)benzenesulfonamide
N-(7-(4-((4′-chlorobiphenyl-2-yl)methyl)piperazin-1-yl)quinazolin-4-yl)-4-(2-methyl-1-(thiophen-2-ylthio)propan-2-ylamino)-3-nitrobenzenesulfonamide
(R)—N-(7-(4-((4′-chlorobiphenyl-2-yl)methyl)piperazin-1-yl)quinazolin-4-yl)-4-(4-morpholino-1-(thiazol-2-ylthio)butan-2-ylamino)-3-nitrobenzenesulfonamide
(R)-3-(4-(N-(7-(4-((4′-chlorobiphenyl-2-yl)methyl)piperazin-1-yl)quinazolin-4-yl)sulfamoyl)-2-nitrophenylamino)-N,N-dimethyl-4-(pyrimidin-2-ylthio)butanamide
(R)—N-(7-(4-((4′-chlorobiphenyl-2-yl)methyl)piperazin-1-yl)quinazolin-4-yl)-4-(4-morpholino-4-oxo-1-(thiophen-2-ylthio)butan-2-ylamino)-3-nitrobenzenesulfonamide
N-(7-(4-((4′-chlorobiphenyl-2-yl)methyl)piperazin-1-yl)quinazolin-4-yl)-4-(2-methyl-1-(pyrimidin-2-ylthio)propan-2-ylamino)-3-nitrobenzenesulfonamide
(R)-3-(4-(N-(7-(4-((4′-chlorobiphenyl-2-yl)methyl)piperazin-1-yl)quinazolin-4-yl)sulfamoyl)-2-nitrophenylamino)-N,N-dimethyl-4-(thiazol-2-ylthio)butanamide
(R)—N-(7-(4-((4′-chlorobiphenyl-2-yl)methyl)piperazin-1-yl)quinazolin-4-yl)-4-(4-morpholino-1-(thiophen-2-ylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(4-(trifluoromethoxy)phenylthio)butan-2-ylamino)-3-nitrobenzenesulfonamide
N-(7-(4-((4′-chlorobiphenyl-2-yl)methyl)piperazin-1-yl)quinazolin-4-yl)-3-nitro-4-(2-phenoxyethylamino)benzenesulfonamide
(R)—N-(7-(4-((4′-chlorobiphenyl-2-yl)methyl)piperazin-1-yl)quinazolin-4-yl)-4-(4-morpholino-1-(4-(trifluoromethoxy)phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(1-(4-methoxyphenylthio)-4-morpholinobutan-2-ylamino)-3-nitrobenzenesulfonamide
(R)—N-(7-(4-((4′-chlorobiphenyl-2-yl)methyl)piperazin-1-yl)quinazolin-4-yl)-4-(4-morpholino-1-(p-tolylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(thiophen-2-ylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-4-(1-(4-chlorophenylthio)-4-morpholinobutan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(4-fluorophenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(1-(4-fluorophenylthio)-4-morpholinobutan-2-ylamino)-3-nitrobenzenesulfonamide
(R)—N-(7-(4-((4′-chlorobiphenyl-2-yl)methyl)piperazin-1-yl)-5-fluoroquinazolin-4-yl)-4-(4-(dimethylamino)-1-(phenylthio)butan-2-ylamino)-3-nitrobenzenesulfonamide
(R)—N-(7-(1-((4′-chlorobiphenyl-2-yl)methyl)-1,2,3,6-tetrahydropyridin-4-yl)quinazolin-4-yl)-4-(4-(dimethylamino)-1-(phenylthio)butan-2-ylamino)-3-nitrobenzenesulfonamide
(R)—N-(7-(1-((4′-chlorobiphenyl-2-yl)methyl)-1,2,3,6-tetrahydropyridin-4-yl)quinazolin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)-5-fluoroquinazolin-4-yl)-4-(4-(dimethylamino)-1-(phenylthio)butan-2-ylamino)-3-(trifluoromethyl)benzenesulfonamide
(R)—N-(7-(4-((4′-chlorobiphenyl-2-yl)methyl)piperazin-1-yl)pyrido[4,3-d]pyrimidin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)piperazin-1-yl)pyrido[2,3-d]pyrimidin-4-yl)-4-(4-(dimethylamino)-1-(phenylthio)butan-2-ylamino)-3-nitrobenzenesulfonamide
(R)—N-(7-(1-((4′-chlorobiphenyl-2-yl)methyl)piperidin-4-yl)quinazolin-4-yl)-4-(4-(dimethylamino)-1-(phenylthio)butan-2-ylamino)-3-nitrobenzenesulfonamide
(R)—N-(7-(1-((4′-chlorobiphenyl-2-yl)methyl)piperidin-4-yl)quinazolin-4-yl)-4-(4-morpholino-1-(phenylthio)butan-2-ylamino)-3-nitrobenzenesulfonamide
(R)—N-(7-(1-((4′-chlorobiphenyl-2-yl)methyl)-1,2,3,6-tetrahydropyridin-4-yl)quinazolin-4-yl)-4-(4-(dimethylamino)-1-(phenylthio)butan-2-ylamino)-3-(trifluoromethyl)benzenesulfonamide
(R)—N-(7-(4-((4′-chlorobiphenyl-2-yl)methyl)piperazin-1-yl)pyrido[4,3-d]pyrimidin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)pyrido[2,3-d]pyrimidin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)pyrido[2,3-d]pyrimidin-4-yl)-4-(4-(dimethylamino)-1-(phenylthio)butan-2-ylamino)-3-(trifluoromethyl)benzenesulfonamide
(R)—N-(7-(4-((4′-chlorobiphenyl-2-yl)methyl)piperazin-1-yl)pyrido[4,3-d]pyrimidin-4-yl)-4-(4-(dimethylamino)-1-(phenylthio)butan-2-ylamino)-3-(trifluoromethyl)benzenesulfonamide
(R)—N-(7-(4-((4′-chlorobiphenyl-2-yl)methyl)piperidin-1-yl)quinazolin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)piperidin-1-yl)quinazolin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)pyrido[3,2-d]pyrimidin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)piperazin-1-yl)pyrido[3,2-d]pyrimidin-4-yl)-4-(4-morpholino-1-(phenylthio)butan-2-ylamine)-3-nitrobenzenesulfonamide
(R)—N-(7-(1-((2-(4-chlorophenyl)-5,5-dimethylcyclohex-1-enyl)methyl)-1,2,3,6-tetrahydropyridin-4-yl)quinazolin-4-yl)-4-(4-(dimethylamine)-1-(phenylthio)butan-2-ylamine)-3-nitrobenzenesulfonamide
(R)—N-(7-(1-((2-(4-chlorophenyl)cyclohex-1-enyl)methyl)-1,2,3,6-tetrahydropyridin-4-yl)quinazolin-4-yl)-4-(4-(dimethylamine)-1-(phenylthio)butan-2-ylamine)-3-nitrobenzenesulfonamide
(R)—N-(7-(1-((2-(4-chlorophenyl)cyclohex-1-enyl)methyl)-1,2,3,6-tetrahydropyridin-4-yl)quinazolin-4-yl)-4-(4-morpholine-1-(phenylthio)butan-2-ylamine)-3-nitrobenzenesulfonamide
(R)—N-(7-(1-((2-(4-chlorophenyl)-5,5-dimethylcyclohex-1-enyl)methyl)-1,2,3,6-tetrahydropyridin-4-yl)quinazolin-4-yl)-4-(4-morpholine-1-(phenylthio)butan-2-ylamine)-3-nitrobenzenesulfonamide
N-(7-(4-((4′-chlorobiphenyl-2-yl)methyl)cyclohex-1-enyl)quinazolin-4-yl)-4-((R)-4-(dimethylamine)-1-(phenylthio)butan-2-ylamino)-3-nitrobenzenesulfonamide
N-(7-(4-((4′-chlorobiphenyl-2-yl)methyl)cyclohex-1-enyl)quinazolin-4-yl)-4-((R)-4-morpholino-1-(phenylthio)butan-2-ylamino)-3-nitrobenzenesulfonamide
N-(7-((3aR,6aS)-5-((4′-chlorobiphenyl-2-yl)methyl)hexahydropyrrolo[3,4-c]pyrrol-2(1H)-yl)quinazolin-4-yl)-4-((R)-4-(dimethylamino)-1-(phenylthio)butan-2-ylamino)-3-nitrobenzenesulfonamide
N-(7-(4-(biphenyl-2-ylmethyl)piperazin-1-yl)quinazolin-4-yl)-4-(1,3-dimethyl-3-(4-(trifluoromethoxy)phenyl)ureido)-3-nitrobenzenesulfonamide
N-(7-(4-(biphenyl-2-ylmethyl)piperazin-1-yl)quinazolin-4-yl)-4-(3-(4-methoxyphenyl)-1,3-dimethylureido)-3-nitrobenzenesulfonamide
4-(3-benzhydryl-1,3-dimethylureido)-N-(7-(4-(biphenyl-2-ylmethyl)piperazin-1-yl)quinazolin-4-yl)-3-nitrobenzenesulfonamide
(S)—N-(7-(4-(biphenyl-2-ylmethyl)piperazin-1-yl)quinazolin-4-yl)-4-(1,3-dimethyl-3-(1-phenylethyl)ureido)-3-nitrobenzenesulfonamide
N-(7-(4-(biphenyl-2-ylmethyl)piperazin-1-yl)quinazolin-4-yl)-4-(1,3-dimethyl-3-(2-(4-methylpiperazin-1-yl)-1-phenylethyl)ureido)-3-nitrobenzenesulfonamide
N-(7-(4-(biphenyl-2-ylmethyl)piperazin-1-yl)quinazolin-4-yl)-4-(1,3-dimethyl-3-(2-morpholino-1-phenylethyl)ureido)-3-nitrobenzenesulfonamide
N-(7-(4-(biphenyl-2-ylmethyl)piperazin-1-yl)quinazolin-4-yl)-4-(3-(1,2-diphenylethyl)-1,3-dimethylureido)-3-nitrobenzenesulfonamide
N-(7-(4-(biphenyl-2-ylmethyl)piperazin-1-yl)quinazolin-4-yl)-4-(3-(2-(dimethylamino)-1-phenylethyl)-1,3-dimethylureido)-3-nitrobenzenesulfonamide
3-amino-N-(7-(4-((4′-chlorobiphenyl-2-yl)methyl)piperazin-1-yl)quinazolin-4-yl)-4-(2-(phenylthio) ethylamino)benzenesulfonamide
N-(7-(4-((4′-chlorobiphenyl-2-yl)methyl)piperazin-1-yl)quinazolin-4-yl)-1-(2-(phenylthio)ethyl)-1H-benzo[d][1,2,3]triazole-5-sulfonamide
N-(7-(4-((4′-chlorobiphenyl-2-yl)methyl)piperazin-1-yl)quinazolin-4-yl)-1-(2-(phenylthio)ethyl)-1H-benzo[d]imidazole-5-sulfonamide
N-(7-(4-(biphenyl-2-ylmethyl)-4-methoxypiperidin-1-yl)quinazolin-4-yl)-4-(cyclohexylmethylamino)-3-nitrobenzenesulfonamide
N-(7-(4-(biphenyl-2-ylmethyl)-4-methoxypiperidin-1-yl)quinazolin-4-yl)-4-(cyclohexylamino)-3-nitrobenzenesulfonamide
N-(7-(4-(biphenyl-2-ylmethyl)-4-methoxypiperidin-1-yl)quinazolin-4-yl)-4-(2-methyl-1-(phenylthio)propan-2-ylamino)-3-nitrobenzenesulfonamide
N-(7-(4-(biphenyl-2-ylmethyl)piperazin-1-yl)quinazolin-4-yl)-3-nitro-4-(1-(phenylthiomethyl)cyclopentylamino)benzenesulfonamide
N-(7-(4-(biphenyl-2-ylmethyl)-4-methoxypiperidin-1-yl)quinazolin-4-yl)-3-nitro-4-(1-(phenylthiomethyl)cyclopentylamino)benzenesulfonamide
N-(7-(4-((4′-chlorobiphenyl-2-yl)methyl)piperazin-1-yl)quinazolin-4-yl)-3-nitro-4-(1-(phenylthiomethyl)cyclopentylamino)benzenesulfonamide
(S)—N-(7-(4-((4′-chlorobiphenyl-2-yl)methyl)piperazin-1-yl)quinazolin-4-yl)-3-nitro-4-(1-(phenylthio)butan-2-ylamino)benzenesulfonamide
(S)—N-(7-(4-(biphenyl-2-ylmethyl)-4-methoxypiperidin-1-yl)quinazolin-4-yl)-3-nitro-4-(1-(phenylthio)butan-2-ylamino)benzenesulfonamide
(R)—N-(7-(4-((4′-chlorobiphenyl-2-yl)methyl)piperazin-1-yl)quinazolin-4-yl)-4-(4-morpholino-1-(phenylthio)butan-2-ylamino)-3-nitrobenzenesulfonamide
(S)—N-(7-(4-((4′-chlorobiphenyl-2-yl)methyl)piperazin-1-yl)quinazolin-4-yl)-4-(4-methyl-1-(phenylthio)pentan-2-ylamino)-3-nitrobenzenesulfonamide
(S)—N-(7-(4-(biphenyl-2-ylmethyl)-4-methoxypiperidin-1-yl)quinazolin-4-yl)-4-(4-methyl-1-(phenylthio)pentan-2-ylamino)-3-nitrobenzenesulfonamide
N-(7-(4-((4′-chlorobiphenyl-2-yl)methyl)piperazin-1-yl)quinazolin-4-yl)-3-nitro-4-(1-(phenylthiomethyl)cyclopropylamino)benzenesulfonamide
N-(7-(4-((4′-chlorobiphenyl-2-yl)methyl)piperazin-1-yl)quinazolin-4-yl)-3-nitro-4-(1-(phenylthiomethyl)cyclohexylamino)benzenesulfonamide
(R)—N-(7-(4-((4′-chlorobiphenyl-2-yl)methyl)piperazin-1-yl)quinazolin-4-yl)-3-nitro-4-(1-(phenylthio)propan-2-ylamino)benzenesulfonamide
(S)—N-(7-(4-(biphenyl-2-ylmethyl)piperazin-1-yl)quinazolin-4-yl)-3-nitro-4-(1-(phenylthio)propan-2-ylamino)benzenesulfonamide
N-(7-(4-(biphenyl-2-ylmethyl)piperazin-1-yl)quinazolin-4-yl)-3-nitro-4-((1R,2S)-2-(phenylthio)cyclohexylamino)benzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-(trifluoromethyl)benzenesulfonamide
(R)-4-(6-amino-1-(phenylthio)hexan-2-ylamino)-N-(7-(4-((4′-chlorobiphenyl-2-yl)methyl)piperazin-1-yl)quinazolin-4-yl)-3-nitrobenzenesulfonamide
(S)—N-(7-(4-(biphenyl-2-ylmethyl)piperazin-1-yl)quinazolin-4-yl)-3-nitro-4-(1-(phenylthio)-3-(pyridin-3-yl)propan-2-ylamino)benzenesulfonamide
(S)—N-(7-(4-(biphenyl-2-ylmethyl)-4-methoxypiperidin-1-yl)quinazolin-4-yl)-3-nitro-4-(1-(phenylthio)-3-(pyridin-3-yl)propan-2-ylamino)benzenesulfonamide
N-(7-(4-(biphenyl-2-ylmethyl)piperazin-1-yl)quinazolin-4-yl)-3-nitro-4-((1S,2R)-2-(phenylthio)cyclohexylamino)benzenesulfonamide
N-(7-(4-(biphenyl-2-ylmethyl)piperazin-1-yl)quinazolin-4-yl)-4-(1-((2-methylfuran-3-ylthio)methyl)cyclopentylamino)-3-nitrobenzenesulfonamide
N-(7-(4-((4′-chlorobiphenyl-2-yl)methyl)piperazin-1-yl)quinazolin-4-yl)-4-(1-((2-methylfuran-3-ylthio)methyl)cyclopentylamino)-3-nitrobenzenesulfonamide
(S)—N-(7-(4-((4′-chlorobiphenyl-2-yl)methyl)piperazin-1-yl)quinazolin-4-yl)-3-nitro-4-(1-(phenylthio)-3-(pyridin-3-yl)propan-2-ylamino)benzenesulfonamide
NA
N-(7-(4-((2-(4-chlorophenyl)pyridin-3-yl)methyl)piperazin-1-yl)quinazolin-4-yl)-3-nitro-4-(2-(phenylthio)ethylamino)benzenesulfonamide
(R)—N-(7-(4-((2-(4-chlorophenyl)pyridin-3-yl)methyl)piperazin-1-yl)quinazolin-4-yl)-4-(4-morpholino-1-(phenylthio)butan-2-ylamino)-3-nitrobenzenesulfonamide
3-nitro-N-(7-(4-((2-phenylpyridin-3-yl)methyl)piperazin-1-yl)quinazolin-4-yl)-4-(2-(phenylthio) ethylamino)benzenesulfonamide
(R)-4-(4-morpholino-1-(phenylthio)butan-2-ylamino)-3-nitro-N-(7-(4-((2-phenylpyridin-3-yl)methyl)piperazin-1-yl)quinazolin-4-yl)benzenesulfonamide
(R)-4-(4-(dimethylamino)-1-(phenylthio)butan-2-ylamino)-3-nitro-N-(7-(4-((2-phenylpyridin-3-yl)methyl)piperazin-1-yl)quinazolin-4-yl)benzenesulfonamide
(R)-4-(4-(dimethylamino)-1-(phenylthio)butan-2-ylamino)-N-(7-(4-((2-(4-(methylthio)phenyl)pyridin-3-yl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((2-(4-methoxyphenyl)pyridin-3-yl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((2-(4-(dimethylamino)phenyl)pyridin-3-yl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((2-(4-fluorophenyl)pyridin-3-yl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((2-(4-(methylsulfonyl)phenyl)pyridin-3-yl)methyl)piperazin-1-yl)quinazolin-4-yl)-3-nitrobenzenesulfonamide
N-(7-(4-((4′-chlorobiphenyl-2-yl)methyl)piperazin-1-yl)quinazolin-4-yl)-3-nitro-4-(2-(pyridin-4-ylthio)ethylamino)benzenesulfonamide
(R)-4-(4-(dimethylamino)-1-(phenylthio)butan-2-ylamino)-N-(7-(4-((4′-(methylsulfonyl)biphenyl-2-yl)methyl)piperazin-1-yl)quinazolin-4-yl)-3-nitrobenzenesulfonamide
(R)—N-(7-(4-((4′-(methylsulfonyl)biphenyl-2-yl)methyl)piperazin-1-yl)quinazolin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-morpholino-1-(phenylsulfonyl)butan-2-ylamino)-3-nitrobenzenesulfonamide
(R)-4-(4-(dimethylamino)-1-(phenylthio)butan-2-ylamino)-N-(7-(4-((4′-(dimethylamino)biphenyl-2-yl)methyl)piperazin-1-yl)quinazolin-4-yl)-3-nitrobenzenesulfonamide
(R)-3-(4-(N-(7-(4-((4′-chlorobiphenyl-2-yl)methyl)piperazin-1-yl)quinazolin-4-yl)sulfamoyl)-2-nitrophenylamino)-N,N-dimethyl-4-(phenylsulfonyl)butanamide
(R)—N-(7-(4-((4′-chlorobiphenyl-2-yl)methyl)piperazin-1-yl)quinazolin-4-yl)-3-nitro-4-(1-(phenylthio)pyrrolidin-3-ylamino)benzenesulfonamide
(R)—N-(7-(4-((4′-chlorobiphenyl-2-yl)methyl)piperazin-1-yl)quinazolin-4-yl)-3-nitro-4-(1-(phenylthio)-4-(pyridin-4-ylthio)butan-2-ylamino)benzenesulfonamide
(R)—N-(7-(4-((3-(4-chlorophenyl)pyridin-4-yl)methyl)piperazin-1-yl)quinazolin-4-yl)-4-(4-(dimethylamino)-1-(phenylthio)butan-2-ylamino)-3-nitrobenzenesulfonamide
N-(7-(4-((3-(4-chlorophenyl)pyridin-4-yl)methyl)piperazin-1-yl)quinazolin-4-yl)-3-nitro-4-(2-(phenylthio)ethylamino)benzenesulfonamide
(R)—N-(7-(4-((2-(4-chlorophenyl)cyclopent-1-enyl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((2-bromocyclopent-1-enyl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-morpholino-1-(phenylthio)butan-2-ylamino)-3-nitrobenzenesulfonamide
(R)—N-(7-(4-((2-(4-chlorophenyl)cyclohex-1-enyl)methyl)piperazin-1-yl)quinazolin-4-yl)-4-(4-(dimethylamino)-1-(phenylthio)butan-2-ylamino)-3-(trifluoromethyl)benzenesulfonamide
(R)—N-(7-(4-((2-bromocyclohex-1-enyl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((4-(4-chlorophenyl)-5,6-dihydro-2H-pyran-3-yl)methyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-((2-(4-methoxyphenyl)cyclohex-1-enyl)methyl)piperazin-1-yl)quinazolin-4-yl)-3-nitrobenzenesulfonamide
(R)-4-(4-(dimethylamino)-1-(phenylthio)butan-2-ylamino)-N-(7-(4-((2-(4-fluorophenyl)cyclohex-1-enyl)methyl)piperazin-1-yl)quinazolin-4-yl)-3-nitrobenzenesulfonamide
(R)-4-(4-(dimethylamino)-1-(phenylthio)butan-2-ylamino)-3-nitro-N-(7-(4-((2-phenylcyclohex-1-enyl)methyl)piperazin-1-yl)quinazolin-4-yl)benzenesulfonamide
(R,Z)—N-(7-(4-((2-(4-chlorophenyl)cyclooct-1-enyl)methyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-((2-(4-(methylthio)phenyl)cyclohex-1-enyl)methyl)piperazin-1-yl)quinazolin-4-yl)-3-nitrobenzenesulfonamide
(R,Z)—N-(7-(4-((2-(4-chlorophenyl)cyclohept-1-enyl)methyl)piperazin-1-yl)quinazolin-4-yl)-4-(4-(dimethylamino)-1-(phenylthio)butan-2-ylamino)-3-nitrobenzenesulfonamide
(R,Z)—N-(7-(4-((2-(4-chlorophenyl)cyclohept-1-enyl)methyl)piperazin-1-yl)quinazolin-4-yl)-4-(4-morpholino-1-(phenylthio)butan-2-ylamino)-3-nitrobenzenesulfonamide
(R)—N-(7-(4-((2-(4-chlorophenyl)-5,5-dimethylcyclohex-1-enyl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((2-(4-chlorophenyl)-5,5-dimethylcyclohex-1-enyl)methyl)piperazin-1-yl)quinazolin-4-yl)-4-(4-morpholino-1-(phenylthio)butan-2-ylamino)-3-nitrobenzenesulfonamide
N-(7-(4-(biphenyl-2-ylmethyl)-4-(2-morpholinoethoxy)piperidin-1-yl)quinazolin-4-yl)-4-(2-methyl-1-(phenylthio)propan-2-ylamino)-3-nitrobenzenesulfonamide
N-(7-(4-(biphenyl-2-ylmethyl)-4-(2-morpholinoethoxy)piperidin-1-yl)quinazolin-4-yl)-3-nitro-4-(2-(phenylthio)ethylamino)benzenesulfonamide
N-(7-(4-(biphenyl-2-ylmethyl)-4-(2-(pyrrolidin-1-yl)ethoxy)piperidin-1-yl)quinazolin-4-yl)-4-(2-methyl-1-(phenylthio)propan-2-ylamino)-3-nitrobenzenesulfonamide
N-(7-(4-(biphenyl-2-ylmethyl)-4-(2-(pyrrolidin-1-yl)ethoxy)piperidin-1-yl)quinazolin-4-yl)-3-nitro-4-(2-(phenylthio)ethylamino)benzenesulfonamide
N-(7-(4-(biphenyl-2-ylmethyl)-4-(2-(pyrrolidin-1-yl)ethoxy)piperidin-1-yl)quinazolin-4-yl)-3-nitro-4-(1-(phenylthiomethyl)cyclopentylamino)benzenesulfonamide
N-(7-(4-(biphenyl-2-ylmethyl)-4-(2-(dimethylamino)ethoxy)piperidin-1-yl)quinazolin-4-yl)-4-(2-methyl-1-(phenylthio)propan-2-ylamino)-3-nitrobenzenesulfonamide
N-(7-(4-(biphenyl-2-ylmethyl)-4-(2-(dimethylamino)ethoxy)piperidin-1-yl)quinazolin-4-yl)-3-nitro-4-(2-(phenylthio) ethylamino)benzenesulfonamide
N-(7-(4-(biphenyl-2-ylmethyl)-4-(2-(dimethylamino)ethoxy)piperidin-1-yl)quinazolin-4-yl)-3-nitro-4-(1-(phenylthiomethyl)cyclopentylamino)benzenesulfonamide
N-(7-(4-(biphenyl-2-ylmethyl)-4-(2-(piperidin-1-yl)ethoxy)piperidin-1-yl)quinazolin-4-yl)-4-(2-methyl-1-(phenylthio)propan-2-ylamino)-3-nitrobenzenesulfonamide
N-(7-(4-(biphenyl-2-ylmethyl)-4-(2-(piperidin-1-yl)ethoxy)piperidin-1-yl)quinazolin-4-yl)-3-nitro-4-(2-(phenylthio)ethylamino)benzenesulfonamide
N-(7-(4-(biphenyl-2-ylmethyl)-4-(2-(piperidin-1-yl)ethoxy)piperidin-1-yl)quinazolin-4-yl)-3-nitro-4-(1-(phenylthiomethyl)cyclopentylamino)benzenesulfonamide
(S)—N-(7-(4-(biphenyl-2-ylmethyl)piperazin-1-yl)quinazolin-4-yl)-4-(4-morpholino-1-(phenylthio)butan-2-ylamino)-3-nitrobenzenesulfonamide
(R)-4-(4-(dimethylamino)-1-(phenylthio)butan-2-ylamino)-N-(7-(4-((4′-(2-(dimethylamino)ethoxy)biphenyl-2-yl)methyl)piperazin-1-yl)quinazolin-4-yl)-3-nitrobenzenesulfonamide
(R)—N-(7-(4-((4′-(2-(dimethylamino)ethoxy)biphenyl-2-yl)methyl)piperazin-1-yl)quinazolin-4-yl)-4-(4-morpholino-1-(phenylthio)butan-2-ylamino)-3-nitrobenzenesulfonamide
N-(7-(4-((4′-(2-(dimethylamino)ethoxy)biphenyl-2-yl)methyl)piperazin-1-yl)quinazolin-4-yl)-4-(2-methyl-1-(phenylthio)propan-2-ylamino)-3-nitrobenzenesulfonamide
N-(7-(4-((4′-(2-(dimethylamino)ethoxy)biphenyl-2-yl)methyl)piperazin-1-yl)quinazolin-4-yl)-3-nitro-4-(2-(phenylthio)ethylamino)benzenesulfonamide
(R)-4-(4-(dimethylamino)-1-(phenylthio)butan-2-ylamino)-N-(7-(4-((4′-(2-morpholinoethoxy)biphenyl-2-yl)methyl)piperazin-1-yl)quinazolin-4-yl)-3-nitrobenzenesulfonamide
(R)-4-(4-morpholino-1-(phenylthio)butan-2-ylamino)-N-(7-(4-((4′-(2-morpholinoethoxy)biphenyl-2-yl)methyl)piperazin-1-yl)quinazolin-4-yl)-3-nitrobenzenesulfonamide
4-(2-methyl-1-(phenylthio)propan-2-ylamino)-N-(7-(4-((4′-(2-morpholinoethoxy)biphenyl-2-yl)methyl)piperazin-1-yl)quinazolin-4-yl)-3-nitrobenzenesulfonamide
N-(7-(4-((4′-(2-morpholinoethoxy)biphenyl-2-yl)methyl)piperazin-1-yl)quinazolin-4-yl)-3-nitro-4-(2-(phenylthio)ethylamino)benzenesulfonamide
(R)-4-(4-(1H-imidazol-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)-4-(4-(1H-imidazol-1-yl)-1-(phenylthio)butan-2-ylamino)-N-(7-(4-(biphenyl-2-ylmethyl)piperazin-1-yl)quinazolin-4-yl)-3-nitrobenzenesulfonamide
(R)-4-(4-(1H-imidazol-1-yl)-1-(phenylthio)butan-2-ylamino)-N-(7-(4-(biphenyl-2-ylmethyl)-4-methoxypiperidin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-4-(5-(4-methylpiperazin-1-yl)-1-(phenylthio)pentan-2-ylamino)-3-nitrobenzenesulfonamide
(R)—N-(7-(4-((4′-chlorobiphenyl-2-yl)methyl)piperazin-1-yl)quinazolin-4-yl)-4-(1-((2-(dimethylamino)ethyl)(methyl)amino)-3-(phenylthio)propan-2-ylamino)-3-nitrobenzenesulfonamide
(R)-4-(4-(N-(7-(4-((4′-chlorobiphenyl-2-yl)methyl)piperazin-1-yl)quinazolin-4-yl)sulfamoyl)-2-nitrophenylamino)-N,N-dimethyl-5-(phenylthio)pentanamide
(R)—N-(7-(4-((4′-chlorobiphenyl-2-yl)methyl)piperazin-1-yl)quinazolin-4-yl)-4-(5-(dimethylamino)-1-(phenylsulfonyl)pentan-2-ylamino)-3-nitrobenzenesulfonamide
(R)-2-((3-(4-(N-(7-(4-((4′-chlorobiphenyl-2-yl)methyl)piperazin-1-yl)quinazolin-4-yl)sulfamoyl)-2-nitrophenylamino)-4-(phenylthio)butyl) (methyl)amino)-N,N-dimethylacetamide
(R)—N-tert-butyl-3-(4-(N-(7-(4-((4′-chlorobiphenyl-2-yl)methyl)piperazin-1-yl)quinazolin-4-yl)sulfamoyl)-2-nitrophenylamino)-4-(phenylthio)butanamide
(R)-3-(4-(N-(7-(4-((4′-chlorobiphenyl-2-yl)methyl)piperazin-1-yl)quinazolin-4-yl)sulfamoyl)-2-nitrophenylamino)-N,N-diisopropyl-4-(phenylthio)butanamide
(R)—N-tert-butyl-3-(4-(N-(7-(4-((4′-chlorobiphenyl-2-yl)methyl)piperazin-1-yl)quinazolin-4-yl)sulfamoyl)-2-nitrophenylamino)-N-methyl-4-(phenylthio)butanamide
(R)-3-(4-(N-(7-(4-((4′-chlorobiphenyl-2-yl)methyl)piperazin-1-yl)quinazolin-4-yl)sulfamoyl)-2-nitrophenylamino)-N-isopropyl-N-methyl-4-(phenylthio)butanamide
(R)—N-(7-(4-((4′-chlorobiphenyl-2-yl)methyl)piperazin-1-yl)quinazolin-4-yl)-3-nitro-4-(4-oxo-1-(phenylthio)-4-(piperidin-1-yl)butan-2-ylamino)benzenesulfonamide
(R)—N-(5-(4-(N-(7-(4-((4′-chlorobiphenyl-2-yl)methyl)piperazin-1-yl)quinazolin-4-yl)sulfamoyl)-2-nitrophenylamino)-6-(phenylthio)hexyl)-2-(dimethylamino) acetamide
(R)-3-(4-(N-(7-(4-((4′-chlorobiphenyl-2-yl)methyl)piperazin-1-yl)quinazolin-4-yl)sulfamoyl)-2-nitrophenylamino)-N,N-dimethyl-4-(phenylthio)butanamide
(R)-3-(4-(N-(7-(4-((4′-chlorobiphenyl-2-yl)methyl)piperazin-1-yl)quinazolin-4-yl)sulfamoyl)-2-nitrophenylamino)-4-(phenylthio)butanamide
(R)-3-(4-(N-(7-(4-((4′-chlorobiphenyl-2-yl)methyl)piperazin-1-yl)quinazolin-4-yl)sulfamoyl)-2-nitrophenylamino)-N-cyclopropyl-4-(phenylthio)butanamide
(R)-3-(4-(N-(7-(4-((4′-chlorobiphenyl-2-yl)methyl)piperazin-1-yl)quinazolin-4-yl)sulfamoyl)-2-nitrophenylamino)-N-cyclobutyl-4-(phenylthio)butanamide
(R)—N-(7-(4-((4′-chlorobiphenyl-2-yl)methyl)piperazin-1-yl)quinazolin-4-yl)-4-(4-(4-methylpiperazin-1-yl)-4-oxo-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-morpholino-4-oxo-1-(phenylthio)butan-2-ylamino)-3-nitrobenzenesulfonamide
(R)-4-(4-(azetidin-1-yl)-4-oxo-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)-3-(4-(N-(7-(4-((4′-chlorobiphenyl-2-yl)methyl)piperazin-1-yl)quinazolin-4-yl)sulfamoyl)-2-nitrophenylamino)-N-(2-morpholinoethyl)-4-(phenylthio)butanamide
(R)-3-(4-(N-(7-(4-((4′-chlorobiphenyl-2-yl)methyl)piperazin-1-yl)quinazolin-4-yl)sulfamoyl)-2-nitrophenylamino)-N-methyl-4-(phenylthio)butanamide
(R)-4-(4-amino-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-4-(4-cyano-1-(phenylthio)butan-2-ylamino)-3-nitrobenzenesulfonamide
(R)-4-(4-(tert-butylamino)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-4-(4-(cyclopropylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(cyclobutylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(diethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(isopropyl(methyl)amino)-1-(phenylthio)butan-2-ylamino)-3-nitrobenzenesulfonamide
(R)-4-(4-(tert-butyl (methyl)amino)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(piperidin-1-yl)butan-2-ylamino)benzenesulfonamide
(R)—N-(7-(4-((4′-chlorobiphenyl-2-yl)methyl)piperazin-1-yl)quinazolin-4-yl)-4-(4-(4-hydroxypiperidin-1-yl)-1-(phenylthio)butan-2-ylamino)-3-nitrobenzenesulfonamide
(R)-4-(4-(4-acetylpiperazin-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-thiomorpholinobutan-2-ylamino)benzenesulfonamide
(R)—N-(7-(4-((4′-chlorobiphenyl-2-yl)methyl)piperazin-1-yl)quinazolin-4-yl)-4-(4-(2-morpholinoethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(piperazin-1-yl)butan-2-ylamino)benzenesulfonamide
N-(7-(4-((4′-chlorobiphenyl-2-yl)methyl)piperazin-1-yl)quinazolin-4-yl)-4-((R)-4-((R)-3-hydroxypyrrolidin-1-yl)-1-(phenylthio)butan-2-ylamino)-3-nitrobenzenesulfonamide
4-((R)-4-((R)-3-aminopyrrolidin-1-yl)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-4-(4-(3-hydroxyazetidin-1-yl)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(4-methylpiperazin-1-yl)-1-(phenylthio)butan-2-ylamino)-3-nitrobenzenesulfonamide
(R)-4-(4-(benzo[d][1,3]dioxol-5-ylamino)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)-4-(4-(benzo[d][1,3]dioxol-4-ylmethylamino)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(pyridin-2-ylmethylamino)butan-2-ylamino)benzenesulfonamide
(R)—N-(7-(4-((4′-chlorobiphenyl-2-yl)methyl)piperazin-1-yl)quinazolin-4-yl)-3-nitro-4-(1-(phenylthio)-4-(2-(pyridin-2-yl)ethylamino)butan-2-ylamino)benzenesulfonamide
(R)—N-(7-(4-((4′-chlorobiphenyl-2-yl)methyl)piperazin-1-yl)quinazolin-4-yl)-3-nitro-4-(1-(phenylthio)-4-(pyridin-4-ylmethylamino)butan-2-ylamino)benzenesulfonamide
(R)—N-(7-(4-((4′-chlorobiphenyl-2-yl)methyl)piperazin-1-yl)quinazolin-4-yl)-4-(4-(morpholinoamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(methyl(pyridin-4-yl)amino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(pyridin-3-ylamino)butan-2-ylamino)benzenesulfonamide
N-(7-(4-((4′-chlorobiphenyl-2-yl)methyl)piperazin-1-yl)quinazolin-4-yl)-4-((2R)-4-(2,6-dimethylpiperidin-1-yl)-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-((R)-4-((2R,6S)-2,6-dimethylpiperidin-1-yl)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(pyrrolidin-1-ylamino)butan-2-ylamino)benzenesulfonamide
(R)—N-(7-(4-((4′-chlorobiphenyl-2-yl)methyl)piperazin-1-yl)quinazolin-4-yl)-4-(4-(4-(methoxyimino)piperidin-1-yl)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-3-nitro-4-(1-(phenylthio)-4-(2H-tetrazol-5-yl)butan-2-ylamino)benzenesulfonamide
(R)—N-(7-(4-((4′-chlorobiphenyl-2-yl)methyl)piperazin-1-yl)quinazolin-4-yl)-4-(4-(diisopropylamino)-1-(phenylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-4-(4-(isopropylamino)-1-(phenylthio)butan-2-ylamino)-3-(trifluoromethyl)benzenesulfonamide
(R)-4-(4-(bis(2-hydroxyethyl)amino)-1-(phenylthio)butan-2-ylamino)-N-(7-(4-((4′-chlorobiphenyl-2-yl)methyl)piperazin-1-yl)quinazolin-4-yl)-3-nitrobenzenesulfonamide
(R)—N-(7-(4-((4′-chlorobiphenyl-2-yl)methyl)piperazin-1-yl)quinazolin-4-yl)-3-(4-(dimethylamino)-1-(phenylthio)butan-2-ylamino)-4-(trifluoromethoxy)benzenesulfonamide
(R)—N-(7-(4-((4′-chlorobiphenyl-2-yl)methyl)piperazin-1-yl)quinazolin-4-yl)-4-(4-(isopropyl(methyl)amino)-1-(phenylthio)butan-2-ylamino)-3-(trifluoromethyl)benzenesulfonamide
(R)—N-(7-(4-((4′-chlorobiphenyl-2-yl)methyl)piperazin-1-yl)quinazolin-4-yl)-4-(4-(diethylamino)-1-(phenylthio)butan-2-ylamino)-3-(trifluoromethyl)benzenesulfonamide
N-(7-(4-((4′-chlorobiphenyl-2-yl)methyl)piperazin-1-yl)quinazolin-4-yl)-4-((2R)-4-(2,5-dimethylpyrrolidin-1-yl)-1-(phenylthio)butan-2-ylamino)-3-nitrobenzenesulfonamide
(R)-4-(4-amino-1-(phenylthio)butan-2-ylamino)-N-(7-(4-((4′-chlorobiphenyl-2-yl)methyl)piperazin-1-yl)quinazolin-4-yl)-3-(trifluoromethyl)benzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-2-(trifluoromethyl)benzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-fluorobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-2-(trifluoromethoxy)benzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-2,5-difluorobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-methylbenzenesulfonamide
(R)—N-(7-(4-((2-(4-chlorophenyl)cyclohex-1-enyl)methyl)piperazin-1-yl)quinazolin-4-yl)-4-(4-(diisopropylamino)-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-((R)-4-((2R,5R)-2,5-dimethylpyrrolidin-1-yl)-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-((R)-4-((2S,5S)-2,5-dimethylpyrrolidin-1-yl)-1-(phenylthio)butan-2-ylamino)-3-nitrobenzenesulfonamide
N-(7-(4-((4′-chlorobiphenyl-2-yl)methyl)piperazin-1-yl)quinazolin-4-yl)-4-((R)-4-((2R,5S)-2,5-dimethylpyrrolidin-1-yl)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-nitro-5-(trifluoromethyl)benzenesulfonamide
(R)—N-(7-(4-((2-(4-chlorophenyl)cyclohex-1-enyl)methyl)piperazin-1-yl)quinazolin-4-yl)-4-(4-(dimethylamino)-1-(phenylthio)butan-2-ylamino)-3-nitro-5-(trifluoromethyl)benzenesulfonamide
(R,Z)—N-(7-(4-((2-(4-chlorophenyl)cyclohept-1-enyl)methyl)piperazin-1-yl)quinazolin-4-yl)-4-(4-(dimethylamino)-1-(phenylthio)butan-2-ylamino)-3-nitro-5-(trifluoromethyl)benzenesulfonamide
(R)—N-(7-(4-((4′-chlorobiphenyl-2-yl)methyl)piperazin-1-yl)quinazolin-4-yl)-3-(4-(dimethylamino)-1-(phenylthio)butan-2-ylamino)-4-nitrobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3,5-difluorobenzenesulfonamide
(R)-methyl 5-(N-(7-(4-((4′-chlorobiphenyl-2-yl)methyl)piperazin-1-yl)quinazolin-4-yl)sulfamoyl)-2-(4-(dimethylamino)-1-(phenylthio)butan-2-ylamino)-3-nitrobenzoate
(R)-5-(N-(7-(4-((4′-chlorobiphenyl-2-yl)methyl)piperazin-1-yl)quinazolin-4-yl)sulfamoyl)-2-(4-(dimethylamino)-1-(phenylthio)butan-2-ylamino)-3-nitrobenzoic acid
(R)-5-(N-(7-(4-((4′-chlorobiphenyl-2-yl)methyl)piperazin-1-yl)quinazolin-4-yl)sulfamoyl)-2-(4-(dimethylamino)-1-(phenylthio)butan-2-ylamino)-3-nitrobenzoic acid
(R)-5-(N-(7-(4-((2-(4-chlorophenyl)cyclohex-1-enyl)methyl)piperazin-1-yl)quinazolin-4-yl)sulfamoyl)-2-(4-(dimethylamino)-1-(phenylthio)butan-2-ylamino)-3-nitrobenzoic acid
(R)-5-(N-(7-(4-((2-(4-chlorophenyl)cyclohex-1-enyl)methyl)piperazin-1-yl)quinazolin-4-yl)sulfamoyl)-2-(4-(dimethylamino)-1-(phenylthio)butan-2-ylamino)-3-nitrobenzamide
(R)-5-(N-(7-(4-((4′-chlorobiphenyl-2-yl)methyl)piperazin-1-yl)quinazolin-4-yl)sulfamoyl)-2-(4-(dimethylamino)-1-(phenylthio)butan-2-ylamino)-3-nitrobenzamide
(R)-methyl 5-(N-(7-(4-((2-(4-chlorophenyl)cyclohex-1-enyl)methyl)piperazin-1-yl)quinazolin-4-yl)sulfamoyl)-2-(4-(dimethylamino)-1-(phenylthio)butan-2-ylamino)-3-(trifluoromethyl)benzoate
(R)-methyl 5-(N-(7-(4-((4-(4-chlorophenyl)-5,6-dihydro-2H-pyran-3-yl)methyl)piperazin-1-yl)quinazolin-4-yl)sulfamoyl)-2-(4-(dimethylamino)-1-(phenylthio)butan-2-ylamino)-3-(trifluoromethyl)benzoate
(R)-methyl 5-(N-(7-(4-((4′-chlorobiphenyl-2-yl)methyl)piperazin-1-yl)quinazolin-4-yl)sulfamoyl)-2-(4-(dimethylamino)-1-(phenylthio)butan-2-ylamino)-3-(trifluoromethyl)benzoate
N-(7-(4-((2-(4-chlorophenyl)cyclohex-1-enyl)methyl)piperazin-1-yl)quinazolin-4-yl)-4-((R)-5-((2R,5S)-2,5-dimethylpyrrolidin-1-yl)-1-(phenylthio)pentan-2-ylamino)-3-nitrobenzenesulfonamide
N-(7-(4-((4-(4-chlorophenyl)-5,6-dihydro-2H-pyran-3-yl)methyl)piperazin-1-yl)quinazolin-4-yl)-4-((R)-5-((2R,5S)-2,5-dimethylpyrrolidin-1-yl)-1-(phenylthio)pentan-2-ylamino)-3-nitrobenzenesulfonamide
(R)-tert-butyl 3-(4-(4-(4-(4-(dimethylamino)-1-(phenylthio)butan-2-ylamino)-3-nitrophenylsulfonamido)quinazolin-7-yl)piperazine-1-carbonyl)phenylcarbamate
(R)-4-(4-(dimethylamino)-1-(phenylthio)butan-2-ylamino)-N-(7-(4-(3-(dimethylamino)benzoyl)piperazin-1-yl)quinazolin-4-yl)-3-nitrobenzenesulfonamide
(R)—N-(7-(4-(biphenyl-2-ylmethyl)piperazin-1-yl)quinazolin-4-yl)-4-(4-(dimethylamino)-2-methyl-1-(phenylthio)butan-2-ylamino)-3-nitrobenzenesulfonamide
(S)—N-(7-(4-(biphenyl-2-ylmethyl)piperazin-1-yl)quinazolin-4-yl)-4-(4-(dimethylamino)-2-methyl-1-(phenylthio)butan-2-ylamino)-3-nitrobenzenesulfonamide
(R)-4-(4-(dimethylamino)-1-(phenylthio)butan-2-ylamino)-N-(7-(4-(2-(isoindolin-2-yl)benzyl)piperazin-1-yl)quinazolin-4-yl)-3-nitrobenzenesulfonamide
(R)—N-(7-(4-(2-(cyclohexylamino)benzyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-N-(7-(4-(2-(isopropylamino)benzyl)piperazin-1-yl)quinazolin-4-yl)-3-nitrobenzenesulfonamide
(R)—N-(7-(4-(2-(benzylamino)benzyl)piperazin-1-yl)quinazolin-4-yl)-4-(4-(dimethylamino)-1-(phenylthio)butan-2-ylamino)-3-nitrobenzenesulfonamide
(R)-4-(4-(dimethylamino)-1-(phenylthio)butan-2-ylamino)-3-nitro-N-(7-(4-(2-(piperidin-1-yl)benzyl)piperazin-1-yl)quinazolin-4-yl)benzenesulfonamide
N-(7-(4-(biphenyl-2-ylmethyl)piperazin-1-yl)quinazolin-4-yl)-3-nitro-4-(2-(phenylthio) ethylamino)benzenesulfonamide
N-(7-(4-(biphenyl-2-ylmethyl)piperazin-1-yl)quinazolin-4-yl)-4-(cyclohexylmethylamino)-3-nitrobenzenesulfonamide
(R)—N-(7-(4-(biphenyl-2-ylmethyl)-4-methoxypiperidin-1-yl)quinazolin-4-yl)-4-(4-morpholino-1-(phenylthio)butan-2-ylamino)-3-nitrobenzenesulfonamide
N-(7-(4-(biphenyl-2-ylmethyl)-4-methoxypiperidin-1-yl)quinazolin-4-yl)-3-nitro-4-(2-(phenylthio) ethylamino)benzenesulfonamide
(S)—N-(7-(4-((4′-chlorobiphenyl-2-yl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(1-(phenylthio)-4-(pyrrolidin-1-yl)butan-2-ylamino)-3-(trifluoromethyl)benzenesulfonamide
(R)—N-(7-(4-((4-(4-chlorophenyl)pyridin-3-yl)methyl)piperazin-1-yl)quinazolin-4-yl)-4-(4-(dimethylamino)-1-(phenylthio)butan-2-ylamino)-3-nitrobenzenesulfonamide
(R)—N-(7-(4-((4-(4-chlorophenyl)pyridin-3-yl)methyl)piperazin-1-yl)quinazolin-4-yl)-4-(4-morpholino-1-(phenylthio)butan-2-ylamino)-3-nitrobenzenesulfonamide
(R)—N-(7-(4-((4′-chlorobiphenyl-2-yl)methyl)piperazin-1-yl)quinazolin-4-yl)-4-(4-(dimethylamino)-1-(phenylthio)butan-2-ylamino)-2-fluoro-3-(trifluoromethyl)benzenesulfonamide

Still another embodiment pertains to compositions for treating diseases during which are expressed one or more than one of antiapoptotic Bcl-X_{L} protein, antiapoptotic Bcl-2 protein or antiapoptotic Bcl-w protein, said compositions comprising an excipient and a therapeutically effective amount of the compound having Formula I.

Still another embodiment pertains to methods of treating disease in a patient during which is expressed one or more than one of antiapoptotic Bcl-X_{L} protein, antiapoptotic Bcl-2 protein or antiapoptotic Bcl-w protein, said methods comprising administering to the patient a therapeutically effective amount of a compound having Formula I.

Still another embodiment pertains to compositions for treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer, said compositions comprising an excipient and a therapeutically effective amount of the compound having Formula I.

Still another embodiment pertains to methods of treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer in a patient, said methods comprising administering to the patient a therapeutically effective amount of a compound having Formula I.

Still another embodiment pertains to compositions for treating diseases during which are expressed one or more than one of antiapoptotic Bcl-X_{L} protein, antiapoptotic Bcl-2 protein or antiapoptotic Bcl-w protein, said compositions comprising an excipient and a therapeutically effective amount of the compound having Formula I and a therapeutically effective amount of one additional therapeutic agent or more than one additional therapeutic agent.

Still another embodiment pertains to methods of treating disease in a patient during which is expressed one or more than one of antiapoptotic Bcl-X_{L} protein, antiapoptotic Bcl-2 protein or antiapoptotic Bcl-w protein, said methods comprising administering to the patient a therapeutically effective amount of a compound having Formula I and a therapeutically effective amount of one additional therapeutic agent or more than one additional therapeutic agent.

Still another embodiment pertains to compositions for treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer, said compositions comprising an excipient and a therapeutically effective amount of the compound having Formula I and a therapeutically effective amount of one additional therapeutic agent or more than one additional therapeutic agent.

Still another embodiment pertains to methods of treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer in a patient, said methods comprising administering to the patient a therapeutically effective amount of the compound having Formula I and a therapeutically effective amount of one additional therapeutic agent or more than one additional therapeutic agent.

Still another embodiment pertains to treating inflammation related to autoimmune hypersensitivity using compounds of Formula I.

### DETAILED DESCRIPTION OF THE INVENTION

### Definitions

Variable moieties herein are represented by identifiers (capital letters with numerical and/or alphabetical superscripts) and may be specifically embodied.

It is meant to be understood that proper valences are maintained for all moieties and combinations thereof, that monovalent moieties having more than one atom are drawn from left to right and are attached through their left ends, and that divalent moieties are also drawn from left to right.

It is also meant to be understood that a specific embodiment of a variable moiety herein may be the same or different as another specific embodiment having the same identifier.

The term “cyclic moiety,” as used herein in reference to compounds of the invention, means arene, aryl, cycloalkane, cycloalkyl, cycloalkene, cycloalkenyl, heteroarene, heteroaryl, heterocycloalkane, heterocycloalkyl, heterocycloalkene, heterocycloalkenyl, spiroalkyl, spiroalkenyl, spiroheteroalkyl and spiroheteroalkenyl.

The term “arene,” as used herein in reference to compounds of the invention, means benzene.

The term “aryl,” as used herein in reference to compounds of the invention, means phenyl.

The term “cycloalkane,” as used herein in reference to compounds of the invention, means C₃-cycloalkane, C₄-cycloalkane, C₅-cycloalkane, C₆-cycloalkane, C₇-cycloalkane, C₈-cycloalkane, C₉-cycloalkane, C₁₀-cycloalkane, C₁₁-cycloalkane, C₁₂-cycloalkane, C₁₃-cycloalkane and C₁₄-cycloalkane.

The term “cycloalkyl,” as used herein in reference to compounds of the invention, means C₃-cycloalkyl, C₄-cycloalkyl, C₅-cycloalkyl C₆-cycloalkyl, C₇-cycloalkyl, C₈-cycloalkyl, C₉-cycloalkyl, C₁₀-cycloalkyl, C₁₁-cycloalkyl, C₁₂-cycloalkyl, C₁₃-cycloalkyl and C₁₄-cycloalkyl.

The term “cycloalkene,” as used herein in reference to compounds of the invention, means C₄-cycloalkene, C₅-cycloalkene, C₆-cycloalkene, C₇-cycloalkene, C₈-cycloalkene, C₉-cycloalkene, C₁₀-cycloalkene, C₁₁-cycloalkene, C₁₂-cycloalkene, C₁₃-cycloalkene and C₁₄-cycloalkene.

The term “cycloalkenyl,” as used herein in reference to compounds of the invention, means C₃-cycloalkenyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, C₁₁-cycloalkenyl, C₁₂-cycloalkenyl, C₁₃-cycloalkenyl and C₁₄-cycloalkenyl.

The term “heteroarene,” as used herein in reference to compounds of the invention, means furan, imidazole, isothiazole, isoxazole, 1,2,3-oxadiazole, 1,2,5-oxadiazole, oxazole, pyrazine, pyrazole, pyridazine, pyridine, pyrimidine, pyrrole, triazole, thiophene, triazine and 1,2,3-triazole.

The term “heteroaryl,” as used herein in reference to compounds of the invention, means furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl and 1,2,3-triazolyl.

The term “heterocycloalkane,” as used herein in reference to compounds of the invention, means cycloalkane having one or two or three CH₂ moieties replaced with independently selected O, C(O), CNOH, CNOCH₃, S, S(O), SO₂ or NH and one or two CH moieties unreplaced or replaced with N and also means cycloalkane having one or two or three CH₂ moieties unreplaced or replaced with independently selected O, C(O), CNOH, CNOCH₃, S, S(O), SO₂ or NH and one or two CH moieties replaced with N.

The term “heterocycloalkyl,” as used herein in reference to compounds of the invention, means cycloalkyl having one or two or three CH₂ moieties replaced with independently selected O, C(O), CNOH, CNOCH₃, S, S(O), SO₂ or NH and one or two CH moieties unreplaced or replaced with N and also means cycloalkyl having one or two or three CH₂ moieties unreplaced or replaced with independently selected O, C(O), CNOH, CNOCH₃, S, S(O), SO₂ or NH and one or two CH moieties replaced with N.

The term “heterocycloalkene,” as used herein in reference to compounds of the invention, means cycloalkene having one or two or three CH₂ moieties replaced with independently selected O, C(O), CNOH, CNOCH₃, S, S(O), SO₂ or NH and one or two CH moieties unreplaced or replaced with N and also means cycloalkene having one or two or three CH₂ moieties unreplaced or replaced with independently selected O, C(O), CNOH, CNOCH₃, S, S(O), SO₂ or NH and one or two CH moieties replaced with N.

The term “heterocycloalkenyl,” as used herein in reference to compounds of the invention, means cycloalkenyl having one or two or three CH₂ moieties replaced with independently selected O, C(O), CNOH, CNOCH₃, S, S(O), SO₂ or NH and one or two CH moieties unreplaced or replaced with N and also means cycloalkenyl having one or two or three CH₂ moieties unreplaced or replaced with independently selected O, C(O), CNOH, CNOCH₃, S, S(O), SO₂ or NH and one or two CH moieties replaced with N.

The term “spiroalkyl,” as used herein in reference to compounds of the invention, means C₂-spiroalkyl, C₃-spiroalkyl, C₄-spiroalkyl, C₅-spiroalkyl, C₆-spiroalkyl, C₇-spiroalkyl, C₈-spiroalkyl and C₉-spiroalkyl.

The term “spiroalkenyl,” as used herein in reference to compounds of the invention, means C₂-spiroalkenyl, C₃-spiroalkenyl, C₄-spiroalkenyl, C₅-spiroalkenyl, C₆-spiroalkenyl, C₇-spiroalkenyl, C₈-spiroalkenyl and C₉-spiroalkenyl.

The term “spiroheteroalkyl,” as used herein in reference to compounds of the invention, means spiroalkyl having one or two CH₂ moieties replaced with independently selected O, C(O), CNOH, CNOCH₃, S, S(O), SO₂ or NH.

The term “spiroheteroalkenyl,” as used herein in reference to compounds of the invention, means spiroalkenyl having one or two CH₂ moieties replaced with independently selected O, C(O), CNOH, CNOCH₃, S, S(O), SO₂ or NH and one or two CH moieties unreplaced or replaced with N and also means spiroalkenyl having one or two CH₂ moieties unreplaced or replaced with independently selected O, C(O), CNOH, CNOCH₃, S, S(O), SO₂ or NH and one or two CH moieties replaced with N.

The term “alkenyl,” as used herein in reference to compounds of the invention, means C₂-alkenyl, C₃-alkenyl, C₄-alkenyl, C₅-alkenyl and C₆-alkenyl.

The term “alkyl,” as used herein in reference to compounds of the invention, means C₁-alkyl, C₂-alkyl, C₃-alkyl, C₄-alkyl, C₅-alkyl and C₆-alkyl.

The term “alkynyl,” as used herein in reference to compounds of the invention, means C₂-alkynyl, C₃-alkynyl, C₄-alkynyl, C₅-alkynyl and C₆-alkynyl.

The term “C₂-alkenyl,” as used herein in reference to compounds of the invention, means ethenyl (vinyl).

The term “C₃-alkenyl,” as used herein in reference to compounds of the invention, means 1-propen-1-yl, 1-propen-2-yl (isopropenyl) and 1-propen-3-yl (allyl).

The term “C₄-alkenyl,” as used herein in reference to compounds of the invention, means 1-buten-1-yl, 1-buten-2-yl, 1,3-butadien-1-yl, 1,3-butadien-2-yl, 2-buten-1-yl, 2-buten-2-yl, 3-buten-1-yl, 3-buten-2-yl, 2-methyl-1-propen-1-yl and 2-methyl-2-propen-1-yl.

The term “C₅-alkenyl,” as used herein in reference to compounds of the invention, means 2-methylene-3-buten-1-yl, 2-methylenebut-1-yl, 2-methyl-1-buten-1-yl, 2-methyl-1,3-butadien-1-yl, 2-methyl-2-buten-1-yl, 2-methyl-3-buten-1-yl, 2-methyl-3-buten-2-yl, 3-methyl-1-buten-1-yl, 3-methyl-1-buten-2-yl, 3-methyl-1,3-butadien-1-yl, 3-methyl-1,3-butadien-2-yl, 3-methyl-2-buten-1-yl, 3-methyl-2-buten-2-yl, 3-methyl-3-buten-1-yl, 3-methyl-3-buten-2-yl, 1-penten-1-yl, 1-penten-2-yl, 1-penten-3-yl, 1,3-pentadien-1-yl, 1,3-penta-dien-2-yl, 1,3-pentadien-3-yl, 1,4-pentadien-1-yl, 1,4-pentadien-2-yl, 1,4-pentadien-3-yl, 2-penten-1-yl, 2-penten-2-yl, 2-penten-3-yl, 2,4-pentadien-1-yl, 2,4-pentadien-2-yl, 3-penten-1-yl, 3-penten-2-yl, 4-penten-1-yl and 4-penten-2-yl.

The term “C₆-alkenyl,” as used herein in reference to compounds of the invention, means 2,2-dimethyl-3-buten-1-yl, 2,3-dimethyl-1-buten-1-yl, 2,3-dimethyl-1,3-butadien-1-yl, 2,3-dimethyl-2-buten-1-yl, 2,3-dimethyl-3-buten-1-yl, 2,3-dimethyl-3-buten-2-yl, 3,3-dimethyl-1-buten-1-yl, 3,3-dimethyl-1-buten-2-yl, 2-ethenyl-1,3-butadien-1-yl, 2-ethenyl-2-buten-1-yl, 2-ethyl-1-buten-1-yl, 2-ethyl-1,3-butadien-1-yl, 2-ethyl-2-buten-1-yl, 2-ethyl-3-buten-1-yl, 1-hexen-1-yl, 1-hexen-2-yl, 1-hexen-3-yl, 1,3-hexadien-1-yl, 1,3-hexadien-2-yl, 1,3-hexadien-3-yl, 1,3,5-hexatrien-1-yl, 1,3,5-hexatrien-2-yl, 1,3,5-hexatrien-3-yl, 1,4-hexadien-1-yl, 1,4-hexadien-2-yl, 1,4-hexadien-3-yl, 1,5-hexadien-1-yl, 1,5-hexadien-2-yl, 1,5-hexadien-3-yl, 2-hexen-1-yl, 2-hexen-2-yl, 2-hexen-3-yl, 2,4-hexadien-1-yl, 2,4-hexadien-2-yl, 2,4-hexadien-3-yl, 2,5-hexadien-1-yl, 2,5-hexadien-2-yl, 2,5-hexadien-3-yl, 3-hexen-1-yl, 3-hexen-2-yl, 3-hexen-3-yl, 3,5-hexadien-1-yl, 3,5-hexadien-2-yl, 3,5-hexadien-3-yl, 4-hexen-1-yl, 4-hexen-2-yl, 4-hexen-3-yl, 5-hexen-1-yl, 5-hexen-2-yl, 5-hexen-3-yl, 2-methylene-3-methyl-3-buten-1-yl, 2-methylene-3-methylbut-1-yl, 2-methylene-3-penten-1-yl, 2-methylene-4-penten-1-yl, 2-methylenepent-1-yl, 2-methylenepent-3-yl, 3-methylene-1-penten-1-yl, 3-methylene-1-penten-2-yl, 3-methylenepent-1-yl, 3-methylene-1,4-pentadien-1-yl, 3-methylene-1,4-pentadien-2-yl, 3-methylene-pent-2-yl, 2-methyl-1-penten-1-yl, 2-methyl-1-penten-3-yl, 2-methyl-1,3-pentadien-1-yl, 2-methyl-1,3-pentadien-3-yl, 2-methyl-1,4-pentadien-1-yl, 2-methyl-1,4-pentadien-3-yl, 2-methyl-2-penten-1-yl, 2-methyl-2-penten-3-yl, 2-methyl-2,4-pentadien-1-yl, 2-methyl-2,4-pentadien-3-yl, 2-methyl-3-penten-1-yl, 2-methyl-3-penten-2-yl, 2-methyl-3-penten-3-yl, 2-methyl-4-penten-1-yl, 2-methyl-4-penten-2-yl, 2-methyl-4-penten-3-yl, 3-methyl-1-penten-1-yl, 3-methyl-1-penten-2-yl, 3-methyl-1,3-pentadien-1-yl, 3-methyl-1,3-pentadien-2-yl, 3-methyl-1,4-pentadien-1-yl, 3-methyl-1,4-pentadien-2-yl, 3-methyl-2-penten-1-yl, 3-methyl-2-penten-2-yl, 3-methyl-2,4-pentadien-1-yl, 3-methyl-3-penten-1-yl, 3-methyl-3-penten-2-yl, 3-methyl-4-penten-1-yl, 3-methyl-4-penten-2-yl, 3-methyl-4-penten-3-yl, 4-methyl-1-penten-1-yl, 4-methyl-1-penten-2-yl, 4-methyl-1-penten-3-yl, 4-methyl-1,3-pentadien-1-yl, 4-methyl-1,3-pentadien-2-yl, 4-methyl-1,3-pentadien-3-yl, 4-methyl-1,4-pentadien-1-yl, 4-methyl-1,4-pentadien-2-yl, 4-methyl-1,4-pentadien-3-yl, 4-methylene-2-penten-3-yl, 4-methyl-2-penten-1-yl, 4-methyl-2-penten-2-yl, 4-methyl-2-penten-3-yl, 4-methyl-2,4-pentadien-1-yl, 4-methyl-2,4-pentadien-2-yl, 4-methyl-3-penten-1-yl, 4-methyl-3-penten-2-yl, 4-methyl-3-penten-3-yl, 4-methyl-4-penten-1-yl and 4-methyl-4-penten-2-yl.

The term “C₁-alkyl,” as used herein in reference to compounds of the invention, means methyl.

The term “C₂-alkyl,” as used herein in reference to compounds of the invention, means ethyl.

The term “C₃-alkyl,” as used herein in reference to compounds of the invention, means prop-1-yl and prop-2-yl (isopropyl).

The term “C₄-alkyl,” as used herein in reference to compounds of the invention, means but-1-yl, but-2-yl, 2-methylprop-1-yl and 2-methylprop-2-yl (tert-butyl).

The term “C₅-alkyl,” as used herein in reference to compounds of the invention, means 2,2-dimethylprop-1-yl (neo-pentyl), 2-methylbut-1-yl, 2-methylbut-2-yl, 3-methylbut-1-yl, 3-methylbut-2-yl, pent-1-yl, pent-2-yl and pent-3-yl.

The term “C₆-alkyl,” as used herein in reference to compounds of the invention, means 2,2-dimethylbut-1-yl, 2,3-dimethylbut-1-yl, 2,3-dimethylbut-2-yl, 3,3-dimethylbut-1-yl, 3,3-dimethylbut-2-yl, 2-ethylbut-1-yl, hex-1-yl, hex-2-yl, hex-3-yl, 2-methylpent-1-yl, 2-methylpent-2-yl, 2-methylpent-3-yl, 3-methylpent-1-yl, 3-methylpent-2-yl, 3-methylpent-3-yl, 4-methylpent-1-yl and 4-methylpent-2-yl.

The term “C₂-alkynyl,” as used herein in reference to compounds of the invention, means ethynyl (acetylenyl).

The term “C₂-alkynyl,” as used herein in reference to compounds of the invention, means 1-propyn-1-yl and 2-propyn-1-yl (propargyl).

The term “C₄-alkynyl,” as used herein in reference to compounds of the invention, means 1-butyn-1-yl, 1,3-butadiyn-1-yl, 2-butyn-1-yl, 3-butyn-1-yl and 3-butyn-2-yl.

The term “C₅-alkynyl,” as used herein in reference to compounds of the invention, means 2-methyl-3-butyn-1-yl, 2-methyl-3-butyn-2-yl, 3-methyl-1-butyn-1-yl, 1,3-pentadiyn-1-yl, 1,4-pentadiyn-1-yl, 1,4-pentadiyn-3-yl, 2,4-pentadiyn-1-yl, 1-pentyn-1-yl, 1-pentyn-3-yl, 2-pentyn-1-yl, 3-pentyn-1-yl, 3-pentyn-2-yl, 4-pentyn-1-yl and 4-pentyn-2-yl.

The term “C₆-alkynyl,” as used herein in reference to compounds of the invention, means 2,2-dimethyl-3-butyn-1-yl, 3,3-dimethyl-1-butyn-1-yl, 2-ethyl-3-butyn-1-yl, 2-ethynyl-3-butyn-1-yl, 1-hexyn-1-yl, 1-hexyn-3-yl, 1,3-hexadiyn-1-yl, 1,3,5-hexatriyn-1-yl, 1,4-hexadiyn-1-yl, 1,4-hexadiyn-3-yl, 1,5-hexadiyn-1-yl, 1,5-hexadiyn-3-yl, 2-hexyn-1-yl, 2,5-hexadiyn-1-yl, 3-hexyn-1-yl, 3-hexyn-2-yl, 3,5-hexadiyn-2-yl, 4-hexyn-1-yl, 4-hexyn-2-yl, 4-hexyn-3-yl, 5-hexyn-1-yl, 5-hexyn-2-yl, 5-hexyn-3-yl, 2-methyl-3-pentyn-1-yl, 2-methyl-3-pentyn-2-yl, 2-methyl-4-pentyn-1-yl, 2-methyl-4-pentyn-2-yl, 2-methyl-4-pentyn-3-yl, 3-methyl-1-pentyn-1-yl, 3-methyl-4-pentyn-1-yl, 3-methyl-4-pentyn-2-yl, 3-methyl-1,4-pentadiyn-1-yl, 3-methyl-1,4-pentadiyn-3-yl, 3-methyl-4-pentyn-1-yl, 3-methyl-4-pentyn-3-yl, 4-methyl-1-pentyn-1-yl and 4-methyl-2-pentyn-1-yl.

The term “C₄-cycloalkane,” as used herein in reference to compounds of the invention, means cyclobutane.

The term “C₅-cycloalkane,” as used herein in reference to compounds of the invention, means cyclopentane.

The term “C₆-cycloalkane,” as used herein in reference to compounds of the invention, means cyclohexane.

The term “C₇-cycloalkane,” as used herein in reference to compounds of the invention, means cycloheptane.

The term “C₈-cycloalkane,” as used herein in reference to compounds of the invention, means cyclooctane.

The term “C₉-cycloalkane,” as used herein in reference to compounds of the invention, means cyclononane.

The term “C₁₀-cycloalkane,” as used herein in reference to compounds of the invention, means cyclodecane.

The term “C₁₁-cycloalkane,” as used herein in reference to compounds of the invention, means cycloundecane.

The term “C₁₂-cycloalkane,” as used herein in reference to compounds of the invention, means cyclododecane.

The term “C₁₃-cycloalkane,” as used herein in reference to compounds of the invention, means cyclotridecane.

The term “C₁₄-cycloalkane,” as used herein in reference to compounds of the invention, means cyclotetradecane.

The term “C₄-cycloalkene,” as used herein in reference to compounds of the invention, means cyclobutene and 1,3-cyclobutadiene.

The term “C₅-cycloalkene,” as used herein in reference to compounds of the invention, means cyclopentene and 1,3-cyclopentadiene.

The term “C₆-cycloalkene,” as used herein in reference to compounds of the invention, means cyclohexene, 1,3-cyclohexadiene and 1,4-cyclohexadiene.

The term “C₇-cycloalkene,” as used herein in reference to compounds of the invention, means cycloheptene and 1,3-cycloheptadiene.

The term “C₈-cycloalkene,” as used herein in reference to compounds of the invention, means cyclooctene, 1,3-cyclooctadiene, 1,4-cyclooctadiene, 1,5-cyclooctadiene, 1,3,5-cyclooctatriene and 1,3,6-cyclooctatriene.

The term “C₉-cycloalkene,” as used herein in reference to compounds of the invention, means cyclononene, 1,3-cyclononadiene, 1,4-cyclononadiene, 1,5-cyclononadiene, 1,3,5-cyclononatriene, 1,3,6-cyclononatriene, 1,3,7-cyclononatriene and 1,3,5,7-cyclononatetraene.

The term “C₁₀-cycloalkene,” as used herein in reference to compounds of the invention, means cyclodecene, 1,3-cyclodecadiene, 1,4-cyclodecadiene, 1,5-cyclodecadiene, 1,6-cyclodecadiene, 1,3,5-cyclodecatriene, 1,3,6-cyclodecatriene, 1,3,5,7-cyclodecatetraene, 1,3,5,8-cyclodecatetraene and 1,3,6,8-cyclodecatetraene.

The term “C₁₁-cycloalkene,” as used herein in reference to compounds of the invention, means cycloundecene, 1,3-cycloundecadiene, 1,4-cycloundecadiene, 1,5-cycloundecadiene, 1,6-cycloundecadiene, 1,3,5-cycloundecatriene, 1,3,6-cycloundecatriene, 1,3,7-cycloundecatriene, 1,4,7-cycloundecatriene, 1,4,8-cycloundecatriene, 1,3,5,7-cycloundecatetraene, 1,3,5,8-cycloundecatetraene, 1,3,6,8-cycloundecatetraene and 1,3,5,7,9-cycloundecapentaene.

The term “C₁₂-cycloalkene,” as used herein in reference to compounds of the invention, means cyclododecene, 1,3-cyclododecadiene, 1,4-cyclododecadiene, 1,5-cyclododecadiene, 1,6-cyclododecadiene, 1,7-cyclododecadiene, 1,3,5-cyclododecatriene, 1,3,6-cyclododecatriene, 1,3,7-cyclododecatriene, 1,3,8-cyclododecatriene, 1,4,7-cyclododecatriene, 1,4,8-cyclododecatriene, 1,5,9-cyclododecatriene, 1,3,5,7-cyclododecatetraene, 1,3,5,8-cyclododecatetraene, 1,3,5,9-cyclododecatetraene, 1,3,6,8-cyclododecatetraene, 1,3,6,9-cyclododecatetraene, 1,3,6,10-cyclododecatetraene, 1,3,7,9-cyclododecatetraene, 1,4,7,10-cyclododecatetraene, 1,3,5,7,9-cyclododecapentaene, 1,3,5,7,10-cyclododecapentaene and 1,3,5,8,10-cyclododecapentaene.

The term “C₁₃-cycloalkene,” as used herein in reference to compounds of the invention, means 1,3-cyclotridecadiene, 1,4-cyclotridecadiene, 1,5-cyclotridecadiene, 1,6-cyclotridecadiene, 1,7-cyclotridecadiene, 1,3,5-cyclotridecatriene, 1,3,6-cyclotridecatriene, 1,3,7-cyclotridecatriene, 1,3,8-cyclotridecatriene, 1,4,7-cyclotridecatriene, 1,4,8-cyclotridecatriene, 1,4,9-cyclotridecatriene, 1,5,9-cyclotridecatriene, 1,3,5,7-cyclotridecatetraene, 1,3,5,8-cyclotridecatetraene, 1,3,5,9-cyclotridecatetraene, 1,3,6,8-cyclotridecatetraene, 1,3,6,9-cyclotridecatetraene, 1,3,6,10-cyclotridecatetraene, 1,3,6,11-cyclotridecatetraene, 1,3,7,9-cyclotridecatetraene, 1,3,7,10-cyclotridecatetraene, 1,4,7,10-cyclotridecatetraene, 1,3,6,11-cyclotridecatetraene, 1,3,5,7,9-cyclotridecapentaene, 1,3,5,7,10-cyclotridecapentaene, 1,3,5,8,10-cyclotridecapentaene, 1,3,5,8,11-cyclotridecapentaene, 1,3,6,8,11-cyclotridecapentaene and 1,3,5,7,9,11-cyclotridecahexaene.

The term “C₁₄-cycloalkene,” as used herein in reference to compounds of the invention, means cyclotetradecene, 1,3-cyclotetradecadiene, 1,4-cyclotetradecadiene, 1,5-cyclotetradecadiene, 1,6-cyclotetradecadiene, 1,7-cyclotetradecadiene, 1,8-cyclotetradecadiene, 1,3,5-cyclotetradecatriene, 1,3,6-cyclotetradecatriene, 1,3,7-cyclotetradecatriene, 1,3,8-cyclotetradecatriene, 1,3,9-cyclotetradecatriene, 1,4,7-cyclotetradecatriene, 1,4,8-cyclotetradecatriene, 1,4,9-cyclotetradecatriene, 1,5,9-cyclotetradecatriene, 1,5,10-cyclotetradecatriene, 1,3,5,7-cyclotetradecatetraene, 1,3,5,8-cyclotetradecatetraene, 1,3,5,9-cyclotetradecatetraene, 1,3,5,10-cyclotetradecatetraene, 1,3,6,8-cyclotetradecatetraene, 1,3,6,9-cyclotetradecatetraene, 1,3,6,10-cyclotetradecatetraene, 1,3,6,11-cyclotetradecatetraene, 1,3,6,12-cyclotetradecatetraene, 1,3,7,9-cyclotetradecatetraene, 1,3,7,10-cyclotetradecatetraene, 1,3,7,11-cyclotetradecatetraene, 1,3,8,10-cyclotetradecatetraene, 1,4,7,10-cyclotetradecatetraene, 1,4,7,11-cyclotetradecatetraene, 1,4,8,11-cyclotetradecatetraene, 1,3,5,7,9-cyclotetradecapentaene, 1,3,5,7,10-cyclotetradecapentaene, 1,3,5,7,11-cyclotetradecapentaene, 1,3,5,8,10-cyclotetradecapentaene, 1,3,5,8,11-cyclotetradecapentaene, 1,3,5,8,12-cyclotetradecapentaene, 1,3,5,9,11-cyclotetradecapentaene, 1,3,5,8,11-cyclotetradecapentaene, 1,3,6,8,11-cyclotetradecapentaene, 1,3,6,9,11-cyclotetradecapentaene, 1,3,6,9,12-cyclotetradecapentaene, 1,3,5,8,11-cyclotetradecapentaene, 1,3,5,8,12-cyclotetradecapentaene, 1,3,5,7,9,11-cyclotetradecahexaene, 1,3,5,7,9,12-cyclotetradecahexaene, 1,3,5,7,10,12-cyclotetradecahexaene, 1,3,5,8,10,12-cyclotetradecahexaene and 1,3,5,7,9,11,13-cyclotetradecaheptaene.

The term “C₃-cycloalkenyl,” as used herein in reference to compounds of the invention, means cycloprop-1-en-1-yl and cycloprop-2-en-1-yl.

The term “C₄-cycloalkenyl,” as used herein in reference to compounds of the invention, means cyclobut-1-en-1-yl and cyclobut-2-en-1-yl.

The term “C₅-cycloalkenyl,” as used herein in reference to compounds of the invention, means cyclopent-1-en-1-yl, cyclopent-2-en-1-yl, cyclopent-3-en-1-yl and cyclopenta-1,3-dien-1-yl.

The term “C₆-cycloalkenyl,” as used herein in reference to compounds of the invention, means cyclohex-1-en-1-yl, cyclohex-2-en-1-yl, cyclohex-3-en-1-yl, cyclohexa-1,3-dien-1-yl, cyclohexa-1,4-dien-1-yl, cyclohexa-1,5-dien-1-yl, cyclohexa-2,4-dien-1-yl and cyclohexa-2,5-dien-1-yl.

The term “C₇-cycloalkenyl,” as used herein in reference to compounds of the invention, means bicyclo[2.2.1]hept-2-en-1-yl, bicyclo[2.2.1]hept-2-en-2-yl, bicyclo[2.2.1]hept-2-en-5-yl, bicyclo[2.2.1]hept-2-en-7-yl, bicyclo[2.2.1]hepta-2,5-dien-1-yl, bicyclo[2.2.1]hepta-2,5-dien-2-yl, bicyclo[2.2.1]hepta-2,5-dien-7-yl, cyclohept-1-en-1-yl, cyclohept-2-en-1-yl, cyclohept-3-en-1-yl, cyclohept-4-en-1-yl, cyclohepta-1,3-dien-1-yl, cyclohepta-1,4-dien-1-yl, cyclohepta-1,5-dien-1-yl, cyclohepta-1,6-dien-1-yl, cyclohepta-2,4-dien-1-yl, cyclohepta-2,5-dien-1-yl, cyclohepta-2,6-dien-1-yl, cyclohepta-3,5-dien-1-yl, cyclohepta-1,3,5-trien-1-yl, cyclohepta-1,3,6-trien-1-yl, cyclohepta-1,4,6-trien-1-yl and cyclohepta-2,4,6-trien-1-yl.

The term “C₈-cycloalkenyl,” as used herein in reference to compounds of the invention, means bicyclo[2.2.2]oct-2-en-1-yl, bicyclo[2.2.2]oct-2-en-2-yl, bicyclo[2.2.2]oct-2-en-5-yl, bicyclo[2.2.2]oct-2-en-7-yl, bicyclo[2.2.2]octa-2,5-dien-1-yl, bicyclo[2.2.2]octa-2,5-dien-2-yl, bicyclo[2.2.2]octa-2,5-dien-7-yl, bicyclo[2.2.2]octa-2,5,7-trien-1-yl, bicyclo[2.2.2]octa-2,5,7-trien-2-yl cyclooct-1-en-1-yl, cyclooct-2-en-1-yl, cyclooct-3-en-1-yl, cyclooct-4-en-1-yl, cycloocta-1,3-dien-1-yl, cycloocta-1,4-dien-1-yl, cycloocta-1,5-dien-1-yl, cycloocta-1,6-dien-1-yl, cyclooctal, 7-dien-1-yl, cycloocta-2,4-dien-1-yl, cycloocta-2,5-dien-1-yl, cycloocta-2,6-dien-1-yl, cycloocta-2,7-dien-1-yl, cycloocta-3,5-dien-1-yl, cycloocta-3,6-dien-1-yl, cycloocta-1,3,5-trien-1-yl, cycloocta-1,3,6-trien-1-yl, cycloocta-1,3,7-trien-1-yl, cycloocta-1,4,6-trien-1-yl, cycloocta-1,4,7-trien-1-yl, cycloocta-1,5,7-trien-1-yl, cycloocta-2,4,6-trien-1-yl, cycloocta-2,4,7-trien-1-yl, cycloocta-2,5,7-trien-1-yl and cycloocta-1,3,5,7-tetraen-1-yl.

The term “C₉-cycloalkenyl,” as used herein in reference to compounds of the invention, means cyclonon-1-en-1-yl, cyclonon-2-en-1-yl, cyclonon-3-en-1-yl, cyclonon-4-en-1-yl, cyclonon-5-en-1-yl, cyclonona-1,3-dien-1-yl, cyclonona-1,4-dien-1-yl, cyclonona-1,5-dien-1-yl, cyclonona-1,6-dien-1-yl, cyclonona-1,7-dien-1-yl, cyclonona-1,8-dien-1-yl, cyclonona-2,4-dien-1-yl, cyclonona-2,5-dien-1-yl, cyclonona-2,6-dien-1-yl, cyclonona-2,7-dien-1-yl, cyclonona-2,8-dien-1-yl, cyclonona-3,5-dien-1-yl, cyclonona-3,6-dien-1-yl, cyclonona-3,7-dien-1-yl, cyclonona-4,6-dien-1-yl, cyclonona-1,3,5-trien-1-yl, cyclonona-1,3,6-trien-1-yl, cyclonona-1,3,7-trien-1-yl, cyclonona-1,3,8-trien-1-yl, cyclonona-1,4,6-trien-1-yl, cyclonona-1,4,7-trien-1-yl, cyclonona-1,4,8-trien-1-yl, cyclonona-1,5,7-trien-1-yl, cyclonona-1,5,8-trien-1-yl, cyclonona-1,6,8-trien-1-yl, cyclonona-2,4,8-trien-1-yl, cyclonona-2,4,6-trien-1-yl, cyclonona-2,4,7-trien-1-yl, cyclonona-2,4,8-trien-1-yl, cyclonona-2,5,7-trien-1-yl, cyclonona-2,5,8-trien-1-yl, cyclonona-1,3,5,7-tetraen-1-yl, cyclonona-1,3,5,8-tetraen-1-yl, cyclonona-1,3,6,8-tetraen-1-yl, cyclonona-1,4,6,8-tetraen-1-yl and cyclonona-2,4,6,8-tetraen-1-yl.

The term “C₁₀-cycloalkenyl,” as used herein in reference to compounds of the invention, means cyclodec-1-en-1-yl, cyclodec-2-en-1-yl, cyclodec-3-en-1-yl, cyclodec-4-en-1-yl, cyclodec-5-en-1-yl, cyclodeca-1,3-dien-1-yl, cyclodeca-1,4-dien-1-yl, cyclodeca-1,5-dien-1-yl, cyclodeca-1,6-dien-1-yl, cyclodeca-1,7-dien-1-yl, cyclodeca-1,8-dien-1-yl, cyclodeca-1,9-dien-1-yl, cyclodeca-2,4-dien-1-yl, cyclodeca-2,5-dien-1-yl, cyclodeca-2,6-dien-1-yl, cyclodeca-2,7-dien-1-yl, cyclodeca-2,8-dien-1-yl, cyclodeca-2,9-dien-1-yl, cyclodeca-3,5-dien-1-yl, cyclodeca-3,6-dien-1-yl, cyclodeca-3,7-dien-1-yl, cyclodeca-3,8-dien-1-yl, cyclodeca-4,6-dien-1-yl, cyclodeca-4,7-dien-1-yl, cyclodeca-1,3,5-trien-1-yl, cyclodeca-1,3,6-trien-1-yl, cyclodeca-1,3,7-trien-1-yl, cyclodeca-1,3,8-trien-1-yl, cyclodeca-1,3,9-trien-1-yl, cyclodeca-1,4,6-trien-1-yl, cyclodeca-1,4,7-trien-1-yl, cyclodeca-1,4,8-trien-1-yl, cyclodeca-1,4,9-trien-1-yl, cyclodeca-1,5,7-trien-1-yl, cyclodeca-1,5,8-trien-1-yl, cyclodeca-1,5,9-trien-1-yl, cyclodeca-1,6,8-trien-1-yl, cyclodeca-1,6,9-trien-1-yl, cyclodeca-1,7,9-trien-1-yl, cyclodeca-2,4,6-trien-1-yl, cyclodeca-2,4,7-trien-1-yl, cyclodeca-2,4,8-trien-1-yl, cyclodeca-2,4,9-trien-1-yl, cyclodeca-2,5,7-trien-1-yl, cyclodeca-2,5,8-trien-1-yl, cyclodeca-2,5,9-trien-1-yl, cyclodeca-2,6,8-trien-1-yl, cyclodeca-3,5,7-trien-1-yl, cyclodeca-3,5,8-trien-1-yl, cyclodeca-1,3,5,7-tetraen-1-yl, cyclodeca-1,3,5,8-tetraen-1-yl, cyclodeca-1,3,5,9-tetraen-1-yl, cyclodeca-1,3,6,8-tetraen-1-yl, cyclodeca-1,3,6,9-tetraen-1-yl, cyclodeca-1,3,7,9-tetraen-1-yl, cyclodeca-1,4,6,8-tetraen-1-yl, cyclodeca-1,4,6,9-tetraen-1-yl, cyclodeca-1,4,7,9-tetraen-1-yl, cyclodeca-1,5,7,9-tetraen-1-yl, cyclodeca-2,4,6,8-tetraen-1-yl, cyclodeca-2,4,6,9-tetraen-1-yl, cyclodeca-2,4,7,9-tetraen-1-yl and cyclodeca-1,3,5,7,9-pentaen-1-yl.

The term “C₁₁-cycloalkenyl,” as used herein in reference to compounds of the invention, means cycloundec-1-en-1-yl, cycloundec-2-en-1-yl, cycloundec-3-en-1-yl, cycloundec-4-en-1-yl, cycloundec-5-en-1-yl, cycloundec-6-en-1-yl, cycloundeca-1,3-dien-1-yl, cycloundeca-1,4-dien-1-yl, cycloundeca-1,5-dien-1-yl, cycloundeca-1,6-dien-1-yl, cycloundeca-1,7-dien-1-yl, cycloundeca-1,8-dien-1-yl, cycloundeca-1,9-dien-1-yl, cycloundeca-1,10-dien-1-yl, cycloundeca-2,4-dien-1-yl, cycloundeca-2,5-dien-1-yl, cycloundeca-2,6-dien-1-yl, cycloundeca-2,7-dien-1-yl, cycloundeca-2,8-dien-1-yl, cycloundeca-2,9-dien-1-yl, cycloundeca-2,10-dien-1-yl, cycloundeca-3,5-dien-1-yl, cycloundeca-3,6-dien-1-yl, cycloundeca-3,7-dien-1-yl, cycloundeca-3,8-dien-1-yl, cycloundeca-3,9-dien-1-yl, cycloundeca-4,6-dien-1-yl, cycloundeca-4,7-dien-1-yl, cycloundeca-4,8-dien-1-yl, cycloundeca-5,7-dien-1-yl, cycloundeca-1,3,5-trien-1-yl, cycloundeca-1,3,6-trien-1-yl, cycloundeca-1,3,7-trien-1-yl, cycloundeca-1,3,8-trien-1-yl, cycloundeca-1,3,9-trien-1-yl, cycloundeca-1,3,10-trien-1-yl, cycloundeca-1,4,6-trien-1-yl, cycloundeca-1,4,7-tri-en-1-yl, cycloundeca-1,4,8-trien-1-yl, cycloundeca-1,4,9-trien-1-yl, cycloundeca-1,4,10-trien-1-yl, cycloundeca-1,5,7-trien-1-yl, cycloundeca-1,5,8-trien-1-yl, cycloundeca-1,5,9-trien-1-yl, cycloundeca-1,5,10-trien-1-yl, cycloundeca-1,6,8-trien-1-yl, cycloundeca-1,6,9-trien-1-yl, cycloundeca-1,6,10-trien-1-yl, cycloundeca-1,7,9-trien-1-yl, cycloundeca-1,7,10-trien-1-yl, cycloundeca-1,8,10-trien-1-yl, cycloundeca-2,4,6-trien-1-yl, cycloundeca-2,4,7-trien-1-yl, cycloundeca-2,4,8-trien-1-yl, cycloundeca-2,4,9-trien-1-yl, cycloundeca-2,4,10-trien-1-yl, cycloundeca-2,5,7-trien-1-yl, cycloundeca-2,5,8-trien-1-yl, cycloundeca-2,5,9-trien-1-yl, cycloundeca-2,5,10-tri-en-1-yl, cycloundeca-2,6,8-trien-1-yl, cycloundeca-2,6,9-trien-1-yl, cycloundeca-2,6,10-trien-1-yl, cycloundeca-2,7,9-trien-1-yl, cycloundeca-3,5,7-trien-1-yl, cycloundeca-3,5,8-trien-1-yl, cycloundeca-3,5,9-trien-1-yl, cycloundeca-3,6,8-trien-1-yl, cycloundeca-3,6,9-trien-1-yl, cycloundeca-4,6,8-trien-1-yl, cycloundeca-1,3,5,7-tetraen-1-yl, cycloundeca-1,3,5,8-tetraen-1-yl, cycloundeca-1,3,5,9-tetraen-1-yl, cycloundeca-1,3,5,10-tetraen-1-yl, cycloundeca-1,3,6,8-tetraen-1-yl, cycloundeca-1,3,6,9-tetraen-1-yl, cycloundeca-1,3,6,10-tetraen-1-yl, cycloundeca-1,3,7,9-tetraen-1-yl, cycloundeca-1,3,7,10-tetraen-1-yl, cycloundeca-1,3,8,10-tetraen-1-yl, cycloundeca-1,4,6,8-tetraen-1-yl, cycloundeca-1,4,6,9-tetraen-1-yl, cycloundeca-1,4,6,10-tetraen-1-yl, cycloundeca-1,4,8,10-tetraen-1-yl, cycloundeca-1,5,7,9-tetraen-1-yl, cycloundeca-1,5,7,10-tetraen-1-yl, cycloundeca-1,5,8,10-tetraen-1-yl, cycloundeca-1,6,8,10-tetraen-1-yl, cycloundeca-2,4,6,8-tetraen-1-yl, cycloundeca-2,4,6,9-tetraen-1-yl, cycloundeca-2,4,6,10-tetraen-1-yl, cycloundeca-2,4,7,9-tetraen-1-yl, cycloundeca-2,5,7,9-tetraen-1-yl, cycloundeca-3,5,7,9-tetraen-1-yl, cycloundeca-1,3,5,7,9-pentaenyl, cycloundeca-1,3,5,7,10-pentaenyl, cycloundeca-1,3,5,8,10-pentaenyl, cycloundeca-1,3,6,8,10-pentaenyl, cycloundeca-1,4,6,8,10-pentaenyl and cycloundeca-2,4,6,8,10-pentaenyl.

The term “C₁₂-cycloalkenyl,” as used herein in reference to compounds of the invention, means cyclododec-1-en-1-yl, cyclododec-2-en-1-yl, cyclododec-3-en-1-yl, cyclododec-4-en-1-yl, cyclododec-5-en-1-yl, cyclododec-6-en-1-yl, cyclododeca-1,3-dien-1-yl, cyclododeca-1,4-dien-1-yl, cyclododeca-1,5-dien-1-yl, cyclododeca-1,6-dien-1-yl, cyclododeca-1,7-dien-1-yl, cyclododeca-1,8-dien-1-yl, cyclododeca-1,9-dien-1-yl, cyclododeca-1,10-dien-1-yl, cyclododeca-1,1′-dien-1-yl, cyclododeca-2,4-dien-1-yl, cyclododeca-2,5-dien-1-yl, cyclododeca-2,6-dien-1-yl, cyclododeca-2,7-dien-1-yl, cyclododeca-2,8-dien-1-yl, cyclododeca-2,9-dien-1-yl, cyclododeca-2,10-dien-1-yl, cyclododeca-2,11-dien-1-yl, cyclododeca-3,5-dien-1-yl, cyclododeca-3,6-dien-1-yl, cyclododeca-3,7-dien-1-yl, cyclododeca-3,8-dien-1-yl, cyclododeca-3,9-dien-1-yl, cyclododeca-3,10-dien-1-yl, cyclododeca-3,11-dien-1-yl, cyclododeca-4,6-dien-1-yl, cyclododeca-4,7-dien-1-yl, cyclododeca-4,8-dien-1-yl, cyclododeca-4,9-dien-1-yl, cyclododeca-5,7-dien-1-yl, cyclododeca-5,8-dien-1-yl, cyclododeca-1,3,5-trien-1-yl, cyclododeca-1,3,6-trien-1-yl, cyclododeca-1,3,7-trien-1-yl, cyclododeca-1,3,8-trien-1-yl, cyclododeca-1,3,9-trien-1-yl, cyclododeca-1,3,10-trien-1-yl, cyclododeca-1,3,11-trien-1-yl, cyclododeca-1,4,6-trien-1-yl, cyclododeca-1,4,7-trien-1-yl, cyclododeca-1,4,8-trien-1-yl, cyclododeca-1,4,9-trien-1-yl, cyclododeca-1,4,10-trien-1-yl, cyclododeca-1,4,11-trien-1-yl, cyclododeca-1,5,7-trien-1-yl, cyclododeca-1,5,8-trien-1-yl, cyclododeca-1,5,9-trien-1-yl, cyclododeca-1,5,10-trien-1-yl, cyclododeca-1,5,11-trien-1-yl, cyclododeca-1,6,8-trien-1-yl, cyclododeca-1,6,9-trien-1-yl, cyclododeca-1,6,10-trien-1-yl, cyclododeca-1,6,11-trien-1-yl, cyclododeca-1,7,9-trien-1-yl, cyclododeca-1,7,10-trien-1-yl, cyclododeca-1,7,11-trien-1-yl, cyclododeca-1,8,10-trien-1-yl, cyclododeca-1,8,11-trien-1-yl, cyclododeca-1,9,11-trien-1-yl, cyclododeca-2,4,6-trien-1-yl, cyclododeca-2,4,7-trien-1-yl, cyclododeca-2,4,8-trien-1-yl, cyclododeca-2,4,9-trien-1-yl, cyclododeca-2,4,10-trien-1-yl, cyclododeca-2,4,11-trien-1-yl, cyclododeca-2,5,7-trien-1-yl, cyclododeca-2,5,8-trien-1-yl, cyclododeca-2,5,9-trien-1-yl, cyclododeca-2,5,10-trien-1-yl, cyclododeca-2,5,11-trien-1-yl, cyclododeca-2,6,8-trien-1-yl, cyclododeca-2,6,9-trien-1-yl, cyclododeca-2,6,10-trien-1-yl, cyclododeca-2,6,11-trien-1-yl, cyclododeca-2,7,9-trien-1-yl, cyclododeca-2,7,10-trien-1-yl, cyclododeca-2,8,10-trien-1-yl, cyclododeca-3,5,7-trien-1-yl, cyclododeca-3,5,8-trien-1-yl, cyclododeca-3,5,9-trien-1-yl, cyclododeca-3,5,10-trien-1-yl, cyclododeca-3,6,8-trien-1-yl, cyclododeca-3,6,9-trien-1-yl, cyclododeca-3,6,10-trien-1-yl, cyclododeca-3,7,9-trien-1-yl, cyclododeca-4,6,8-trien-1-yl, cyclododeca-4,6,9-trien-1-yl, cyclododeca-1,3,5,7-tetraen-1-yl, cyclododeca-1,3,5,8-tetraen-1-yl, cyclododeca-1,3,5,9-tetraen-1-yl, cyclododeca-1,3,5,10-tetraen-1-yl, cyclododeca-1,3,5,11-tetraen-1-yl, cyclododeca-1,3,6,8-tetraen-1-yl, cyclododeca-1,3,6,9-tetraen-1-yl, cyclododeca-1,3,6,10-tetraen-1-yl, cyclododeca-1,3,6,11-tetraen-1-yl, cyclododeca-1,3,7,9-tetraen-1-yl, cyclododeca-1,3,7,10-tetraen-1-yl, cyclododeca-1,3,7,11-tetraen-1-yl, cyclododeca-1,3,8,10-tetraen-1-yl, cyclododeca-1,3,8,11-tetraen-1-yl, cyclododeca-1,3,9,11-tetraen-1-yl, cyclododeca-1,4,6,8-tetraen-1-yl, cyclododeca-1,4,6,9-tetraen-1-yl, cyclododeca-1,4,6,10-tetraen-1-yl, cyclododeca-1,4,6,11-tetraen-1-yl, cyclododeca-1,4,7,9-tetraen-1-yl, cyclododeca-1,4,7,10-tetraen-1-yl, cyclododeca-1,4,7,11-tetraen-1-yl, cyclododeca-1,4,8,10-tetraen-1-yl, cyclododeca-1,4,8,11-tetraen-1-yl, cyclododeca-1,4,9,11-tetraen-1-yl, cyclododeca-1,5,7,9-tetraen-1-yl, cyclododeca-1,5,7,10-tetraen-1-yl, cyclododeca-1,5,7,11-tetraen-1-yl, cyclododeca-1,5,8,10-tetraen-1-yl, cyclododeca-1,5,8,11-tetraen-1-yl, cyclododeca-1,5,9,11-tetraen-1-yl, cyclododeca-1,6,8,10-tetraen-1-yl, cyclododeca-1,6,8,11-tetraen-1-yl, cyclododeca-1,6,9,11-tetraen-1-yl, cyclododeca-1,7,9,11-tetraen-1-yl, cyclododeca-2,4,6,8-tetraen-1-yl, cyclododeca-2,4,6,9-tetraen-1-yl, cyclododeca-2,4,6,10-tetraen-1-yl, cyclododeca-2,4,6,11-tetraen-1-yl, cyclododeca-2,4,7,9-tetraen-1-yl, cyclododeca-2,4,7,10-tetraen-1-yl, cyclododeca-2,4,7,11-tetraen-1-yl, cyclododeca-2,4,8,10-tetraen-1-yl, cyclododeca-2,4,8,11-tetraen-1-yl, cyclododeca-2,4,9,11-tetraen-1-yl, cyclododeca-2,5,7,9-tetraen-1-yl, cyclododeca-2,5,7,10-tetraen-1-yl, cyclododeca-2,5,7,11-tetraen-1-yl, cyclododeca-2,5,8,10-tetraen-1-yl, cyclododeca-2,5,8,11-tetraen-1-yl, cyclododeca-2,6,8,10-tetraen-1-yl, cyclododeca-3,5,7,9-tetraen-1-yl, cyclododeca-3,5,7,10-tetraen-1-yl, cyclododeca-3,5,8,10-tetraen-1-yl, cyclododeca-1,3,5,7,9-pentaen-1-yl, cyclododeca-1,3,5,7,10-pentaen-1-yl, cyclododeca-1,3,5,7,11-pentaen-1-yl, cyclododeca-1,3,5,8,10-pentaen-1-yl, cyclododeca-1,3,5,8,11-pentaen-1-yl, cyclododeca-1,3,5,9,11-pentaen-1-yl, cyclododeca-1,3,6,8,10-pentaen-1-yl, cyclododeca-1,3,6,8,11-pentaen-1-yl, cyclododeca-1,3,6,9,11-pentaen-1-yl, cyclododeca-1,3,7,9,11-pentaen-1-yl, cyclododeca-1,4,6,8,10-pentaen-1-yl, cyclododeca-1,4,6,8,11-pentaen-1-yl, cyclododeca-1,4,6,9,11-pentaen-1-yl, cyclododeca-1,4,7,9,11-pentaen-1-yl, cyclododeca-1,5,7,9,11-pentaen-1-yl, cyclododeca-2,4,6,8,10-pentaen-1-yl, cyclododeca-2,4,6,8,11-pentaen-1-yl, cyclododeca-2,4,6,9,11-pentaen-1-yl and cyclododeca-1,3,5,7,9,11-hexaen-1-yl.

The term “C₁₃-cycloalkenyl,” as used herein in reference to compounds of the invention, means cyclotridec-1-en-1-yl, cyclotridec-2-en-1-yl, cyclotridec-3-en-1-yl, cyclotridec-4-en-1-yl, cyclotridec-5-en-1-yl, cyclotridec-6-en-1-yl, cyclotridec-7-en-1-yl, cyclotrideca-1,3-dien-1-yl, cyclotrideca-1,4-dien-1-yl, cyclotrideca-1,5-dien-1-yl, cyclotrideca-1,6-dien-1-yl, cyclotrideca-1,7-dien-1-yl, cyclotrideca-1,8-dien-1-yl, cyclotrideca-1,9-dien-1-yl, cyclotrideca-1,10-dien-1-yl, cyclotrideca-1,1′-dien-1-yl, cyclotrideca-1,12-dien-1-yl, cyclotrideca-2,4-dien-1-yl, cyclotrideca-2,5-dien-1-yl, cyclotrideca-2,6-dien-1-yl, cyclotrideca-2,7-dien-1-yl, cyclotrideca-2,8-dien-1-yl, cyclotrideca-2,9-dien-1-yl, cyclotrideca-2,10-dien-1-yl, cyclotrideca-2,11-dien-1-yl, cyclotrideca-2,12-dien-1-yl, cyclotrideca-3,5-dien-1-yl, cyclotrideca-3,6-dien-1-yl, cyclotrideca-3,7-dien-1-yl, cyclotrideca-3,8-dien-1-yl, cyclotrideca-3,9-dien-1-yl, cyclotrideca-3,10-dien-1-yl, cyclotrideca-3,11-dien-1-yl, cyclotrideca-4,6-dien-1-yl, cyclotrideca-4,7-dien-1-yl, cyclotrideca-4,8-dien-1-yl, cyclotrideca-4,9-dien-1-yl, cyclotrideca-4,10-dien-1-yl, cyclotrideca-5,7-dien-1-yl, cyclotrideca-5,8-dien-1-yl, cyclotrideca-5,9-dien-1-yl, cyclotrideca-6,8-dien-1-yl, cyclotrideca-1,3,5-trien-1-yl, cyclotrideca-1,3,6-trien-1-yl, cyclotrideca-1,3,7-trien-1-yl, cyclotrideca-1,3,8-trien-1-yl, cyclotrideca-1,3,9-trien-1-yl, cyclotrideca-1,3,10-trien-1-yl, cyclotrideca-1,3,11-trien-1-yl, cyclotrideca-1,3,12-trien-1-yl, cyclotrideca-1,4,6-trien-1-yl, cyclotrideca-1,4,7-trien-1-yl, cyclotrideca-1,4,8-trien-1-yl, cyclotrideca-1,4,9-trien-1-yl, cyclotrideca-1,4,10-trien-1-yl, cyclotrideca-1,4,11-trien-1-yl, cyclotrideca-1,4,12-trien-1-yl, cyclotrideca-1,5,7-trien-1-yl, cyclotrideca-1,5,8-trien-1-yl, cyclotrideca-1,5,9-trien-1-yl, cyclotrideca-1,5,10-trien-1-yl, cyclotrideca-1,5,11-trien-1-yl, cyclotrideca-1,5,12-trien-1-yl, cyclotrideca-1,6,8-trien-1-yl, cyclotrideca-1,6,9-trien-1-yl, cyclotrideca-1,6,10-trien-1-yl, cyclotrideca-1,6,11-trien-1-yl, cyclotrideca-1,6,12-trien-1-yl, cyclotrideca-1,7,9-trien-1-yl, cyclotrideca-1,7,10-trien-1-yl, cyclotrideca-1,7,11-trien-1-yl, cyclotrideca-1,7,12-trien-1-yl, cyclotrideca-1,8,10-trien-1-yl, cyclotrideca-1,8,11-trien-1-yl, cyclotrideca-1,8,12-trien-1-yl, cyclotrideca-1,9,11-trien-1-yl, cyclotrideca-1,9,12-trien-1-yl, cyclotrideca-1,10,12-trien-1-yl, cyclotrideca-2,4,6-trien-1-yl, cyclotrideca-2,4,7-trien-1-yl, cyclotrideca-2,4,8-trien-1-yl, cyclotrideca-2,4,9-trien-1-yl, cyclotrideca-2,4,10-trien-1-yl, cyclotrideca-2,4,11-trien-1-yl, cyclotrideca-2,4,12-trien-1-yl, cyclotrideca-2,5,7-trien-1-yl, cyclotrideca-2,5,8-trien-1-yl, cyclotri-deca-2,5,9-trien-1-yl, cyclotri-deca-2,5,10-trien-1-yl, cyclotrideca-2,5,11-trien-1-yl, cyclotrideca-2,5,12-trien-1-yl, cyclotrideca-2,6,8-trien-1-yl, cyclotrideca-2,6,9-trien-1-yl, cyclotrideca-2,6,10-trien-1-yl, cyclotrideca-2,6,11-trien-1-yl, cyclotrideca-2,6,12-trien-1-yl, cyclotrideca-2,7,9-trien-1-yl, cyclotrideca-2,7,10-trien-1-yl, cyclotrideca-2,7,11-trien-1-yl, cyclotrideca-2,7,12-trien-1-yl, cyclotrideca-2,8,10-trien-1-yl, cyclotrideca-2,8,11-trien-1-yl, cyclotrideca-2,8,12-trien-1-yl, cyclotrideca-2,9,11-trien-1-yl, cyclotrideca-2,9,12-trien-1-yl, cyclotrideca-2,10,12-trien-1-yl, cyclotrideca-3,5,7-trien-1-yl, cyclotrideca-3,5,8-trien-1-yl, cyclotrideca-3,5,9-trien-1-yl, cyclotrideca-3,5,10-trien-1-yl, cyclotrideca-3,5,11-trien-1-yl, cyclotrideca-3,6,8-trien-1-yl, cyclotrideca-3,6,9-trien-1-yl, cyclotrideca-3,6,10-trien-1-yl, cyclotrideca-3,6,11-trien-1-yl, cyclotrideca-3,7,9-trien-1-yl, cyclotrideca-3,7,10-trien-1-yl, cyclotrideca-3,7,11-trien-1-yl, cyclotrideca-3,8,10-trien-1-yl, cyclotrideca-4,6,8-trien-1-yl, cyclotrideca-4,6,9-trien-1-yl, cyclotrideca-4,6,10-trien-1-yl, cyclotrideca-4,7,9-trien-1-yl, cyclotrideca-4,7,10-trien-1-yl, cyclotrideca-1,3,5,7-tetraen-1-yl, cyclotrideca-1,3,5,8-tetraen-1-yl, cyclotrideca-1,3,5,9-tetraen-1-yl, cyclotrideca-1,3,5,10-tetraen-1-yl, cyclotrideca-1,3,5,11-tetraen-1-yl, cyclotrideca-1,3,5,12-tetraen-1-yl, cyclotrideca-1,3,6,8-tetraen-1-yl, cyclotrideca-1,3,6,9-tetraen-1-yl, cyclotrideca-1,3,6,10-tetraen-1-yl, cyclotrideca-1,3,6,11-tetraen-1-yl, cyclotrideca-1,3,6,12-tetraen-1-yl, cyclotrideca-1,3,7,9-tetraen-1-yl, cyclotrideca-1,3,7,10-tetraen-1-yl, cyclotrideca-1,3,7,11-tetraen-1-yl, cyclotrideca-1,3,7,12-tetraen-1-yl, cyclotri-deca-1,3,8,10-tetraen-1-yl, cyclotrideca-1,3,8,11-tetraen-1-yl, cyclotrideca-1,3,8,12-tetraen-1-yl, cyclotrideca-1,3,9,11-tetraen-1-yl, cyclotrideca-1,3,9,12-tetraen-1-yl, cyclotrideca-1,3,10,12-tetraen-1-yl, cyclotrideca-1,4,6,8-tetraen-1-yl, cyclotrideca-1,4,6,9-tetraen-1-yl, cyclotri-deca-1,4,6,10-tetraen-1-yl, cyclotrideca-1,4,6,11-tetraen-1-yl, cyclotrideca-1,4,6,12-tetraen-1-yl, cyclotrideca-1,4,7,9-tetraen-1-yl, cyclotrideca-1,4,7,10-tetraen-1-yl, cyclotrideca-1,4,7,11-tetraen-1-yl, cyclotrideca-1,4,7,12-tetraen-1-yl, cyclotri-deca-1,4,8,10-tetraen-1-yl, cyclotrideca-1,4,8,11-tetraen-1-yl, cyclotrideca-1,4,8,12-tetraen-1-yl, cyclotrideca-1,4,9,11-tetraen-1-yl, cyclotrideca-1,4,9,12-tetraen-1-yl, cyclotrideca-1,4,10,12-tetraen-1-yl, cyclotrideca-1,5,7,9-tetraen-1-yl, cyclotrideca-1,5,7,10-tetraen-1-yl, cyclotri-deca-1,5,7,11-tetraen-1-yl, cyclotrideca-1,5,7,12-tetraen-1-yl, cyclotrideca-1,5,8,10-tetraen-1-yl, cyclotrideca-1,5,8,11-tetraen-1-yl, cyclotrideca-1,5,8,12-tetraen-1-yl, cyclotrideca-1,5,9,11-tetraen-1-yl, cyclotrideca-1,5,9,12-tetraen-1-yl, cyclotrideca-1,5,10,12-tetraen-1-yl, cyclotrideca-1,6,8,10-tetraen-1-yl, cyclotrideca-1,6,8,11-tetraen-1-yl, cyclotrideca-1,6,8,12-tetraen-1-yl, cyclotrideca-1,6,9,11-tetraen-1-yl, cyclotrideca-1,6,9,12-tetraen-1-yl, cyclotrideca-1,6,10,12-tetraen-1-yl, cyclotrideca-1,7,9,11-tetraen-1-yl, cyclotrideca-1,7,9,12-tetraen-1-yl, cyclotri-deca-1,7,10,12-tetraen-1-yl, cyclotrideca-1,8,10,12-tetraen-1-yl, cyclotrideca-2,4,6,8-tetraen-1-yl, cyclotrideca-2,4,6,9-tetraen-1-yl, cyclotrideca-2,4,6,10-tetraen-1-yl, cyclotrideca-2,4,6,11-tetraen-1-yl, cyclotrideca-2,4,6,12-tetraen-1-yl, cyclotrideca-2,4,7,9-tetraen-1-yl, cyclotri-deca-2,4,7,10-tetraen-1-yl, cyclotrideca-2,4,7,11-tetraen-1-yl, cyclotrideca-2,4,7,12-tetraen-1-yl, cyclotrideca-2,4,8,10-tetraen-1-yl, cyclotri-deca-2,4,8,11-tetraen-1-yl, cyclotrideca-2,4,8,12-tetraen-1-yl, cyclotrideca-2,4,9,11-tetraen-1-yl, cyclotrideca-2,4,9,12-tetraen-1-yl, cyclotrideca-2,4,10,12-tetraen-1-yl, cyclotrideca-2,5,7,9-tetraen-1-yl, cyclotrideca-2,5,7,10-tetraen-1-yl, cyclotrideca-2,5,7,11-tetraen-1-yl, cyclotrideca-2,5,7,12-tetraen-1-yl, cyclotrideca-2,5,8,10-tetraen-1-yl, cyclotrideca-2,5,8,11-tetraen-1-yl, cyclotrideca-2,5,8,12-tetraen-1-yl, cyclotri-deca-2,5,9,11-tetraen-1-yl, cyclotrideca-2,5,9,12-tetraen-1-yl, cyclotrideca-2,5,10,12-tetraen-1-yl, cyclotrideca-2,6,8,10-tetraen-1-yl, cyclotrideca-2,6,8,11-tetraen-1-yl, cyclotrideca-2,6,8,12-tetraen-1-yl, cyclotrideca-2,6,9,11-tetraen-1-yl, cyclotrideca-2,6,9,12-tetraen-1-yl, cyclotri-deca-2,6,10,12-tetraen-1-yl, cyclotrideca-2,7,9,11-tetraen-1-yl, cyclotrideca-2,7,9,12-tetraen-1-yl, cyclotrideca-2,7,10,12-tetraen-1-yl, cyclotri-deca-3,5,7,9-tetraen-1-yl, cyclotrideca-3,5,7,10-tetraen-1-yl, cyclotrideca-3,5,7,11-tetraen-1-yl, cyclotrideca-3,5,8,10-tetraen-1-yl, cyclotrideca-3,5,8,11-tetraen-1-yl, cyclotrideca-3,5,9,11-tetraen-1-yl, cyclotrideca-3,6,8,10-tetraen-1-yl, cyclotrideca-3,6,8,11-tetraen-1-yl, cyclotrideca-3,7,9,11-tetraen-1-yl, cyclotrideca-1,3,5,7,9-pentaen-1-yl, cyclotrideca-1,3,5,7,10-pentaen-1-yl, cyclotrideca-1,3,5,7,11-pentaen-1-yl, cyclotrideca-1,3,5,7,12-pentaen-1-yl, cyclotrideca-1,3,5,8,10-pentaen-1-yl, cyclotrideca-1,3,5,8,11-pentaen-1-yl, cyclotrideca-1,3,5,8,12-pentaen-1-yl, cyclotrideca-1,3,5,9,11-pentaen-1-yl, cyclotrideca-1,3,5,9,12-pentaen-1-yl, cyclotrideca-1,3,6,8,10-pentaen-1-yl, cyclotrideca-1,3,6,8,11-pentaen-1-yl, cyclotrideca-1,3,6,8,12-pentaen-1-yl, cyclotrideca-1,3,6,9,11-pentaen-1-yl, cyclotrideca-1,3,6,9,12-pentaen-1-yl, cyclotrideca-1,3,7,9,11-pentaen-1-yl, cyclotrideca-1,3,7,9,12-pentaen-1-yl, cyclotrideca-1,4,6,8,10-pentaen-1-yl, cyclotrideca-1,4,6,8,11-pentaen-1-yl, cyclotrideca-1,4,6,8,12-pentaen-1-yl, cyclotrideca-1,4,6,9,11-pentaen-1-yl, cyclotrideca-1,4,6,9,12-pentaen-1-yl, cyclotrideca-1,4,7,9,11-pentaen-1-yl, cyclotrideca-1,4,7,9,12-pentaen-1-yl, cyclotrideca-1,5,7,9,11-pentaen-1-yl, cyclotrideca-1,5,7,9,12-pentaen-1-yl, cyclotrideca-2,4,6,8,10-pentaen-1-yl, cyclotrideca-2,4,6,8,11-pentaen-1-yl, cyclotrideca-2,4,6,8,12-pentaen-1-yl, cyclotrideca-2,4,6,9,11-pentaen-1-yl, cyclotrideca-2,5,7,9,11-pentaen-1-yl, cyclotrideca-2,5,7,9,12-pentaen-1-yl, cyclotrideca-1,3,5,7,9,11-hexaen-1-yl and cyclotrideca-2,4,6,8,10,12-hexaen-1-yl.

The term “C₁₄-cycloalkenyl,” as used herein in reference to compounds of the invention, means cyclotetradec-1-en-1-yl, cyclotetradec-2-en-1-yl, cyclotetradec-3-en-1-yl, cyclotetradec-4-en-1-yl, cyclotetradec-5-en-1-yl, cyclotetradec-6-en-1-yl, cyclotetradec-7-en-1-yl, cyclotetradec-8-en-1-yl, cyclotetradeca-1,3-dien-1-yl, cyclotetradeca-1,4-dien-1-yl, cyclotetradeca-1,5-dien-1-yl, cyclotetradeca-1,6-dien-1-yl, cyclotetradeca-1,7-dien-1-yl, cyclotetradeca-1,8-dien-1-yl, cyclotetradeca-1,9-dien-1-yl, cyclotetradeca-1,10-dien-1-yl, cyclotetradeca-1,1′-di-en-1-yl, cyclotetradeca-1,12-dien-1-yl, cyclotetradeca-1,13-dien-1-yl, cyclotetradeca-2,4-dien-1-yl, cyclotetradeca-2,5-dien-1-yl, cyclotetradeca-2,6-dien-1-yl, cyclotetradeca-2,7-dien-1-yl, cyclotetradeca-2,8-dien-1-yl, cyclotetradeca-2,9-dien-1-yl, cyclotetradeca-2,10-dien-1-yl, cyclotetradeca-2,11-dien-1-yl, cyclotetradeca-2,12-dien-1-yl, cyclotetradeca-2,13-dien-1-yl, cyclotetradeca-3,5-dien-1-yl, cyclotetradeca-3,6-dien-1-yl, cyclotetradeca-3,7-dien-1-yl, cyclotetradeca-3,8-dien-1-yl, cyclotetradeca-3,9-dien-1-yl, cyclotetradeca-3,10-dien-1-yl, cyclotetradeca-3,11-dien-1-yl, cyclotetradeca-3,12-dien-1-yl, cyclotetradeca-4,6-dien-1-yl, cyclotetradeca-4,7-dien-1-yl, cyclotetradeca-4,8-dien-1-yl, cyclotetradeca-4,9-dien-1-yl, cyclotetradeca-4,10-dien-1-yl, cyclotetradeca-4,11-dien-1-yl, cyclotetradeca-5,7-dien-1-yl, cyclotetradeca-5,8-dien-1-yl, cyclotetradeca-5,9-dien-1-yl, cyclotetradeca-5,10-dien-1-yl, cyclotetradeca-6,8-dien-1-yl, cyclotetra-deca-6,9-dien-1-yl, cyclotetradeca-1,3,5-tetraen-1-yl, cyclotetradeca-1,3,6-tetraen-1-yl, cyclotetradeca-1,3,7-tetraen-1-yl, cyclotetradeca-1,3,8-tetraen-1-yl, cyclotetradeca-1,3,9-tetraen-1-yl, cyclotetradeca-1,3,10-tetraen-1-yl, cyclotetradeca-1,3,11-tetraen-1-yl, cyclotetradeca-1,3,12-tetraen-1-yl, cyclotetradeca-1,3,13-tetraen-1-yl, cyclotetradeca-1,4,6-tetraen-1-yl, cyclotetradeca-1,4,7-tetraen-1-yl, cyclotetradeca-1,4,8-tetraen-1-yl, cyclotetradeca-1,4,9-tetraen-1-yl, cyclotetradeca-1,4,10-tetraen-1-yl, cyclotetradeca-1,4,11-tetraen-1-yl, cyclotetradeca-1,4,12-tetraen-1-yl, cyclotetradeca-1,4,13-tetraen-1-yl, cyclotetradeca-1,5,7-tetraen-1-yl, cyclotetradeca-1,5,8-tetraen-1-yl, cyclotetradeca-1,5,9-tetraen-1-yl, cyclotetradeca-1,5,10-tetraen-1-yl, cyclotetradeca-1,5,11-tetraen-1-yl, cyclotetradeca-1,5,12-tetraen-1-yl, cyclotetradeca-1,5,13-tetraen-1-yl, cyclotetradeca-1,6,8-tetraen-1-yl, cyclotetradeca-1,6,9-tetraen-1-yl, cyclotetradeca-1,6,10-tetraen-1-yl, cyclotetradeca-1,6,11-tetraen-1-yl, cyclotetradeca-1,6,12-tetraen-1-yl, cyclotetradeca-1,6,13-tetraen-1-yl, cyclotetradeca-1,7,9-tetraen-1-yl, cyclotetradeca-1,7,10-tetraen-1-yl, cyclotetradeca-1,7,11-tetraen-1-yl, cyclotetradeca-1,7,12-tetraen-1-yl, cyclotetradeca-1,7,13-tetraen-1-yl, cyclotetradeca-1,8,10-tetraen-1-yl, cyclotetradeca-1,8,11-tetraen-1-yl, cyclotetradeca-1,8,12-tetraen-1-yl, cyclotetradeca-1,8,13-tetraen-1-yl, cyclotetradeca-1,9,11-tetraen-1-yl, cyclotetradeca-1,9,12-tetraen-1-yl, cyclotetradeca-1,9,13-tetraen-1-yl, cyclotetradeca-1,10,12-tetraen-1-yl, cyclotetradeca-1,10,13-tetraen-1-yl, cyclotetradeca-1,11,13-tetraen-1-yl, cyclotetradeca-2,4,6-tetraen-1-yl, cyclotetradeca-2,4,7-tetraen-1-yl, cyclotetradeca-2,4,8-tetraen-1-yl, cyclotetradeca-2,4,9-tetraen-1-yl, cyclotetradeca-2,4,10-tetraen-1-yl, cyclotetradeca-2,4,11-tetraen-1-yl, cyclotetradeca-2,4,12-tetraen-1-yl, cyclotetradeca-2,4,13-tetraen-1-yl, cyclotetradeca-2,5,7-tetraen-1-yl, cyclotetradeca-2,5,8-tetraen-1-yl, cyclotetradeca-2,5,9-tetraen-1-yl, cyclotetradeca-2,5,10-tetraen-1-yl, cyclotetradeca-2,5,11-tetraen-1-yl, cyclotetradeca-2,5,12-tetraen-1-yl, cyclotetradeca-2,5,13-tetraen-1-yl, cyclotetradeca-2,6,8-tetraen-1-yl, cyclotetradeca-2,6,9-tetraen-1-yl, cyclotetradeca-2,6,10-tetraen-1-yl, cyclotetradeca-2,6,11-tetraen-1-yl, cyclotetradeca-2,6,12-tetraen-1-yl, cyclotetradeca-2,6,13-tetraen-1-yl, cyclotetradeca-2,7,9-tetraen-1-yl, cyclotetradeca-2,7,10-tetraen-1-yl, cyclotetradeca-2,7,11-tetraen-1-yl, cyclotetradeca-2,7,12-tetraen-1-yl, cyclotetradeca-2,7,13-tetraen-1-yl, cyclotetradeca-2,8,10-tetraen-1-yl, cyclotetradeca-2,8,11-tetraen-1-yl, cyclotetradeca-2,8,12-tetraen-1-yl, cyclotetradeca-2,8,13-tetraen-1-yl, cyclotetradeca-2,9,11-tetraen-1-yl, cyclotetradeca-2,9,12-tetraen-1-yl, cyclotetradeca-2,9,13-tetraen-1-yl, cyclotetradeca-2,10,12-tetraen-1-yl, cyclotetradeca-2,10,13-tetraen-1-yl, cyclotetradeca-2,11,13-tetraen-1-yl, cyclotetradeca-3,5,7-tetraen-1-yl, cyclotetradeca-3,5,8-tetraen-1-yl, cyclotetradeca-3,5,9-tetraen-1-yl, cyclotetradeca-3,5,10-tetraen-1-yl, cyclotetradeca-3,5,11-tetraen-1-yl, cyclotetradeca-3,5,12-tetraen-1-yl, cyclotetradeca-3,6,8-tetraen-1-yl, cyclotetradeca-3,6,9-tetraen-1-yl, cyclotetradeca-3,6,10-tetraen-1-yl, cyclotetradeca-3,6,11-tetraen-1-yl, cyclotetradeca-3,6,12-tetraen-1-yl, cyclotetradeca-3,7,9-tetraen-1-yl, cyclotetradeca-3,7,10-tetraen-1-yl, cyclotetradeca-3,7,11-tetraen-1-yl, cyclotetradeca-3,7,12-tetraen-1-yl, cyclotetradeca-3,8,10-tetraen-1-yl, cyclotetradeca-3,8,11-tetraen-1-yl, cyclotetradeca-3,9,11-tetraen-1-yl, cyclotetradeca-4,6,8-tetraen-1-yl, cyclotetradeca-4,6,9-tetraen-1-yl, cyclotetradeca-4,6,10-tetraen-1-yl, cyclotetradeca-4,6,11-tetraen-1-yl, cyclotetradeca-4,7,9-tetraen-1-yl, cyclotetradeca-4,7,10-tetraen-1-yl, cyclotetradeca-4,7,11-tetraen-1-yl, cyclotetradeca-4,8,10-tetraen-1-yl, cyclotetradeca-1,3,5,7-tetraen-1-yl, cyclotetradeca-1,3,5,8-tetraen-1-yl, cyclotetradeca-1,3,5,9-tetraen-1-yl, cyclotetradeca-1,3,5,10-tetraen-1-yl, cyclotetradeca-1,3,5,11-tetraen-1-yl, cyclotetradeca-1,3,5,12-tetraen-1-yl, cyclotetradeca-1,3,5,13-tetraen-1-yl, cyclotetradeca-1,3,6,8-tetraen-1-yl, cyclotetradeca-1,3,6,9-tetraen-1-yl, cyclotetradeca-1,3,6,10-tetraen-1-yl, cyclotetradeca-1,3,6,11-tetraen-1-yl, cyclotetradeca-1,3,6,12-tetraen-1-yl, cyclotetradeca-1,3,6,13-tetraen-1-yl, cyclotetradeca-1,3,7,9-tetraen-1-yl, cyclotetradeca-1,3,7,10-tetraen-1-yl, cyclotetradeca-1,3,7,11-tetraen-1-yl, cyclotetradeca-1,3,7,12-tetraen-1-yl, cyclotetradeca-1,3,7,13-tetraen-1-yl, cyclotetradeca-1,3,8,10-tetraen-1-yl, cyclotetradeca-1,3,8,11-tetraen-1-yl, cyclotetradeca-1,3,8,12-tetraen-1-yl, cyclotetradeca-1,3,8,13-tetraen-1-yl, cyclotetradeca-1,3,9,11-tetraen-1-yl, cyclotetradeca-1,3,9,12-tetraen-1-yl, cyclotetradeca-1,3,9,13-tetraen-1-yl, cyclotetradeca-1,3,10,12-tetraen-1-yl, cyclotetradeca-1,3,10,13-tetraen-1-yl, cyclotetradeca-1,3,11,13-tetraen-1-yl, cyclotetradeca-1,4,6,8-tetraen-1-yl, cyclotetradeca-1,4,6,9-tetraen-1-yl, cyclotetradeca-1,4,6,10-tetraen-1-yl, cyclotetradeca-1,4,6,11-tetraen-1-yl, cyclotetradeca-1,4,6,12-tetraen-1-yl, cyclotetradeca-1,4,6,13-tetraen-1-yl, cyclotetradeca-1,4,7,9-tetraen-1-yl, cyclotetradeca-1,4,7,10-tetraen-1-yl, cyclotetradeca-1,4,7,11-tetraen-1-yl, cyclotetradeca-1,4,7,12-tetraen-1-yl, cyclotetradeca-1,4,7,13-tetraen-1-yl, cyclotetradeca-1,4,8,10-tetraen-1-yl, cyclotetradeca-1,4,8,11-tetraen-1-yl, cyclotetradeca-1,4,8,12-tetraen-1-yl, cyclotetradeca-1,4,8,13-tetraen-1-yl, cyclotetradeca-1,4,9,11-tetraen-1-yl, cyclotetradeca-1,4,9,12-tetraen-1-yl, cyclotetradeca-1,4,9,13-tetraen-1-yl, cyclotetradeca-1,4,10,12-tetraen-1-yl, cyclotetradeca-1,4,10,13-tetraen-1-yl, cyclotetradeca-1,4,11,13-tetraen-1-yl, cyclotetradeca-1,5,7,9-tetraen-1-yl, cyclotetradeca-1,5,7,10-tetraen-1-yl, cyclotetradeca-1,5,7,11-tetraen-1-yl, cyclotetradeca-1,5,7,12-tetraen-1-yl, cyclotetradeca-1,5,7,13-tetraen-1-yl, cyclotetradeca-1,5,8,10-tetraen-1-yl, cyclotetradeca-1,5,8,11-tetraen-1-yl, cyclotetradeca-1,5,8,12-tetraen-1-yl, cyclotetradeca-1,5,8,13-tetraen-1-yl, cyclotetradeca-1,5,9,11-tetraen-1-yl, cyclotetradeca-1,5,9,12-tetraen-1-yl, cyclotetradeca-1,5,9,13-tetraen-1-yl, cyclotetradeca-1,5,10,12-tetraen-1-yl, cyclotetradeca-1,5,10,13-tetraen-1-yl, cyclotetradeca-1,5,11,13-tetraen-1-yl, cyclotetradeca-1,6,8,10-tetraen-1-yl, cyclotetradeca-1,6,8,11-tetraen-1-yl, cyclotetradeca-1,6,8,12-tetraen-1-yl, cyclotetradeca-1,6,8,13-tetraen-1-yl, cyclotetradeca-1,6,9,11-tetraen-1-yl, cyclotetradeca-1,6,9,12-tetraen-1-yl, cyclotetradeca-1,6,9,13-tetraen-1-yl, cyclotetradeca-1,6,10,12-tetraen-1-yl, cyclotetradeca-1,6,10,13-tetraen-1-yl, cyclotetradeca-1,6,11,13-tetraen-1-yl, cyclotetradeca-1,7,9,11-tetraen-1-yl, cyclotetradeca-1,7,9,12-tetraen-1-yl, cyclotetradeca-1,7,9,13-tetraen-1-yl, cyclotetradeca-1,7,10,12-tetraen-1-yl, cyclotetradeca-1,7,10,13-tetraen-1-yl, cyclotetradeca-1,7,11,13-tetraen-1-yl, cyclotetradeca-1,8,10,12-tetraen-1-yl, cyclotetradeca-1,8,10,13-tetraen-1-yl, cyclotetradeca-1,8,11,13-tetraen-1-yl, cyclotetradeca-2,4,6,8-tetraen-1-yl, cyclotetradeca-2,4,6,9-tetraen-1-yl, cyclotetradeca-2,4,6,10-tetraen-1-yl, cyclotetradeca-2,4,6,11-tetraen-1-yl, cyclotetradeca-2,4,6,12-tetraen-1-yl, cyclotetradeca-2,4,6,13-tetraen-1-yl, cyclotetradeca-2,4,7,9-tetraen-1-yl, cyclotetradeca-2,4,7,10-tetraen-1-yl, cyclotetradeca-2,4,7,11-tetraen-1-yl, cyclotetradeca-2,4,7,12-tetraen-1-yl, cyclotetradeca-2,4,7,13-tetraen-1-yl, cyclotetradeca-2,4,8,10-tetraen-1-yl, cyclotetradeca-2,4,8,11-tetraen-1-yl, cyclotetradeca-2,4,8,12-tetraen-1-yl, cyclotetradeca-2,4,8,13-tetraen-1-yl, cyclotetradeca-2,4,9,11-tetraen-1-yl, cyclotetradeca-2,4,9,12-tetraen-1-yl, cyclotetradeca-2,4,9,13-tetraen-1-yl, cyclotetradeca-2,4,10,12-tetraen-1-yl, cyclotetradeca-2,4,10,13-tetraen-1-yl, cyclotetradeca-2,4,11,13-tetraen-1-yl, cyclotetradeca-2,5,7,9-tetraen-1-yl, cyclotetradeca-2,5,7,10-tetraen-1-yl, cyclotetradeca-2,5,7,11-tetraen-1-yl, cyclotetradeca-2,5,7,12-tetraen-1-yl, cyclotetradeca-2,5,7,13-tetraen-1-yl, cyclotetradeca-2,5,8,10-tetraen-1-yl, cyclotetradeca-2,5,8,11-tetraen-1-yl, cyclotetradeca-2,5,8,12-tetraen-1-yl, cyclotetradeca-2,5,8,13-tetraen-1-yl, cyclotetradeca-2,5,9,11-tetraen-1-yl, cyclotetradeca-2,5,9,12-tetraen-1-yl, cyclotetradeca-2,5,9,13-tetraen-1-yl, cyclotetradeca-2,5,10,12-tetraen-1-yl, cyclotetradeca-2,5,10,13-tetraen-1-yl, cyclotetradeca-2,6,8,10-tetraen-1-yl, cyclotetradeca-2,6,8,11-tetraen-1-yl, cyclotetradeca-2,6,8,12-tetraen-1-yl, cyclotetradeca-2,6,8,13-tetraen-1-yl, cyclotetradeca-2,6,9,11-tetraen-1-yl, cyclotetradeca-2,6,9,12-tetraen-1-yl, cyclotetradeca-2,6,9,13-tetraen-1-yl, cyclotetradeca-2,6,10,12-tetraen-1-yl, cyclotetradeca-2,6,10,13-tetraen-1-yl, cyclotetradeca-2,6,11,13-tetraen-1-yl, cyclotetradeca-2,7,9,11-tetraen-1-yl, cyclotetradeca-2,7,9,12-tetraen-1-yl, cyclotetradeca-2,7,10,12-tetraen-1-yl, cyclotetradeca-3,5,7,9-tetraen-1-yl, cyclotetradeca-3,5,7,10-tetraen-1-yl, cyclotetradeca-3,5,7,11-tetraen-1-yl, cyclotetradeca-3,5,7,12-tetraen-1-yl, cyclotetradeca-3,5,8,10-tetraen-1-yl, cyclotetradeca-3,5,8,11-tetraen-1-yl, cyclotetradeca-3,5,8,12-tetraen-1-yl, cyclotetradeca-3,5,9,11-tetraen-1-yl, cyclotetradeca-3,5,9,12-tetraen-1-yl, cyclotetradeca-3,5,10,12-tetraen-1-yl, cyclotetradeca-3,6,8,10-tetraen-1-yl, cyclotetradeca-3,6,8,11-tetraen-1-yl, cyclotetradeca-3,6,8,12-tetraen-1-yl, cyclotetradeca-3,7,9,11-tetraen-1-yl, cyclotetradeca-1,3,5,7,9-pentaen-1-yl, cyclotetradeca-1,3,5,7,10-pentaen-1-yl, cyclotetradeca-1,3,5,7,11-pentaen-1-yl, cyclotetradeca-1,3,5,7,12-pentaen-1-yl, cyclotetradeca-1,3,5,7,13-pentaen-1-yl, cyclotetradeca-1,3,5,8,10-pentaen-1-yl, cyclotetradeca-1,3,5,8,11-pentaen-1-yl, cyclotetradeca-1,3,5,8,12-pentaen-1-yl, cyclotetradeca-1,3,5,8,13-pentaen-1-yl, cyclotetradeca-1,3,5,9,11-pentaen-1-yl, cyclotetradeca-1,3,5,9,12-pentaen-1-yl, cyclotetradeca-1,3,5,9,13-pentaen-1-yl, cyclotetradeca-1,3,5,10,12-pentaen-1-yl, cyclotetradeca-1,3,5,10,13-pentaen-1-yl, cyclotetradeca-1,3,5,11,13-pentaen-1-yl, cyclotetradeca-1,3,6,8,10-pentaen-1-yl, cyclotetradeca-1,3,6,8,11-pentaen-1-yl, cyclotetradeca-1,3,6,8,12-pentaen-1-yl, cyclotetradeca-1,3,6,8,13-pentaen-1-yl, cyclotetradeca-1,3,6,9,11-pentaen-1-yl, cyclotetradeca-1,3,6,9,12-pentaen-1-yl, cyclotetradeca-1,3,6,9,13-pentaen-1-yl, cyclotetradeca-1,3,7,9,11-pentaen-1-yl, cyclotetradeca-1,3,7,9,12-pentaen-1-yl, cyclotetradeca-1,3,7,9,13-pentaen-1-yl, cyclotetradeca-1,4,6,8,10-pentaen-1-yl, cyclotetradeca-1,4,6,8,11-pentaen-1-yl, cyclotetradeca-1,4,6,8,12-pentaen-1-yl, cyclotetradeca-1,4,6,8,13-pentaen-1-yl, cyclotetradeca-1,4,6,9,11-pentaen-1-yl, cyclotetradeca-1,4,6,9,12-pentaen-1-yl, cyclotetradeca-1,4,6,9,13-pentaen-1-yl, cyclotetradeca-1,4,7,9,11-pentaen-1-yl, cyclotetradeca-1,4,7,9,12-pentaen-1-yl, cyclotetradeca-1,4,7,9,13-pentaen-1-yl, cyclotetradeca-1,5,7,9,11-pentaen-1-yl, cyclotetradeca-1,5,7,9,12-pentaen-1-yl, cyclotetradeca-1,5,7,9,13-pentaen-1-yl, cyclotetradeca-2,4,6,8,10-pentaen-1-yl, cyclotetradeca-2,4,6,8,11-pentaen-1-yl, cyclotetradeca-2,4,6,8,12-pentaen-1-yl, cyclotetradeca-2,4,6,8,13-pentaen-1-yl, cyclotetradeca-2,4,6,9,11-pentaen-1-yl, cyclotetradeca-2,4,6,9,12-pentaen-1-yl, cyclotetradeca-2,4,6,9,13-pentaen-1-yl, cyclotetradeca-2,4,6,10,12-pentaen-1-yl, cyclotetradeca-2,4,6,10,13-pentaen-1-yl, cyclotetradeca-2,4,6,11,13-pentaen-1-yl, cyclotetradeca-2,4,7,9,11-pentaen-1-yl, cyclotetradeca-2,4,7,9,12-pentaen-1-yl, cyclotetradeca-2,4,7,9,13-pentaen-1-yl, cyclotetradeca-2,4,7,10,12-pentaen-1-yl, cyclotetradeca-2,4,7,10,13-pentaen-1-yl, cyclotetradeca-2,4,7,11,13-pentaen-1-yl, cyclotetradeca-2,4,8,10,12-pentaen-1-yl, cyclotetradeca-2,4,8,10,13-pentaen-1-yl, cyclotetradeca-2,5,7,9,11-pentaen-1-yl, cyclotetradeca-2,5,7,9,12-pentaen-1-yl, cyclotetradeca-2,5,7,9,13-pentaen-1-yl, cyclotetradeca-2,5,7,10,12-pentaen-1-yl, cyclotetradeca-2,5,7,10,13-pentaen-1-yl, cyclotetradeca-1,3,5,7,9,11-hexaen-1-yl, cyclotetradeca-1,3,5,7,9,12-hexaen-1-yl, cyclotetradeca-1,3,5,7,9,13-hexaen-1-yl, cyclotetradeca-2,4,6,8,10,12-hexaen-1-yl, cyclotetradeca-2,4,6,8,10,13-hexaen-1-yl, cyclotetradeca-2,4,6,8,11,13-hexaen-1-yl and cyclotetradeca-1,3,5,7,9,11,13-heptaen-1-yl.

The term “C₃-cycloalkyl,” as used herein in reference to compounds of the invention, means cycloprop-1-yl.

The term “C₄-cycloalkyl,” as used herein in reference to compounds of the invention, means cyclobut-1-yl.

The term “C₅-cycloalkyl,” as used herein in reference to compounds of the invention, means cyclopent-1-yl.

The term “C₆-cycloalkyl,” as used herein in reference to compounds of the invention, means cyclohex-1-yl.

The term “C₇-cycloalkyl,” as used herein in reference to compounds of the invention, means bicyclo[2.2.1]hept-1-yl, bicyclo[2.2.1]hept-2-yl, cyclohept-1-yl, bicyclo[2.2.1]hept-7-yl and cyclohept-1-yl.

The term “C₈-cycloalkyl,” as used herein in reference to compounds of the invention, means bicyclo[2.2.2]oct-1-yl, bicyclo[2.2.2]oct-2-yl, bicyclo[2.2.2]oct-7-yl, cyclooct-1-yl.

The term “C₉-cycloalkyl,” as used herein in reference to compounds of the invention, means cyclonon-1-yl.

The term “C₁₀-cycloalkyl,” as used herein in reference to compounds of the invention, means adamant-1-yl, adamant-2-yl and cyclodec-1-yl.

The term “C₁₁-cycloalkyl,” as used herein in reference to compounds of the invention, means cycloundec-1-yl, tricyclo[4.3.1.1^{3,8}]undec-1-yl (homoadamant-1-yl), tricyclo[4.3.1.1^{3,8}]undec-2-yl (homoadamant-2-yl), tricyclo[4.3.1.1^{3,8}]undec-3-yl (homoadamant-3-yl), tricyclo[4.3.1.1^{3,8}]undec-4-yl (homoadamant-4-yl), and tricyclo[4.3.1.1^{3,8}]undec-9-yl (homoadamant-9-yl).

The term “C₁₂-cycloalkyl,” as used herein in reference to compounds of the invention, means cyclododec-1-yl.

The term “C₁₃-cycloalkyl,” as used herein in reference to compounds of the invention, means cyclotridec-1-yl.

The term “C₁₄-cycloalkyl,” as used herein in reference to compounds of the invention, means cyclotetradec-1-yl.

The term “C₂-spiroalkenyl,” as used herein in reference to compounds of the invention, means ethen-1,2-ylene, both ends of which replace hydrogen atoms of the same CH₂ moiety.

The term “C₃-spiroalkenyl,” as used herein in reference to compounds of the invention, means prop-1-en-1,3-ylene, both ends of which replace hydrogen atoms of the same CH₂ moiety.

The term “C₄-spiroalkenyl,” as used herein in reference to compounds of the invention, means but-1-en-1,4-ylene, but-2-en-1,4-ylene and buta-1,3-dien-1,4-ylene, both ends of which replace hydrogen atoms of the same CH₂ moiety.

The term “C₅-spiroalkenyl,” as used herein in reference to compounds of the invention, means pent-1-en-1,5-yl-ene, pent-2-en-1,5-ylene, penta-1,3-dien-1,5-ylene and penta-1,4-dien-1,5-ylene, both ends of which replace hydrogen atoms of the same CH₂ moiety.

The term “C₆-spiroalkenyl,” as used herein in reference to compounds of the invention, means hex-1-en-1,6-ylene, hex-2-en-1,6-ylene, hexa-1,3-dien-1,6-ylene, hexa-1,4-di-en-1,6-ylene and hexa-1,3,5-trien-1,6-ylene, both ends of which replace hydrogen atoms of the same CH₂ moiety.

The term “C₇-spiroalkenyl,” as used herein in reference to compounds of the invention, means hept-1-en-1,7-yl-ene, hept-2-en-1,7-ylene, hept-3-en-1,7-ylene, hepta-1,3-dien-1,7-ylene, hepta-1,4-dien-1,7-ylene, hepta-1,5-dien-1,7-ylene, hepta-2,4-dien-1,7-ylene, hepta-2,5-dien-1,7-ylene, hepta-1,3,5-trien-1,7-ylene and hepta-1,3,6-trien-1,7-ylene, both ends of which replace hydrogen atoms of the same CH₂ moiety.

The term “C₈-spiroalkenyl,” as used herein in reference to compounds of the invention, means oct-1-en-1,8-ylene, oct-2-en-1,8-ylene, oct-3-en-1,8-ylene, octa-1,3-dien-1,8-ylene, octa-1,4-dien-1,8-ylene, octa-1,5-dien-1,8-ylene, octa-1,6-dien-1,8-ylene, octa-2,4-dien-1,8-ylene, octa-2,5-dien-1,8-ylene, octa-3,5-dien-1,8-ylene, octa-1,3,5-trien-1,8-ylene, octa-1,3,6-trien-1,8-ylene and octa-2,4,6-trien-1,8-ylene, both ends of which replace hydrogen atoms of the same CH₂ moiety.

The term “C₉-spiroalkenyl,” as used herein in reference to compounds of the invention, means nona-1-en-1,9-yl-ene, nona-2-en-1,9-ylene, nona-3-en-1,9-ylene, nona-4-en-1,9-ylene, nona-1,3-dien-1,9-ylene, nona-1,4-dien-1,9-ylene, nona-1,5-dien-1,9-ylene, nona-1,6-dien-1,9-ylene, nona-1,7-dien-1,9-ylene, nona-1,8-dien-1,9-ylene, nona-2,4-dien-1,9-ylene, nona-2,5-dien-1,9-ylene, nona-2,6-dien-1,9-ylene, nona-2,7-dien-1,9-ylene, nona-3,5-dien-1,9-ylene, nona-3,6-dien-1,9-ylene, nona-4,6-dien-1,9-ylene, nona-1,3,5-trien-1,9-ylene, nona-1,3,6-trien-1,9-ylene, nona-1,3,7-trien-1,9-ylene, nona-1,3,8-trien-1,9-ylene, nona-1,4,6-trien-1,9-ylene, nona-1,4,7-trien-1,9-ylene, nona-1,4,8-trien-1,9-ylene, nona-1,5,7-trien-1,9-ylene, nona-2,4,6-trien-1,9-ylene, nona-2,4,7-trien-1,9-ylene, nona-1,3,5,7-tetraen-1,9-ylene, nona-1,3,5,8-tetraen-1,9-ylene and nova-1,3,6,9-tetraen-1,9-ylene, both ends of which replace hydrogen atoms of the same CH₂ moiety.

The term “C₂-spiroalkyl,” as used herein in reference to compounds of the invention, means eth-1,2-ylene, both ends of which replace hydrogen atoms of the same CH₂ moiety.

The term “C₃-spiroalkyl,” as used herein in reference to compounds of the invention, means prop-1,3-ylene, both ends of which replace hydrogen atoms of the same CH₂ moiety.

The term “C₄-spiroalkyl,” as used herein in reference to compounds of the invention, means but-1,4-ylene, both ends of which replace hydrogen atoms of the same CH₂ moiety.

The term “C₅-spiroalkyl,” as used herein in reference to compounds of the invention, means pent-1,5-ylene, both ends of which replace hydrogen atoms of the same CH₂ moiety.

The term “C₆-spiroalkyl,” as used herein in reference to compounds of the invention, means hex-1,6-ylene, both ends of which replace hydrogen atoms of the same CH₂ moiety.

The term “C₇-spiroalkyl,” as used herein in reference to compounds of the invention, means hept-1,7-ylene, both ends of which replace hydrogen atoms of the same CH₂ moiety.

The term “C₈-spiroalkyl,” as used herein in reference to compounds of the invention, means oct-1,8-ylene, both ends of which replace hydrogen atoms of the same CH₂ moiety.

The term “C₉-spiroalkyl,” as used herein in reference to compounds of the invention, means non-1,9-ylene, both ends of which replace hydrogen atoms of the same CH₂ moiety.

The terms used to describe the compounds of Formula I′, which are excluded from the presently claimed compounds of Formula I are defined below as follows.

As used herein in reference to compounds described by Formula I′, the term “alkyl” refers to a straight chain or branched saturated hydrocarbon group having 1 to 10 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

As used herein in reference to compounds described by Formula I′, the term “alkenyl” refers to a straight-chain or branched hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl and decenyl.

As used herein in reference to compounds described by Formula I′, the term “alkynyl” refers to a straight-chain or branched hydrocarbon group having one or more triple bonds between carbon atoms and having 2 to 10 carbon atoms. Where appropriate, the alkynyl group may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to, ethynyl, propynyl, butynyl, pentynyl, hexynyl, octynyl, nonynyl and decynyl.

As used herein in reference to compounds described by Formula I′, the term “cycloalkyl” refers to a saturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentanyl, cyclohexanyl, cycloheptanyl and cyclooctanyl.

As used herein in reference to compounds described by Formula I′, the term “cycloalkenyl” refers to a cyclic hydrocarbon having at least one double bond. The cycloalkenyl ring may include a specified number of carbon atoms. For example, a 4 to 8 membered cycloalkenyl group contains at least one double bond and 4, 5, 6, 7 or 8 carbon atoms. Examples of suitable cycloalkenyl groups include, but are not limited to cyclopentenyl, cyclopenta-1,3-dienyl, cyclohexenyl, cyclohexen-1,3-dienyl, or cyclohexen-1,4-dienyl.

The term “acyl” used herein in reference to compounds described by Formula I′, refers to an alkanoyl or aroyl group as defined by (C═O)R where suitable R groups include, but are not limited to, —C₁₋₇alkyl, —C₁₋₇alkenyl, —C₁₋₇alkynyl, —C₃₋₈-cycloalkyl, —C₃₋₈cycloalkenyl aryl, heterocyclyl, heteroaryl, —C₁₋₇alkylaryl, —C₁₋₇alkylcycloalkyl, —C₁₋₇alkylcycloalkenyl, —C₁₋₇alkylheterocyclyl, —C₁₋₇alkylheteroaryl, —C₁₋₇alkoxyalkyl, —C₁₋₇alkylthioalkyl, —C₁₋₇alkylthioaryl, —C₁₋₇alkoxyaryl and the like.

The terms “alkyloxy” or “alkoxy”, “alkenyloxy”, “alkynyloxy”, “cycloalkyloxy”, “cycloalkenyloxy”, “aryloxy”, “heterocyclyloxy”, “heteroaryloxy”, “Oalkyl”, “Oalkenyl”, “Oalkynyl”, “Ocycloalkyl”, “Ocycloalkenyl”, “Oaryl”, “Oheterocyclyl” and “Oheteroaryl” as used herein in reference to compounds described by Formula I′, represent an alkyl, alkenyl, alkynyl, cycloalkyl, cycloalkenyl, aryl, heterocyclyl or heteroaryl group as defined attached through an oxygen bridge. Examples of suitable alkyloxy, alkenyloxy, alkynyloxy, cycloalkyloxy, cycloalkenyloxy, aryloxy, heterocyclyloxy and heteroaryloxy groups include, but are not limited to, methoxy, ethoxy, n-propyloxy, n-butyloxy, n-pentyloxy, n-hexyloxy, ethenyloxy, propenyloxy, butenyloxy, pentenyloxy, hexenyloxy, ethynyloxy, propynyloxy, butynyloxy, pentynyloxy, hexynyloxy, cyclopentyloxy, cyclohexyloxy, cyclopentenyloxy, cyclohexenyloxy, phenoxy, naphthoxy, pyrrolidinyloxy, tetrahydrofuranyloxy, furanyloxy and pyridinyloxy.

The terms “alkylthio”, “alkenylthio”, “alkynylthio”, “Salkyl” and “Salkenyl”, as used herein in reference to compounds described by Formula I′, represent an alkyl, alkenyl or alkynyl group as defined above attached through a sulfur bridge. Examples of suitable alkylthio, alkenylthio and alkynylthio include, but are not limited to, methylthio, ethylthio, propylthio, butylthio, pentylthio, hexylthio, ethenylthio, propenylthio, butenylthio, pentenylthio, hexenylthio, ethynylthio, propynylthio, butynylthio, pentynylthio and hexynylthio.

As used herein in reference to compounds described by Formula I′, the term “aryl” is intended to mean any stable, monocyclic or bicyclic carbon ring of up to 7 atoms in each ring, wherein at least one ring is aromatic. Examples of such aryl groups include, but are not limited to, phenyl, naphthyl, tetrahydronaphthyl, indanyl, biphenyl and binaphthyl.

As used herein in reference to compounds described by Formula I′, the term “halogen” or “halo” refers to fluorine (fluoro), chlorine (chloro), bromine (bromo) and iodine (iodo).

The term “heterocyclic” or “heterocyclyl” as used herein in reference to compounds described by Formula I′, refers to a cyclic hydrocarbon in which one to four carbon atoms have been replaced by heteroatoms independently N, S or O. A heterocyclic ring may be saturated or unsaturated. Examples of suitable heterocyclyl groups include tetrahydrofuranyl, tetrahydrothiophenyl, pyrrolidinyl, pyrrolinyl, pyranyl, piperidinyl, piperazinyl, pyrazolinyl, dithiolyl, oxathiolyl, dioxanyl, dioxinyl, morpholino, thiomorpholino and oxazinyl.

The term “heteroaryl” as used herein in reference to compounds described by Formula I′, represents a stable monocyclic or bicyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, pyrazolyl, indolyl, benzotriazolyl, furanyl, thienyl, thiophenyl, benzothienyl, benzofuranyl, quinolinyl, isoquinolinyl, oxazolyl, isoxazolyl, imidazolyl, pyrazinyl, pyridazinyl, pyridinyl, pyrimidinyl, pyrrolyl, tetrahydroquinoline, thiazolyl, isothiazolyl, 1,2,4-triazolyl, 1,2,4-oxadiazolyl and 1,2,4-thiadiazolyl. Particular heteroaryl groups have 5- or 6-membered rings, such as pyrazolyl, furanyl, thienyl, oxazolyl, isoxazolyl, imidazolyl, pyrazinyl, pyridazinyl, pyridinyl, pyrimidinyl, pyrrolyl, thiazolyl, isothiazolyl, 1,2,4-triazolyl and 1,2,4-oxadiazolyl and 1,2,4-thiadiazolyl.

Each alkyl, alkenyl, alkynyl, cycloalkyl, cycloalkenyl, aryl, heterocyclyl and heteroaryl, as used herein in reference to compounds described by Formula I′, whether an individual entity or as part of a larger entity may be optionally substituted with one or more optional substituents selected from the group consisting of C₁₋₆alkyl, C₂₋₆alkenyl, C₂₋₆alkynyl, C₃₋₆cycloalkyl, C₁₋₆alkyloxy(CH₂)ₚ—, C₂₋₆alkenyloxy(CH₂)ₚ—, C₂₋₆alkynyloxy(CH₂)ₚ—, C₃₋₆cycloalkoxy(CH₂)ₚ—, C₁₋₆alkylthio(CH₂)ₚ—, C₂₋₆alkenylthio(CH₂)ₚ—, C₂₋₆alkynylthio(CH₂)_{p I C}₃₋₆cycloalkylthio(CH₂)ₚ—, hydroxy(CH₂)ₚ—, (CH₂)ₚSH, —(CH₂)ₚCO₂H, —(CH₂)ₚCO₂C₁₋₆alkyl, (CH₂)ₚCON(R¹⁰)₂, C₂₋₆acyl(CH₂)ₚ—, C₂₋₆acyloxy(CH₂)ₚ—, C₂₋₆alkylSO₂(CH₂)ₚ, C₂₋₆alkenylSO₂(CH₂)_{p I C}₂₋₆alkynylSO₂ (CH₂)ₚ—, arylSO₂ (CH₂)ₚ—, heteroarylSO₂ (CH₂)ₚ—, heterocyclylSO₂ (CH₂)ₚ—, —(CH₂)ₚNH₂, —(CH₂)ₚNH(C₁₋₆alkyl). —(CH₂)ₚN(C₁₋₆alkyl)₂, —(CH₂)ₚNH(phenyl), —(CH₂)ₚN(phenyl)₂, —(CH₂)ₚNH(acyl), —(CH₂)ₚN(acyl)(phenyl), —(CH₂)ₚNH—(CH₂)ₚ—S-aryl, —(CH₂)ₚN═NHC(O)NH₂, —(CH₂)_{P}C(R¹¹)₂, —(CH₂)ₚOC(R¹¹)₂, —(CH₂)ₚSC(R¹¹)₂, —(CH₂)ₚCN, —(CH₂)ₚNO₂, —(CH₂)ₚhalogen, —(CH₂)ₚheterocyclyl, heterocyclyloxy(CH₂)ₚ—, —(CH₂)ₚheteroaryl, heteroaryloxy(CH₂)ₚ—, —(CH₂)ₚaryl, —(CH₂)ₚC(O)aryl and aryloxy(CH₂)ₚ— wherein each R¹¹ is independently hydrogen or halogen; each R¹⁰ is independently H, C₁₋₆alkyl, phenyl or cycloalkyl or the two R¹⁰ taken together with the nitrogen to which they are attached can form a heterocyclyl or heteroaryl ring; and p is 0 or an integer from 1 to 6. Examples of suitable substituents include, but are not limited to, methyl, ethyl, propyl, isopropyl, butyl, sec-butyl, tert-butyl, vinyl, methoxy, ethoxy, propoxy, isopropoxy, butoxy, methylthio, ethylthio, propylthio, isopropylthio, butylthio, hydroxy, hydroxymethyl, hydroxyethyl, hydroxypropyl, hydroxybutyl, fluoro, chloro, bromo, iodo, cyano, nitro, CO₂H, CO₂CH₂, CH₂CO₂CH₃, trifluoromethyl, trifluoromethoxy, trifluoromethylthio, acetyl, morpholino, amino, methylamino, dimethylamino, phenyl, phenylcarbonyl, NHCOphenyl, NHCObenzyl in which the phenyl ring is optionally substituted with methyl or methoxy, NHCOethylphenyl, NHCOCH₂Sphenyl-N═NHC(O)NH₂, —CH═C(CN)₂ and phenoxy. Particular substituents include fluoro, chloro, methyl, ethyl, propyl, isopropyl, butyl, tert-butyl, methoxy, ethoxy, propoxy, isopropoxy, trifluoromethyl, trifluoromethoxy, cyano, nitro, acetyl, amino, methylamino, dimethylamino, phenyl and benzyl in which the phenyl or benzyl ring is optionally substituted with halo, methyl or methoxy.

The term “lymphoma,” as used herein, means malignant neoplasms of lymphatic or reticular endothelial tissues that occur as circumscribed solid tumors and are composed of cells resembling lymphocytes, plasma cells or histocytes. Specific examples of lymphoma include, but are not limited to, B-cell lymphoma, Hodgkin's lymphoma, non-Hodgkin's lymphoma and disseminated lymphoma.

The term “antitumorigenesis,” as used herein, means reduction of tumor growth.

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration present in the higher amount, preferably an excess of about 85%-90%, more preferably an excess of about 95%-99%, and still more preferably an excess greater than about 99%. Accordingly, this invention includes racemic mixtures, relative and absolute stereoisomers, and mixtures of relative and absolute stereoisomers.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds may also exist as an equilibrium mixture of Z or E configurations.

Compounds of this invention containing NH, C(O)OH, OH or SH moieties may have attached thereto prodrug-forming moieties. The prodrug-forming moieties are removed by metabolic processes and release the compounds having the freed hydroxyl, amino or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance. For example, examples of prodrug-forming moieties for compounds having C(O)OH moieties are pivalate (CH₂OC(O)C(CH₃)₃) or phosphonooxy (CH₂OP(O)(OH)₂) esters.

Metabolites of compounds having Formula I, produced by in vitro or in vivo metabolic processes, may also have utility for treating diseases associated with expression of an anti-apoptotic protein family member such as of BCl-X_{L} protein, and Bcl-2 protein or Bcl-w protein.

Certain precursor compounds which may be metabolized in vitro or in vivo to form compounds having Formula I may also have utility for treating diseases associated with expression of an anti-apoptotic protein family member such as of BCl-X_{L} protein, and Bcl-2 protein or Bcl-w protein.

Compounds having Formula I may exist as an acid addition salts, basic addition salts or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the compounds and prodrugs thereof are contemplated as being embraced by this invention. Basic addition salts of the compounds are those derived from the reaction of the compounds with the hydroxide, carbonate or bicarbonate of cations such as lithium, sodium, potassium, calcium, and magnesium.

Synthesis of Compounds:

Compounds having Formula I may be made by synthetic chemical processes, examples of which are shown herein. It is meant to be understood that the order of the steps in the processes may be varied, reagents, solvents, and reaction conditions may be substituted for those specifically mentioned, and vulnerable moieties may be protected and deprotected, as necessary, by NH, C(O)OH, OH, SH protecting groups.

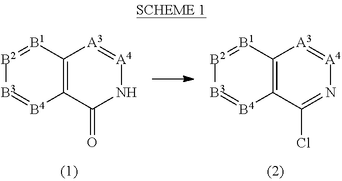

Compounds having Formula 1 may be converted to compounds having Formula 2 by reacting the former with a chlorinating agent such as, for example, POCl₃, PCl₅ or SOCl₂, in a solvent such as, for example, N,N-dimethylformamide or N-methylpyrrolidinone.

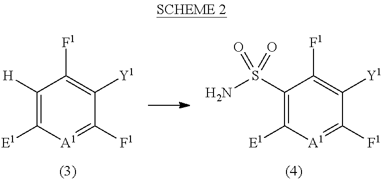

Compounds having Formula 3 may be converted to compounds having Formula 4 by reacting the former, chlorosulfonic acid, and ammonia.

Additionally, compounds of Formula 4 suitable for reaction with compounds of Formula 2 can be prepared according to the methods described the Examples section found in U.S. patent application Ser. No. 11/600,445, and International Application No. PCT/US01/29432, the teaching of each is incorporated herein by reference.

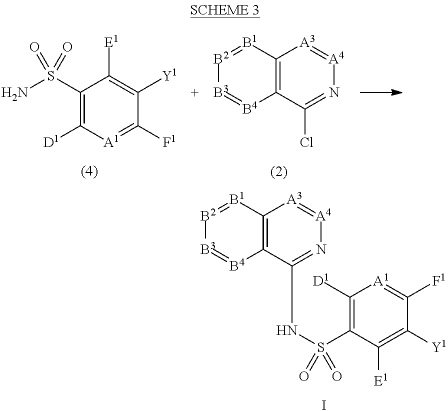

Compounds having Formula 4 may be converted to compounds having Formula I by reacting the former and compounds having Formula 2 in the presence or absence of a first base and optionally in the presence of a coupling agent. Examples of coupling agents include EDCI, CDI, and PyBop. Examples of first bases include TEA, DIEA, DMAP, and mixtures thereof.

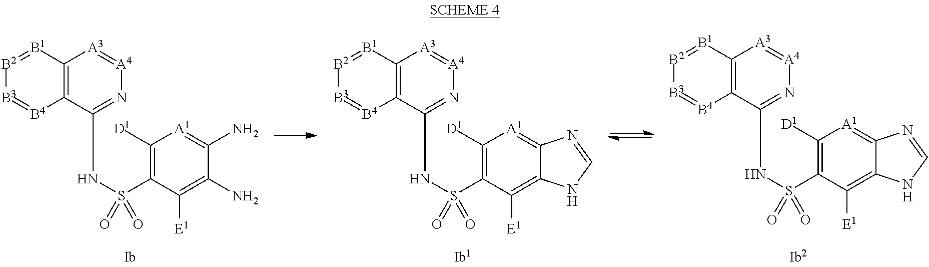

Compounds having Formula Ib may be converted to compounds having Formula Ib¹, wherein F¹ and Y¹ together are imidazole, by reacting the former, sodium nitrite, hydrochloric acid, and acetic acid. Compounds having Formula Ib¹, wherein F¹ and Y¹ together are imidazole, may be reacted with a second base and the appropriate electrophile to provide compounds having Formula Ib², wherein F¹ and Y¹ together are substituted imidazole. Examples of second bases include sodium hydride, potassium hydride, lithium diisopropylamide and sodium bis(trimethylsilyl)amide.

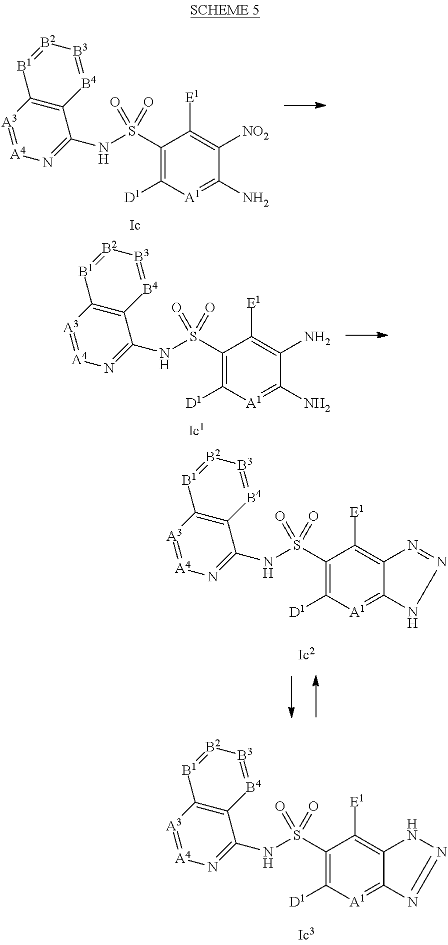

Compounds having Formula Ic, may be converted to compounds having Formula Ic¹ by reacting the former, hydrogen and a hydrogenation catalyst. Examples of hydrogenation catalysts include Pd on carbon, platinum on carbon, and Raney nickel.

Compounds having Formula Ic¹ may be converted to compounds having Formula Ic², wherein F¹ and Y¹ together are triazole, by reacting the former, sodium nitrite, hydrochloric acid, and acetic acid. Compounds having Formula Ic², wherein F¹ and Y¹ together are triazole, may be reacted with the second base and the appropriate electrophile to provide compounds having Formula Ic^{a}, wherein F¹ and Y¹ together are substituted triazole.

Compositions and Methods of Use:

The compounds having Formula I may be administered, for example, bucally, ophthalmically, orally, osmotically, parenterally (intramuscularly, intraperintoneally intrasternally, intravenously, subcutaneously), rectally, topically, transdermally, or vaginally.

Therapeutically effective amounts of compounds having Formula I depend on recipient of treatment, disorder being treated and severity thereof, composition containing it, time of administration, route of administration, duration of treatment, its potency, its rate of clearance and whether or not another drug is co-administered. The amount of a compound of this invention having Formula I used to make a composition to be administered daily to a patient in a single dose or in divided doses is from about 0.03 to about 200 mg/kg body weight. Single dose compositions contain these amounts or a combination of submultiples thereof.

Compounds having Formula I may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

Excipients for preparation of compositions comprising a compound having Formula I to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof. Excipients for preparation of compositions comprising a compound of this invention having Formula I to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising a compound of this invention having Formula I to be administered osmotically include, for example, chlorofluorohydrocarbons, ethanol, water and mixtures thereof. Excipients for preparation of compositions comprising a compound of this invention having Formula I to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising a compound of this invention having Formula I to be administered rectally or vaginally include, for example, cocoa butter, polyethylene glycol, wax and mixtures thereof.

Compounds having Formula I may also be administered with one or more than one additional therapeutic agents, wherein additional therapeutic agents include radiation or chemotherapeutic agents, wherein chemotherapeutic agents include, but are not limited to, carboplatin, cisplatin, cyclophosphamide, dacarbazine, dexamethasone, docetaxel, doxorubicin, etoposide, fludarabine, irinotecan, CHOP(C: Cytoxan® (cyclophosphamide); H: Adiamycin® (hydroxydoxorubicin); O: Vincristine (Oncovin®); P: prednisone), paclitaxel, rapamycin, Rituxin® (rituximab) and vincristine.

The compounds of Formula I have utility as inhibitors of anti-apoptotic Bcl-X_{L} and anti-apoptotic Bcl-2. It is expected that, because compounds having Formula I bind to and inhibit the activity of BCl-X_{L} and Bcl-2, they would also have utility as inhibitors of anti-apopotic protein family members having close structural homology to BCl-X_{L} and Bcl-2, such as, for example, anti-apopotic Bcl-w, Mcl-1 and Bfl-1/A1 proteins.

Accordingly, compounds having Formula I are expected to have utility in treatment of diseases during which anti-apopotic Bcl-X_{L} protein, anti-apopotic Bcl-2 protein, anti-apopotic Bcl-w protein or a combination thereof, are expressed.

Diseases during which anti-apopotic protein family members such as BCl-X_{L} protein, Bcl-2 protein and Bcl-w protein are expressed include cancer, neoplastic disease and autoimmune disorders, wherein cancer and neoplastic disease include, but are not limited to, cancer and autoimmune disorders, wherein cancer includes, but is not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung carcinoma, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, renal cell carcinoma, retinoblastoma, rhabdomyosarcoma, sarcoma, sebaceous gland carcinoma, seminoma, small cell lung carcinoma, solid tumors (carcinomas and sarcomas), small cell lung cancer squamous cell carcinoma, synovioma, sweat gland carcinoma, Waldenstrom's macroglobulinemia, testicular tumors, uterine cancer and Wilms' tumor, (Cancer Res., 2000, 60, 6101-10 and Medicine, 2d Ed., J.B. Lippincott Co., Philadelphia (1985)); autoimmune disorders include, but are not limited to, acquired immunodeficiency disease syndrome, autoimmune lymphoproliferative syndrome, hemolytic anemia, inflammatory diseases, and thrombocytopenia (Current Allergy and Asthma Reports 2003, 3:378-384; Br. J. Haematol. 2000 September; 110(3): 584-90; Blood 2000 Feb. 15; 95(4):1283-92; and New England Journal of Medicine 2004 September; 351(14): 1409-1418).

It is also expected that compounds having Formula I would inhibit the growth of cells derived from a cancer or neoplasm such as breast cancer (including estrogen-receptor positive breast cancer), colorectal cancer, endometrial cancer, lung cancer (including small cell lung cancer), lymphoma (including follicular or Diffuse Large B-cell), lymphoma (including non-Hodgkin's lymphoma), neuroblastoma, ovarian cancer, prostate cancer (including hormone-insensitive prostate cancer), testicular cancer (including germ cell testicular cancer).

It is also expected that compounds having Formula I would inhibit the growth of cells derived from a pediatric cancer or neoplasm such as embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous syatem, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer (commonly-owned U.S. application Ser. No. 10/988,338), Cancer Res., 2000, 60, 6101-10); autoimmune disorders include, but are not limited to, acquired immunodeficiency disease syndrome, autoimmune lymphoproliferative syndrome, hemolytic anemia, inflammatory diseases, and thrombocytopenia (Current Allergy and Asthma Reports 2003, 3:378-384; Br. J. Haematol. 2000 September; 110(3): 584-90; Blood 2000 Feb. 15; 95(4):1283-92; and New England Journal of Medicine 2004 September; 351(14): 1409-1418).

The compounds of the invention having Formula I are expected to be useful as chemotherapeutic agents alone or in combination with additional therapeutic agents including, but not limited to, angiogenesis inhibitors, antiproliferative agents, kinase inhibitors, receptor tyrosine kinase inhibitors, aurora kinase inhibitors, polo-like kinase inhibitors, bcr-abl kinase inhibitors, growth factor inhibitors, COX-2 inhibitors, non-steroidal anti-inflammatory drugs (NSAIDS), antimitotic agents, alkylating agents, antimetabolites, intercalating antibiotics, platinum containing agents, growth factor inhibitors, ionizing radiation, cell cycle inhibitors, enzymes, topoisomerase inhibitors, biologic response modifiers, immunologicals, antibodies, hormonal therapies, retinoids/deltoids plant alkaloids, proteasome inhibitors, HSP-90 inhibitors, histone deacetylase inhibitors (HDAC) inhibitors, purine analogs, pyrimidine analogs, MEK inhibitors, CDK inhibitors, ErbB2 receptor inhibitors, mTOR inhibitors as well as other antitumor agents.

Angiogenesis inhibitors include, but are not limited to, EGFR inhibitors, PDGFR inhibitors, VEGFR inhibitors, TIE2 inhibitors, IGF1R inhibitors, matrix metalloproteinase 2 (MMP-2) inhibitors, matrix metalloproteinase 9 (MMP-9) inhibitors and thrombospondin analogs.

Examples of EGFR inhibitors include, but are not limited to, Iressa (gefitinib), Tarceva (erlotinib or OSI-774), Erbitux (cetuximab), EMD-7200, ABX-EGF, HR3, IgA antibodies, TP-38 (IVAX), EGFR fusion protein, EGF-vaccine, anti-EGFr immunoliposomes and Tykerb (lapatinib).

Examples of PDGFR inhibitors include, but are not limited to, CP-673,451 and CP-868596.

Examples of VEGFR inhibitors include, but are not limited to, Avastin (bevacizumab), Sutent (sunitinib, SU11248), Nexavar (sorafenib, BAY43-9006), CP-547,632, axitinib (AG13736), Zactima (vandetanib, ZD-6474), AEE788, AZD-2171, VEGF trap, Vatalanib (PTK-787, ZK-222584), Macugen, IM862, Pazopanib (GW786034), ABT-869 and angiozyme.

Examples of thrombospondin analogs include, but are not limited to, TSP-1, ABT-510, ABT-567 and ABT-898.

Examples of aurora kinase inhibitors include, but are not limited to, VX-680, AZD-1152 and MLN-8054.

An example of a polo-like kinase inhibitor includes, but is not limited to BI-2536.

Examples of bcr-abl kinase inhibitors include, but are not limited to, Gleevec (imatinib) and Dasatinib (BMS354825).

Examples of platinum containing agents includes, but are not limited to, cisplatin, Paraplatin (carboplatin), eptaplatin, lobaplatin, nedaplatin, Eloxatin (oxaliplatin) and satraplatin.

Examples of mTOR inhibitors includes, but are not limited to, CCI-779, rapamycin, temsirolimus, everolimus, RAD001, and AP-23573.

Examples of HSP-90 inhibitors includes, but are not limited to, geldanamycin, radicicol, 17-AAG, KOS-953, 17-DMAG, CNF-101, CNF-1010,17-AAG-nab, NCS-683664, Mycograb, CNF-2024, PU3, PU24FC1, VER49009, IPI-504, SNX-2112 and STA-9090.

Examples of histone deacetylase inhibitors (HDAC) includes, but are not limited to, Suberoylanilide hydroxamic acid (SAHA), MS-275, Valproic acid, TSA, LAQ-824, Trapoxin, and Depsipeptide.

Examples of MEK inhibitors include, but are not limited to, PD325901, ARRY-142886, ARRY-438162 and PD98059.

Examples of CDK inhibitors include, but are not limited to, flavopyridol, MCS-5A, CVT-2584, seliciclib (CYC-202, R-roscovitine), ZK-304709, PHA-690509, BMI-1040, GPC-286199, BMS-387,032, PD0332991 and AZD-5438.

Examples of useful COX-2 inhibitors include, but are not limited to, CELEBREX™ (celecoxib), parecoxib, deracoxib, ABT-963, MK-663 (etoricoxib), COX-189 Lumiracoxib), BMS347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl-1H-pyrrole, T-614, JTE-522, S-2474, SVT-2016, CT-3, SC-58125 and Arcoxia (etoricoxib).

Examples of non-steroidal anti-inflammatory drugs (NSAIDs) include, but are not limited to, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol) and Oxaprozin (Daypro).

Examples of ErbB2 receptor inhibitors include, but are not limited to, CP-724-714, CI-1033 (canertinib), Herceptin (trastuzumab), Omitarg (2C4, petuzumab), TAK-165, GW-572016 (Ionafarnib), GW-282974, EKB-569, PI-166, dHER2 (HER2 Vaccine), APC8024 (HER2Vaccine), anti-HER/2neu bispecific antibody, B7.her2IgG3, AS HER2 trifunctional bispecfic antibodies, mAB AR-209 and mAB 2B-1.

Examples of alkylating agents include, but are not limited to, nitrogen mustard N-oxide, cyclophosphamide, ifosfamide, trofosfamide, chlorombucil, melphalan, busulfan, mitobronitol, carboquone, thiotepa, ranimustine, nimustine, Cloretazine (VNP 40101M), temozolomide, AMD-473, altretamine, AP-5280, apaziquone, brostallicin, bendamustine, carmustine, estramustine, fotemustine, glufosfamide, KW-2170, mafosfamide, and mitolactol, carmustine (BCNU), lomustine (CCNU), Busulfan, Treosulfan, Decarbazine and Temozolomide.

Examples of antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabine, 5-azacitidine, capecitabine, cladribine, clofarabine, decitabine, eflornithine, ethnylcytidine, cytosine arabinoside, hydroxyurea, TS-1, melphalan, nelarabine, nolatrexed, ocfosate, disodium premetrexed, pentostatin, pelitrexol, raltitrexed, triapine, trimetrexate, vidarabine, mycophenolic acid, tiazofurin, Ribavirin, EICAR, hydroxyurea and deferoxamine.

Examples of antibiotics include intercalating antibiotics but are not limited to, aclarubicin, actinomycin D, amrubicin, annamycin, adriamycin, bleomycin, daunorubicin, doxorubicin, elsamitrucin, epirbucin, glarbuicin, idarubicin, mitomycin C, nemorubicin, neocarzinostatin, peplomycin, pirarubicin, rebeccamycin, stimalamer, streptozocin, valrubicin, zinostatin and combinations thereof.

Examples of topoisomerase inhibiting agents include, but are not limited to, one or more agents selected from the group consisting of aclarubicin, amonafide, belotecan, camptothecin, 10-hydroxycamptothecin, 9-aminocamptothecin, Amsacrine, Cardioxane (Dexrazoxine), diflomotecan, irinotecan HCL (Camptosar), edotecarin, epirubicin (Ellence), etoposide, exatecan, Becatecarin, gimatecan, lurtotecan, orathecin (Supergen), BN-80915, mitoxantrone, pirarbucin, pixantrone, rubitecan, sobuzoxane, SN-38, tafluposide and topotecan.

Examples of antibodies include, but are not limited to, Rituximab, Cetuximab, Bevacizumab, Trastuzimab, specific CD40 antibodies and specific IGF1R antibodies, chTNT-1/B, Denosumab, Panorex (Edrecolomab), Rencarex (WX G250), Zanolimumab, Lintuzumab, Ticilimumab.

Examples of hormonal therapies include, but are not limited to, exemestane (Aromasin), leuprolide acetate, Buserelin, Cetrorelix, Deslorelin, Vantas, anastrozole (Arimidex), fosrelin (Zoladex), goserelin, Degarelix, doxercalciferol, fadrozole, formestane, tamoxifen citrate (tamoxifen), Arzoxifene, Casodex, Abarelix, Trelstar, finasteride, fulvestrant, toremifene, raloxifene, Trilostane (Modrastane, Desopan), lasofoxifene, letrozole, flutamide, bicalutamide, megesterol, mifepristone, nilutamide, dexamethasone, predisone and other glucocorticoids.

Examples of retinoids/deltoids include, but are not limited to, seocalcitol (EB 1089, CB 1093), lexacalcitrol (KH 1060), fenretinide, Panretin (aliretinoin), Atragen, Bexarotene and LGD-1550.

Examples of plant alkaloids include, but are not limited to, vincristine, vinblastine, vindesine and vinorelbine.

Examples of proteasome inhibitors include, but are not limited to, bortezomib (Velcade), MG132, NPI-0052 and PR-171.

Examples of immunologicals include, but are not limited to, interferons and numerous other immune enhancing agents. Interferons include interferon alpha, interferon alpha-2a, interferon alpha-2b, interferon beta, interferon gamma-1a, interferon gamma-1b (Actimmune), or interferon gamma-n1 and combinations thereof. Other agents include Alfaferone (Leukocyte alpha interferon, Cliferon), filgrastim, lentinan, sizofilan, TheraCys, ubenimex, WF-10, aldesleukin, alemtuzumab, BAM-002, decarbazine, daclizumab, denileukin, gemtuzumab ozogamicin, ibritumomab, imiquimod, lenograstim, lentinan, melanoma vaccine (Corixa), molgramostim, OncoVAC-CL, sargaramostim, tasonermin, tecleukin, thymalasin, tositumomab, Virulizin, Z-100, epratuzumab, mitumomab, oregovomab, pemtumomab (Y-muHMFG1), Provenge (Dendreon), CTLA4 (cytotoxic lymphocyte antigen 4) antibodies and agents capable of blocking CTLA4 such as MDX-010.

Examples of biological response modifiers are agents that modify defense mechanisms of living organisms or biological responses, such as survival, growth, or differentiation of tissue cells to direct them to have anti-tumor activity. Such agents include krestin, lentinan, sizofuran, picibanil PF-3512676 (CpG-8954) and ubenimex.

Examples of pyrimidine analogs include, but are not limited to, 5-Fluorouracil, Floxuridine, Doxifluridine, Ratitrexed, cytarabine (ara C), Cytosine arabinoside, Fludarabine, triacetyluridine Troxacitabine (Troxatyl) and Gemcitabine.

Examples of purine analogs include but are not limited to, Mercaptopurine and thioguanine.

Examples of antimitotic agents include, but are not limited to, N-(2-((4-hydroxyphenyl)amino)pyridin-3-yl)-4-methoxybenzenesulfonamide, paclitaxel, docetaxel, epothilone D (KOS-862), PNU100940 (109881), Batabulin, Ixabepilone (BMS 247550), Patupilone, XRP-9881, Vinflunine and ZK-EPO.

Compounds of the present invention are also intended to be used as a radiosensitizer that enhances the efficacy of radiotherapy. Examples of radiotherapy include but are not limited to, external beam radiotherapy (XBRT), or teletherapy, brachtherapy or sealed source radiotherapy, unsealed source radiotherapy.

Additionally, compounds having Formula I may be combined with other antitumor agents selected from the following agents, Genasense, Panitumumab, Zevalin, Bexxar (Corixa), Arglabin, Abarelix, Alimta, EP0906, discodermolide, Neovastat, enzastaurin, Combrestatin A4P, ZD-6126, AVE-8062, DMXAA, Thymitaq, Temodar, Revlimid, Cypat, Histerelin, Plenaizis, Atrasentan, Celeuk (celmoleukin), Satraplatin, thalomide (Thalidomide), theratope, Temilifene, ABI-007, Evista, Atamestane, Xyotax, Targretin, Triazone, Aposyn, Nevastat, Ceplene, Lanreotide, Aredia (pamidronic acid), Orathecin, Virulizin, Gastrimmune, DX-8951f, Mepact (Liposome muramyl tripeptide phophatidylethanolamine, Junovan), Dimericine (Liposome T4 endonuclase V), Onconase, BEC2, Xcytrin, CeaVac, NewTrexin, OvaRex, Osidem, Advexin, RSR13 (efaproxiral, Cotara, NBI-3001 (IL-4), Canvaxin, GMK vaccine, PEG Interferon A, Taxoprexin, gene therapy agents such as TNFerade (GeneVac) or GVAX, Interferon-alpha, Interferon-gamma, Gardasil, Eniluracil (GW 776C85), Lonafarnib, ABT-100, Tumor necrosis factor, Lovastatin, staurosporine, dactinomycin, zorubicin, Bosentan, OncoVAX, Cervarix, Cintredekin besudotox (IL-13-PE38, IL-13-PE38QQR, Interleukin 13-pseudomonas exotoxin), Oncophage (HSPPC 96), Phenoxodiol (NV 06), IGN 101, PANVAC (CEA, MUC-1 vaccinia), ampligen, ibandronic acid, miltefosine, L-asparaginase, procarbazine, Trabectedin (ET-743, Ecteinascidin 743, Yondelis), 5,10-methylenetetrahydrofolate, hydroxycarbamide, pegaspargase, pentostatin, tazarotne, TransMID 107R (KSB 311), Trisenox, Telcyta, tretinoin, acitretin, Zometa (zolendronic acid), Pandimex (Aglycon protopanaxadiol, PBD-2131), Talabostat (PT100), Tesmilifene, Tetrandrine, halofuginone, rebimastat, removab, squalamine, ukrain, paditaxel, Zinecard and Vitaxin.

The following Examples are provided merely to illustrate certain aspects of the invention and do not by any means limit the scope of the invention.

### EXAMPLES

### Example 1

### Preparation of 7-(piperazin-1-yl)quinazolin-4-ol

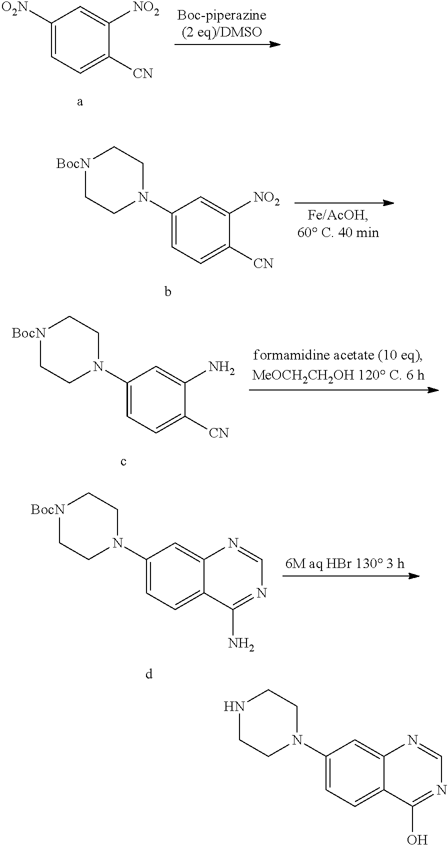

2,4-dinitrobenzonitrile (10g) and Boc-piperazine (20g, ca 2 eq) in DMSO (60 ml) was stirred for 3 days at room temperature. The dark brown reaction mixture was then partitioned between ethyl acetate (ca 400 ml) and water (ca 2×100 mL) [note—warming was sometime needed in order to prevent product from crystallizing out of organic layer]. The separated and dried organic layer was concentrated and the residue triturated with ether and filtered to give intermediate b (1st crop 8g, 47% yield,) as a deep yellow powder. A 2^{nd} crop was obtained from the ethereal ethyl acetate supernatant on standing after some evaporation had taken place (2^{nd} crop. 1.7 g, 10%).

Intermediate b(3g) was reduced to the aniline c using iron powder (2g) in acetic acid (20 ml) at 60 deg. with rapid stirring. The reaction mixture was diluted with ethyl acetate (ca 60 ml), filtered twice through celite, and the acetic acid removed with a base wash using ca 6M aq. NaOH. The washed organic layer was separated and dried and concentrated to give intermediate c (52-72% yield) as a pale yellow powder.

Intermediate c (1.5 g) in MeOCH₂CH₂OH (10 ml) at 120 deg. was treated portionwise (4×2.5 eq over the period of an hour) with formamidine acetate and the whole heated for a further 6 hours, during which time the reaction mixture became heterogenous due to product formation. After standing overnight at room temperature, the reaction mixture was shaken with ether (ca 40 mL) and filtered and the filtercake washed further with ether, then slurried with water (ca 40 ml) and re-filtered to give intermediate d as a colourless powder (ca 1.1 g, 69%). On standing, the filtrate after evaporation of the organic layer gave a further crop of crude product (ca 22%).

Intermediate d (4 mmol) was then added carefully with rapid stirring [note—vigorous effervescence occurred] to ca 6M aq. HBr (ca 5-6 mL) to initially remove the Boc group giving the intermediate V. Further stirring and heating at 130° C. in a capped vessel for 3 h gave the hydrolysed product 7-(piperazin-1-yl)quinazolin-4-ol dihydrobromide as colourless needles (97%) after the hot reaction mixture was added to hot methanol (ca 50 mL) and the whole allowed to cool overnight and filtered [note—cooling too rapidly led to an intractable gel]. M⁺ 231.

### Example 2

### Preparation of 2-bromomethyl-4′-chloro-biphenyl

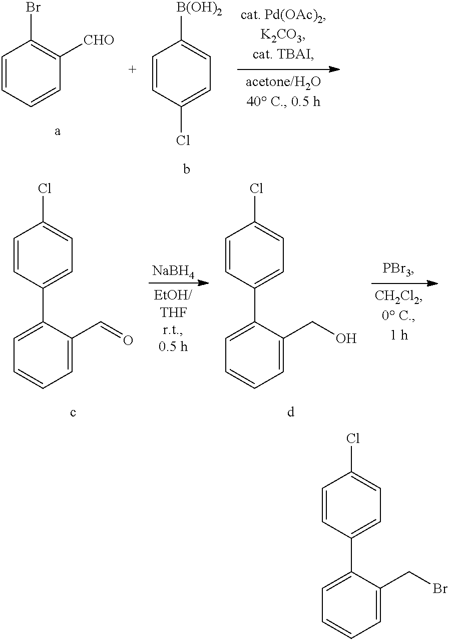

2-Bromobenzaldehyde a (19 mmol), 4-cholorophenyl boronic acid b (19 mmol), tetrabutylammonium iodide (0.19 mmol), potassium carbonate (57 mmol) and palladium acetate (0.12 mmol) in mixture of acetone/water (25 ml/25 ml) was stirred at 40° C. for 30 mins. The mixture was partitioned between ethyl acetate and water and the layers were separated. The organic layer was dried (MgSO₄) and concentrated in vacuo. The resulting residue was applied to silica chromatography gradient eluting with 100% petroleum ether to 5% ethyl acetate/petroleum ether to yield intermediate c 4′-chloro-4′-chloro-biphenyl-2-carbaldehyde as a colourless oil (76%). ¹H NMR (300 MHz, CDCl₃) δ 9.96 (1H, s), 8.02 (1H, dd, J 7.8 and 1.0 Hz), 7.64-7.30 (7H, m, ArH).

Sodium borohydride (11.5 mmol) was added to a mixture of the aldehyde (2.3 mmol) in a mixture of tetrahydrofuran and ethanol (7.5 ml/7.5 ml) at room temperature. The mixture was stirred for 30 mins and was then quenched by addition of cold water. The pH was adjusted to pH 5-6 and the solution stirred for 15 mins. Diethyl ether (20 ml) was added to the solution and layers were then separated. The aqueous solution was extracted once more with diethyl ether (20 ml). The combined organic layers were dried (MgSO₄) and concentrated in vacuo to yield intermediate d 4′-chloro-biphenyl-2-yl)-methanol as a colourless oil (95%). The compound was of sufficient purity to be used in the next step without further purification. ¹H NMR (300 MHz, DMSO) δ 7.56-7.18 (8H, m, ArH) 5.1 (1H, bs, OH) and 4.36 (2H, s, ArCH₂).

Phosphorous tribromide (4.6 mmol) in dichloromethane (10 ml) was added slowly to a solution of the alcohol d (4.6 mmol) in dry dichloromethane (40 ml) at 0° C. The solution was allowed to stir for 1 h at 0° C. and was then quenched by addition of cold water. The layers were separated and then aqueous was extracted with dichloromethane (20 ml). The combined organic layers were dried (MgSO₄) and concentrated in vacuo. Further drying yielded intermediate 2-bromomethyl-4′-chloro-biphenyl as a white solid (80%). The compound was of sufficient purity to be used in the next step without further purification. ¹H NMR (300 MHz, CDCl₃) δ 7.56-7.23 (8H, m, ArH) and 4.44 (2H, s, ArCH₂).

A solution of phosphorous chloride (0.5 ml) and N,N-dimethylformamide (0.058 mmol) in 1,2-dichloroethane (2 ml) was added dropwise over 15 mins to a stirred solution of the quinazolinone c (1.16 mmol) in 1,2-dichloroethane (30 ml) at 70° C. under an atmosphere of nitrogen. Additional phosphorous chloride was added in increments (1 ml) of 15 mins over the next hour. The solution was allowed to stir at 70° C. for 20 h. The solution was then concentrated in vacuo to dryness and then diluted with a solution of 10% sodium hydrogencarbonate (40 ml) and dichloromethane (40 ml). The layers were separated, the organic layer was dried (MgSO₄) and concentrated in vacuo. The resulting residue was then applied to alumina column chromatography gradient eluting from 100% dichloromethane to 0.5% methanol/dichloromethane to afford 4-chloro-7-(4-((2-(4-chlorophenyl)-5,5-dimethylcyclohex-1-enyl)methyl)piperazin-1-yl)quinazoline as a yellow foam (58%). LCMS—r.t. 6.41, M+H 463.

### Example 3

### Preparation of 1 N-{7-[4-(4′-Chloro-biphenyl-2-ylmethyl)piperazin-1-yl]-quinazolin-4-yl}-4-((R)-3-dimethylamino-1-phenylsulfanylmethyl-propylamino)-3-nitro-benzenesulfonamide

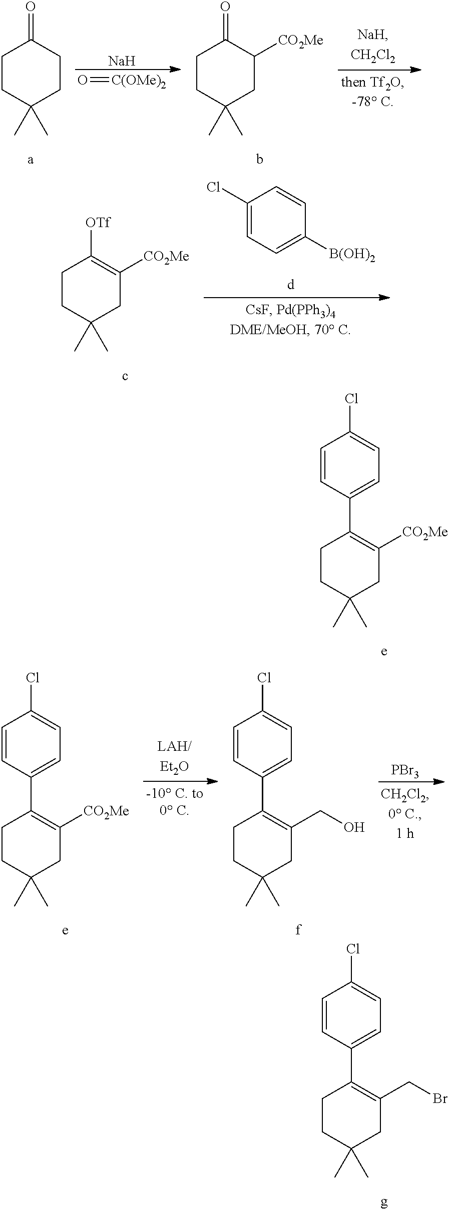

To a solution of 21 g of dimethyl carbonate (0.23 mol) in dry THF (400 ml) was added sodium hydride (9.6 g, 0.24 mol) by portion at 0° C. The resulting mixture was stirred at 0° C. for 30 min and then was added a solution of 10 g of compound a (79 mmol) in THF (100 ml) dropwise over 30 min. The resultant mixture was heated to 60° C.-80° C. for 3 h before cooled to room temperature. The reaction mixture was poured into saturated NaHCO₃ solution and extracted with ether. The organic layer was washed with water, brine, dried over Na₂SO₄ and concentrated to give 25g of intermediate b methyl 5,5-dimethyl-2-oxocyclohexanecarboxylate (yield: 84%). MS (ESI) m/e (M+H⁺): 185.

To a solution of b (10 g, 54 mmol) in dry DCM (100 ml) was added sodium hydride (6.6 g, 0.16 mol) by portion at 0° C. The resulting mixture was stirred at 0° C. for 30 min and then was cooled down to −78° C. 46.6 g of trifluoromethanesulfonic anhydride was added to the slurry dropwise over 1 h. The resultant mixture was warmed to r. t. and stirred overnight. The reaction mixture was poured into saturated NaHCO₃ solution and extracted with DCM. The organic layer was washed with water, brine, dried over Na₂SO₄ and concentrated to give the crude product, which was purified by column to afford 9.5 g of intermediate c methyl 5,5-dimethyl-2-(trifluoromethylsulfonyloxy)cyclohex-1-ene-carboxylate (yield: 55%). MS (ESI) m/e (M+H⁺): 317.

A mixture of compound c (5.1 g, 16 mmol), compound d (3.0 g, 19 mmol), cesium fluoride (6.1 g, 40 mmol) and tetrakis(triphenylphosphine)palladium(0) (0.8 mmol) in 2:1 DME/methanol (100 ml) was heated to 70° C. under N₂ atmosphere overnight. The mixture was filtered through celite and concentrated to give crude product, which was purified by column to afford 4g of intermediate e methyl 2-(4-chlorophenyl)-5,5-dimethylcyclohex-1-enecarboxylate e (yield: 89%). MS (ESI) m/e (M+H⁺): 279.

To a suspension of LiAlH₄ (0.95g, 25 mmol) in ether (100 ml) was added intermediate e (2.79 g, 10 mmol) at −10° C. over 30 min. The resultant mixture was stirred for 1 h 30 min at −10° C.-0° C. Then the reaction mixture was quenched with 1 ml water and 1 ml 10% NaOH aqueous solution at 0° C. The resulting mixture was filtered and the filtrate was diluted with ether, then the ether layer wash washed with water, brine, and dried over anhydrous Na₂SO₄ and concentrated to afford 2.3 g of intermediate f (2-(4-chlorophenyl)-5,5-dimethylcyclohex-1-enyl)methanol (yield: 95%). MS (ESI) m/e (M+H⁺): 251/233. ¹H-NMR (DMSO, 400 MHz): δ 7.35 (d, J=8.4 Hz, 2H), 7.20 (d, J=8.4 Hz, 2H), 4.52 (t, J=5.2 Hz, 1H), 3.67 (d, J=4.8 Hz, 1H), 2.21 (t, J=6.0 Hz, 1H), 1.92 (s, 2H), 1.40 (t, J=6.4 Hz, 2H), 0.94 (s, 6H),

Phosphorous tribromide (4.6 mmol) in dichloromethane (10 ml) was added slowly to a solution of the intermediate f (4.6 mmol) in dichloromethane (40 ml) at 0° C. The solution was allowed to stir for 1 h at 0° C. and was then quenched by addition of cold water. The layers were separated and then the aqueous was extracted with dichloromethane (20 ml). The combined organic layers were dried (MgSO₄) and concentrated in vacuo to afford 1-(2-bromomethyl-4,4-dimethyl-cyclohex-1-enyl)-4-chloro-benzene g as a colourless oil (95%). The compound was of sufficient purity to be used in the next step without further purification. ¹H NMR (300 MHz, CDCl₃) δ 7.26 (4H, q, J=17.2 Hz), 3.83 (2H, s), 2.31-2.27 (2H, m), 2.09 (3H, t, J=2.1 Hz), 1.49 (2H, t, J=6.5 Hz) and 1.01 (6H, s).

### Example 4

### Preparation of 4-chloro-7-[4-(4′-chloro-biphenyl-2-ylmethyl)piperazin-1-yl]-quinazoline

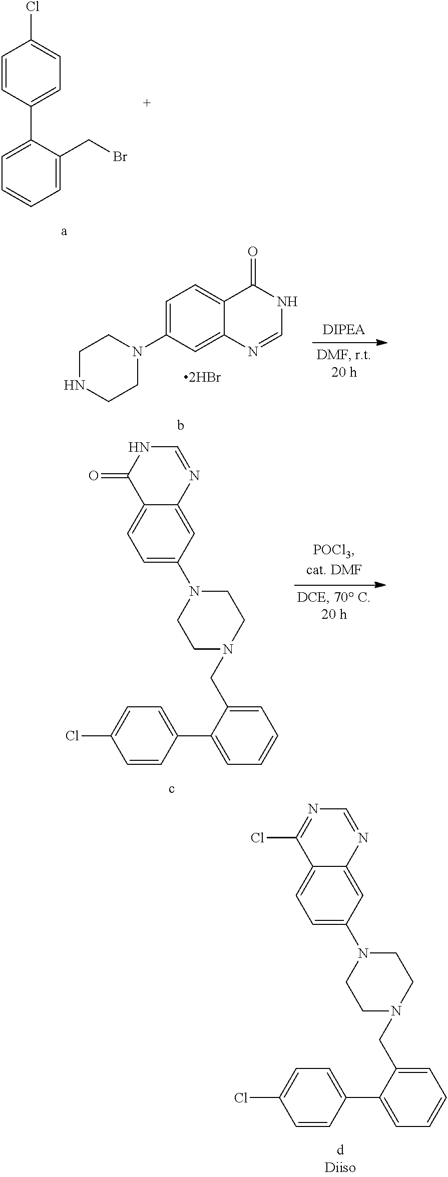

propylethylamine (2.55 mmol) was added to a stirred solution of the quinazolinone b (1.28 mmol) in N,N-dimethylformamide (10 ml). To this solution the bromide intermediate a (1.28 mmol) in N,N-dimethylformamide (4 ml) was added dropwise over 30 mins. The solution was allowed to stir at room temperature for 20 h. A solution of 10% sodium hydrogencarbonate (50 ml) was added to the stirred solution. The resulting precipitate was filtered off and dried in a vacuum oven to yield intermediate c 7-[4-(4′-Chloro-biphenyl-2-ylmethyl)-piperazin-1-yl]-quinazolin-4-ol as a white solid (80%). The compound was of sufficient purity to be used in the next step without further purification. ¹H NMR (300 MHz, DMSO) δ 7.92 (1H, s), 7.86 (1H, d, J=9.0 Hz) 7.52-7.34 (7H, m), 7.23 (1H, dd, J 6.9 and 1.9 Hz), 7.11 (1H, dd, J 9.0 and 2.2 Hz), 6.88 (1H, d, J=2.3 Hz), 3.38 (2H, s), 3.25 (4H, bs) and 2.41 (4H, bs). LCMS-r.t. 5.77, M+H 431.

A solution of phosphorous chloride (0.5 ml) and N,N-dimethylformamide (0.058 mmol) in 1,2-dichloroethane (2 ml) was added dropwise over 15 mins to a stirred solution of the quinazolinone c (1.16 mmol) in 1,2-dichloroethane (30 ml) at 70° C. under an atmosphere of nitrogen. Additional phosphorous chloride was added in increments (1 ml) of 15 mins over the next hour. The solution was allowed to stir at 70° C. for 20 h. The solution was then concentrated in vacuo to dryness and then diluted with a solution of 10% sodium hydrogencarbonate (40 ml) and dichloromethane (40 ml). The layers were separated, the organic layer was dried (MgSO₄) and concentrated in vacuo. The resulting residue was then applied to alumina column chromatography gradient eluting from 100% dichloromethane to 0.5% methanol/dichloromethane to afford 4-chloro-7-[4-(4′-chloro-biphenyl-2-ylmethyl)-piperazin-1-yl]-quinazoline d as a yellow foam (55%). ¹H NMR (300 MHz, CDCl₃) δ 8.80 (1H, s), 8.02 (1H, d, J=9.4 Hz) 7.37-7.24 (7H, m), 7.12 (1H, d, J=2.5 Hz), 3.45 (6H, bs), 2.55 (4H, s). LCMS-r.t. 3.67, M+H 449.

### Example 5

### Preparation of 4-chloro-7-(4-((2-(4-chlorophenyl)-5,5-dimethylcyclohex-1-enyl)methyl)piperazin-1-yl)quinazoline

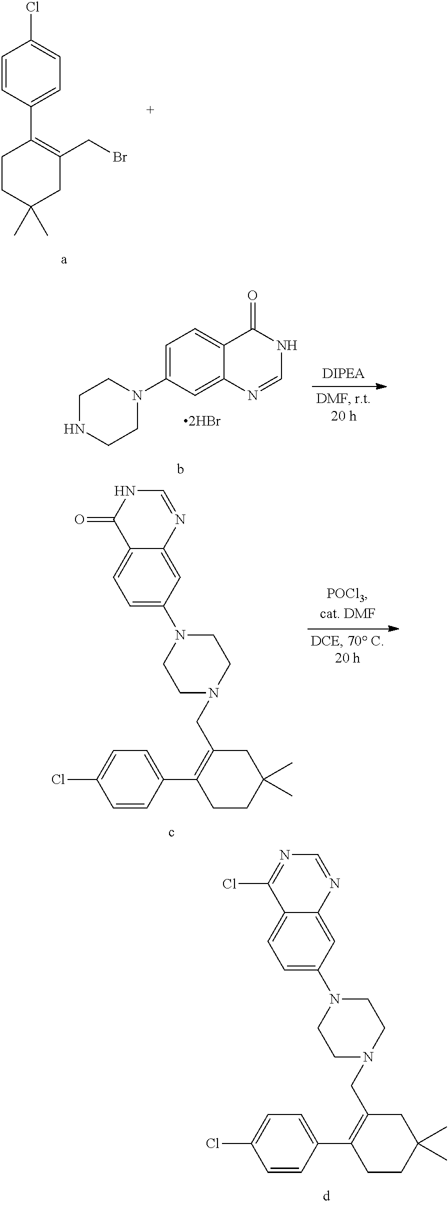

Diisopropylethylamine (2.55 mmol) was added to a stirred solution of the quinazolinone b (1.28 mmol) in N,N-dimethylformamide (10 ml). To this solution the bromide a (1.28 mmol) in N,N-dimethylformamide (4 ml) was added dropwise over 30 mins. The solution was allowed to stir at room temperature for 20 h. A solution of 10% sodium hydrogencarbonate (50 ml) was added to the stirred solution. The resulting precipitate was filtered off and dried in a vacuum oven to yield intermediate c as a white solid (84%). The compound was of sufficient purity to be used in the next step without further purification. ¹H NMR (300 MHz, DMSO) δ 7.92 (1H, s), 7.83 (1H, d, J=9.0 Hz) 7.36 (2H, d, J=6.5 Hz), 7.15 (2H, d, J=6.5 Hz), 7.06 (1H, dd, J 9.0 and 2.4 Hz), 6.82 (1H, d, J=2.3 Hz), 3.25 (4H, bs), 2.74 (2H, bs), 2.27-2.21 (6H, m), 1.98 (2H, s), 1.42 (2H, t, J=6.4 Hz) and 0.96 (6H, s). LCMS—r.t. 5.95, M+H 463.

A solution of phosphorous chloride (0.5 ml) and N,N-dimethylformamide (0.058 mmol) in 1,2-dichloroethane (2 ml) was added dropwise over 15 mins to a stirred solution of the quinazolinone c (1.16 mmol) in 1,2-dichloroethane (30 ml) at 70° C. under an atmosphere of nitrogen. Additional phosphorous chloride was added in increments (1 ml) of 15 mins over the next hour. The solution was allowed to stir at 70° C. for 20 h. The solution was then concentrated in vacuo to dryness and then diluted with a solution of 10% sodium hydrogencarbonate (40 ml) and dichloromethane (40 ml). The layers were separated, the organic layer was dried (MgSO₄) and concentrated in vacuo. The resulting residue was then applied to alumina column chromatography gradient eluting from 100% dichloromethane to 0.5% methanol/dichloromethane to afford 4-chloro-7-(4-((2-(4-chlorophenyl)-5,5-dimethylcyclohex-1-enyl)methyl)piperazin-1-yl)quinazoline d as a yellow foam (58%). LCMS—r.t. 6.41, M+H 463.

### Example 6

### Preparation of tert-butyl 4-(4-cyano-3-nitrophenyl)piperazine-1-carboxylate

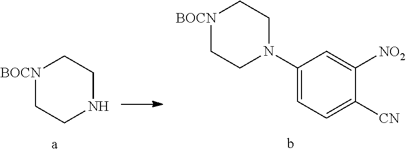

Boc-piperazine a (20 mmol) in DMSO (20 mL) was treated with 2,4-dinitrobenzonitrile (10 mmol) and the reaction mixture, which immediately became deep orange/red, was stirred overnight at room temperature. The reaction mixture was partitioned between ethyl acetate and 10% citric acid, the ethyl acetate layer further washed, evaporated and the residue triturated with ether to give a piperizinyl product b (yield 33%) as a yellow powder. If contaminated with starting nitrile, crystallization from ethyl acetate/ether was effective (M⁺ [ES⁺] 333, ¹H δ: (ppm, d6-DMSO) 7.82, d (J₁ 8.86 Hz), 1H, ArH; 7.66, d (J₂, 2.52 Hz), 1H, ArH; 7.28, dd (J₁ 8.86 Hz, J₂ 2.52 Hz), 1H, ArH; 3.4-3.5, m, 8H, 4×CH₂; 1.39, m, 9H, CMe₃.

### Example 7

### Preparation of 2-nitro-4-(piperazin-1-yl)benzonitrile bistosylate salt

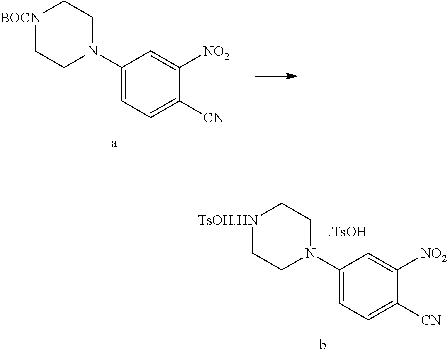

The piperazinyl compound a (15 mmol) was deprotected by dissolving in acetonitrile (40 mL) and treating with 5 equivalents of p-toluenesulfonic acid in acetonitrile (20 mL) and standing 2 hours. The product in the form of a bis-tosylate was then filtered off as course prisms (yield 83%). (M⁺ [ES⁺] 233; ¹H δ: (ppm, d6-DMSO) 8.80, bs, 1H, N⁺H, 7.88, d (J₁ 8.82 Hz), 1H, ArH; 7.76, d (J₂ 2.58 Hz), 1H, ArH; 7.46, d (J₁ 8.07 Hz), 4H, 4×ArH; 7.37, dd (J₁ 8.82 Hz, J₂ 2.58 Hz), 1H, ArH; 7.09, d (J₁ 8.07 Hz), 4H, 4×ArH; 3.6-3.7, m, 4H, 2×CH₂; 3.1-3.3, m, 4H, 2×CH₂; 2.25, s, 6H, 2×Me.

### Example 8

### Preparation of 4-(4-(2-bromobenzyl)piperazin-1-yl)-2-nitrobenzonitrile

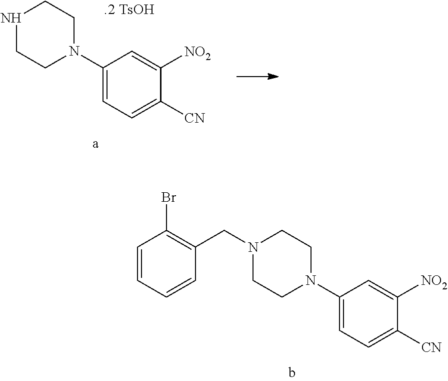

To 4 mmol of this bis-tosylate a and 6 mmol of 2-bromobenzylbromide in isopropanol (15 mL) was added triethylamine (14 mmol) and the whole stirred for 3 hours. Methanol was then added (20 mL), the mixture allowed to stand a few minutes, and the aryl bromide product b, was filtered off pure as an orange powder (93%). (M⁺ [ES⁺] 401, 403; ¹H δ: (ppm, d6-DMSO) 7.80, d (J₁ 8.9 Hz), 1H, ArH; 7.67, d (J₂ 2.47 Hz), 1H, ArH; 7.58, d (J₁ 7.6 Hz), 1H, ArH; 7.49, d (J₁ 7.6 Hz), 1H, ArH; 7.36, dd (J₁ 7.6 Hz, J₁ 7.6 Hz), 1H, ArH; 7.30, dd (J₁ 8.9 Hz, J₂ 2.47 Hz); 7.19, dd (J₁ 7.6 Hz, J₁ 7.6 Hz); 3.58, s, 2H, CH₂; 3.4-3.5, m 4H, 2×CH₂; 2.5-2.6, m 4H, 2×CH₂.

### Example 9

### Preparation of 4-(4-((4′-chlorobiphenyl-2-yl)methyl)piperazin-1-yl)-2-nitrobenzonitrile tosylate salt

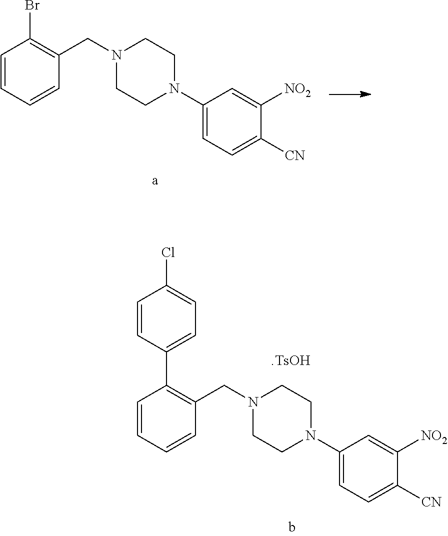

To a mixture of 3.43 mmol of the aryl bromide, 703 mg of p-chlorophenylboronic acid, and 50 mg PdCl₂(PPh₃)₂ stirring in 1:1:1 dimethoxyethane:ethanol:water (20 mL) under nitrogen was added 2M aqueous sodium carbonate solution (2.25 mL) and the solution heated at 90° C. for 4 hours. The reaction mixture was partitioned between ethyl acetate and water, filtered through celite, the organic layer dried, evaporated and the residue treated with 10 mmol of p-toluenesulfonic acid in acetonitrile (20 mL) with ether (40 mL) then added. On standing in the freezer, the nitroarene product precipitated as a yellow powder: yield 1.68 g (81%). (M⁺ [ES⁺] 433, 435; ¹H δ: (ppm, d6-DMSO) 9.57, bs, 1H, N⁺H, 7.85, d (J₁ 8.8 Hz), 1H, ArH; 7.7-7.8, m, 1H, ArH; 7.69, d (J₂ 2.52 Hz), 1H, ArH; 7.4-7.6, m, 6H, 6×ArH; 7.2-7.4, m, 4H, 4×ArH; 7.08, d (J₁ 7.9 Hz), 2H, 2×ArH; 4.36, m, 2H, CH₂; 4.07, m, 2H, CH₂; 3.22, m, 4H, 2×CH₂; 2.88, m, 2H, CH₂; 2.25, 2, 3H, Me.

### Example 10

### Preparation of 2-amino-4-(4-((4′-chlorobiphenyl-2-yl)methyl)piperazin-1-yl)benzonitrile

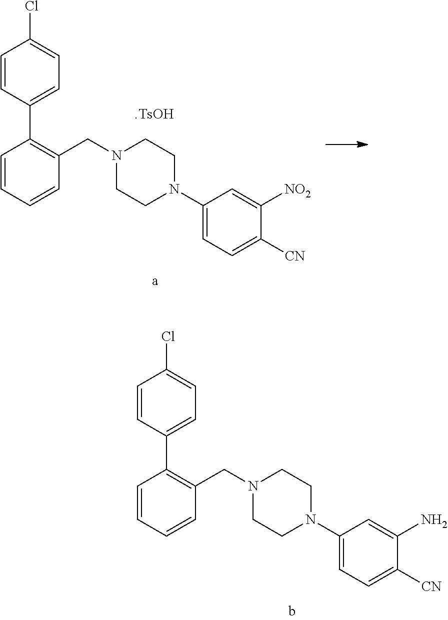

The nitroarene compound a (62 mg) and iron powder (50 mg) in glacial acetic acid (0.2 ml) was heated at 90° C. with stirring for 10 minutes, partitioned between ethyl acetate and saturated aqueous sodium bicarbonate solution, the organic layer separated, washed and evaporated to dryness to give the crude aniline, as a brownish residue. This was repeated on a 1.3 g scale. The crude residue was purified by triturating with ether. This gave the aniline product b in ca 60% yield, and a further 25% could be recovered from the ethereal supernatant if so desired. (M⁺ [ES⁺] 403,405; ¹H δ: (ppm, d6-DMSO) 7.1-7.6, m, 9H, 9×ArH; 6.23, d (J₁ 9.2 Hz), 1H, ArH; 6.04, s, 1H, ArH; 4.23, bs, 2H, CH₂; 3.40, bs, 2H, NH₂; 3.19, bs, 4H, 2×CH₂; 2.34, bs, 4H, 2×CH₂.

### Example 11

### Preparation of 7-(4-((4′-chlorobiphenyl-2-yl)methyl)piperazin-1-yl) quinazolin-4-amine

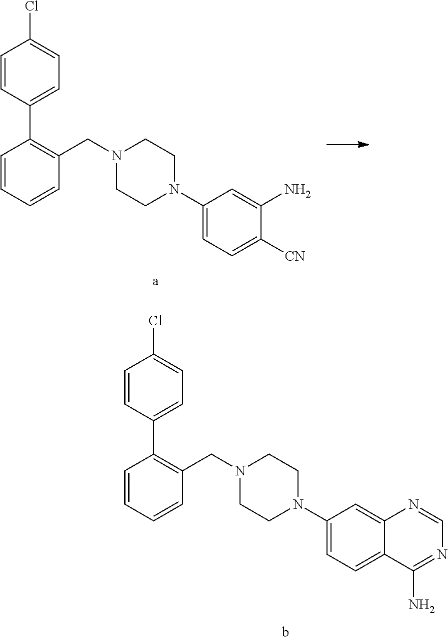

The aniline compound a (216 mg) was on-reacted with formamidine acetate (10 eq) in MeOCH₂CH₂OH (5 mL) at reflux for 3 hours under nitrogen, and the product precipitated from the dark, cooled reaction mixture by the addition of a little water. This was filtered and dried to give the 4-aminoquinazoline compound b, as a buff solid that could be recrystallized from aqueous DMSO after neutralization with aqueous ammonia (198 mg, yield 81%). M⁺ [ES⁺] 430, 432. ¹H δ: (ppm, d6-DMSO) 8.18, s, 1H, ArH (H2); 7.95, d, (J 9.2 Hz), 1H, ArH (H5); 7.47-7.50, m, 1H, ArH; 7.45, bs, 4H, 4×ArH; 7.29-7.36, m, 4H, 2×ArH+NH₂; 7.20-7.23, m, 1H, ArH; 7.16, dd (J, 9.2 Hz, J₂ 2.4 Hz) 1H, ArH; 6.80, d, (J 2.3 Hz), 1H, ArH; 3.37, s, 2H, CH₂; 3.23, m, 4H, 2×CH₂ (piperazine); 2.40, m, 4H, 2×CH₂ (piperazine).

### Example 12

### Preparation of 7-(4-((4′-chlorobiphenyl-2-yl)methyl)piperazin-1-yl)quinazolin-4(3H)-one

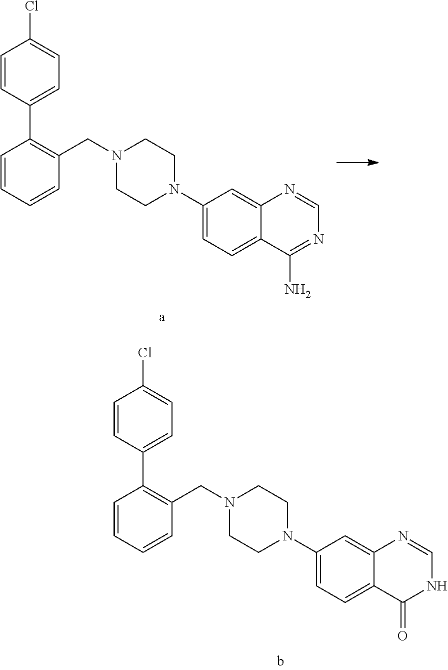

The 4-aminoquinazoline compound a (176 mg) was heated at ca 130° C. for 9 hours in glacial acetic acid (2 mL) and concentrated aqueous (25%) hydrochloric acid solution (2 mL) in a small flask fitted with an air condenser. The solvent was removed and the residue recrystallized from aqueous DMSO after neutralization with minimal aqueous ammonia. This gave the hydrolysed product b as a buff powder (87% yield). M⁺ [ES⁺] 431, 433. ¹H δ: (ppm, d6-DMSO) 11.8, bs, 1H, OH; 7.90, s, 1H, ArH (H2); 7.84, d (J 9.0 Hz), 1H, ArH (H5); 7.46-7.51, m, 1H, ArH; 7.44, bs, 4H, 4×ArH; 7.30-7.38, m, 2H, 2×ArH; 7.20-7.23, m, 1H, ArH, 7.09, dd, (J, 9.0 Hz, J₂ 2.0 Hz) 1H, ArH; 6.86, d, (J 2.0 Hz) 1H, ArH; 4.44-3.37, bs, 2H, NCH₂Ph; 3.27, m, 4H, 2×CH₂ (piperazine) 2.38, bm, 4H, 2×CH₂. (piperazine).

### Example 13

### Preparation of 4-chloro-7-(4-((4′-chlorobiphenyl-2-yl)methyl)piperazin-1-yl)quinazoline

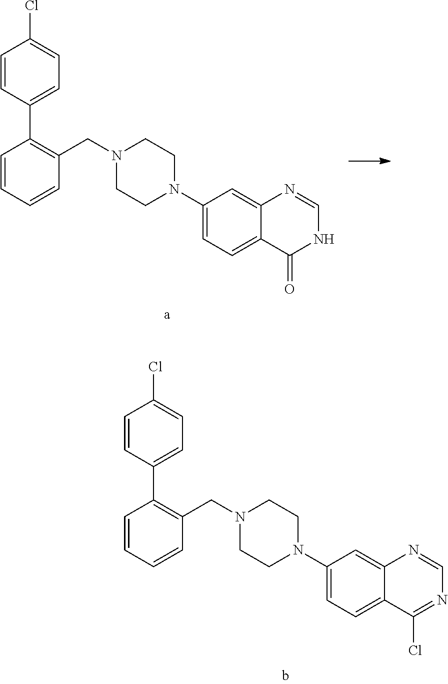

The hydrolysed product a was chlorinated by treating 30 mg in 1 mL dry chloroform and 1 mL thionyl chloride with a catalytic amount of DMF (10 uL), refluxing 1 hour, pouring onto ice and extracting product with ethyl acetate, to give the chlorinated product b which was on-reacted without characterization.

### Example 14

### Preparation of 3-nitro-4-(2-(phenylthio)ethylamino)benzenesulfonamide

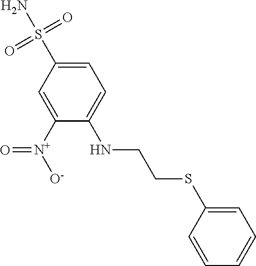

The sulfonamide, 3-nitro-4-(2-(phenylthio)ethylamino)benzenesulfonamide, was prepared using techniques described in WO 2002/24636, the teaching of which is incorporated herein by reference.

### Example 15

### Preparation of N-(7-(4-((4′-chlorobiphenyl-2-yl)methyl)piperazin-1-yl)quinazolin-4-yl)-3-nitro-4-(2-(phenylthio)ethylamino)benzenesulfonamide

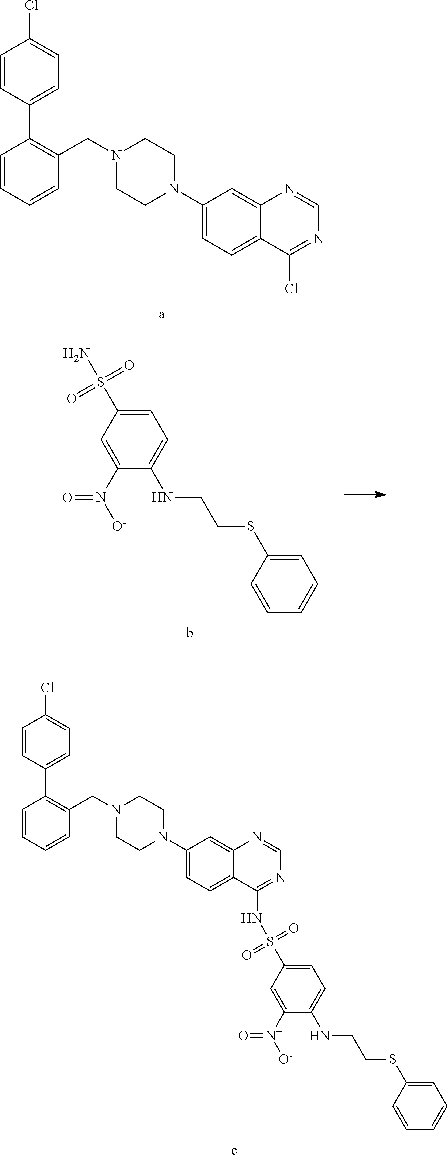

A portion of the chloroquinaline compound a is coupled to the sulfonamide b by heating at 85° C. in DMF (0.2 mL) with potassium carbonate (40 mg) overnight. The reaction mixture is partitioned between ethyl acetate (2 mL) and water (2 mL), and the organic layer is separated, dried and evaporated to give a yellow residue. This was purified by HPLC to give 2 mg of the compound c.

### Example 16

### Preparation of N-(7-(4-((4′-chlorobiphenyl-2-yl)methyl)piperazin-1-yl)quinazolin-4-yl)-3-nitro-4-(2-(phenylthio)ethylamino)benzenesulfonamide

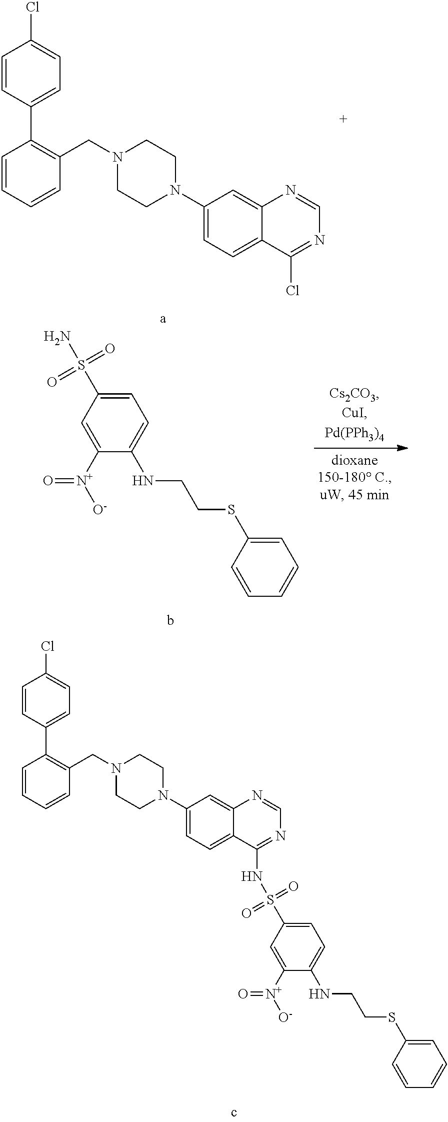

A solution of the chloroquinazoline a (0.22 mmol), the sulfonamide b (0.22 mmol), cesium carbonate (0.31 mmol), palladium tetrakis(triphenylphosphine) (0.015 mmol), copper iodide (0.03 mmol) in dioxane (4 ml) is degassed for 5 mins before being subject to microwave irradiation (300 W, 150-180° C., CEM Discover Labmate) for 45 mins. The mixture is filtered washing with ethyl acetate and then is washed with solution of 10% sodium hydrogencarbonate (10 ml). The organic layer is dried (MgSO₄) and concentrated in vacuo to give a crude residue (90%). This residue is then subject to preparative reverse phase HPLC for purification of final compound c.

### Example 17

### Preparation of 3-nitro-4-(2-(phenylthio)ethylamino)benzenesulfonamide (b)

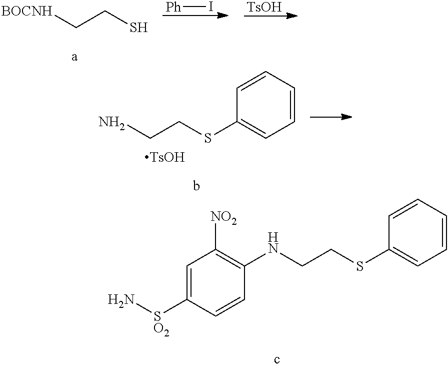

As per the procedure of Kwong and Buchwald (Org. Lett. 2002, 4:3517-3520), CuI (160 mg), K₂CO₃ (8 mmol), PhI (4 mmol) and BOCNHCH₂CH₂SH (a) (4 mmol) in DME (1 ml) stirred 80 deg. 3 days in capped screw-top vial. At the end of this time, ca 5 ml DCM was added and the reaction mixture filtered through a silica plug to remove low rf impurities. After removal of the solvent in vacuo, the residue was dissolved in MeCN containing ca 20 mmol TsOH and stood 2 h. The resulting product b was filtered off as colourless plates in 45% overall yield (590 mg). ES⁺ 154; ¹H NMR (d6-DMSO): 7.81, bs, 3H, NH₃; 7.45, d, 2H, Ts; 7.35, m, 4H, 4×ArH; 7.24, m, 1H, 1×ArH; 7.08, d, 2H, Ts; 3.15, m, 2H, CH₂; 2.93, m, 2H, CH₂; 2.25, s, 3H, CH₃.

Compound b (1.1 equivalents) and DIPEA (1.5 mmol, 270 uL) in MeCN (1 ml) was treated with the activated aryl chloride, NH₂SO₂Ph(3-NO₂) (4-Cl) and stirred at ca 60 degrees celcius for 3 days. At the end of this period, ca 2 ml water was added and the product c precipitated out as yellow solid. This was filtered and washed with 3×1 ml 50:50 MeOH:H₂O) and dried to give 150 mg (85%) of c which was pure by tic and good enough to on-react, though a sample was taken and recrystallised from acetone/toluene. MS confirmed mw of 353. ¹H NMR (d6-DMSO): 8.63, t, J=5.9 Hz, 1H, NH; 8.41, d, J=2.2 Hz, 1H, ArH; 7.76, dd, J₁ 9.1 Hz, J₂ 2.2 Hz, 1H, ArH; 7.25-7.38, m, 6H, 4×ArH+NH₂; 7.13-7.19, m, 2H, 2×ArH; 3.62, “q”, 2H, CH₂; 3.25, “t”, 2H, CH₂.

### Example 18

### Preparation of 4-chloro-7-(4-fluorophenyl)quinazoline (d)

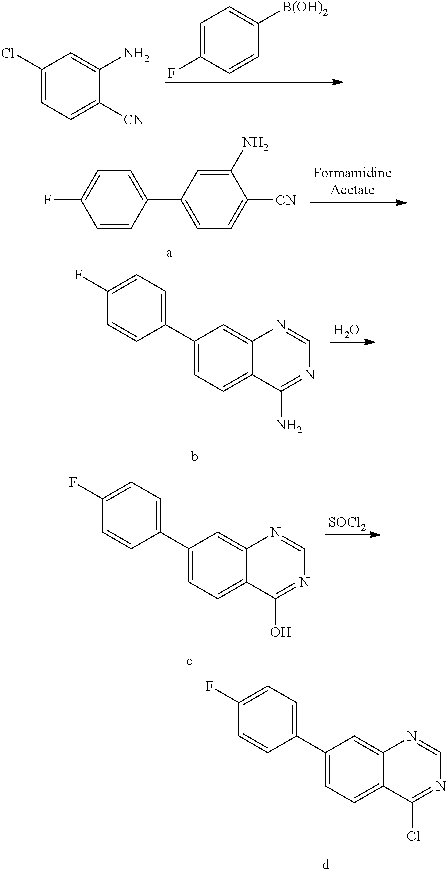

The aryl chloride (2-amino-4-chlorobenzonitrile, 4 mmol), arylboronic acid (4-fluorophenylboronic acid, 6 mmol), K₂CO₃ (8 mmol), TBAB (4 mmol), the catalyst ** 2 mol % (which was prepared as described below) in water (ca 15 ml) refluxed in air 2 hours and the product extracted into 4×30 ml warm toluene, the organic layer separated, dried (MgSO₄), and decanted through a silica plug, eluting product with DCM, which was recrystallised from 50% cyclohexane toluene to give a as off-white solid (480 mg, 56%). Sometimes this was contaminated with some unreacted aryl chloride, but this disappeared in the subsequent step. This compound was not further characterized other than confirmation by MS of the expected mw of 212.

To 1 mmol of a in ethylene glycol monomethyl ether, MeOCH₂CH₂OH (ca 1-2 ml) was added 6 mmol formamidine acetate and the mixture refluxed under nitrogen for 5 h, stood, and the product filtered off from the cooled reaction mixture (180 mg, 75%) to give b as colourless (or sometimes darkened) plates, analytically pure. MS confirmed expected mw of 239. ¹H NMR (d6-DMSO): 8.38, s, 1H, ArH (H2); 8.26, d, J=8.6 Hz, 1H, ArH (H5); 7.74-7.87, m, 6H, 4×ArH+NH₂; 7.31, “t”, 2H, 2×ArH.

The aromatic amine in b was hydrolysed to the OH by refluxing 85 mg in 5 ml 5M HCl for 30 minutes, and the product filtered after standing to give c in 92% yield. This compound was not further characterized other than confirmation by MS of the expected mw of 240.

Preparation of Catalyst (**): the Palladium Catalyst ** was made via a procedure adapted from Baleizao et al, J. Org. Chem 69 p 439. Thus, to a solution of NH₂OH.HCl (5.13 g, 74 mmol) and NaOAc.3H₂O (17 g, 125 mmol) in water (20 ml) was added p-hydroxyacetophenone (3 g, 22 mmol) and the solution refluxed 1.5h, stood overnight and product filtered off and washed with 3×20 ml cold water to afford 2.78 g oxime as buff needles (83%). Then, to a solution of Li₂PdCl₄ (2 mmol) in methanol (4 ml) was added a methanolic solution (2 ml) of the above oxime (2 mmol) and sodium acetate (2 mmol) and the mixture stirred 72h rt, filtered, and after water (5 ml) was added, the palladium catalyst started to precipitate as a yellow solid (60%).

Compound c was chlorinated by treating 103 mg in 1 ml dry chloroform and 1 ml SOCl₂ with 1 drop DMF and refluxing 30mins with CaCl₂ drying tube, to give, after pouring onto ice, extracting with ethyl acetate and evaporating, d as a creamy solid (110 mg, 99%). This compound was used without further purification (See, Example 19, below).

### Example 19

### Preparation of N-(7-(4-fluorophenyl)quinazolin-4-yl)-3-nitro-4-(2-(phenylthio)ethylamino)benzenesulfonamide (c)

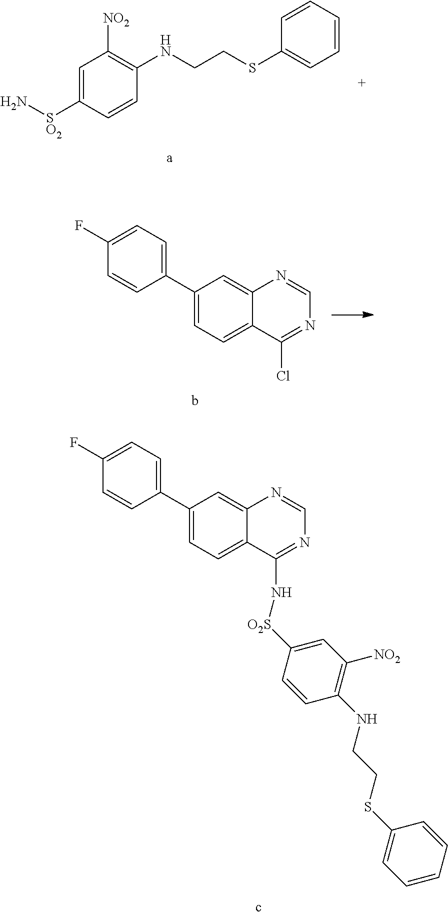

Compounds a and b were combined with and K₂CO₃ (180 mg) in DMF (ca 1 ml) and heated at 60 deg. o/n. The resultant reaction mixture was diluted with EtOAc/citric acid and the organic layer separated and evaporated to provide a yellow solid, which after trituration with 2×5 ml hot toluene, then 2×2 ml MeCN, gave c, 53 mg, 68% by starting sulfonamide) as a yellow powder. Crystalline granules were obtained from the d6-DMSO NMR solution by precipitation with minimal water. ES⁺576. ¹H NMR (d6-DMSO): 8.63, t, 1H, NH; 8.59, d, J=2.0 Hz, 1H, ArH (H2′); 8.41, bs, 1H, NH; 8.23, d, J 8.4, 1H, ArH (H2); 7.79-7.96, m, 5H, 5×ArH; 7.07-7.40, m, 9H, 9×ArH; 3.56-3.63, m, CH₂; 3.21-3.28, 2H, CH₂. When tested in the Alphascreen assay, compound c had an IC₅₀ of 3.0 uM against Bcl-X_{L}.

### Example 20

### Bcl-2 Binding Assay

The following example describes the measurement of competition of compounds of the invention with Bim26-mer for a Bcl-2 homologue binding site.

Alphascreen (Amplified Luminescent Proximity Homogenous Assay) is a bead based technology which measures the interaction between molecules. The assay consists of two hydrogel coated beads which, when brought into close proximity by a binding interaction, results in the transfer of singlet oxygen from a donor bead to an acceptor bead.

Upon binding and excitation with laser light at 680 nm, a photosensitizer in the donor bead converts ambient oxygen to a more excited singlet state. This singlet oxygen then diffuses and reacts with a chemiluminescer in the acceptor bead. Fluorophores within the same bead are activated and result in the emission of light at 580-620 nm.

Screening of the compounds of the invention is performed using the Alphascreen GST (glutathione s-transferase) detection kit system. Test compounds are titrated into the assay which consists of GST tagged Bcl_{w} ΔC29 protein (0.05 nM Final concentration) and Biotinylated Bim BH3-26 peptide, Biotin-DLRPEIRIAQELRRIGDEFNETYTRR (3.0 nM Final concentration). For the GST tagged Bcl-X_{L} assay, GST tagged Bcl-X_{L} ΔC25 protein (0.6 nM Final concentration) and Biotinylated Bim BH3-26 peptide, Biotin-DLRPEIRIAQELRRIGDEFNETYTRR (5.0 nM final concentration) are used. To this reaction mix, anti-GST coated acceptor beads and Streptavidin coated donor beads, both at 15 μg/ml Final concentration, are added and the assay mixture is incubated for 4 hours at room temperature before reading. Similarly when the Bcl-2 protein is Mcl-1, GST tagged Mcl-1 protein (0.4 nM Final concentration) and Biotinylated Bak BH3 peptide, Biotin-PSSTMGQVGRQLAIIGDDINRRYDSE-OH (4.0 nM Final concentration) are used.

### Detailed Protocol:

1) Prepare a 384 well with 4.75 μL of buffer and 0.25 μL of compounds (20 mM in DMSO) per well.
2) Mix the binding partners, in one tube add Bcl-w, Bcl-X_{L} or Mcl-1 and the acceptor beads, in the second tube add Biotinylated BH3 peptide and the donor beads.
3) Pre-incubate the two pairs of binding partners for 30 minutes.
4) Add 1 μl, of acceptor beads:Bcl-w, Bcl-X_{L} or Mcl-1 protein mix to each well.
5) Seal the plate and incubate at room temperature for 30 minutes.
6) Add 10 μl, of donor bead:BH3 peptide mix to each well.
7) Seal the plate, cover with foil and incubate for 4 hours.

Assay buffer contains 50 mM Hepes pH 7.4, 10 mM DTT, 100 mM NaCl, 0.05% Tween and 0.1 mg/ml casein. Bead dilution buffer contains 50 mM Tris, pH 7.5, 0.01% Tween and 0.1 mg/ml casein. The final DMSO concentration in the assay is 0.5%. Assays are performed in 384 well white Optiplates and is analyzed on the PerkinElmer Fusion alpha plate reader (Ex680, Em520-620 nM).

The GST Alphascreen detection kit and Optiplates are purchased from PerkinElmer.

### Example 21

### Cell Viability Assay

The efficacy of the compounds of the present invention can also be determined in cell based killing assays using a variety of cell lines and mouse tumor models. For example, their activity on cell viability can be assessed on a panel of cultured tumorigenic and non-tumorigenic cell lines, as well as primary mouse or human cell populations, e.g. lymphocytes. For these assays, 5,000-20,000 cells are cultured at 37° C. and 10% CO₂ in appropriate growth media, eg: 100 μl, Dulbecco's Modified Eagle's medium supplemented with 10% foetal calf serum, asparaginase and 2-mercaptoethanol in the case of pre-B Eμ-Myc mouse tumors in 96 well plates. Cell viability and total cell numbers can be monitored over 1-7 days of incubation with 1 nM-1001.IM of the compounds to identify those that kill at IC50<10 μM. Cell viability is determined by the ability of the cells to exclude propidum iodide (10 μg/mL by immunofluorescence analysis of emission wavelengths of 660-675 nm on a flow cytometer (BD FACScan). Alternatively, a high throughput colorimetric assay such as the Cell Titre 96 can be used. Aqueous Non-Radioactive Cell Proliferation Assay (Promega) may be used. Cell death by apoptosis is confirmed by pre-incubation of the cells with 50 μM of a caspase inhibitor such as zVAD-fmk.

Neutralization of both Bcl-xL and Mcl-1 anti-apoptotic proteins in normal cells is required before a cell undergoes apoptosis via the downstream Bax/Bak pathway [Chen et al., 2005; Willis et al., 2005]. A compound that only targets Bc1-xL should not affect normal cells, but could kill certain cancer cells if they rely more on Bcl-xL and less on Mcl-1 for survival. To mirror this, compounds of the invention can be tested for its effect on survival of wild type (wt) mouse embryo fibroblasts (MEFs), Bax/Bak double knockout (BB DKO) MEFs, MEFs that expressed Noxa, and MEFs that expressed Bad. Noxa specifically neutralizes Mcl-1. Hence, MEFs that express Noxa mirror cancer cell types that are reliant on Bcl-xL for survival and should be much more sensitive to killing by a Bcl-xL targeting compound than MEFs where both Bcl-xL and Mcl-1 are protective.

### Example 22

### CellTitre-Glow Luminescent Cytotoxity Assay

Cytoxicity of compounds of the invention are evaluated on SCLC cell lines NCI-H889, NCI-H1963 and NCI-H146 using Promega CellTitre-Glow luminescent assay kit G7571 according to the following procedures:

### Culture Medium and Cell Lines:

1. SCLC cell lines NCI-H889, NCI-H1963, and NCI-H146 are purchased from American Type Culture Collection. Cells are maintained in RPMI 1640 (Invitrogen Corp., Grand Island, N.Y.) that is supplemented with 10% fetal bovine serum (FBS, Invitrogen), 1% sodium pyruvate, 25 mM HEPES, 4.5 g/L glucose and 1% penicillin/streptomycin (Sigma) in a humidified chamber at 37° C. containing 5% CO₂.
2. Cells are grown as suspension aggregates in a T162 flask with 25 ml medium and kept at concentration of 1 million/mL.

### Test Compound Stocks:

1. Test compounds are prepared as 10 mM stocks in DMSO and stored at −20° C.

### Test Compound Serial Dilutions:

1. Prewarm medium to 37° C.
2. Thaw compounds to room temperature.
3. Determine what will be the highest concentration to be tested. (i.e. 10 μM).
4. Prepare a 2× stock in culture medium of the first dose (i.e. 2×10 μM=20 μM) in an Eppendorf tube (i.e. 4 μl of 5 mM stock into 1000 μl=20 μM).
5. Invert tube several times to mix.

### Serial Dilution in 96 Well Plates:

Compounds are tested in triplicate at concentrations of 10, 5, 2.5, 1.3, 0.63, 0.32, 0.16, 0.08, 0.04 and 0.02 μM. Columns 1-10 comprise the serial test compound treatments, column 11 is the untreated control and column 12 is the ‘no cell’ control for determining the background.
1. In a 96 well plate is added 50 μl medium/well in columns 2 thru 11 and 100 μl is added to column 12.
2. In column 1 is added 100 μl of 2×compound.
3. Make serial dilutions by transferring 50 μl to column 2, and so on up to column 10. After adding 50 μl to column 10 and mixing, discard 50 μl.
4. Store plates at 37° C. until ready to add cells.

### Cell Preparation:

1. Cells are washed one time in the culture medium and are prepared as a suspension.
   a. Cells are first spun down to remove the medium and then -1 ml 0.25% trypsin is added and gently mixed and is incubated for no more than three minutes at room temperature.
   b. ˜10 ml of the medium is added and the cells are gently pipetted several times.
   c. Cells are then counted and spun down to the volume necessary for the total cell number that is needed, then is then resuspended in the medium to 200 cells/μl concentration (50,000 cells/well).
2. 50 μl of cell prep is added to the appropriate wells.
3. Incubate cells for 48 hr at 37° C.

### CellTiter-Glow Luminescent Assay (Promega—Kit G7571):

1. Buffer thaws in a 37° C. water bath until thawing completed and then left for at least ½ hr at room temperature
2. Substrate and buffer are mixed together and inverted gently several times to dissolve substrate.
3. Cell culture plates are removed from the incubator and are allowed to adjust to room temperature for at least 15 min.
4. 100 μl of reagent is added to 100 μl culture medium and is mixed on plate shaker for 2 min at RT.
5. Incubate for 15 min on bench
6. Luminescence is read on BioTek plate reader (sensitivity=95).
7. Average background value is calculated (column 12)
8. Average background counts is subtracted from all other wells (columns 1-11)
9. Average untreated control value is calculated (column 11)
10. Test compound treated well values (rows 1-10) are divided by the average control value and expressed as an EC50.

We claim:

1. A compound of Formula I: 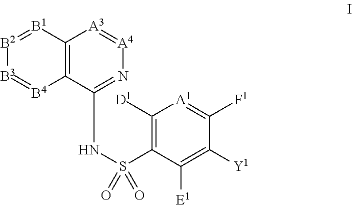
wherein
A¹ is N or C(A²);
one or two or three or each of A², F¹, D¹ and E¹ are independently selected R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NR¹C(O)NHR¹, NR¹C(O)N(R¹)₂, SO₂NHR¹, SO₂N(R¹)₂, NHSO₂R¹, NHSO₂NHR¹ or N(CH₃)SO₂N(CH₃)R¹, and the remainder are independently selected H, F, Cl, Br, I, CN, CF₃, C(O)OH, C(O)NH₂ or C(O)OR^{1A}; and
Y¹ is H, CN, NO₂, C(O)OH, F, Cl, Br, I, CF₃, OCF₃, CF₂CF₃, OCF₂CF₃, R¹⁷, OR¹⁷, C(O)R¹⁷, C(O)OR¹⁷, SR¹⁷, NH₂, NHR¹⁷, N(R¹⁷)₂, NHC(O)R¹⁷, C(O)NH₂, C(O)NHR¹⁷, C(O)N(R¹⁷)₂, NHS(O)R¹⁷ or NHSO₂R¹⁷;
or
F¹ and Y¹, together with the atoms to which they are attached, are imidazole or triazole; and
one or two or each of A², D¹ and E¹ are independently selected R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NHC(O)NHR¹, N(CH₃) C(O)N(CH₃) R¹, SO₂NHR¹, SO₂N(R¹)₂, NHSO₂R¹, NHSO₂NHR¹ or N(CH₃)SO₂N(CH₃)R¹, and the remainder are independently selected H, F, Cl, Br, I, CF₃, C(O)OH, C(O)NH₂ or C(O)OR^{1A};
R¹ is R², R³, R⁴ or R⁵;
R^{1A} is C₁-C₆-alkyl, C₃-C₆-alkenyl or C₃-C₆-alkynyl;
R² is phenyl which is unfused or fused with arene, heteroarene or R^{2A}; R^{2A} is cycloalkane or heterocycloalkane;
R³ is heteroaryl which is unfused or fused with benzene, heteroarene or R^{3A}; R^{3A} is cycloalkane or heterocycloalkane;
R⁴ is cycloalkyl, cycloalkenyl, heterocycloalkyl or heterocycloalkenyl, each of which is unfused or fused with arene, heteroarene or R^{4A}; R^{4A} is cycloalkane, cycloalkene, heterocycloalkane or heterocycloalkene;
R⁵ is alkyl, alkenyl or alkynyl, each of which is unsubstituted or substituted with one or two or three independently selected R₆, NC(R^{6A})(R^{6B}), R⁷, OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂, NHC(O)NHR⁷, NHC(O)CH(CH₃) NHC(O)CH(CH₃) NH₂, NHC(O)CH(CH₃)NHC(O)CH(CH₃)NHR¹, OH, (O), C(O)OH, (O), N₃, CN, NH₂, CF₃, CF₂CF₃, F, Cl, Br or I substituents;
R⁶ is C₂-C₅-spiroalkyl, each of which is unsubstituted or substituted with OH, (O), N₃, CN, CF₃, CF₂CF₃, F, Cl, Br, I, NH₂, NH(CH₃) or N(CH₃)₂;
R^{6A} and R^{6B} are independently selected alkyl or, together with the N to which they are attached is R^{6C};
R^{6C} is aziridin-1-yl, azetidin-1-yl, pyrrolidin-1-yl or piperidin-1-yl, each having one CH₂ moiety unreplaced or replaced with O, C(O), CNOH, CNOCH₃, S, S(O), SO₂ or NH;
R⁷ is R⁸, R⁹, R¹⁰ or R¹¹;
R⁸ is phenyl which is unfused or fused with arene, heteroarene or R^{8A};
R^{8A} is cycloalkane, cycloalkene, heterocycloalkane or heterocycloalkene;
R⁹ is heteroaryl which is unfused or fused with arene, heteroarene or R^{9A}; R^{9A} is cycloalkane, cycloalkene, heterocycloalkane or heterocycloalkene;
R¹⁰ is C₃-C₁₀-cycloalkyl or C₄-C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{10A}; R^{10A} is cycloalkane, cycloalkene, heterocycloalkane or heterocycloalkene;
R¹¹ is alkyl, alkenyl or alkynyl, each of which is unsubstituted or substituted with one or two or three independently selected R¹², OR¹², NHR¹², N(R¹²)₂, C(O)NH₂, C(O)NHR¹², C(O)N(R¹²)₂, OH, (O)C(O)OH, N₃, CN, NH₂, CF₃, CF₂CF₃, F, Cl, Br or I substituents;
R¹² is R¹³, R¹⁴, R¹⁵ or R¹⁶;
R¹³ is phenyl which is unfused or fused with arene, heteroarene or R^{13A}; R^{13A} is cycloalkane, cycloalkene, i heterocycloalkane or heterocycloalkene;
R¹⁴ is heteroaryl, each of which is unfused or fused with arene, heteroarene or R^{14A}; R^{14A} is cycloalkane, cycloalkene, heterocycloalkane or heterocycloalkene;
R¹⁵ is cycloalkane, cycloalkene, heterocycloalkane or heterocycloalkene, each of which is unfused or fused with arene, heteroarene or R^{15A}; R^{15A} is cycloalkane, cycloalkene, heterocycloalkane or heterocycloalkene;
R¹⁶ is alkyl, alkenyl or alkynyl;
R¹⁷ is R¹⁸, R¹⁹, R²⁰ or R²¹;
R¹⁸ is phenyl which is unfused or fused with arene, heteroarene or R^{18A}; R^{18A} is cycloalkane, cycloalkene, heterocycloalkane or heterocycloalkene;
R¹⁹ is heteroaryl which is unfused or fused with arene, heteroarene or R^{19A}; R^{19A} is cycloalkane, cycloalkene, heterocycloalkane or heterocycloalkene;
R²⁰ is C₃-C₁₀-cycloalkyl or C₄-C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{20A}; R^{20A} is cycloalkane, cycloalkene, heterocycloalkane or heterocycloalkene;
R²¹ is alkyl, alkenyl or alkynyl, each of which is unsubstituted or substituted with one or two or three independently selected R²², OR²², NHR²², N(R²²)₂, C(O)NH₂, C(O)NHR²², C(O)N(R²²)₂, OH, (O)C(O)OH, N₃, CN, NH₂, CF₃, CF₂CF₃, F, Cl, Br or I substituents;
R²² is R²³, R²⁴ or R²⁵;
R²³ is phenyl which is unfused or fused with arene, heteroarene or R^{23A}; R^{23A} is cycloalkane, cycloalkene, i heterocycloalkane or heterocycloalkene;
R²⁴ is heteroarene which is unfused or fused with arene, heteroarene or R^{24A}; R^{24A} is cycloalkane, cycloalkene, i
R²⁵ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{25A}; R^{25A} is cycloalkane, cycloalkene, heterocycloalkane or heterocycloalkene;
A³ and A⁴ are each independently N or C(R²⁶); each R²⁶ is independently hydrogen, halogen, C₁₋₆-alkyl, C₂₋₆-alkenyl, C₂₋₆-alkynyl, hydroxy, C₁₋₆-alkoxy, C₂₋₆-alkenyloxy, C₂₋₆-alkynyloxy, C(O)N(R^{26A})₂, acyl, N(R^{26B})₂, C(R^{26C})₃, wherein R^{26A} is independently C₁₋₆-alkyl, C₂₋₆-alkenyl or C₂₋₆-alkynyl; R^{26B} is independently hydrogen, C₁₋₆-alkyl, C₂₋₆-alkenyl, C₂₋₆-alkynyl or acyl; R^{26C} is independently hydrogen or halogen;
B¹, B² B³ and B⁴ are each independently N, C(Z) or C(R²⁷) wherein one of B¹, B², B³ or B⁴ is C(Z); each R²⁷ is independently selected H, F, Cl, Br, I, CH₂R^{27C}, CH(R^{27B})(R^{27C}), C(R^{27B})(R^{27A})(R^{27C}), C(O)R^{27C}, OR^{27C}, SR^{27C}, S(O)R^{27C}, SO₂R^{27C}, NHR^{27C} or N(R^{27D})R^{27C};
R^{27A} and R^{27B} are independently F, Cl, Br or alkyl or are taken together and are C₂-C₅-spiroalkyl;
R^{27D} is R^{27E}, C(O)R^{27E} or C(O)OR^{27E};
R^{27F} is R^{27F} or R^{27G};
R^{27F} is phenyl which is unfused or fused with aryl, heteroaryl or R^{27FF}; R^{27FF} is cycloalkane, cycloalkene, heterocycloalkane or heterocycloalkene;
R^{27G} is alkyl which is unsubstituted or substituted with R^{27H};
R^{27H} is phenyl which is unfused or fused with arene, heteroarene or R_{27HH}; R^{27HH} is cycloalkane, cycloalkene, heterocycloalkane or heterocycloalkene;
R^{27C} is R^{27I}, R^{27J} or R^{27K}, each of which unsubstituted or is substituted with F, Cl, Br, I, R^{27L}, OR^{27L}, NHR^{27L}, N(R^{27L})₂, NHC(O)OR^{27L}SR^{27L}S(O)R^{27L} or SO₂R^{27L};
R^{27I} is phenyl which is unfused or fused with arene, heteroarene or R^{27II}; R^{27II} is cycloalkane, cycloalkene, heterocycloalkane or heterocycloalkene;
R^{27J} is heteroaryl which is unfused or fused with arene, heteroarene or R^{27JJ}; R^{27JJ} is cycloalkane, cycloalkene, heterocycloalkane or heterocycloalkene;
R^{27K} is C₃-C₈-cycloalkyl or C₄-C₈-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{27KK}; R^{27KK} cycloalkane, cycloalkene, heterocycloalkane or heterocycloalkene;
R^{27L} is R^{27M}, R^{27N}, R^{27P} or R^{27Q};
R^{27M} is phenyl which is unfused or fused with arene, heteroarene or R^{27MM}; R^{27MM} is cycloalkane, cycloalkene, heterocycloalkane or heterocycloalkene;
R^{27N} is heteroaryl which is unfused or fused with arene, heteroarene or R^{27NN}; R^{27NN} is cycloalkane, cycloalkene, heterocycloalkane or heterocycloalkene;
R^{27P} is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{27PP}; R^{27PP} is cycloalkane, cycloalkene, heterocycloalkane or heterocycloalkene;
R^{27Q} is alkyl, alkenyl or alkynyl, each of which is unsubstituted or substituted with one or two independently selected R^{27QQ}, OR^{27QQ}, NHR^{27QQ}, N(R^{27QQ})₂, C(O)NH₂, C(O)NHR^{27QQ}, C(O)N(R^{27QQ})₂, OH, (O), C(O)OH, N₃, CN, NH₂, CF₃, CF₂CF₃, F, Cl, Br or I substituents;
R^{27QQ} is R^{27R}, R^{27S} or R^{27T};
R^{27R} is phenyl which is unfused or fused with arene, heteroarene or R^{27RR}; R^{27RR} is cycloalkane, cycloalkene, heterocycloalkane or heterocycloalkene;
R^{27S} is heteroaryl or R^{27SS}; R^{27SS} is cycloalkane, cycloalkene, heterocycloalkane or heterocycloalkene;
R^{27T} is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{27TT}; R^{27TT} is cycloalkane, cycloalkene, heterocycloalkane or heterocycloalkene;
Z is R²⁸, R²⁹ or R³⁰, wherein
R²⁸ is phenyl which is unfused or fused with arene, heteroarene or R^{28A}; R^{28A} is cycloalkane, cycloalkene, heterocycloalkane or heterocycloalkene
R²⁹ is heteroaryl or R^{29A}; R^{29A} is cycloalkane, cycloalkene, heterocycloalkane or heterocycloalkene;
R³⁰ is cycloalkyl or cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{30A}; R^{30A} is cycloalkane, cycloalkene, heterocycloalkane or heterocycloalkene; and
each of R²⁸, R²⁹ and R³⁰ is unsubstituted or is substituted with F, Cl, Br, I, CH₂R³⁷, CH(R³¹)(R³⁷), C(R³¹)(R^{31A})(R³⁷), C(O)R³⁷, OR³⁷, SR³⁷, S(O)R³⁷, SO₂R³⁷, NHR³⁷ or N(R³²) R³⁷;
R³¹ and R^{31A} are independently F, Cl, Br or alkyl or are taken together and are C₂-C₅-spiroalkyl;
R³² is R³³, C(O)R³³ or C(O)OR³³;
R³³ is R³⁴ or R³⁵;
R³⁴ is phenyl which is unfused or fused with aryl, heteroaryl or R^{34A}; R^{34A} is cycloalkane, cycloalkene, heterocycloalkane or heterocycloalkene;
R³⁵ is alkyl which is unsubstituted or substituted with R³⁶;
R³⁶ is phenyl which is unfused or fused with arene, heteroarene or R^{36A}; R^{36A} is cycloalkane, cycloalkene, heterocycloalkane or heterocycloalkene;
R³⁷ is R³⁸, R³⁹ or R⁴⁰ each of which is unsubstituted or is substituted with F, Cl, Br, I, R⁴¹, OR⁴¹, NHR⁴¹, N(R⁴¹)₂, NHC(O)OR⁴¹, SR⁴¹, S(O)R⁴¹ or SO₂R⁴¹;
R³⁸ is phenyl which is unfused or fused with arene, heteroarene or R^{38A}; R^{38A} is cycloalkane, cycloalkene, i heterocycloalkane or heterocycloalkene;
R³⁹ is heteroaryl which is unfused or fused with arene, heteroarene or R^{39A}; R^{39A} is cycloalkane, cycloalkene, heterocycloalkane or heterocycloalkene;
R⁴⁰ is C₃-C₈-cycloalkyl or C₄-C₈-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{40A}; R^{40A} cycloalkane, cycloalkene, heterocycloalkane or heterocycloalkene;
R⁴¹ is R⁴², R⁴³, R⁴⁴ or R⁴⁵;
R⁴² is phenyl which is unfused or fused with arene, heteroarene or R^{42A}; R^{42A} is cycloalkane, cycloalkene, i heterocycloalkane or heterocycloalkene;
R⁴³ is heteroaryl which is unfused or fused with arene, heteroarene or R^{43A}; R^{43A} is cycloalkane, cycloalkene, heterocycloalkane or heterocycloalkene;
R⁴⁴ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{44A}; R^{44A} is cycloalkane, cycloalkene, heterocycloalkane or heterocycloalkene;
R⁴⁵ is alkyl, alkenyl or alkynyl, each of which is unsubstituted or substituted with one or two independently selected R⁴⁶, OR⁴⁶, NHR⁴⁶, N(R⁴⁶)₂, C(O)NH₂, C(O)NHR⁴⁶, C(O)N(R⁴⁶)₂, OH, (O)C(O)OH, N₃, CN, NH₂, CF₃, CF₂CF₃, F, Cl, Br or I substituents;
R⁴⁶ is R⁴⁷, R⁴⁸ or R⁴⁹;
R⁴⁷ is phenyl which is unfused or fused with arene, heteroarene or R^{47A}; R^{47A} is cycloalkane, cycloalkene, i heterocycloalkane or heterocycloalkene;
R⁴⁸ is heteroaryl or R^{48A}; R^{48A} is cycloalkane, cycloalkene, heterocycloalkane or heterocycloalkene;
R⁴⁹ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{49A}; R^{49A} is cycloalkane, cycloalkene, heterocycloalkane or heterocycloalkene;
wherein each foregoing cyclic moiety is independently unsubstituted, further unsubstituted, substituted or further substituted with one or two or three or four or five independently selected from R⁵⁰, OR⁵⁰, SR⁵⁰S(O)R⁵⁰, SO₂R⁵⁰, C(O)R⁵⁰CO(O)R⁵⁰OC(O)R⁵⁰OC(O)OR⁵⁰NH₂, NHR⁵⁰N 50) C(O)NH₂, C(O)NHR⁵⁰, C(O)N(R⁵⁰)₂, C(O)NHOH, C(O)NHOR⁵⁰, C(O)NHSO₂R⁵⁰, C(O)NR⁵⁵SO₂R⁵⁰, SO₂NH₂, SO₂NHR⁵⁰, SO₂N(R⁵⁰)₂, CF₃, CF₂CF₃, C(O) FI, C(O)OH, C(N)NH₂, C(N)NHR⁵⁰, C(N)N(R⁵⁰)₂, OH, (O) N₃, NO₂, CF₃, CF₂CF₃, OCF₃, OCF₂CF₃, F, Cl, Br or I substituents;
R⁵⁰ is R⁵¹, R⁵², R⁵³ or R⁵⁴;
R⁵¹ is phenyl which is unfused or fused with arene, heteroarene or R^{51A}; R^{51A} is cycloalkane, cycloalkene, heterocycloalkane or heterocycloalkene;
R⁵² is heteroaryl or R^{52A}; R^{52A} is cycloalkane, cycloalkene, heterocycloalkane or heterocycloalkene;
R⁵³ is C₃-C₆-cycloalkyl or C₄-C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with arene, heteroarene or R^{53A}; R^{53A} is cycloalkane, cycloalkene, heterocycloalkane or heterocycloalkene;
R⁵⁴ is alkyl, alkenyl or alkynyl, each of which is unsubstituted or substituted with one or two or three independently selected R⁵⁵, OR⁵⁵, SR⁵⁵, S(O)R⁵⁵, SO₂R⁵⁵, NHR⁵⁵, N(R⁵⁵)₂, C(O)R⁵⁵, C(O)NH₂, C(O)NHR⁵⁵, NHC(O)R⁵⁵, NHSO₂R⁵⁵, NHC(O)OR⁵⁵, SO₂NH₂, SO₂NHR⁵⁵, SO₂N(R⁵⁵)₂, NHC(O)NH₂, NHC(O)NHR⁵⁵, OH, (O)C(O)OH, (O)N₃, CN, NH₂, CF₃, OCF₃, CF₂CF₃, OCF₂CF₃, F, Cl, Br or I substituents;
R⁵⁵ is alkyl, alkenyl, alkynyl, phenyl, heteroaryl or R⁵⁶;
R⁵⁶ is C₃-C₆-cycloalkyl or C₄-C₆-cycloalkyl, each having one or two CH₂ moieties unreplaced or replaced with independently selected O, C(O), CNOH, CNOCH₃, S, S(O), SO₂ or NH and one or two CH moieties unreplaced or replaced with N; and
with the proviso that excluded from the presently claimed compounds of Formula I are the compounds of Formula I′: 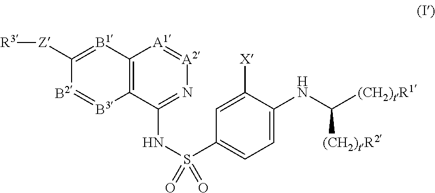
wherein X′ is NO₂ or —SO₂—C(X″)₂ wherein X″ is H or halo; A^{1′}, A^{2′}, B^{1′}, B^{2′} and B^{3′} are independently N or CR^{4′}; Z′ is a cycloalkyl, cycloalkenyl, aryl, heterocyclic or heteroaryl group; R^{1′} and R^{2′} are independently aryl, heteroaryl, —NR^{5′}R^{6′}, —CONR^{5′}R^{6′}, —O(CH₂), aryl, —O(CH₂), heteroaryl, —CO(CH₂), aryl, —CO(CH₂), heteroaryl, —CO₂(CH₂), aryl, —CO₂(CH₂), heteroaryl, —OCO(CH₂), aryl, —OCO(CH₂), heteroaryl, —S(CH₂), aryl, —S(CH₂), heteroaryl, —SO(CH₂), aryl, —SO(CH₂), heteroaryl, —SO₂(CH₂), aryl or —SO₂(CH₂), heteroaryl; R^{3′} is alkyl, alkenyl, —(CH₂)_{t′}cycloalkyl, —(CH₂)ₜ,cycloalkenyl, —(CH₂)ₜ,aryl, —(CH₂)_{t′}heterocyclyl or —(CH₂)_{t′}heteroaryl, wherein each cycloalkyl, cycloalkenyl, aryl, heterocyclyl and heteroaryl may be optionally substituted with alkyl, alkenyl, halo, nitro, haloalkyl, or phenyl optionally substituted with 1, 2 or 3 alkyl, alkenyl, alkoxy, halo or nitro groups; R^{4′} is hydrogen, halogen, —C₂₋₆alkyl, —C₂₋₆alkenyl, —C₂₋₆alkynyl, hydroxy, —OC₂₋₆alkyl, —OC₂₋₆alkenyl, —OC₂₋₆alkynyl, —N(R^{7′})₂, acyl, —C(R^{8′})₂ or —CON(R^{9′})₂; R^{5′} and R^{6′} are independently hydrogen, alkyl or alkenyl or R^{5′} and R^{6′} taken together with the nitrogen to which they are attached form a heterocyclic or heteroaryl ring; each R^{7′} is independently hydrogen, —C₁₋₆alkyl, —C₂₋₆alkenyl, —C₂₋₆alkynyl or acyl; each R^{8′} is independently hydrogen or halogen; each R^{9′} is independently hydrogen, 6alkyl, —C₂₋₆alkenyl or —C₂₋₆alkynyl, t′ is 0 or an integer 1 to 6; and r′ is 0 or an integer 1 to 6; wherein each alkyl, alkenyl, alkynyl, cycloalkyl, cycloalkenyl, aryl, heterocyclyl and heteroaryl group may be optionally substituted.

2. The compound of claim 1, wherein A¹ is N or C(A²);
A² is H, F, CN, C(O)OH, C(O)NH₂ or C(O)OR^{1A};
F¹ is R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NR¹C(O)NHR¹, NR¹C(O)N(R¹)₂, SO₂NHR¹, SO₂N(R¹)₂, NHSO₂R¹, NHSO₂NHR¹ or N(CH₃)SO₂N(CH₃)R¹;
D¹ is H, F, C₁ or CF₃;
E¹ is H, F or Cl;
Y¹ is H, CN, NO₂, C(O)OH, F, Cl, Br, I, CF₃, OCF₃, CF₂CF₃, OCF₂CF₃, R¹⁷, OR¹⁷, C(O)R¹⁷, C(O)OR¹⁷, SR¹⁷, NH₂, NHR¹⁷, N(R¹⁷)₂. NHC(O)R¹⁷, C(O)NH₂, C(O)NHR¹⁷, C(O)N(R¹⁷)₂, NHS(O)R¹⁷ or NHSO₂R¹⁷;
or
F¹ and Y¹, together with the atoms to which they are attached, are imidazole or triazole;
R¹R², R³, R⁴ or R⁵;
R^{1A} is C₁-alkyl, C₂-alkyl, C₃-alkyl, C₄-alkyl, C₅-alkyl, C₆-alkyl;
R² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;
R⁵ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁶, R⁷, OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂, NHC(O)NHR⁷, NHC(O)CH(CH₃)NHC(O)CH(CH₃)NH₂, OH, (O), C(O)OH, (O), N₃, CN, NH₂, CF₃, CF₂CF₃, F, Cl, Br or I substituents;
R⁶ is C₂-spiroalkyl, C₃-spiroalkyl, C₄-spiroalkyl or C₅-spiroalkyl, each of which is unsubstituted or substituted with OH, (O), N₃, CN, CF₃, CF₂CF₃, F, Cl, Br, I, NH₂, NH(CH₃) or N(CH₃)₂;
R⁷ is R⁸, R⁹, R¹⁰ or R¹¹;
R⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{8A};
R^{8A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), CNOH, CNOCH₃, S, S(O), SO₂ or NH and one or two CH moieties unreplaced or replaced with N;
R⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;
R¹¹ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two or three independently selected R¹², OR¹², NHR¹², N(R¹²)_{2f} C(O)NH₂, C(O)NHR¹², C(O)N(R¹²)₂, OH, (O), C(O)OH, N₃, CN, NH₂, CF₃, CF₂CF₃, F, Cl, Br or I substituents;
R¹² is R¹³, R¹⁴, R¹⁵ or R¹⁶;
R¹³ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{13A};
R^{13A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), CNOH, CNOCH₃, S, S(O), SO₂ or NH and one or two CH moieties unreplaced or replaced with N;
R¹⁴ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl, 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁵ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;
R¹⁶ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₃-alkynyl, C₄-alkynyl, C₅-alkynyl or C₆-alkynyl;
R¹⁷ is R¹⁸, R¹⁹, R²⁰ or R²¹;
R¹⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R¹⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;
R²¹ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two or three independently selected R²², OR²², NHR²², N(R²²)₂, C(O)NH₂, C(O)NHR²², C(O)N(R²²)₂, OH, (O), C(O)OH, N₃, CN, NH₂, CF₃, CF₂CF₃, F, Cl, Br or I substituents;
R²² is R²³, R²⁴ or R²⁵;
R²³ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²⁴ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl, 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²⁵ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;
A³ and A⁴ are each independently N or C(R²⁶); each R²⁶ is independently hydrogen, halogen, C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl, hydroxy, C₁-alkoxy, C₂-alkoxy, C₃-alkoxy, C₄-alkoxy, C₅-alkoxy, C₆-alkoxy, C₂-alkenyloxy, C₃-alkenyloxy, C₄-alkenyloxy, C₅-alkenyloxy, C₆-alkenyloxy, C₂-alkynyloxy, C₃-alkynyloxy, C₄-alkynyloxy, C₅-alkynyloxy, C₆-alkynyloxy, —C(O)N(R^{26A})₂, acyl, —N(R^{26B})₂, —C(R^{26C})³, wherein R^{26A} is independently C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl; R^{26B} is independently hydrogen, C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl or acyl; R^{26C} is independently hydrogen, F, Cl, Br or I;
B¹, B² B³ and B⁴ are each independently N, C(Z) or C(R²⁷) wherein one of B¹, B², B³ or B⁴ is C(Z); each R²⁷ is independently selected from H, F, Cl, Br, I, CH₂R^{27C}, CH(R^{27B})(R^{27C}) C(R^{27B})(R^{27A})(R^{27C}) C(O)R^{27C}, OR^{27C}, SR^{27C}, S(O)R^{27C}, SO₂R^{27C}, NHR^{27C} or N(R^{27D})R^{27C};
R^{27A} and R^{27B} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;
R^{27D} is R^{27E}, C(O)R^{27E} or C(O)OR^{27E};
R^{27E} is R^{27F} or R^{27G};
R^{27F} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27G} is C₁-alkyl, C₂-alkyl, C₃-alkyl, C₄-alkyl, C₅-alkyl or C₆-alkyl, each of which is unsubstituted or substituted with R^{27H};
R^{27H} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27C} is R^{27I}, R^{27J} or R^{27K}, each of which is substituted with F, Cl, Br, I, R^{27L}, OR^{27L}, NHR^{27L}, N(R^{27L})₂, NHC(O)OR^{27L}, SR^{27L},(O)R^{27L} or SO₂R^{27L};
R^{27I} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27J} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27K} is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;
R^{27L} is R^{27M}, R^{27N}, R^{27P} or R^{27Q};
R^{27M} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{27MM};
R^{27MM} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;
R^{27N} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27P} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27Q} is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R^{27QQ}, OR^{27QQ}, NHR^{27QQ}, N(R^{27QQ})₂, C(O)NH₂, C(O)NHR^{27QQ}, C(O)N(R^{27QQ})₂, OH, (O), C(O)OH, N₃, CN, NH₂, CF₃, CF₂CF₃, F, Cl, Br or I substituents;
R^{27QQ} is R^{27R}, R^{27S} or R^{T};
R^{27R} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27S} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27T} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
Z is R²⁸, R²⁹ or R³⁰;
R²⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₁₁-cycloalkyl, C₁₂-cycloalkyl, C₁₃-cycloalkyl, C₁₄-cycloalkyl, C₄-cycloalkenyl, C₅-cycloalkenyl, C₆-cycloalkenyl, C₇-cycloalkenyl, C₈-cycloalkenyl, C₉-cycloalkenyl or C₁₀-cycloalkenyl, each having one or two CH₂ moieties unreplaced or replaced with independently selected O, C(O), S, S(O), SO₂ or NH and one or two CH moieties unreplaced or replaced with N;
each of R²⁸, R²⁹ or R³⁰ is unsubstituted or substituted with F, Cl, Br, I, CH₂R³⁷, CH(R³¹)(R³⁷), C(R³¹)(R^{31A})(R³⁷) C(O)R³⁷, OR³⁷, SR³⁷, S(O)R³⁷, SO₂R³⁷, NHR³⁷ or N(R³²) R³⁷;
R³¹ and R^{31A} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;
R³² is R³³, C(O)R³³ or C(O)OR³³;
R³³ is R³⁴ or R³⁵;
R³⁴ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³⁵ is C₁-alkyl, C₅-alkyl or C₆-alkyl, each of which is unsubstituted or substituted with R³⁶;
R³⁶ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³⁷ is R³⁸, R³⁹ or R⁴⁰, each of which is unsubstituted or is substituted with F, Cl, Br, I, R⁴¹, OR⁴¹, NHR⁴¹, N(R⁴¹)₂, NHC(O)OR⁴¹, SR⁴¹, S(O)R⁴¹ or SO₂R⁴¹;
R³⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁰ is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;
R⁴¹ is R⁴², R⁴³, R⁴⁴ or R⁴⁵;
R⁴² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{42-A};
R^{42A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;
R⁴³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁵ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R⁴⁶, OR⁴⁶, NHR⁴⁶, N(R⁴⁶)₂, C(O)NH₂, C(O)NHR⁴⁶, C(O)N(R⁴⁶)₂, OH, (O), C(O)OH, N₃, CN, NH₂, CF₃, CF₂CF₃, F, Cl, Br or I substituents;
R⁴⁶ is R⁴⁷, R⁴⁸ or R⁴⁹;
R⁴⁷ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁸ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁹ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
wherein the moieties represented by F¹ and Y¹ together are substituted with C₁-alkyl, C₂-alkyl, C₃-alkyl, C₄-alkyl, C₅-alkyl or C₆-alkyl, each of which is substituted with one or two independently selected SR⁵⁵ or N(R⁵⁵)₂ substituents, wherein R⁵⁵ is independently selected phenyl, C₁-alkyl, i C₂-alkyl, C₃-alkyl, C₄-alkyl, C₅-alkyl or C₆-alkyl;
the moieties represented by R², R³ and R⁴ are unsubstituted or substituted with one or two independently selected R⁵⁰, OR⁵⁰, SR⁵⁰, SO₂R⁵⁰, CO(O)R⁵⁰ or OCF₃ substituents, wherein R⁵⁹ is phenyl, C₁-alkyl, C₂-alkyl, C₃-alkyl, C₄-alkyl, C₅-alkyl or C₆-alkyl;
the moieties represented by R⁸, R⁹ and R¹⁰ are unsubstituted or substituted with one or two independently selected R⁵⁰, OR⁵⁰, C(O)NHSO₂R⁵⁰, CO(O)R⁵⁰, C(O)R⁵⁰, C(O)OH, C(O)NHOH, OH, NH₂, F, Cl, Br or I substituents, wherein R⁵⁰ is phenyl, tetrazolyl or R⁵⁴, wherein R⁵⁴ is C₁-alkyl, C₂-alkyl, C₃-alkyl, C₄-alkyl, C₅-alkyl or C₆-alkyl, each of which is unsubstituted or substituted with phenyl;
the moieties represented by R²⁶ and R²⁷ are further unsubstituted or substituted with one or two independently selected F, Br, Cl or I substituents;
the moieties represented by R²⁸, R²⁹ and R³⁰ are further unsubstituted or substituted with OR⁵⁴, wherein R⁵⁴ is C₁-alkyl, C₂-alkyl, C₃-alkyl, C₄-alkyl, C₅-alkyl or C₆-alkyl, each of which is unsubstituted or substituted with R⁵⁶, wherein R⁵⁶ is C₃-cycloalkyl, C₄-cycloalkyl C₅-cycloalkyl or C₆-cycloalkyl, each having one or two CH₂ moieties replaced with independently selected O or NH and one CH moiety unreplaced or replaced with N;
the moieties represented by R³⁸, R³⁹ and R⁴⁰ are further unsubstituted or substituted with one or two independently selected R⁵⁴, F, Br, Cl or I substituents, wherein R⁵⁴ is C₁-alkyl, C₂-alkyl, C₃-alkyl, C₄-alkyl, C₅-alkyl or C₆-alkyl; and
the moieties represented by R⁴², R⁴³, R⁴⁴ and R⁴⁵ are unsubstituted or substituted with one or two independently selected R⁵⁰, OR⁵⁰, SR⁵⁰, N(R⁵⁰)₂, SO₂R⁵⁰, CN, CF₃, F, Cl, Br or I substituents, wherein R⁵⁰ is phenyl or R⁵⁴, wherein R⁵⁴ is C₁-alkyl, C₂-alkyl, C₃-alkyl, C₄-alkyl, C₅-alkyl or C₆-alkyl, each of which is unsubstituted or substituted with N(R⁵⁵)₂ or R⁵⁶, wherein R⁵⁵ is C₁-alkyl, C₂-alkyl, C₃-alkyl, C₄-alkyl, C₅-alkyl or C₆-alkyl and R⁵⁶ is C₃-cycloalkyl, C₄-cycloalkyl, C₅-cycloalkyl or C₆-cycloalkyl, each having one CH₂ moiety unreplaced or replaced with independently selected O, C(O), S, S(O), SO₂ or NH and one or two CH moieties unreplaced or replaced with N.

3. The compound of claim 2, wherein A¹ is C(A²);
A² is H, F, CN, C(O)OH, C(O)NH₂ or C(O)OR^{1A};
F¹ is R¹, OR¹, SR¹, S(O)R¹, SO₂R¹, C(O)R¹, C(O)OR¹, OC(O)R¹, NHR¹, N(R¹)₂, C(O)NHR¹, C(O)N(R¹)₂, NHC(O)R¹, NHC(O)OR¹, NR¹C(O)NHR¹, NR¹C(O)N(R¹)₂, SO₂NHR¹, SO₂N(R¹)₂, NHSO₂R¹, NHSO₂NHR¹ or N(CH₃) SO₂N(CH₃) R¹;
D¹ is H, F, C₁ or CF₃;
E¹ is H, F or Cl;
Y¹ is H, CN, NO₂, C(O)OH, F, Cl, Br, CF₃, OCF₃, NH₂, C(O)NH₂, or
F¹ and Y¹, together with the atoms to which they are attached, are imidazole or triazole;
R¹ is R², R³, R⁴ or R⁵;
R^{1A} is C₁-alkyl, C₂-alkyl, C₃-alkyl, C₄-alkyl, C₅-alkyl, C₆-alkyl;
R² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
R⁵ is C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₃-alkynyl, C₄-alkynyl, C₅-alkynyl or C₆-alkynyl, each of which is unsubstituted or substituted with one or two or three independently selected R⁶, R⁷, OR⁷, SR⁷, S(O)R⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, SO₂NH₂, SO₂NHR⁷, SO₂N(R⁷)₂, NHC(O)NH₂, NHC(O)NHR⁷, NHC(O)CH(CH₃)NHC(O)CH(CH₃)NH₂, OH, (O), C(O)OH, (O), N₃, CN, NH₂, CF₃, CF₂CF₃, F, Cl, Br or I substituents;
R⁶ is C₂-spiroalkyl, C₃-spiroalkyl, C₄-spiroalkyl or C₅-spiroalkyl, each of which is unsubstituted or substituted with OH, (O), N₃, CN, CF₃, CF₂CF₃, F, Cl, Br, I, NH₂, NH(CH₃) or N(CH₃)₂;
R⁷ is R⁸, R⁹, R¹⁰ or R¹¹;
R⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{BA};
R^{8A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;
R⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R¹⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₄-cycloalkenyl, C₅-cycloalkenyl, C₆-cycloalkenyl, C₇-cycloalkenyl, C₈-cycloalkenyl, C₉-cycloalkenyl, C₁₀-cycloalkenyl, each having one or two CH₂ moieties unreplaced or replaced with independently selected O, C(O), CNOCH₃, S, S(O), SO₂ or NH and one or two CH moieties unreplaced or replaced with N;
R¹¹ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two or three independently selected R¹², OR¹², NHR¹², N(R¹²)₂, C(O)NH₂, C(O)NHR¹², C(O)N(R¹²)₂, OH, (O)C(O)OH, N₃, CN, NH₂, CF₃, CF₂CF₃, F, Cl, Br or I substituents;
R¹² is R¹³, R¹⁴, R¹⁵ or R¹⁶;
R¹³ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{13A};
R^{13A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;
R¹⁴ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl, 1,2,3-triazolyl, each of which is unfused or fused with benzene;
R¹⁵ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N;
R¹⁶ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₃-alkynyl, C₄-alkynyl, C₅-alkynyl or C₆-alkynyl;
A³ and A⁴ are each independently N or C(R²⁶); each R²⁶ is independently hydrogen, halogen, C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl, hydroxy, C₁-alkoxy, C₂-alkoxy, C₃-alkoxy, C₄-alkoxy, C₅-alkoxy, C₆-alkoxy, C₂-alkenyloxy, C₃-alkenyloxy, C₄-alkenyloxy, C₅-alkenyloxy, C₆-alkenyloxy, C₂-alkynyloxy, C₃-alkynyloxy, C₄-alkynyloxy, C₅-alkynyloxy, C₆-alkynyloxy, —C(O)N(R^{26A})₂, acyl, —N(R^{26B})², —C(R^{26C})³, wherein R^{26A} is independently C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl or C₆-alkynyl; R^{26B} is independently hydrogen, C₁-alkyl, C₂-alkyl, C₃-alkyl, C₄-alkyl, C₅-alkyl, C₆-alkyl, C₂-alkenyl, C₃-alkenyl, C₄-alkenyl, C₅-alkenyl, C₆-alkenyl, C₂-alkynyl, C₃-alkynyl, C₄-alkynyl, C₅-alkynyl, C₆-alkynyl or acyl; R^{26C} is independently hydrogen, F, Cl or Br;
B¹, B², B³ and B⁴ are each independently N, C(Z) or C(R²⁷), wherein one of B¹, B², B³ or B⁴ is C(Z); each R²⁷ is independently selected from H, Cl, Br, I, CH₂R^{27C}, C(R^{27B})(R^{27A})(R^{27C}), C(O)R^{27C}, OR^{27C}, SR^{27C}, S(O)R^{27C}, SO₂R^{27C} or NHR^{27C};
R^{27A} and R^{27B} are independently F, Cl, Br, C₁-alkyl, C₂-alkyl, C₃-alkyl, C₄-alkyl, C₅-alkyl or C₆-alkyl or are taken together and are C₂-spiroalkyl, C₃-spiroalkyl, C₄-spiroalkyl or C₅-spiroalkyl;
R^{27C} is R^{27I}, R^{27J} or R^{27K}, each of which is substituted with F, Cl, Br, I, R^{27L}, OR^{27L}, NHR^{27L}, N(R^{27L})₂, NHC(O)OR^{27L}, SR^{27C}, S(O)R^{27L} or SO₂R^{27L};
R^{27I} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27J} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R^{27K} is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;
R^{27L} is R^{27M}, R^{27N}, R^{27P} or R^{27Q};
R^{27M} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{27MM};
R^{27MM} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;
R^{27N} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R^{27P} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27Q} is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R^{27QQ}, OR^{27QQ}, NHR^{27QQ}, N(R^{27QQ})₂, C(O)NH₂, C(O)NHR^{27QQ}, C(O)N(R^{27QQ)}₂, OH, (O), C(O)OH, N₃, CN, NH₂, CF₃, CF₂CF₃, F, Cl, Br or I substituents;
R^{27QQ} is R^{27R}, R^{27S} or R^{T};
R^{27R} is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R^{27S} is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R^{27T} is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
Z is R²⁸, R²⁹ or R³⁰;
R²⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R²⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R³⁰ is C₃-cycloalkyl, C₄-cycloalkyl, C₅-cycloalkyl, C₆-cycloalkyl, C₇-cycloalkyl, C₈-cycloalkyl, C₉-cycloalkyl, C₁₀-cycloalkyl, C₁₁-cycloalkyl, C₁₂-cycloalkyl, C₁₃-cycloalkyl, C₁₄-cycloalkyl, C₄-cycloalkenyl, C₅-cycloalkenyl, C₆-cycloalkenyl, C₇-cycloalkenyl, C₈-cycloalkenyl, C₉-cycloalkenyl or C₁₀-cycloalkenyl, each having one or two CH₂ moieties unreplaced or replaced with independently selected O, C(O), S, S(O), SO₂ or NH and one or two CH moieties unreplaced or replaced with N;
each of R²⁸, R²⁹ and R³⁰ is unsubstituted or substituted with Cl, Br, CH₂R³⁷, C(O)R³⁷, C(R³¹)(R^{31A})(R³⁷), C(O)R³⁷, OR³⁷, SR³⁷, S(O)R³⁷, SO₂R³⁷ or NHR³⁷;
R³¹ and R^{31A} are independently F, Cl, Br, C₁-alkyl, C₂-alkyl, C₅-alkyl or C₆-alkyl or are taken together and are C₂-spiroalkyl, C₃-spiroalkyl, C₄-spiroalkyl or C₅-spiroalkyl;
R³⁷ is R³⁸, R³⁹ or R⁴⁹, each of which is unsubstituted or substituted with F, Cl, Br, I, R⁴¹, OR⁴¹, NHR⁴¹, N(R⁴¹)₂, NHC(O)OR⁴¹, SR⁴¹, S(O)R⁴¹ or SO₂R⁴¹;
R³⁸ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R³⁹ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R⁴⁰ is C₃-cycloalkyl, C₄-cycloalkyl, C₅-cycloalkyl, C₆-cycloalkyl, C₇-cycloalkyl, C₈-cycloalkyl, C₄-cycloalkenyl, C₅-cycloalkenyl, C₆-cycloalkenyl, C₇-cycloalkenyl or C₈-cycloalkenyl, each having one or two CH₂ moieties unreplaced or replaced with independently selected O, C(O), S, S(O), SO₂ or NH and one or two CH moieties unreplaced or replaced with N;
R⁴¹ is R⁴², R⁴³, R⁴⁴ or R⁴⁵;
R⁴² is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or R^{42A};
R^{42A} is C₄-cycloalkane, C₅-cycloalkane, C₆-cycloalkane, C₄-cycloalkene, C₅-cycloalkene or C₆-cycloalkene, each having one or two CH₂ moieties unreplaced or replaced with independently selected O, C(O), S, S(O), SO₂ or NH and one or two CH moieties unreplaced or replaced with N;
R⁴³ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R⁴⁴ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), CNOH, CNOCH₃, S, S(O), SO₂ or NH and one or two CH moieties unreplaced or replaced with N, and each of which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁵ is C₁-alkyl, C₂-alkyl, C₃-alkyl, C₄-alkyl, C₅-alkyl C₆-alkyl, C₂-alkenyl, C₃-alkenyl, C₄-alkenyl, C₅-alkenyl C₆-alkenyl, C₂-alkynyl, C₃-alkynyl, C₄-alkynyl, C₅-alkynyl or C₆-alkynyl, each of which is unsubstituted or substituted with one or two independently selected R⁴⁶, OR⁴⁶, NHR⁴⁶, N(R⁴⁶)₂, C(O)NH₂, C(O)NHR⁴⁶, C(O)N(R⁴⁶)₂, OH, (O), C(O)OH, N₃, CN, NH₂. CF₃, CF₂CF₃, F, Cl, Br or I substituents;
R⁴⁶ is R⁴⁷, R⁴⁸ or R⁴⁹;
R⁴⁷ is phenyl which is unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine or 1,2,3-triazole;
R⁴⁸ is furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thienyl, triazinyl or 1,2,3-triazolyl, each of which is unfused or fused with benzene;
R⁴⁹ is C₃-cycloalkyl, C₄-cycloalkyl, C₅-cycloalkyl, C₆-cycloalkyl, C₄-cycloalkenyl, C₅-cycloalkenyl or C₆-cycloalkenyl, each having one or two CH₂ moieties unreplaced or replaced with independently selected O, C(O), S, S(O), SO₂ or NH and one or two CH moieties unreplaced or replaced with N;
wherein the moieties represented by F¹ and Y¹ together are substituted with C₂-alkyl, C₃-alkyl or C₄-alkyl, each of which is substituted with one or two independently selected SR⁵⁵ or N(R⁵⁵)₂ substituents, wherein R⁵⁵ is independently selected phenyl or C₁-alkyl;
the moieties represented by R², R³ and R⁴ are unsubstituted or substituted with one or two independently selected R⁵⁰, OR⁵⁰, SR⁵⁰, SO₂R⁵⁰, CO(O)R⁵⁰ or OCF₃ substituents, wherein R⁵⁰ is phenyl, C₁-alkyl, C₂-alkyl, C₃-alkyl or C₄-alkyl;
the moieties represented by R⁸, R⁹ and R¹⁰ are unsubstituted or substituted with one or two independently selected R⁵⁰, OR⁵⁰, C(O)NHSO₂R⁵⁰, CO(O)R⁵⁰, C(O)R⁵⁰, C(O)OH, C(O)NHOH, OH, NH₂, F, Cl, Br or I substituents, wherein R⁵⁰ is phenyl, tetrazolyl or R⁵⁴, wherein R⁵⁴ is C₁-alkyl, C₂-alkyl or C₃-alkyl, each of which is unsubstituted or substituted with phenyl;
the moieties represented by R²⁸, R²⁹ and R³⁰ are further unsubstituted or substituted with OR⁵⁴, wherein R⁵⁴ is C₁-alkyl or C₂-alkyl, each of which is unsubstituted or substituted with N(R⁵⁵)₂ or R⁵⁶, wherein R⁵⁵ is C₁-alkyl, C₂-alkyl, C₃-alkyl, C₄-alkyl, C₅-alkyl or C₆-alkyl, and R⁵⁶ is C₅-cycloalkyl or C₆-cycloalkyl, each having one or two CH₂ moieties replaced with independently selected O or NH and one CH moiety unreplaced or replaced with N;
the moieties represented by R³⁸, R³⁹ and R⁴⁰ are further unsubstituted or substituted with one or two independently selected C₁-alkyl, F, Br, Cl or I substituents; and
the moieties represented by R⁴², R⁴³, R⁴⁴ and R⁴⁵ are unsubstituted or substituted with one or two independently selected R⁵⁰, OR⁵⁰, SR⁵⁰, N(R⁵⁰)₂, SO₂R⁵⁰, CN, CF₃, F, Cl, Br or I substituents, wherein R⁵⁰ is phenyl or R⁵⁴, wherein R⁵⁴ is C₁-alkyl, C₂-alkyl or C₃-alkyl, each of which is unsubstituted or substituted with N(C₁-alkyl)₂ or C₆-cycloalkyl having one CH₂ moiety replaced with 0 and one CH moiety replaced with N.

4. The compound of claim 3, wherein A¹ is C(A²);
A² is H, F, CN, C(O)OH, C(O)NH₂ or C(O)OCH₃;
F¹ is R¹, OR¹, NHR¹, N(R¹)₂ or NR¹C(O)N(R¹)₂;
D¹ is H, F, C₁ or CF₃;
E¹ is H, F or Cl;
Y¹ is H, CN, NO₂, F, Cl, CF₃, OCF₃, NH₂, C(O)NH₂, or
F¹ and Y¹, together with the atoms to which they are attached, are imidazole or triazole;
R¹ is phenyl, pyrrolyl, C₅-cycloalkyl, C₆-cycloalkyl, piperidinyl, tetrahydrofuranyl, tetrahydropyranyl, tetrahydrothiopyranyl or R⁵;
R⁵ is C₁-alkyl, C₂-alkyl, C₃-alkyl, C₄-alkyl, C₅-alkyl or C₆-alkyl, each of which is unsubstituted or substituted with one or two or three independently selected C₄-spiroalkyl, C₅-spiroalkyl, R⁷, OR⁷, SR⁷, SO₂R⁷, NHR⁷, N(R⁷)₂, C(O)R⁷, C(O)NH₂, C(O)NHR⁷, NHC(O)R⁷, NHSO₂R⁷, NHC(O)OR⁷, NHC(O)NH₂, NHC(O)CH(CH₃) NHC(O)CH(CH₃) NH₂, OH, C(O) 0H or NH₂ substituents;
R⁷ is phenyl, furanyl, imidazolyl, pyridinyl, tetrazolyl, thiazolyl, thienyl, 1,3-benzoxazolyl, 1,3-benzodioxolyl, 1,3-benzothiazole, C₃-cycloalkyl, C₄-cycloalkyl, C₅-cycloalkyl, C₆-cycloalkyl, azetidinyl, morpholinyl, piperazinyl, piperidinyl, thiomorpholinyl, thiomorpholinyl sulfone 7-azabicyclo[2.2.1]heptanyl, 8-azabicyclo[3.2.1]-octanyl, 4,5-dihydro-1H-imidazolyl 2-oxa-5-azabicyclo-[2.2.1]heptanyl, 1,4,5,6-tetrahydropyrimidinyl or R¹¹;
R¹¹ is C₁-alkyl, C₂-alkyl, C₃-alkyl or C₄-alkyl, each of which is unsubstituted or substituted with one or two or three independently selected R¹², OR¹², N(R¹²)₂, C(O)N(R¹²)₂, OH, C(O)OH, NH₂, CF₃, F, Cl, Br or I substituents;
R¹² is 1,3-benzodioxolyl, pyridinyl, morpholinyl or C₁-alkyl;
A³ and A⁴ are each independently N or C(R²⁶); each R²⁶ is independently hydrogen, halogen, C₁-alkyl, C₂-alkyl, C₃-alkyl, hydroxy, C₁-alkoxy, C₂-alkoxy, C₃-alkoxy, C₂-alkenyloxy, C₃-alkenyloxy, C₄-alkenyloxy, C₂-alkynyloxy, C₃-alkynyloxy, C₄-alkynyloxy, acyl, —N(R^{26B})², —C(R^{26C})₃,wherein R^{26A} is independently C₁-alkyl, C₂-alkyl, C₃-alkyl, C₄-alkyl, C₂-alkenyl, C₃-alkenyl, C₄-alkenyl, C₂-alkynyl, C₃-alkynyl or C₄-alkynyl; R^{26B} is independently hydrogen, C₁-alkyl, C₂-alkyl, C₃-alkyl, C₂-alkenyl, C₃-alkenyl, C₄-alkenyl, C₂-alkynyl, C₃-alkynyl, C₄-alkynyl or acyl; R^{26C} is independently hydrogen, F, Cl or Br;
B¹, B², B², B³ and B⁴ are each independently N, C(Z) or C(R²²), wherein one of B², B², B³ or B⁴ is C(Z) and two or three of B¹, B², B³ or B⁴ is C(R²²); R²⁷ is independently H, F, Cl, Br or I; Z is C₆-cycloalkenyl, piperazinyl, piperidinyl, 1,2,3,6-tetrahydropyridinyl or octahydropyrrolo[3,4-c]pyrrolyl, each of which is substituted with CH₂R³⁷, C(C₂-spiroalkyl) (R³⁷) or C(O)R³⁷;
R³⁷ is phenyl, naphthyl, imidazolyl, pyrazolyl, pyridinyl, C₅-cycloalkenyl, C₆-cycloalkenyl, C₇-cycloalkenyl, C₈-cycloalkenyl or 3,6-dihydro-2H-pyranyl, each of which is substituted with F, Cl, Br, I, R⁴¹, NHR⁴¹, N(R⁴¹)₂, NHC(O)OR⁴¹ or SR⁴¹;
R⁴¹ is phenyl, naphthyl, cyclohexyl, morpholinyl, piperidinyl, thienyl, pyridinyl, quinolinyl, benzofuranyl, 1,3-benzodioxolyl, isoindolinyl, 1,3-oxazolidin-2-onyl or R⁴⁵;
R⁴⁵ is C₁-alkyl, C₂-alkyl, C₃-alkyl or C₄-alkyl, each of which is unsubstituted or substituted with phenyl;
wherein the moieties represented by F¹ and Y¹ together are substituted with C₂-alkyl, C₃-alkyl or C₄-alkyl, each of which is substituted with one or two independently selected SR⁵⁵ or N(R⁵⁵)₂ substituents, wherein R⁵⁵ is independently selected phenyl or C₁-alkyl;
the moieties represented by R¹ are unsubstituted or substituted with one or two independently selected R⁵⁰, OR⁵⁰, SR⁵⁰, SO₂R⁵⁰, CO(O)R⁵⁰ or OCF₃ substituents, wherein R⁵⁰ is phenyl, C₁-alkyl, C₂-alkyl, C₃-alkyl or C₄-alkyl;
the moieties represented by R⁷ are unsubstituted or substituted with one or two independently selected R⁵⁰, OR⁵⁰, C(O)NHSO₂R⁵⁰, CO(O)R⁵⁰, C(O)R⁵⁰, C(O)OH, C(O)NHOH, OH, NH₂, F, Cl, Br or I substituents, wherein R⁵⁰ is phenyl, tetrazolyl or R⁵⁴, wherein R⁵⁴ is C₁-alkyl, C₂-alkyl or C₃-alkyl, each of which is unsubstituted or substituted with phenyl;
the C₆-cycloalkenyl, piperazinyl, piperidinyl, 1,2,3,6-tetrahydropyridinyl and octahydropyrrolo[3,4-c]pyrrolyl moieties of Z are further unsubstituted or substituted with OR⁵⁴, wherein R⁵⁴ is C₁-alkyl or C₂-alkyl, each of which is unsubstituted or substituted with N(C₁-alkyl)₂. morpholinyl, piperidinyl or piperidinyl; and
the moieties represented by R⁴¹ are unsubstituted or substituted with one or two independently selected R⁵⁰, OR⁵⁰, SR⁵⁰, N(R⁵⁰)₂, SO₂R⁵⁰, CN, CF₃, F, Cl, Br or I substituents, wherein R⁵⁰ is phenyl or R⁵⁴, wherein R⁵⁴ is C₁-alkyl, C₂-alkyl or C₃-alkyl, each of which is unsubstituted or substituted with N(C₁-alkyl)₂ or morpholinyl.

5. The compound of claim 1, 2, 3 or 4, wherein said compound is of Subformulae Ia:

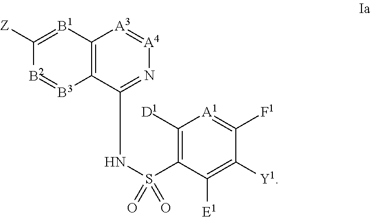

6. The compound of claim 1, 2, 3 or 4, wherein the moiety: 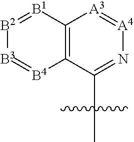
in Formula Ia is selected from the group consisting of:

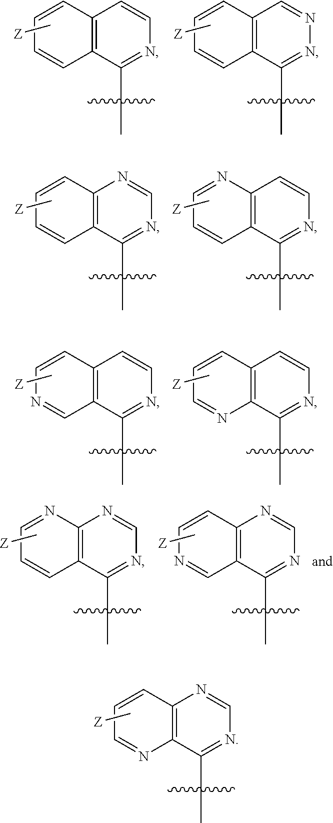

7. A composition for treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer, said composition comprising an excipient and a therapeutically effective amount of the compound of claim 1.

8. A method of treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer in a patient, said method comprising administering to the patient a therapeutically effective amount of a compound of claim 1.

9. A method of treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer in a patient, said method comprising administering to the patient therapeutically effective amount of the compound of claim 1 and a therapeutically effective amount of one additional therapeutic agent or more than one additional therapeutic agent.
